(19) United States
(12) Patent Application Publication
(10) Pub. No.: US 2025/0066344 A1
(43) Pub. Date: Feb. 27, 2025

(54) BENZOTHIAZOLE, BENZOISOXAZOLE, AND BENZODIOXOLE ANALOGS AS RXFP1 RECEPTOR AGONISTS

(71) Applicant: BRISTOL-MYERS SQUIBB COMPANY, Princeton, PA (US)

(72) Inventors: Sreekantha Ratna Kumar, Bangalore (IN); Pitani Veera Venkata Srinivas, Bangalore (IN); Subramanya Hegde, Bangalore (IN); Michael J. Orwat, New Hope, PA (US)

(21) Appl. No.: 18/718,477

(22) PCT Filed: Dec. 14, 2022
(86) PCT No.: PCT/US2022/081519

§ 371 Date: Jun. 11, 2024

Related U.S. Application Data
(60) Provisional application No. 63/289,830, filed on Dec. 15, 2021.

(51) Int. Cl. C07D 417/12; A61K 31/357; A61K 31/397; A61K 31/4025; A61K 31/4155; A61K 31/422; A61K 31/423; A61K 31/424; A61K 31/4245; A61K 31/428; A61K 31/437; A61K 31/438; A61K 31/443; A61K 31/4525; A61K 31/4725; A61K 31/4985; A61K 31/5377; A61K 31/55; A61K 31/675; C07D 261/20; C07D 317/48; C07D 405/12; C07D 413/12; C07D 413/14; C07D 487/04; C07D 491/107; C07D 495/04; C07D 495/10; C07D 498/04; C07D 498/10; C07D 513/04; C07F 9/6558

(52) U.S. Cl. CPC C07D 417/12; A61K 31/357; A61K 31/397; A61K 31/4025; A61K 31/4155; A61K 31/422; A61K 31/423; A61K 31/424; A61K 31/4245; A61K 31/428; A61K 31/437; A61K 31/438; A61K 31/443; A61K 31/4525; A61K 31/4725; A61K 31/4985; A61K 31/5377; A61K 31/55; A61K 31/675; C07D 261/20; C07D 317/48; C07D 405/12; C07D 413/12; C07D 413/14; C07D 487/04; C07D 491/107; C07D 495/04; C07D 495/10; C07D 498/04; C07D 498/10; C07D 513/04; C07F 9/65583

(57) ABSTRACT

The disclosure relates to compounds of Formula (I), which are RXFP1 receptor agonists, compositions containing them, and methods of using them, for example, in the treatment of heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

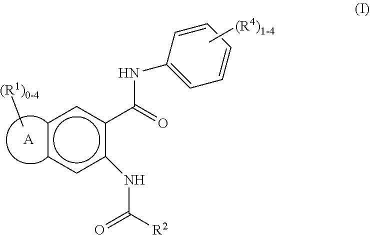

### CROSS-REFERENCE TO RELATED APPLICATIONS

This application claims the benefit of U.S. Provisional Application No. 63/289,830, filed Dec. 15, 2021, the disclosure of which is incorporated herein by reference in its entirety.

### BACKGROUND OF THE INVENTION

The present disclosure relates to novel compounds which are relaxin family peptide receptor 1 (RXFP1) agonists, compositions containing them, and methods of using them, for example in the treatment of heart failure, fibrotic diseases, and related diseases such as lung disease (e.g., idiopathic pulmonary fibrosis), kidney disease (e.g., chronic kidney disease), and hepatic disease (e.g., non-alcoholic steatohepatitis and portal hypertension).

The human relaxin hormone (also called relaxin or H2 relaxin) is a 6-kDa peptide composed of 53 amino acids whose activity was initially discovered when Frederick Hisaw in 1926 injected crude extracts from swine corpus luteum into virgin guinea pigs and observed a relaxation of the fibrocartilaginous pubic symphysis joint (Hisaw F L., Proc. Soc. Exp. Biol. Med., 1926, 23, 661-663). The relaxin receptor was previously known as Lgr7 but is now officially termed the relaxin family peptide receptor 1 (RXFP1) and was deorphanized as a receptor for relaxin in 2002 (Hsu S Y., et al., Science. 2002, 295, 671-674). RXFP1 is reasonably well conserved between mouse and human with 85% amino acid identity and is essentially ubiquitously expressed in humans and in other species (Halls M L., et al., Br. J. Pharmacol., 2007, 150, 677-691). The cell signaling pathways for relaxin and RXFP1 are cell type dependent and quite complex (Halls M L., et al., Br. J. Pharmacol., 2007, 150, 677-691; Halls M L., et al. Ann. N Y Acad. Sci., 2009, 1160, 108-111; Halls M L., Ann N Y Acad Sci., 2007, 1160, 117-120). The best studied pathway is the relaxin-dependent increase in cellular levels of cAMP in which relaxin functions as an RXFP1 agonist to promote GaS coupling and activation of adenylate cyclase (Halls M L., et al., Mol. Pharmacol., 2006, 70, 214-226).

Since the initial discovery of relaxin much experimental work has focused on delineating the role relaxin has played in female reproductive biology and the physiological changes that occur during mammalian pregnancy (Sherwood O D., Endocr. Rev., 2004, 25, 205-234). During human gestation, in order to meet the nutritional demands imposed upon it by the fetus, the female body undergoes a significant ˜30% decrease in systemic vascular resistance (SVR) and a concomitant ˜50% increase in cardiac output (Jeyabalan A C., K. P., Reanl and Electolvte Disorders. 2010, 462-518), (Clapp J F. & Capeless E., Am. J. Cardio., 1997, 80, 1469-1473). Additional vascular adaptations include an ˜30% increase in global arterial compliance that is important for maintaining efficient ventricular-arterial coupling, as well as an ˜50% increase in both renal blood flow (RBF) and glomerular filtration rate (GFR), important for metabolic waste elimination (Jeyabalan A C., K. P., Reanl and Electolyte Disorders. 2010, 462-518), (Poppas A., et al., Circ., 1997, 95, 2407-2415). Both pre-clinical studies in rodents as well as clinical studies performed in a variety of patient settings, provide evidence that relaxin is involved, at least to some extent, in mediating these adaptive physiological changes (Conrad K P., Regul. Integr. Comp. Physiol., 2011, 301, R267-275), (Teichman S L., et al., Heart Fail. Rev., 2009, 14, 321-329). Importantly, many of these adaptive responses would likely be of benefit to HF patients in that excessive fibrosis, poor arterial compliance, and poor renal function are all characteristics common to heart failure patients (Mohammed S F., et al., Circ., 2015, 131, 550-559), (Wohlfahrt P., et al., Eur. J. Heart Fail., 2015, 17, 27-34), (Damman K., et al., Prog. Cardiovasc. Dis., 2011, 54, 144-153).

Heart failure (HF), defined hemodynamically as “systemic perfusion inadequate to meet the body's metabolic demands as a result of impaired cardiac pump function”, represents a tremendous burden on today's health care system with an estimated United States prevalence of 5.8 million and greater than 23 million worldwide (Roger V L., et al., Circ. Res., 2013, 113, 646-659). It is estimated that by 2030, an additional 3 million people in the United States alone will have HF, a 25% increase from 2010. The estimated direct costs (2008 dollars) associated with HF for 2010 was $25 billion, projected to grow to $78 B by 2030 (Heidenreich P A., et al., Circ., 2011, 123, 933-944). Astoundingly, in the United States, 1 in 9 deaths has HF mentioned on the death certificate (Roger V L., et al., Circ., 2012, 125, e2-220) and, while survival after HF diagnosis has improved over time (Matsushita K., er al., Diabetes, 2010, 59, 2020-2026), (Roger V L., er al., JAMA, 2004, 292, 344-350), the death rate remains high with ˜50% of people with HF dying within 5 years of diagnosis (Roger V L., et al., Circ., 2012, 125, e2-220), (Roger V L., et al., JAMA, 2004, 292, 344-350).

The symptoms of HF are the result of inadequate cardiac output and can be quite debilitating depending upon the advanced stage of the disease. Major symptoms and signs of HF include: 1) dyspnea (difficulty in breathing) resulting from pulmonary edema due to ineffective forward flow from the left ventricle and increased pressure in the pulmonary capillary bed; 2) lower extremity edema occurs when the right ventricle is unable to accommodate systemic venous return; and 3) fatigue due to the failing heart's inability to sustain sufficient cardiac output (CO) to meet the body's metabolic needs (Kemp C D., & Conte J V., Cardiovasc. Pathol., 2011, 21, 365-371). Also, related to the severity of symptoms, HF patients are often described as “compensated” or “decompensated”. In compensated heart failure, symptoms are stable, and many overt features of fluid retention and pulmonary edema are absent. Decompensated heart failure refers to a deterioration, which may present as an acute episode of pulmonary edema, a reduction in exercise tolerance, and increasing breathlessness upon exertion (Millane T., et al., BMJ, 2000, 320, 559-562).

In contrast to the simplistic definition of poor cardiac performance not being able to meet metabolic demands, the large number of contributory diseases, multitude of risk factors, and the many pathological changes that ultimately lead to heart failure make this disease exceedingly complex (Jessup M. & Brozena S., N. Engl. J. Med., 2003, 348, 3007-2018). Injurious events thought to be involved in the pathophysiology of HF range from the very acute such as myocardial infarction to a more chronic insult such as life-long hypertension. Historically, HF was primarily described as “systolic HF” in which decreased left-ventricular (LV) contractile function limits the expulsion of blood and hence results in a reduced ejection fraction (EF is stroke volume/end diastolic volume), or “diastolic HF” in which active relaxation is decreased and passive stiffness is increased limiting LV filling during diastole, however overall EF is maintained (Borlaug B A. & Paulus W J., Eur Heart J., 2011, 32, 670-679). More recently, as it became understood that diastolic and systolic LV dysfunction was not uniquely specific to these two groups, new terminology was employed: “heart failure with reduced ejection fraction” (HFrEF), and “heart failure with preserved ejection fraction” (HFpEF) (Borlaug B A. & Paulus W J., Eur Heart J. 2011, 32, 670-679). Although these two patient populations have very similar signs and symptoms, whether HFrEF and HFpEF represent two distinct forms of HF or two extremes of a single spectrum sharing a common pathogenesis is currently under debate within the cardiovascular community (Borlaug B A. & Redfield M M., Circ., 2011, 123, 2006-2013), (De Keulenaer G W., & Brutsaert D L., Circ., 2011, 123, 1996-2004).

Serelaxin, an intravenous (IV) formulation of the recombinant human relaxin peptide with a relatively short first-phase pharmacokinetic half-life of 0.09 hours, is currently being developed for the treatment of HF (Novartis, 2014). Serelaxin has been given to normal healthy volunteers (NHV) and demonstrated to increase RBF (Smith M C., et al., J. Am. Soc. Nephrol. 2006, 17, 3192-3197) and estimated GFR (Dahlke M., et al., J. Clin. Pharmacol., 2015, 55, 415-422). Increases in RBF were also observed in stable compensated HF patients (Voors A A., et al., Cir. Heart Fail., 2014, 7, 994-1002). In large clinical studies, favorable changes in worsening renal function, worsening HF, as well as fewer deaths, were observed in acute decompensated HF (ADHF) patients in response to an in-hospital 48 hour IV infusion of serelaxin (Teerlink J R., et al., Lancet. 2013, 381, 29-39), (Ponikowski P., et al., Eur. Heart, 2014, 35, 431-441). Suggesting that chronic dosing of serelaxin could provide sustained benefit to HF patients, improvement in renal function based on serum creatinine levels was observed in scleroderma patients given serelaxin continuously for 6 months using a subcutaneous pump (Teichman S L., et al., Heart Fail. Rev., 2009, 14, 321-329). In addition to its potential as a therapeutic agent for the treatment of HF, continuous subcutaneous administration of relaxin has also been demonstrated to be efficacious in a variety of animal models of lung (Unemori E N., et al., J. Clin. Invet., 1996, 98, 2739-2745), kidney (Garber S L., et al., Kidney Int., 2001, 59, 876-882), and liver injury (Bennett R G., Liver Int., 2014, 34, 416-426).

In summary, a large body of evidence supports a role for relaxin-dependent agonism of RXFP1 mediating the adaptive changes that occur during mammalian pregnancy, and that these changes translate into favorable physiological effects and outcomes when relaxin is given to HF patients. Additional preclinical animal studies in various disease models of lung, kidney, and liver injury provide evidence that relaxin, when chronically administered, has the potential to provide therapeutic benefit for multiple indications in addition to HF. More specifically, chronic relaxin administration could be of benefit to patients suffering from lung disease (e.g., idiopathic pulmonary fibrosis), kidney disease (e.g., chronic kidney disease), or hepatic disease (e.g., non-alcoholic steatohepatitis and portal hypertension).

### SUMMARY OF THE INVENTION

The present invention provides novel benzothiazole analogs, including stereoisomers, tautomers, pharmaceutically acceptable salts, or solvates thereof, which are useful as RXFP1 receptor agonists.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The compounds of the invention may be used, for example, in the treatment and/or prophylaxis of heart failure, fibrotic diseases, and related diseases, such as; lung disease (e.g., idiopathic pulmonary fibrosis), kidney disease (e.g., chronic kidney disease), or hepatic disease (e.g., non-alcoholic steatohepatitis and portal hypertension).

The compounds of the present invention may be used in therapy.

The compounds of the present invention may be used for the manufacture of a medicament for the treatment and/or prophylaxis of heart failure.

The compounds of the invention can be used alone, in combination with other compounds of the present invention, or in combination with one or more, preferably one to two other agent(s).

These and other features of the invention will be set forth in expanded form as the disclosure continues.

### DESCRIPTION OF THE INVENTION

The invention encompasses compounds of Formula (I), which are RXFP1 receptor agonists, compositions containing them, and methods of using them.

In a first aspect, the present invention provides, inter alia, compounds of Formula (I):

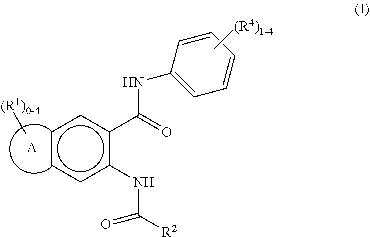

or pharmaceutically acceptable salts thereof, wherein:
Ring A is a 5-membered heterocyclyl comprising 1-3 heteroatoms selected from O, S(═O)ₚ, N, and NR^{1a}, and substituted with 0-4 R¹;
R¹ is halo or C₁₋₄ alkyl substituted with 0-5 halo or —OH;
R^{1a} is H or C₁₋₃ alkyl;
R² is phenyl substituted with 0-3 R3 and 1 R⁵, bicyclic carbocyclyl substituted with 0-3 R³ and 0-1 R⁵, or a 5 to 12-membered heterocyclyl comprising 1-5 heteroatoms selected from O, S(═O)ₚ, N, and NR^{2a} and substituted with 0-3 R³ and 0-1 R⁵;
R^{2a} is H, C₁₋₃ alkyl, or C(═O)R^{b};
R³ is halo, CN, OH, C₁₋₄ alkyl, or —OC₁₋₄ alkyl substituted with 0-5 halo, OH, —OC₁₋₄ alkyl, aryl, or heterocycyl;
R⁴ is halo, CN, C₁₋₄ alkyl substituted with 0-5 halo, OH, or —OC₁₋₄ alkyl substituted with 0-5 halo;
R⁵ is —C(═O)NR¹³R¹³, —S(═O)ₚNR¹³R¹³, C₂₋₆ alkenyl substituted with 0-3 R⁶ and 0-2 R⁵, C₂₋₈ alkynyl substituted with 0-3 R⁶ and 0-2 R⁵, C₃₋₆ carbocyclyl substituted with 0-3 R⁶ and 0-2 R⁷, or a 3- to 12-membered heterocyclyl comprising 1-4 heteroatoms selected from O, S(═O)ₚ, N and NR¹⁰ and substituted with 0-3 R^{b} and 0-1 R⁷; provided when R² is phenyl and R⁵ is heterocyclyl, said heterocyclyl is bonded to the phenyl ring through a carbon or nitrogen atom;
R⁶ is halo, CN, ═O, —OH, —OC₁₋₄ alkyl, or C₁₋₄ alkyl substituted with 0-2 halo or OH;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR^{c}, C₃₋₆ carbocyclyl substituted with 0-5 R^{e}, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{c}, —C(═O)NR^{a}R^{a}, —C(═O)NR^{a}R^{a}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c}, —OP(═O)(OH)₂, —(CH₂)ₙ—C₃₋₆ carbocyclyl substituted with 0-3 R^{e},
   or —(CH₂)ₙ-heterocyclyl comprising 1-4 heteroatoms selected from O, S(═O)ₚ, and N, and NR^{d}, and substituted with 0-5 R^{e};
R¹⁰ is H, C₁₋₄ alkyl substituted with 0-2 R⁷, —C(═O)R^{b}, —C(═O)OR^{b}, —C(═O)NR^{a}R^{a}, —S(O)ₚRₚ, C₃₋₆ carbocyclyl substituted with 0-5 R^{e}, or a 4- to 6-membered heterocyclyl comprising 1-4 heteroatoms selected from O, S(═O)ₚ, N and NR¹² and substituted with 0-5 R^{e};
R¹¹ is —OH, —C(═O)OH, —C(═O)NR^{a}R^{a}, or aryl;
R¹² is H, C₁₋₃ alkyl, or aryl;
R¹³ is H or C₁₋₃ alkyl substituted with 0-2 R⁶ and 0-2 R⁷; R¹³ and R¹³ together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R¹;
R^{a} is H, C₁₋₆ alkyl substituted with 0-5 R^{c}, C₂₋₆ alkenyl substituted with 0-5 R^{c}, C₂₋₆ alkynyl substituted with 0-5 R^{c}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{c}, —(CH₂)ₙ—C₃₋₁₀ carbocyclyl substituted with 0-5 R¹, or —(CH₂)ₙ-heterocyclyl substituted with 0-5 R^{e};
R^{c} is C₁₋₆ alkyl substituted with 0-5 R^{e}, C₂₋₆ alkenyl substituted with 0-5 R^{e}, C₂₋₆ alkynyl substituted with 0-5 R^{e}, C₃₋₆ carbocyclyl, or heterocyclyl;
R^{d} is H or C₁₋₄ alkyl;
R^{e} is halo, CN, NO₂, ═O, C₁₋₆ alkyl substituted with 0-5 R^{g}, C₂₋₆ alkenyl substituted with 0-5 R^{g}, C₂₋₄ alkynyl substituted with 0-5 R^{g}, —(CH₂)ₙ-carbocyclyl substituted with 0-5 R^{g}, —(CH₂)ₙ-heterocyclyl substituted with 0-5 R⁵, —(CH₂)ₙOR^{f}, —C(═O)OR^{f}, —C(═O)NR^{f}R^{f}, —NR^{f}C(═O)R^{f}, —S(═O)ₚR^{f}, —S(═O)ₚNR^{f}R^{f}, —NR^{f}S(═O)ₚR^{f}, —NR^{f}C(═O)OR^{f}, —OC(═O)NR^{f}R^{f}, or —(CH₂)ₙNR^{f}R^{f};
R^{f} is H, C₁₋₆ alkyl substituted with 0-1 —OC₁₋₄ alkyl, C₃₋₆ cycloalkyl, aryl, or heterocyclyl; or R^{f} and R^{f} together with the nitrogen atom to which they are both attached form a heterocyclyl;
R^{g} is halo, CN, OH, S(═O)₂C₁₋₃ alkyl, C₁₋₆ alkyl, C₃₋₆ cycloalkyl, or aryl;
n is zero, 1, 2, or 3; and
p is zero, 1, or 2.

In a second aspect within the scope of the first aspect, the present invention provides compounds of Formula (II):

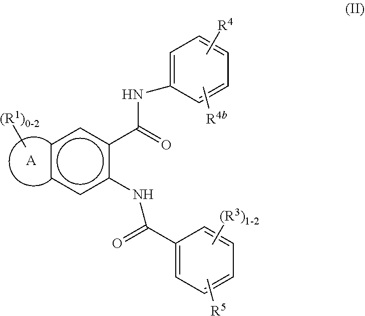

or pharmaceutically acceptable salts thereof, wherein:

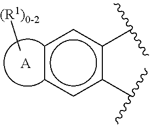

is

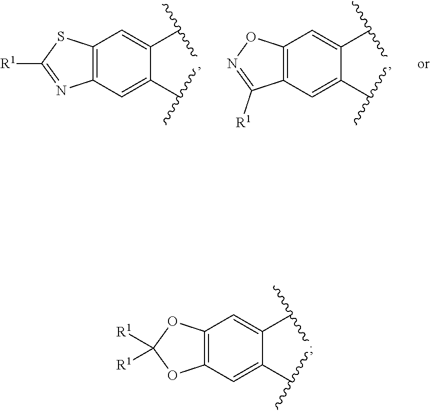

R¹ is halo or C₁₋₃ alkyl;
R³ is halo, C₁₋₃ alkyl or —OC₁₋₄ alkyl substituted with 0-4 halo;
R^{4a} is halo;
R^{4b} is C₁₋₄ alkyl substituted with 0-4 halo;
R⁵ is —C(═O)NR¹³R¹³, —S(═O)ₚNR¹³R¹³, C₂₋₇ alkenyl substituted with 0-3 R⁶ and 0-2 R⁵, C₂₋₇ alkynyl substituted with 0-3 R⁶ and 0-2 R⁵, phenyl substituted with 0-3 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁-4 alkyl, or C₁₋₄ alkyl substituted with 0-2 halo or OH;
R⁷ is C₁₋₄ alkyl substituted with 0-1 R⁶ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl
R¹² is H, C₁₋₃ alkyl, or aryl;
R¹³ is H or C₁₋₃ alkyl substituted with 0-1 R⁶ and 0-1 R⁷; or R¹³ and R¹³ together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷;
R^{a} is H, C₁₋₅ alkyl substituted with 0-5 R^{c}, C₂₋₃ alkenyl substituted with 0-5 R^{e}, C₂₋₅ alkynyl substituted with 0-5 R^{e}, —(CH₂)ₙ—C₃₋₁₀ carbocyclyl substituted with 0-5 Rₑ, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R¹, C₂₋₈ alkenyl substituted with 0-5 R^{e}, C₂₋₅ alkynyl substituted with 0-5 R^{c}, —(CH₂)ₙ—C₃₋₁₀ carbocyclyl substituted with 0-5 R^{e}, or —(CH₂)ₙ-heterocyclyl substituted with 0-5 R^{c};
R^{c} is C₁₋₅ alkyl substituted with 0-5 R^{g}, C₂₋₅ alkenyl substituted with 0-5 R^{g}, C₂₋₅ alkynyl substituted with 0-5 R^{e}, C₃₋₆ carbocyclyl, or heterocyclyl;
R^{d} is H or C₁₋₃ alkyl;
R^{c} is halo, CN, ═O, C₁₋₆ alkyl substituted with 0-5 R⁹, C₂₋₆ alkenyl substituted with 0-5 R⁵, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, —C(═O)OR^{f}, or —S(═O)ₚR^{f};
R^{f} is H or C₁₋₃ alkyl substituted with 0-1 —OC₁₋₄ alkyl;
R⁹ is halo, CN, OH, C₁₋₆ alkyl, C₃₋₆ cycloalkyl, or aryl;
n is zero, 1, 2, or 3; and
p is zero, 1, or 2.

In a third aspect within the scope of the first and second aspects, the present invention provides compounds of Formula (III):

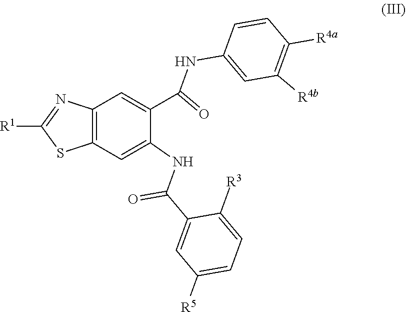

or pharmaceutically acceptable salts thereof, wherein:
R¹ is C₁₋₂ alkyl;
R³ is —OC₁₋₄ alkyl;
R^{4a} is halo;
R^{4b} is C₁₋₃ alkyl substituted with 0-4 F;
R⁵ is —S(═O)ₚNR¹³R¹³, phenyl substituted with 0-3 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R⁶ is halo, CN, C₁₋₃ alkyl, —OH, or —OC₁₋₄ alkyl;
R⁷ is C₁₋₄ alkyl substituted with 0-1 R⁸ and 0-1 R⁵, OR^{b}, —NR^{a}R^{a}, —NR^{a}C(═O)R¹, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{g}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, or —S(═O)ₚNR^{a}R^{a};
R¹⁰ is H, C₁₋₄ alkyl substituted with 0-2 R¹¹, or —C(═O)R^{b};
R¹³ and R¹³ together with the nitrogen atom to which they are both attached form a heterocyclyl comprising additional 0-5 heteroatoms selected from O, S(═O)ₚ, N, and NR¹¹, and substituted with 0-3 R⁶ and 0-2 R⁷;
R^{a} is H, C₁₋₅ alkyl substituted with 0-4 R^{c}, —(CH₂)ₙ—C₃₋₁₀ carbocyclyl substituted with 0-4 R^{c}, or —(CH₂)ₙ-heterocyclyl substituted with 0-4 R^{e}; or R^{a} and R^{a} together with the nitrogen atom to which they are both attached form a heterocyclyl substituted with 0-4 R^{c};
R^{b} is H, C₁₋₅ alkyl substituted with 0-4 R^{e}, —(CH₂)—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₃₋₆ carbocyclyl, or heterocyclyl;
R^{d} is H or C₁₋₂ alkyl;
R^{e} is halo, CN, ═O, C₁₋₆ alkyl substituted with 0-5 R⁹, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR,
R^{f} is H or C₁₋₃ alkyl substituted with 0-1 —OC₁₋₄ alkyl;
R^{g} is halo, CN, OH, C₁₋₆ alkyl, or C₃₋₆ cycloalkyl;
n is zero, 1, 2, or 3; and
p is zero, 1, or 2.

In a fourth aspect within the scope of the third aspect, the present invention provides compounds of Formula (III), or pharmaceutically acceptable salts thereof, wherein:
R¹ is —CH₃;
R³ is —OCH₃;
R^{4a} is F;
R^{4b} is CF₃;
R⁵ is —S(═O)₂NR¹³R¹³,

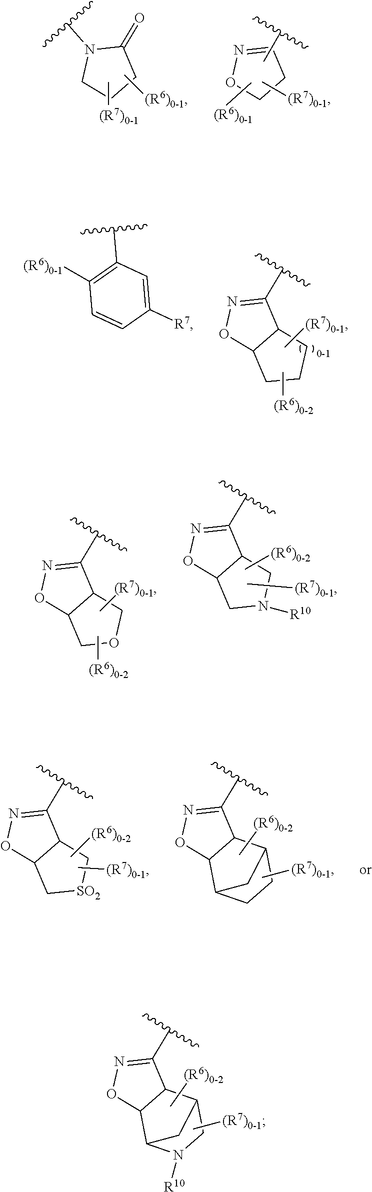

R⁶ is halo, C₁₋₄ alkyl, —OH, or —OC₁₋₄ alkyl;
R⁷ is C₁₋₄ alkyl substituted with 0-1 R⁸ and 0-1 R⁹, —C(═O)NR^{a}R^{a}, or C₃₋₆ cycloalkyl substituted with 0-2 R^{e};
R⁸ is halo, —C(═O)OC₁₋₄ alkyl, or C₁₋₄ alkyl;
R⁹ is —OR^{b}, —NR^{a}R^{a}, —NRIC(═O)R^{b}, or —OC(═O)NR^{a}R^{a};
R¹⁰ is H, C₁₋₃ alkyl substituted with 0-2 R¹⁰, or —C(═O)R^{b};
R¹¹ is —OH;
R¹³ and R¹³ together with the nitrogen atom to which they are both attached form a heterocyclyl comprising additional 0-5 heteroatoms selected from O, S(═O)ₚ, and N, and substituted with 0-3 R⁸ and 0-2 R⁷;
R^{a} is H, C₁₋₃ alkyl, or C₃₋₆ cycloalkyl;
R^{b} is H, C₁₋₄ alkyl substituted with 0-1 R^{e}, or heterocyclyl;
R^{c} is —OR^{f}; and
R^{f} is H or C₁₋₃ alkyl substituted with —OC₁₋₃alkyl.

In a fifth aspect within the scope of the fourth aspect, the present invention provides compounds of Formula (III), or pharmaceutically acceptable salts thereof, wherein:
R⁵ is

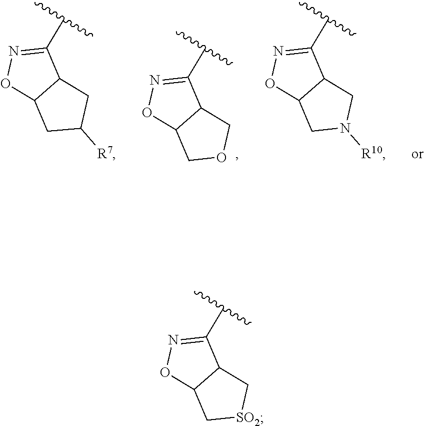

R⁷ is C₁₋₄ alkyl substituted with 0-1 R⁹;
R⁹ is —OR^{b};
R¹⁰ is H or —C(═O)R^{b};
R^{b} is H or C₁₋₄ alkyl substituted with 0-1 R^{e};
R^{e} is —OR;
R^{f} is H or C₁₋₂ alky.

In a sixth aspect within the scope of the second aspect, the present invention provides compounds of Formula (IV):

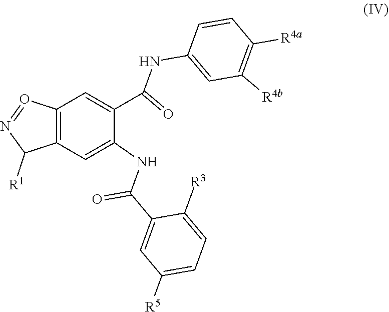

or pharmaceutically acceptable salts, thereof, wherein:
R¹ is C₁₋₂ alkyl;
R³ is —OC₁₋₄ alkyl;
R^{4a} is halo;
R^{4b} is C₁₋₃ alkyl substituted with 0-4 F;
R⁵ is C₂₋₄ alkynyl substituted with OH, or phenyl substituted with 0-3 R⁶ and 0-2 R⁷;
R⁶ is halo, CN, C₁₋₃ alkyl, —OH, or —OC₁₋₄ alkyl;
R⁷ is C₁₋₂ alkyl substituted with 0-1 R⁸ and 0-1 R⁹;
R⁸ is halo or C₄ alkyl substituted with 0-3 halo or OH;
R⁹ is —OR^{b}, —OC(═O)NR^{a}R^{a}, or —OC(═O)NR^{a}OR^{b};
R^{a} is H, C₁₋₃ alkyl, or C₃₋₆ cycloalkyl; and
R^{b} is H or C₁₋₃ alkyl.

In a seventh aspect within the scope of the first aspect, the present invention provides compounds of Formula (V):

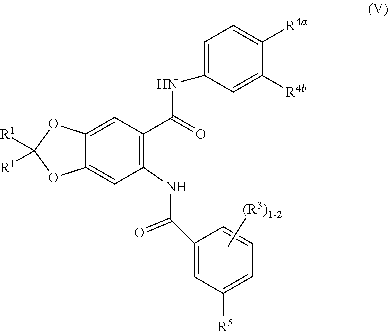

or pharmaceutically acceptable salts thereof, wherein:
R¹ is halo;
R³ is halo, C₁₋₃ alkyl, or —OC₁₋₄ alkyl;
R^{4a} is halo;
R^{4b} is C₁₋₃ alkyl substituted with 0-3 halo;
R⁵ is —C(═O)NR¹³R¹³, C₂₋₆alkenyl substituted with 0-3 R⁶ and 0-2 R⁵, C₂₋₆ alkynyl substituted with 0-3 R⁶ and 0-2 R⁷, phenyl substituted with 0-3 R⁶ and 0-2 R⁵, or a 3- to 10-membered heterocyclyl comprising 1-4 heteroatoms selected from O, S(═O)ₚ, N and NR¹⁰, and substituted with 0-3 R⁶ and 0-1 R⁷;
R⁵ is halo, ═O, —OH, —OC₁₋₄ alkyl, or C₁₋₄ alkyl substituted with 0-2 halo or OH;
R⁷ is C₁₋₄ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR⁵, C₃₋₆ cycloalkyl substituted with 0-4 R^{c}, or a 4- to 6-membered heterocyclyl comprising 1-4 heteroatoms selected from O, S(═O)ₚ, N and NR^{d}, and substituted with 0-4 R^{c};
R^{g} is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{c}, —NR^{a}S(═O)ₚR, —NR^{a}S(O)ₚNR^{a}R^{a}, —OC(═O)NR^{a}R^{a}, —S(═O)ₚNR^{a}R^{a}, or —S(O)ₚR^{c};
R¹¹ is H, C₁₋₄ alkyl substituted with 0-2 R¹⁰, —C(═O)R^{b}, —C(═O)OR^{b}, —C(═O)NR^{a}R^{a}, —S(O)ₚR^{c}, C₃₋₆ cycloalkyl substituted with 0-5 R^{e}, or a 4- to 6-membered heterocyclyl comprising 1-4 heteroatoms selected from O, S(═O)ₚ, N and NR¹² and substituted with 0-5 R^{e};
R¹¹ is —OH, —C(═O)OH, —C(═O)NR^{a}R^{a}, or aryl;
R¹² is H, C₁₋₃ alkyl, or aryl;
R¹³ is H or C₁₋₄ alkyl substituted with 0-3 R⁶ and 0-2 R⁷; or R¹³ and R¹³ together with the nitroen atom to which they are both attached form a heterocyclyl comprising additional 0-4 heteroatoms selected from O, S(═O)ₚ, N, and NR¹⁰, and substituted with 0-3 R⁶ and 0-2 R⁷;
R^{a} is H, C₁₋₅ alkyl substituted with 0-4 R^{e}, —(CH₂)ₙ—C₃₋₁₀ carbocyclyl substituted with 0-4 R^{c}, or —(CH₂)ₙ-heterocyclyl substituted with 0-4 R^{c}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₃₋₆ carbocyclyl, or heterocyclyl;
R^{d} is H or C₁₋₂ alkyl;
R^{c} is halo, CN, ═O, C₁₋₆ alkyl substituted with 0-5 R⁵, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl substituted with 0-1 —OC₁₋₄ alkyl;
R⁸ is halo, CN, OH, C₁₋₆ alkyl, or C₃₋₆ cycloalkyl;
n is zero, 1, 2, or 3; and
p is zero, 1, or 2.

In an eighth aspect within the scope of the seventh aspect, the present invention provides compounds of Formula (VI):

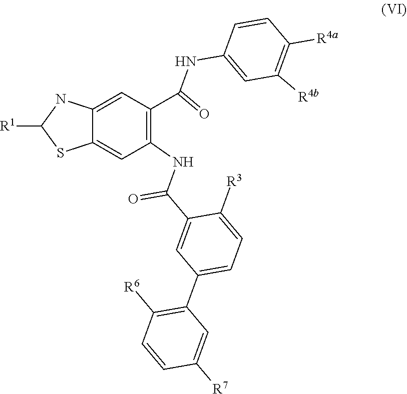

or pharmaceutically acceptable salts thereof, wherein:
R¹ is F;
R³ is —OCH₃;
R^{4a} is F;
R^{4b} is CF₃;
R⁶ is halo;
R⁷ is —C(═O)OR^{b}, —C(═O)NR^{a}R^{a}, —S(═O)ₚNR^{a}R^{a}, or —C(═O)NR^{a}S(═O)ₚC₁₋₄alkyl;
R^{a} is H, C₁₋₃ alkyl substituted with 0-5 R⁵, or heterocyclyl substituted with 0-4 R^{e};
R^{b} is H or C₁₋₃ alkyl substituted with 0-5 R^{c}; and
R^{c} is halo, CN, ═O, or C₁₋₆ alkyl.

In a ninth aspect within the scope of the seventh aspect, the present invention provides compounds of Formula (V), or pharmaceutically acceptable salts thereof, wherein:
R¹ is F;
R³ is —OCH₃;
R^{4a} is F;
R^{4b} is CF₃;
R⁵ is

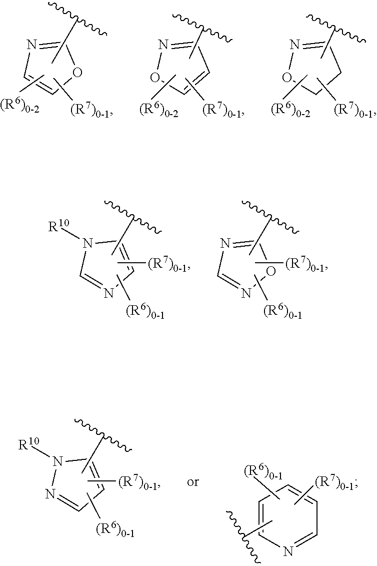

R⁶ is halo, —OH, or C₁₋₄ alkyl substituted with 0-1 OH;
R⁷ is —NR^{a}R^{a} or —S(═O)₂NR^{a}R^{a};
R¹⁰ is H, —C(═O)R¹, or C₁₋₄ alkyl substituted with 0-1 R¹¹,
R¹¹ is —OH, —C(═O)OH, or aryl;
R⁶ is H or C₁₋₃ alkyl; or R^{a} and R^{a} together with the nitrogen atom to which they are both attached form a heterocyclyl; and
R^{b} is H or C₁₋₃ alkyl.

In a tenth aspect within the scope of the seventh aspect, the present invention provides compounds of Formula (V), or pharmaceutically acceptable salts thereof, wherein:
R¹ is F;
R³ is —OCH₃;
R^{4a} is F;
R^{4b} is CF₃;
R⁵ is

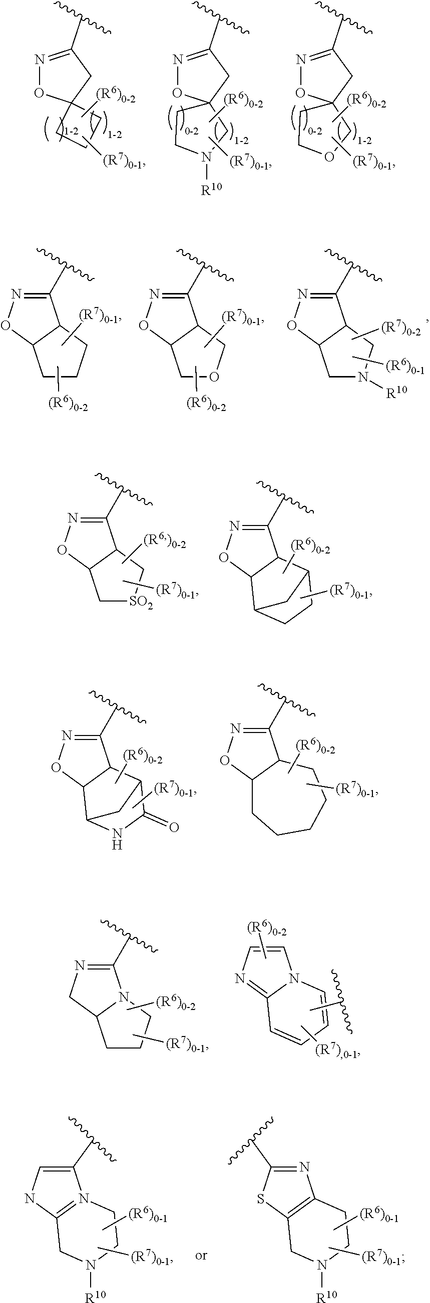

R⁶ is halo, ═O, —OH, —OC₁₋₄ alkyl, or C₁₋₄ alkyl;
R⁷ is C₁₋₃ alkyl substituted with 0-1 R⁸ and 0-1 R⁹ —NR^{a}R^{a}, or —NR^{a}C(═O)R^{b};
R⁸ is —C(═O)OR^{b};
R⁹ is OH;
R¹⁰ is H, C₁₋₃ alkyl substituted with 0-2 R¹¹, —C(═O)R^{b}, —C(═O)OR^{b}, —C(═O)NR^{a}R^{a}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, or C(═O)NR^{a}R^{a};
R¹² is H and C₁₋₃ alkyl;
R^{a} is H or C₁₋₃ alkyl;
R^{b} is H or C₁₋₃ alkyl substituted with 0-1 R^{e}; and
R^{e} is OH.

In an eleventh aspect within the scope of the tenth aspect, the present invention provides compounds of Formula (V), or pharmaceutically acceptable salts thereof, wherein:
R⁵ is

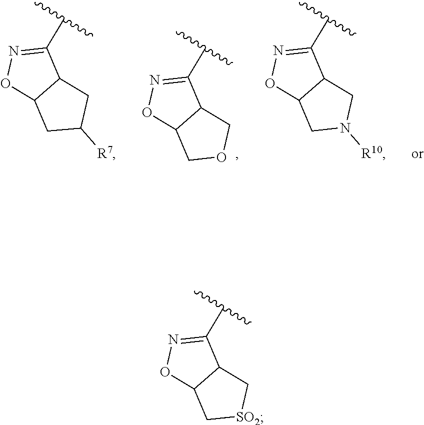

R⁷ is C₁₋₃ alkyl substituted with 0-1 R⁵,
R⁹ is —OH;
R¹⁰ is H, C₁₋₃ alkyl substituted with 0-2 R¹¹, or —C(═O)R^{b};
R¹¹ is —OH;
R^{b} is H or C₁₋₃ alkyl substituted with 0-1 R^{c} and
R^{e} is OH.

In a twelfth aspect within the scope of the seventh aspect, the present invention provides compounds of Formula (V), or pharmaceutically acceptable salts thereof, wherein:
R¹ is F;
R³ is —OCH₃;
R^{4a} is F;
R^{4b} is CF₃;
R⁵ is

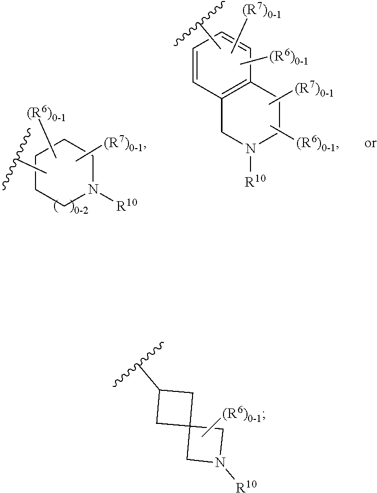

R⁶ is halo, ═O, —OH, —OC₁₋₄ alkyl, or C₁₋₄ alkyl;
R⁷ is C₁₋₂ alkyl substituted with 0-1 R^{g} and 0-1 R⁹, —NR^{a}R^{a}, —NR^{a}C(═O)R^{b}, —NR^{a}C(═O)OR^{b}, or —C(═O)OR^{b};
R⁸ is —C(═O)OR^{b}, —C(═O)NHR^{a}, —C(═O)NHOR^{b}, or C₁₋₅ alkyl substituted with 0-3 halo or OH;
R⁹ is —NR^{a}C(═O)R^{b};
R¹⁰ is H, C₁₋₄ alkyl substituted with 0-2 R¹¹, —C(═O)R^{b}, —C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 Rr;
R¹¹ is —OH, —C(═O)OH, or C(═O)NR^{a}R^{a};
R¹² is H, C₁₋₃ alkyl, or aryl;
R^{a} is H or C₁₋₃ alkyl;
R^{b} is H, or C₁₋₃ alkyl substituted with 0-1 R^{c}; and
R^{e} is OH.

In a thirteenth aspect within the scope of the seventh aspect, the present invention provides compounds of Formula (V), or pharmaceutically acceptable salts thereof, wherein:
R¹ is F;
R³ is —OCH₃;
R^{4a} is F;
R^{4b} is CF₃;
R⁵ is —C(═O)NR¹³R¹³;
R⁶ is halo, —OH, or C₁₋₄ alkyl substituted with 0-1 OH;
R⁷ is —S(═O)₂C₁₋₃ alkyl or C₃₋₆ cycloalkyl substituted with 0-2 R^{e};
R¹⁰ is H, —C(═O)R^{b}, or C₁₋₄ alkyl substituted with 0-1R¹¹;
R¹¹ is —OH, —C(═O)OH, or aryl;
R¹³ is H or C₁₋₃ alkyl substituted with 0-1 R⁶ and 0-1 R⁷; or R¹³ and R¹³ together with the nitrogen atom to which they are both attached form a heterocyclyl selected from

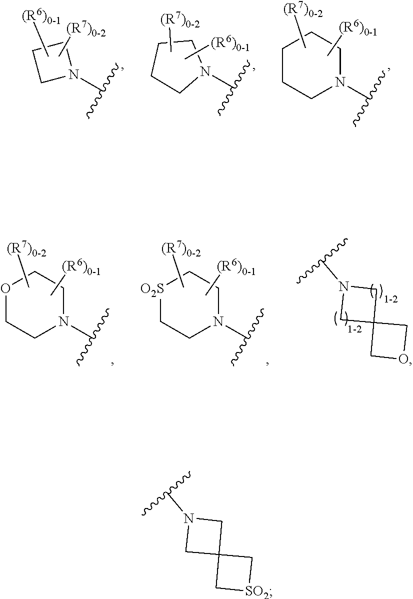

R^{b} is H or C₁₋₆ alkyl; and
R^{e} is —S(═O)₃C₁₋₃ alkyl.

In a fourteenth aspect within the scope of the thirteenth aspect, the present invention provides compounds of Formula (?), or pharmaceutically acceptable salts thereof, wherein:
R⁵ is —C(═O)NHR¹³;
R¹³ is C₁₋₃ alkyl substituted with 0-1 R⁷;
R⁷ is C₃₋₄ cycloalkyl substituted with 0-2 R^{e}; and
R^{c} is —S(═O)₂C₁₋₃ alkyl.

In a fifteenth aspect within the scope of the second aspect, the present invention provides compounds of Formula (II), or pharmaceutically acceptable salts thereof, wherein:

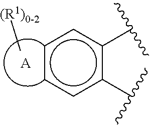

is

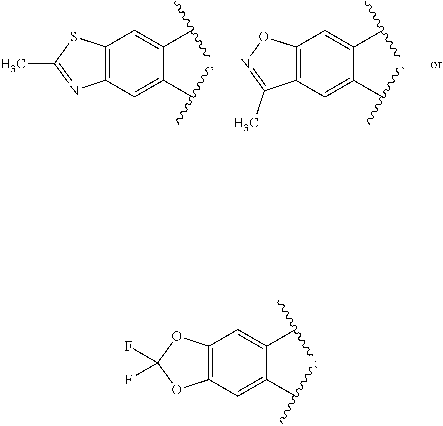 [SMILES: *c1cc2nc(C)sc2cc1* ; *c1cc2onc(C)c2cc1* ; *c1cc2c(cc1*)OC(F)(F)O2]

R³ is —OC₁₋₃ alkyl;
R^{4a} is F;
R^{4b} is CF₃;
R⁵ is —C(═O)NHR¹³,

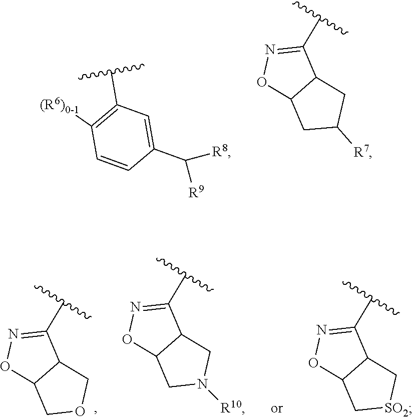

R⁷ is C₁₋₃ alkyl substituted with 0-1 R⁹ or C₃₋₆ cycloalkyl substituted with 0-5 R^{e};
R⁸ is halo, —C(═O)OR^{b}, or C₁₋₄ alkyl substituted with 0-3 halo:
R⁹ is —OH, —NR^{a}C(═O)R^{b} or —OC(═O)NR^{a}R^{a};
R¹⁰ is H, C₁₋₃ alkyl substituted with 0-2 R¹¹, or —C(═O)R^{b};
R¹¹ is —OH;
R¹³ is H or C₁₋₃ alkyl substituted with 0-1 R⁷;
R^{b} is H, C₁₋₃ alkyl substituted with 0-5 R^{e}, or heterocyclyl;
R^{e} is —OR^{f} or —S(═O)₂C₁₋₃ alkyl; and
R^{f} is H or C₁₋₃ alkyl.

In a sixteenth aspect within the scope of the twelfth aspect, the present invention provides compounds of Formula (V), or pharmaceutically acceptable salts thereof, wherein:
R¹ is F;
R³ is —OCH₃;
R¹ is F;
R^{a} is CF₃;
R¹ is C₂₋₆ alkenyl substituted with 0-3 R⁸ and 0-2 R⁵, C₂₋₆ alkynyl substituted with 0-3 R⁶ and 0-2 R⁷;
R⁶ is halo, —OH, or C₁₋₄ alkyl substituted with 0-1 OH;
R⁷ is —OR^{b} or —NR^{a}R^{a};
R⁶ is H or C₁₋₃ alkyl; or R^{a} and R^{a} together with the nitrogen atom to which they are both attached form a heterocyclyl; and
R^{b} is H, C₁₋₃ alkyl, or heterocyclyl.

For a compound of Formula (I), the scope of any instance of a variable substituent, including R¹, R², R³, R⁴ (R^{4a}, R^{4b}), R⁵, R⁶, R⁷, R⁸, R⁹, R¹⁰, R¹¹, R¹², R¹³, R^{a}, R^{b}, R^{c}, R^{d}, R^{e}, R^{f}, and R^{g} can be used independently with the scope of any other instance of a variable substituent. As such, the invention includes combinations of the different aspects.

In one embodiment of Formula (II),

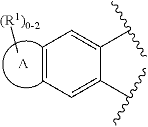

is

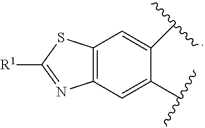

In another embodiment of Formula (II),

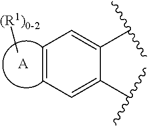

is

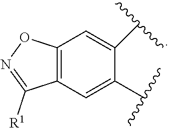

In another embodiment of Formula (II),

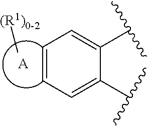

is

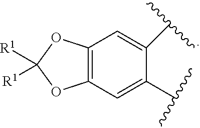

In another embodiment of Formula (II), R^{4a} is F.

In another embodiment of Formula (II), R^{4b} is CF₃.

In another embodiment of Formula (II),

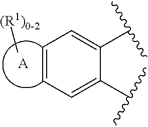

is

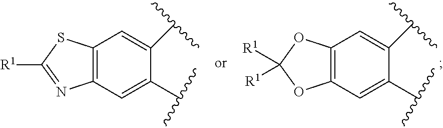

R³ is F or —OCH₃; R^{4a} is F; R^{4b} is CF₃; R⁵ is —C(═O)NR¹³R¹³; R¹³ is H or C₁₋₅ alkyl substituted with 0-1 R⁷; or R¹³ and R¹³ together with the nitrogen atom to which they are both attached form

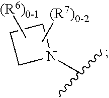

R⁶ is —OH or C₁₋₃ alkyl; R⁷ is C₁₋₃ alkyl substituted with 0-1 R⁹, —S(═O)₂C₁₋₃ alkyl, or C₃₋₆ cycloalkyl substituted with 0-2 R^{e}; R⁹ is —OH; and R^{e} is —S(═O)₂C₁₋₃ alkyl.

In another embodiment of Formula (II),

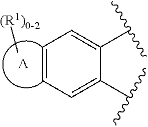

is

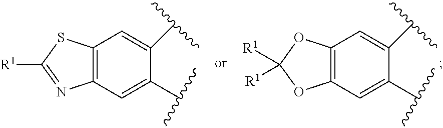

R³ is F or —OCH₃; R^{4a} is F; R^{4b} is CF₃; R⁵ is

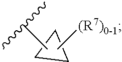

R⁷ is C₁₋₅ alkyl substituted with 0-1 R⁹ or —C(═O)NHR^{a}; R⁹ is —OH, R^{a} is H, C₁₋₃ alkyl, —(CH₂)₀₋₁—C₃₋₆ cycloalkyl, or —(CH₂)₀₋₁-heterocyclyl.

In another embodiment of Formula (II),

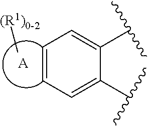

is

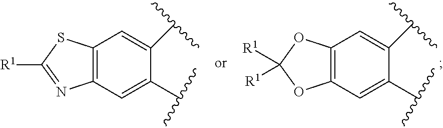

R³ is F or —OCH₃; R^{4a} is F; R^{4b} is CF₃; R⁵ is

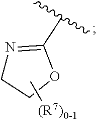

R⁷ is —C(═O)NR^{a}R^{a}; R^{a} and R^{a} together with the nitrogen atom to which they are both attached form

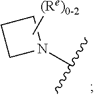

R^{e} is C₁₋₃ alkyl substituted with 0-2 R^{g}; R⁹ is —OH alkyl.

In another embodiment of Formula (II),

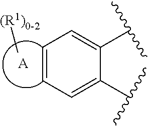

is

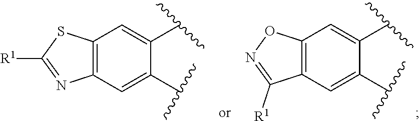

R³ is F or —OCH₃; R^{4a} is F; R^{4b} is CF₃; R⁵ is

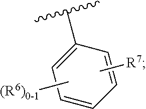

R⁶ is F; R⁷ is —S(═O)₂C₁₋₃; alkyl, —S(═O)₂NHR^{a}, or C₁₋₃ alkyl substituted with 0-1 R⁸ and 0-1 R⁹; R⁸ is —C(═O)OH, or CF₃; R⁹ is —NHR, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{c} is C₁₋₃ alkyl, —(CH₂)₀₋₁OR^{f}; and R^{f} is H or C₁₋₃ alkyl.

In another embodiment of Formula (II),

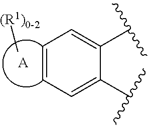

is

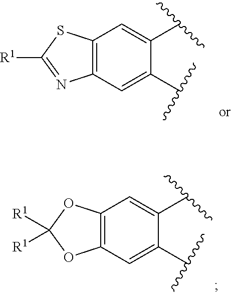

R³ is F or —OCH₃; R^{4a} is F; R^{4b} is CF₃; R⁵ is

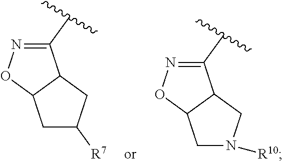

R⁷ is C₁₋₄ alkyl substituted with 0-1 R⁹; R⁹ is —OH; R¹¹ is —C(═O)R^{b}; R^{b} is H or C₁₋₃ alkyl substituted with 0-4 R^{e}; R^{e} is —(CH₂)₀₋₁OR^{f}; and R^{f} is H or C₁₋₃ alkyl.

Unless specified otherwise, these terms have the following meanings.

“Halo” includes fluoro, chloro, bromo, and iodo.

“Alkyl” or “alkylene” is intended to include both branched and straight-chain saturated aliphatic hydrocarbon groups having the specified number of carbon atoms. For example, “C₁ to C₁₀ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁ to C₆ alkyl” or “C₁-C₆ alkyl” denotes alkyl having 1 to 6 carbon atoms. Alkyl group can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). When “Co alkyl” or “Co alkylene” is used, it is intended to denote a direct bond. “Alkyl” also includes deuteroalkyl such as CD₃.

“Alkenyl” or “alkenylene” is intended to include hydrocarbon chains of either straight or branched configuration having one or more, preferably one to three, carbon-carbon double bonds that may occur in any stable point along the chain. For example. “C₂ to C₆ alkenyl” or “C₂₋₆ alkenyl” (or alkenylene), is intended to include C₂, C₃, C₄, C₅, and C₆ alkenyl groups; such as ethenyl, propenyl, butenyl, pentenyl, and hexenyl.

“Alkynyl” or “alkynylene” is intended to include hydrocarbon chains of either straight or branched configuration having one or more, preferably one to three, carbon-carbon triple bonds that may occur in any stable point along the chain. For example. “C₂ to C₆ alkynyl” or “C₂₋₆ alkynyl” (or alkynylene), is intended to include C₂, C₃, C₄, C₅, and C₆ alkynyl groups: such as ethynyl, propynyl, butynyl, pentynyl, and hexynyl.

“Carbocycle”, “carbocyclyl”, or “carbocyclic residue” is intended to mean any stable 3-, 4-, 5-, 6-, 7-, or 8-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocyclyls include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocyclyl (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. When the term “carbocyclyl” is used, it is intended to include “aryl,” “cycloalkyl,” and “spirocycloalkyl.” Preferred carbocyclyls, unless otherwise specified, are cyclopropyl, cyclobutyl, cyclopentyl, cyclohexyl, phenyl, and indanyl.

“Cycloalkyl” is intended to mean cyclized alkyl groups, including mono-, bi- or multicyclic ring systems. “C₃ to C₇ cycloalkyl” or “C₃₋₇ cycloalkyl” is intended to include C₃, C₄, C₅, C₆, and C₇ cycloalkyl groups. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbomyl and adamantyl.

“Spirocycloalkyl” is intended to mean hydrocarbon bicyclic ring systems with both rings connected through a single atom. The ring can be different in size and nature, or identical in size and nature. Examples include spiropentane, spriohexane, spiroheptane, spirooctane, spirononane, or spirodecane.

“Bicyclic carbocyclyl” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated, partially unsaturated, or unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, naphthyl, 1,2-dihydronaphthyl, 1,2,3,4-tetrahydronaphthyl, and indanyl.

“Aryl” groups refer to monocyclic or polycyclic aromatic hydrocarbons, including, for example, phenyl, naphthyl, and phenanthranyl. Aryl moieties are well known and described, for example, in Lewis, R J., ed., Hawley's Condensed Chemical Dictionary, 13th Edition, John Wiley & Sons, Inc., New York (1997).

“Benzyl” is intended to mean a methyl group on which one of the hydrogen atoms is replaced by a phenyl group, wherein said phenyl group may optionally be substituted with 1 to 5 groups, preferably 1 to 3 groups.

“Heterocycle”, “heterocyclyl” or “heterocyclic ring” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocyclyl may optionally be quaternized. It is preferred that when the total number of S and O atoms in the heterocyclyl exceeds 1, then these heteroatoms are not adjacent to one another. It is preferred that the total number of S and O atoms in the heterocyclyl is not more than 1. Bridged rings are also included in the definition of heterocyclyl. When the term “heterocyclyl” is used, it is intended to include heteroaryl.

Examples of heterocyclyls include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocyclyls.

“Bicyclic heterocyclyl” “bicyclic heterocyclyl” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N. O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocyclyl, a 6-membered heterocyclyl or a carbocyclyl (provided the first ring is not benzo when the second ring is a carbocyclyl).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocyclyl exceeds 1, then these heteroatoms are not adjacent to one another. It is preferred that the total number of S and O atoms in the heterocyclyl is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydroquinolinyl, 2,3-dihydrobenzofuranyl, chromanyl, 1,2,3,4-tetrahydroquinoxalinyl, and 1,2,3,4-tetrahydroquinazolinyl.

“Heteroaryl” is intended to mean stable monocyclic and polycyclic aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C=C, C=N, or N=N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The invention includes all tautomeric forms of the compounds, atropisomers and rotational isomers.

All processes used to prepare compounds of the present invention and intermediates made therein are considered to be part of the present invention.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistr, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

The invention is intended to include all isotopes of atoms occurring in the compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt: a salt may be converted into the free compound or another salt, a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

### Biological Methods

RXFP1 Cyclic Adenosine Monophosphate (cAMP) Assays. Human embryonic kidney cells 293 (HEK293) cells and HEK293 cells stably expressing human RXFP1, were cultured in MEM medium supplemented with 10% qualified FBS, and 300 μg/ml hygromycin (Life Technologies). Cells were dissociated and suspended in assay buffer. The assay buffer was HBSS buffer (with calcium and magnesium) containing 20 mM HEPES, 0.05% BSA, and 0.5 mM IBMX. Cells (3000 cells per well, except 1500 cell per well for HEK293 cells stably expressing human RXFP1) were added to 384-well Proxiplates (Perkin-Elmer). Cells were immediately treated with test compounds in DMSO (2% final) at final concentrations in the range of 0.010 nM to 50 μM. Cells were incubated for 30 min at room temperature. The level of intracellular cAMP was determined using the HTRF HiRange cAMP assay reagent kit (Cisbio) according to manufacturer's instructions. Solutions of cryptate conjugated anti-cAMP and d2 fluorophore-labelled cAMP were made in a supplied lysis buffer separately. Upon completion of the reaction, the cells were lysed with equal volume of the d2-cAMP solution and anti-cAMP solution. After a 1 h room temperature incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 2.7 μM to 0.1 μM by plotting the fluorescent intensity ratio from 665 nm emission to the intensity from the 590 nm emission against cAMP concentrations. The potency and activity of a compound to inhibit cAMP production was then determined by fitting to a 4-parametric logistic equation from a plot of cAMP level versus compound concentrations.

The examples disclosed below were tested in the human RXFP1 (hRXFP) HEK293 cAMP assay described above and found to have agonist activity. Tables 1-3, lists EC₅₀ values in the hRXFP1 HEK293 cAMP assay measured for the examples.

Table A lists EC₅₀ values in the hRXFP1 HEK293 cAMP assay measured for the examples.

TABLE A

| | cAMP hRXFP1 HEK293 |
|---|---|
| | Assay |
| Example | EC₅₀ (nM) |
| 2 | 1000 |
| 3 | 8.6 |
| 4 | 30 |
| 5 | 16 |
| 6 | 28 |
| 7 | 31 |
| 8 | 40 |
| 9 | 110 |
| 10 | 130 |
| 11 | 160 |
| 12 | 290 |
| 13 | 540 |
| 14 | 1400 |
| 15 | 180 |
| 16 | 46 |
| 19 | 1000 |
| 21 | 4100 |
| 22 | 8 |
| 23 | 750 |
| 24 | 750 |
| 26 | 1900 |
| 27 | 2200 |
| 28 | 3700 |
| 29 | 2700 |
| 30 | 15 |
| 31 | 116 |
| 32 | 18 |
| 34 | 1345 |
| 35 | 6 |
| 36 | 14 |
| 37 | 56 |
| 38 | 84 |
| 39 | 90 |
| 40 | 196 |
| 41 | 562 |
| 42 | 1202 |
| 43 | 13 |
| 44 | 16 |
| 45 | 6 |
| 46 | 49 |
| 47 | 85 |
| 48 | 196 |
| 49 | 154 |
| 50 | 98 |
| 51 | 21 |
| 52 | 683 |
| 53 | 33 |
| 54 | 683 |
| 55 | 322 |
| 57 | 125 |
| 58 | 1060 |
| 59 | 41 |
| 61 | 17 |
| 62 | 646 |
| 64 | 37 |
| 65 | 285 |
| 66 | 338 |
| 67 | 309 |
| 68 | 173 |
| 69 | 42 |
| 70 | 132 |
| 71 | 43 |
| 72 | 65 |
| 73 | 96 |
| 74 | 127 |
| 75 | 231 |
| 76 | 258 |
| 77 | 487 |
| 78 | 262 |
| 79 | 275 |
| 80 | 260 |
| 81 | 282 |
| 82 | 144 |
| 83 | 399 |
| 84 | 485 |
| 87 | 161 |
| 88 | 274 |
| 92 | 2638 |
| 93 | 1090 |
| 94 | 1397 |
| 95 | 862 |
| 96 | 339 |
| 97 | 666 |
| 98 | 1.3 |
| 99 | 3.9 |
| 100 | 8.4 |
| 101 | 9.8 |
| 102 | 10.6 |

### Pharmaceutical Compositions and Methods of Use

The compounds of Formula (I) are RXFP1 receptor agonists and may find use in the treatment of medical indications such as heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

Another aspect of the invention is a pharmaceutical composition comprising a compound of Formula (I) and a pharmaceutically acceptable carrier.

Another aspect of the invention is a pharmaceutical composition comprising a compound of Formula (I) for the treatment of a relaxin-associated disorder and a pharmaceutically acceptable carrier.

Another aspect of the invention is a method of treating a disease associated with relaxin comprising administering an effective amount of a compound of Formula (I).

Another aspect of the invention is a method of treating a cardiovascular disease comprising administering an effective amount of a compound of Formula (I) to a patient in need thereof.

Another aspect of the invention is a method of treating heart failure comprising administering an effective amount of a compound of Formula (I) to a patient in need thereof.

Another aspect of the invention is a method of treating fibrosis comprising administering a therapeutically effective amount of a compound of Formula (I) to a patient in need thereof.

Another aspect of the invention is a method of treating a disease associated with fibrosis comprising administering a therapeutically effective amount of a compound of Formula (I) to a patient in need thereof.

Another aspect of the invention is a method of treating or preventing kidney failure, comprising administering a therapeutically effective amount of a compound of Formula (I) to a patient in need thereof.

Another aspect of the invention is a method of improving, stabilizing or restoring renal function in a patient in need thereof, comprising administering a therapeutically effective amount of a compound of Formula (I) to the patient.

Unless otherwise specified, the following terms have the stated meanings.

The term “patient” or “subject” refers to any human or non-human organism that could potentially benefit from treatment with a RXFP1 agonist as understood by practioners in this field. Exemplary subjects include human beings of any age with risk factors for cardiovascular disease. Common risk factors include, but are not limited to, age, sex, weight, family history, sleep apnea, alcohol or tobacco use, physical inactivity, arrhythmia, or signs of insulin resistance such as acanthosis nigricans, hypertension, dyslipidemia, or polycystic ovary syndrome (PCOS).

“Treating” or “treatment” cover the treatment of a disease-state as understood by practitioners in this field and include the following: (a) inhibiting the disease-state, i.e., arresting it development; (b) relieving the disease-state, i.e., causing regression of the disease state; and/or (c) preventing the disease-state from occurring in a mammal, in particular, when such mammal is predisposed to the disease-state but has not yet been diagnosed as having it.

“Preventing” or “prevention” cover the preventive treatment (i.e., prophylaxis and/or risk reduction) of a subclinical disease-state aimed at reducing the probability of the occurrence of a clinical disease-state as understood by practitioners in this field. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state. “Risk reduction” or “reducing risk” covers therapies that lower the incidence of development of a clinical disease state. As such, primary and secondary prevention therapies are examples of risk reduction.

“Therapeutically effective amount” is intended to include an amount of a compound of the present invention that is effective when administered alone or in combination with other agents to treat disorders as understood by practitioners in this field. When applied to a combination, the term refers to combined amounts of the active ingredients that result in the preventive or therapeutic effect, whether administered in combination, serially, or simultaneously.

“Disorders of the cardiovascular system” or “cardiovascular disorders” include for example the following disorders: hypertension (high blood pressure), peripheral and cardiac vascular disorders, coronary heart disease, stable and unstable angina pectoris, heart attack, myocardial insufficiency, abnormal heart rhythms (or arrhythmias), persistent ischemic dysfunction (“hibernating myocardium”), temporary postischemic dysfunction (“stunned myocardium”), heart failure, disturbances of peripheral blood flow, acute coronary syndrome, heart failure, heart muscle disease (cardiomyopathy), myocardial infarction and vascular disease (blood vessel disease).

“Heart failure” includes both acute and chronic manifestations of heart failure, as well as more specific or related types of disease, such as advanced heart failure, post-acute heart failure, cardio-renal syndrome, heart failure with impaired kidney function, chronic heart failure, chronic heart failure with mid-range ejection fraction (HFmEF), compensated heart failure, decompensated heart failure, right heart failure, left heart failure, global failure, ischemic cardiomyopathy, dilated cardiomyopathy, heart failure associated with congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, heart failure associated with combined heart valve defects, myocardial inflammation (myocarditis), chronic myocarditis, acute myocarditis, viral myocarditis, diabetic heart failure, alcoholic cardiomyopathy, heart failure associated with cardiac storage disorders, diastolic heart failure, systolic heart failure, acute phases of worsening heart failure, heart failure with preserved ejection fraction (HFpEF), heart failure with reduced ejection fraction (HFrEF), chronic heart failure with reduced ejection fraction (HFrEF), chronic heart failure with preserved ejection fraction (HFpEF), post myocardial remodeling, angina, hypertension, pulmonary hypertension and pulmonary artery hypertension.

“Fibrotic disorders” encompasses diseases and disorders characterized by fibrosis, including among others the following diseases and disorders: hepatic fibrosis, cirrhosis of the liver, NASH, pulmonary fibrosis or lung fibrosis, cardiac fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitreoretinopathy and disorders of the connective tissue (for example sarcoidosis).

Relaxin-associated disorders include but are not limited to disorders of the cardiovascular system and fibrotic disorders.

The compounds of this invention can be administered by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

“Pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, anti-bacterial agents, anti-fungal agents, lubricating agents and dispensing agents, depending on the nature of the mode of administration and dosage forms.

Pharmaceutically acceptable carriers are formulated according to a number of factors well within the purview of those of ordinary skill in the art. These include, without limitation: the type and nature of the active agent being formulated; the subject to which the agent-containing composition is to be administered, the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V., Jr. et al., Remington: The Science and Practice of Pharmacy (2 Volumes), 22nd Edition, Pharmaceutical Press (2012).

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment: the route of administration, the renal and hepatic function of the patient, and the effect desired.

By way of general guidance, the daily oral dosage of each active ingredient, when used for the indicated effects, will range between about 0.01 to about 5000 mg per day, preferably between about 0.1 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition. A typical capsule for oral administration contains at least one of the compounds of the present invention (250 mg), lactose (75 mg), and magnesium stearate (15 mg). The mixture is passed through a 60 mesh sieve and packed into a No. 1 gelatin capsule. A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The compounds may be employed in combination with other suitable therapeutic agents useful in the treatment of diseases or disorders including: anti-atherosclerotic agents, anti-dyslipidemic agents, anti-diabetic agents, anti-hyperglycemic agents, anti-hyperinsulinemic agents, anti-thrombotic agents, anti-retinopathic agents, anti-neuropathic agents, anti-nephropathic agents, anti-ischemic agents, anti-hypertensive agents, anti-obesity agents, anti-hyperlipidemic agents, anti-hypertriglyceridemic agents, anti-hypercholesterolemic agents, anti-restenotic agents, anti-pancreatic agents, lipid lowering agents, anorectic agents, memory enhancing agents, anti-dementia agents, cognition promoting agents, appetite suppressants, agents for treating heart failure, agents for treating peripheral arterial disease, agents for treating malignant tumors, and anti-inflammatory agents.

The additional therapeutic agents may include ACE inhibitors, β-blockers, diuretics, mineralocorticoid receptor antagonists, ryanodine receptor modulators, SERCA2a activators, renin inhibitors, calcium channel blockers, adenosine A1 receptor agonists, partial adenosine A1 receptor, dopamine β-hydroxylase inhibitors, angiotensin II receptor antagonists, angiotensin II receptor antagonists with biased agonism for select cell signaling pathways, combinations of angiotensin II receptor antagonists and neprilysin enzyme inhibitors, neprilysin enzyme inhibitors, soluble guanylate cyclase activators, myosin ATPase activators, rho-kinase 1 inhibitors, rho-kinase 2 inhibitors, apelin receptor agonists, nitroxyl donating compounds, calcium-dependent kinase II inhibitors, antifibrotic agents, galectin-3 inhibitors, vasopressin receptor antagonists, FPR2 receptor modulators, natriuretic peptide receptor agonists, transient receptor potential vanilloid-4 channel blockers, anti-arrhythmic agents, I_{f} “funny current” channel blockers, nitrates, digitalis compounds, inotropic agents and β-receptor agonists, cell membrane resealing agents for example Poloxamer 188, anti-hyperlipidemic agents, plasma HDL-raising agents, anti-hypercholesterolemic agents, cholesterol biosynthesis inhibitors (such as HMG CoA reductase inhibitors), LXR agonist, FXR agonist, probucol, raloxifene, nicotinic acid, niacinamide, cholesterol absorption inhibitors, bile acid sequestrants, anion exchange resins, quaternary amines, cholestyramine, colestipol, low density lipoprotein receptor inducers, clofibrate, fenofibrate, bezafibrate, ciprofibrate, gemfibrizol, vitamin B6, vitamin B12, anti-oxidant vitamins, anti-diabetes agents, platelet aggregation inhibitors, fibrinogen receptor antagonists, aspirin and fibric acid derivatives, PCSK9 inhibitors, aspirin, and P2Y12 Inhibitors such as Clopidogrel.

The additional therapeutic agents may also include nintedanib, Pirfenidone, LPA 1 antagonists, LPA1 receptor antagonists, GLP1 analogs, tralokinumab (IL-13, AstraZeneca), vismodegib (hedgehog antagonist, Roche), PRM-151 (pentraxin-2, TGF beta-1, Promedior), SAR-156597 (bispecific Mab IL-4&IL-13, Sanofi), simtuzumab ((anti-lysyl oxidase-like 2 (anti-LOXL2) antibody, Gilead), CKD-942, PTL-202 (PDE inh./pentoxifylline/NAC oral control, release, Pacific Ther.), omipalisib (oral PI3K/mTOR inhibitor, GSK), IW-001 (oral sol, bovine type V collagen mod., ImmuneWorks), STX-100 (integrin alpha V/beta-6 ant, Stromedix/Biogen), Actimmune (IFN gamma), PC-SOD (midismase, inhaled, LTT Bio-Pharma/CKD Pharm), lebrikizumab (anti-IL-13 SC humanized mAb, Roche). AQX-1125 (SHIP1 activator, Aquinox), CC-539 (JNK inhibitor, Celgene), FG-3019 (FibroGen), SAR-100842 (Sanofi), and obeticholic acid (OCA or INT-747, Intercept).

The above other therapeutic agents, when employed in combination with the compounds of the present invention may be used, for example, in those amounts indicated in the Physicians' Desk Reference, as in the patents set out above, or as otherwise determined by practitioners in the art.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

The compounds of the present invention are also useful as standard or reference compounds, for example as a quality standard or control, in tests or assays involving RXFP1. Such compounds may be provided in a commercial kit, for example, for use in pharmaceutical research involving RXFP1. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness. The compounds of the present invention may also be used in diagnostic assays involving RXFP1.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises a first therapeutic agent, comprising a compound of the present invention or a pharmaceutically acceptable salt form thereof; and, (c) a package insert stating that the pharmaceutical composition can be used for the treatment of dyslipidemias and the sequelae thereof. In another embodiment, the package insert states that the pharmaceutical composition can be used in combination (as defined previously) with a second therapeutic agent for the treatment of dyslipidemias and the sequelae thereof. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### General Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene, T. W. et al., Protecting Groups in Organic Synthesis, 4th Edition, Wiley (2007)).

Benzothiazole compounds of this invention can be synthesized via the general methods outlined in Scheme I-IV. Scheme I describes how appropriately substituted benzothiazole compounds of this invention may be prepared from commercially available intermediate I-I. Starting from amine I-I,

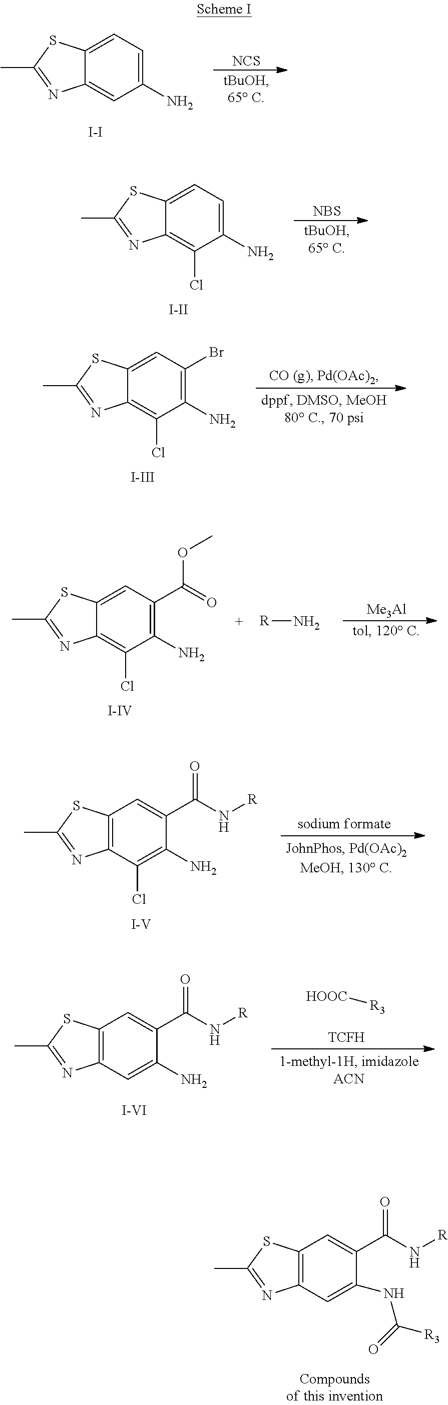

chlorination (NCS) provides intermediate 1-1I. Subsequent bromination (NBS) affords intermediate I-III. Palladium catalysis of intermediate I-IV in the presence of CO in MeOH affords the methyl ester intermediate I-IV which can be converted to a variety of amides in this invention by treatment with appropriate amines in the presence of amide AlMe₃, or via a sequential hydrolysis (LiOH, THF/MeOH/water), followed by standard peptide coupling procedures known in the art to afford intermediate I-V. The chloro moiety of I-V can be further converted to a variety of functional groups or can be de-chlorinated under dehalogenation conditions (e.g. Pd(OAc)₂, JohnPhos) to afford intermediate I-VI. Alternatively, the chlorine may be removed from I-IV followed by amide formation using an appropriate amine and AlMe₃, or coupling of the carboxylic acid (derived from hydrolysis of the ester) with appropriate amines of this invention via standard coupling procedures known in the art. The coupling of intermediate I-VI with appropriately carboxylic acids of this invention via a variety of amide bond forming conditions (e.g., TCFH, 1-methyl-1H-imidazole or BOP, DIEA or other conditions known in the art) provide compounds of this invention.

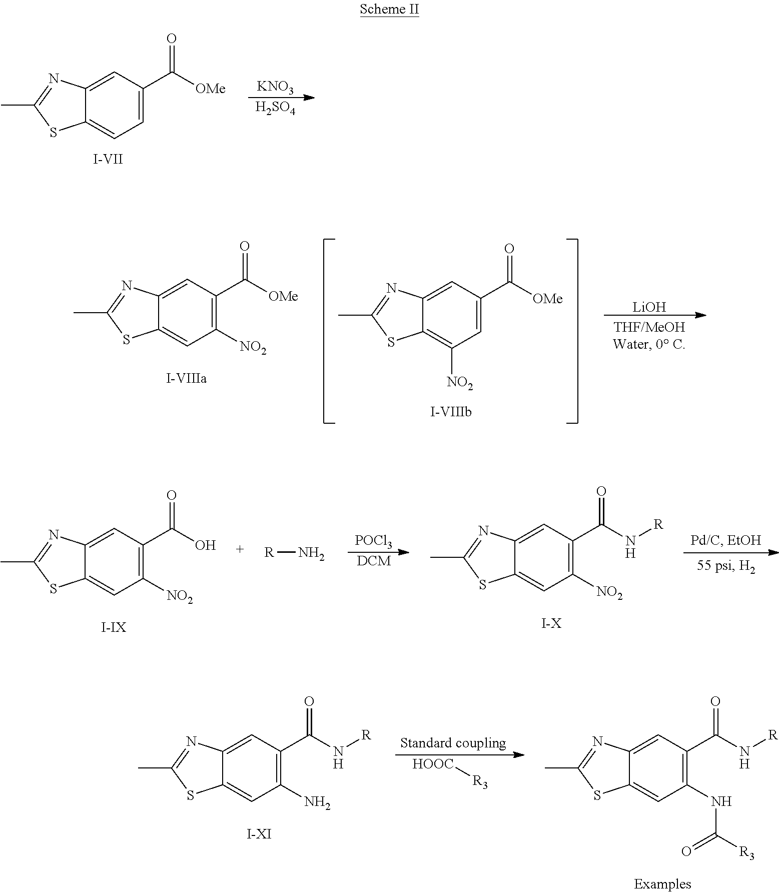

Alternatively, Scheme II describes how benzothiazole examples of this invention may be prepared from commercially available intermediate I-VII. Starting from methyl 2-methylbenzo[d]thiazole-5-carboxylate intermediate I-VII, nitration (HNO₃, H₂SO₄) provides nitro intermediates I-VIIIa and I-VIIIb. Intermediate I-VIIIa can be hydrolyzed (e.g., LiOH, H₂O) to the carboxylic acid derivative I-IX, which can be coupled with various amines of this invention (e.g., POCl₃, or BOP, DIEA, or HATU, DIEA and or other methods known in the art) to provide intermediate I-X. The nitro group reduction of I-X (e.g., H₂, Pd/C, or Zn, NH₄Cl) provides intermediate I-XI, which on coupling with appropriately carboxylic acid intermediates of this invention via a variety of amide bond forming conditions (e.g., TCFH, 1-methyl-1H-imidazole or BOP, DIEA) to provide compounds of this invention.

Alternatively, benzothiazoles (Scheme III) of this invention can also be synthesized via the general methods described in Schemes I and II. The aniline intermediate I-XV can then be coupled under conditions previously described or known in the art to with appropriately substituted benzoic acids to afford benzothiazole compounds of this invention.

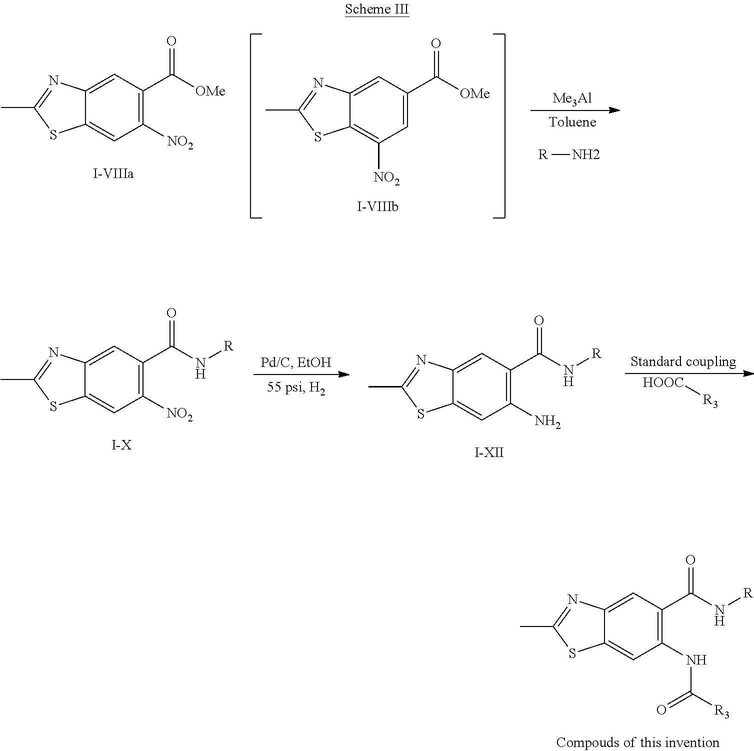

Trifluoromethylbenzothiazole intermediates I-XVI can be accessed according to the general route shown in Scheme IV (Du, G. et. al. Het. 2015, 9, 1723). The intermediate I-XIII can then be converted to the trifluorobenzothiazole intermediate I-XVI as previous shown in Scheme II.

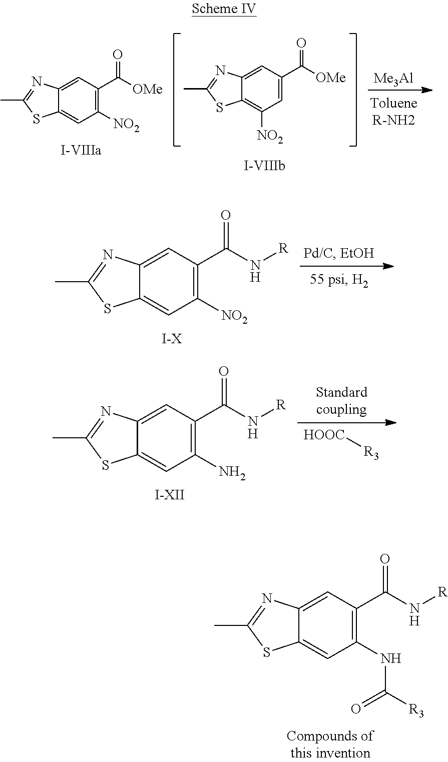

Substituted benzoic acids or heterocyclic acid intermediates used in the preparation of Examples in this invention can be synthesized by the general route shown in Scheme V. The scheme is general for aryl and heteroaryl groups where X═C or N to generate compounds of the general formula I-XIX that were further hydrolyzed or hydrogenated to give compounds of this invention.

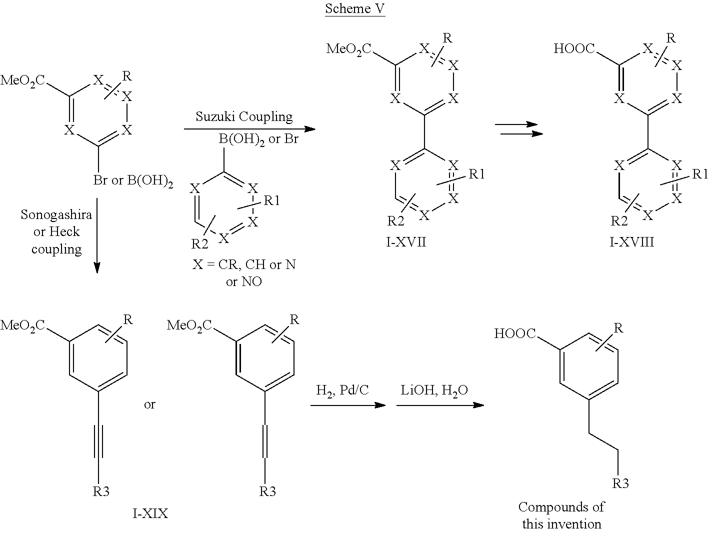

Arylisoxazoline intermediates of this invention can be obtained from commercially available aryl or heteroaryl carboxaldehydes as shown by the general route in Scheme VI. Conversion of the aldehyde I-XX to the oxime 1-XXI followed by treatment with NCS provides chlorooxime I-XXII. Treatment of IXII with an appropriate olefin affords arylisoxazoline intermediates for the general formula I-XXIII, IXXIV. Conversely treatment of I-XXII with an acetylenic intermediate affords arylisoxazole derivatives I-XXV of this invention. Heteroaryl carboxaldehydes can also be subjected to similar conditions to afford heteroaryl isoxazoline or isoxazole intermediates of this invention. Diastereomeric mixtures generated from the condensation can be separated via chiral SFC.

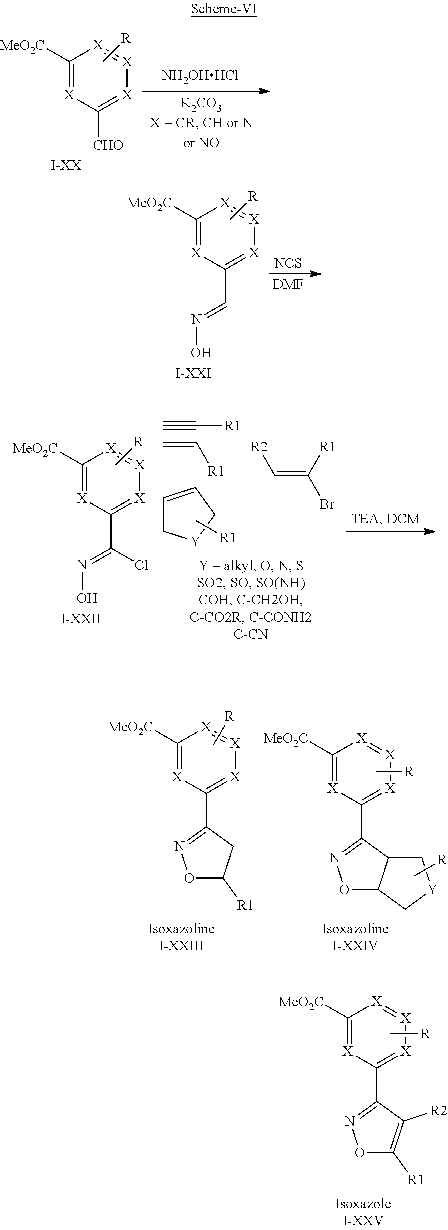

Difluorodioxolane intermediates of this invention can be obtained from commercially available 2,2-difluorobenzo[d][1,3]dioxol-5-amine as shown in Scheme VII. Bromination (NBS) affords intermediate I-XXVI. Palladium catalysis of intermediate I-XXVI in the presence of CO in MeOH affords the methyl ester intermediate I-XXVII which can be converted to a variety of amides of this invention by treatment with XNH₂ in the presence of AlMe₃, or via a sequential hydrolysis (LiOH, THF/MeOH/water), followed by peptide coupling procedures known in the art to afford intermediate I-XXVIII. Intermediate I-XXVII was coupled with arylcarboxylic acids to yield I-XXIX. Deprotection of I-XXXIX with TFA in DCM generated the corresponding carboxylic acid intermediates I_XXX which could be coupled with amines NHR₁R₂ using peptide coupling protocols to yield compounds of this invention.

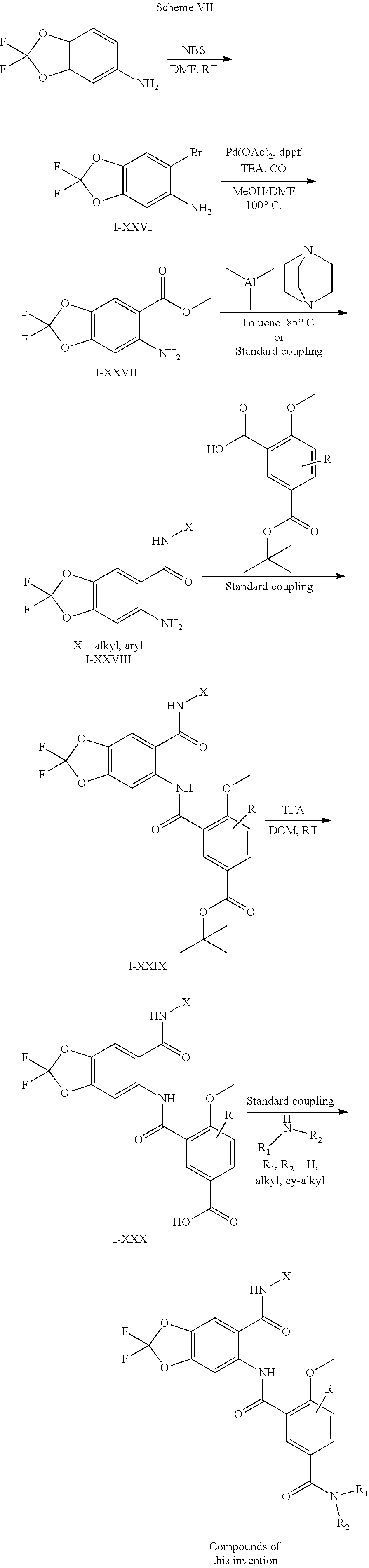

Intermediate I-XXVIII could also be coupled with heterocyclic carboxylic acids as shown in Scheme VIII. Conversion of the carboxylic acid to the corresponding aldehyde I-XXIX followed by reductive amination generated benzylamine examples of this invention.

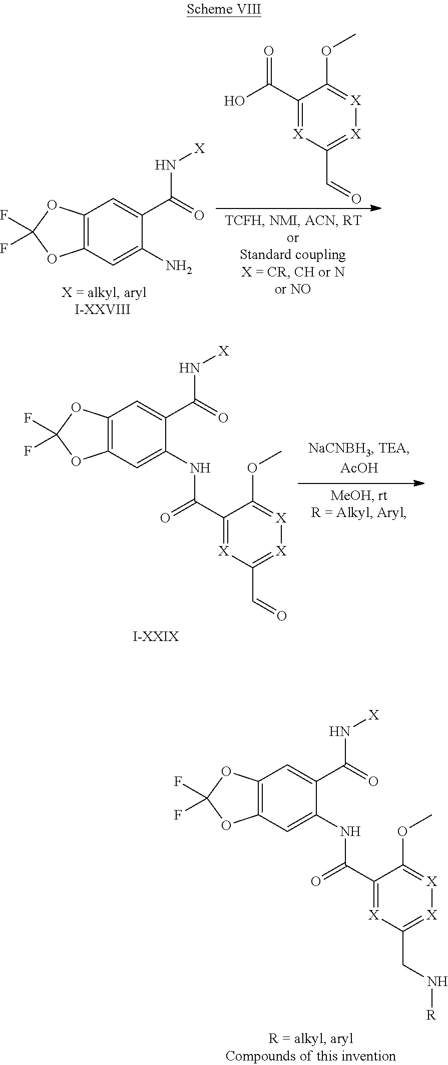

Abbreviations are defined as follows: “1 x” for once, “2 x” for twice. “3 x” for thrice, “° C.” for degrees Celsius, “aq” for aqueous, “eq” or “equiv.” for equivalent or equivalents, “g” for gram or grams, “mg” for milligram or milligrams, “L” for liter or liters, “mL” for milliliter or milliliters, “μL” for microliter or microliters, “N” for normal, “M” for molar, “nM” for nanomolar, “pM” for picomolar, “mol” for mole or moles, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “aq” for “aqueous”, “sat.” for saturated, “MW” for molecular weight, “MS” or “Mass Spec” for mass spectrometry, “ESI” for electrospray ionization mass spectroscopy, “LC-MS” for liquid chromatography mass spectrometry, “HPLC” for high pressure liquid chromatography, “RP HPLC” for reverse phase HPLC, “NMR” for nuclear magnetic resonance spectroscopy, “SFC” for super critical fluid chromatography, “1H” for proton, “S” for delta, “s” for singlet, “d” for doublet, “t” for triplet, “q” for quartet, “m” for multiplet, “br” for broad, “Hz” for hertz, “MHz” for megahertz, and “α”, “β”, “R”, “S”, “E”, and “Z” are stereochemical designations familiar to one skilled in the art.
AcCl acetyl chloride
AcOH acetic acid
AlBN Azobisisobutyronitrile
BHFFT bis(tetramethylene)fluoroformadmidinium hexafluorophosphate
Boc tert-butyloxycarbonyl
BuLi butyllithium
CDI 1,1′-Carbonyldiimidazole
DAST Diethylaminosulfur trifluoride
DCE Dichloroethane
DCM Dichloromethane
DIEA diispropyl ethylamine
DMAP4-dimethylamino pyridine
DMF Dimethylformamide
DPPA Diphenyl phosphorylazide
DPPF 1,1′-Bis(diphenylphosphno)ferrocene
Et₂O diethyl ether
EtOAc Ethyl acetate
EtOH Ethanol
HATU (1-[Bis(dimethylamino)methylene]-1H-1,2,3-triazolo[4,5-b]pyridinium 3-oxid hexafluorophosphate)
HMPA hexamethylphosphoramide
IPA isopropanol
i-Pr Isopropyl
KHMDS potassium bis(trimethylsilyl)amide
LDA lithium diisopropyl amide
ACN Acetonitrile
MeOH Methanol
Me Methyl
NBS N-bromosuccinimide
Pd/C palladium on carbon
pTsOH p-toluenesulfonic acid
PyBroP Bromotripyrrolidinophosphonium hexafluorophosphate
T3P 2,4,6-Tripropyl-1,3,5,2,4,6-trioxatriphosphorinane-2,4,6-trioxide
TBAF tetra-n-butyl ammonium fluoride
t-Bu tert-butyl
TCFH N,N,M,N-tetramethylchlorofornamidinium hexafluorophosphate
Teoc 2-(trimethylsilyl)ethyl carboxylate
TFA trifluoroacetic acid
TFAA trifluoroacetic anhydride
THF Tetrahydrofuran
TsOH Tolulenesulfonic acid
XPhos-Pd-G2 2nd generation XPhos precatalyst CAS no. 1310584-14-5

The following methods were used in the exemplified examples, except where noted otherwise. Purification of intermediates and final products was carried out via either normal or reverse phase chromatography. Normal phase chromatography was carried out using prepacked SiO2 cartridges eluting with either gradients of hexanes and EtOAc or DCM and MeOH unless otherwise indicated. Reverse phase preparative HPLC was carried out using C18 columns with UV 220 nm or prep LCMS detection eluting with gradients of Solvent A (90% water, 10% MeOH, 0.1% TFA) and Solvent B (10% water, 90% MeOH, 0.1% TFA) or with gradients of Solvent A (95% water, 5% ACN, 0.1% TFA) and Solvent B (5% water, 95% ACN, 0.1% TFA) or with gradients of Solvent A (95% water, 2% ACN, 0.1% HCOOH) and Solvent B (98% ACN, 2% water, 0.1% HCOOH) or with gradients of Solvent A (95% water, 5% ACN, 10 mM NH₄OAc) and Solvent B (98% ACN, 2% water, 10 mM NH₄OAc) or with gradients of Solvent A (98% water, 2% ACN, 0.1% NH₄OH) and Solvent B (98% ACN, 2% water, 0.1% NH₄OH). LC/MS methods employed in characterization of examples are listed below.

### Method A

Instrument: Waters Acquity coupled with a Waters MICROMASS® ZQ Mass Spectrometer
Linear gradient of 2 to 98% B over 1 min, with 0.5 min hold time at 98% B
UV visualization at 220 nm
Column: Waters BEH C18, 2.1×50 mm
Flow rate: 0.8 mL/min (Method A)
Mobile Phase A: 0.05% TFA, 100% water
Mobile Phase B: 0.05% TFA, 100% acetonitrile

### Method B

Instrument: Shimadzu Prominence HPLC coupled with a Shimadzu LCMS-2020 Mass Spectrometer
Linear gradient of 0 to 100% B over 3 min, with 0.75 min hold time at 100% B UV visualization at 220 nm
Column: Waters Xbridge C18, 2.1×50 mm, 1.7 um particles
Flow rate: 1 mL/min
Mobile Phase A: 10 mM ammonium acetate, 95:5 water:acetonitrile
Mobile Phase B: 10 mM ammonium acetate, 5:95 water:acetonitrile

### Method C

Instrument: Shimadzu Prominence HPLC coupled with a Shimadzu LCMS-2020 Mass Spectrometer
Linear gradient of 0 to 100% B over 3 min, with 0.75 min hold time at 100% B
UV visualization at 220 nm
Column: Waters Xbridge C18, 2.1×50 mm, 1.7 um particles
Flow rate: 1 mL/min
Mobile Phase A: 0.1% TFA, 95:5 water:acetonitrile
Mobile Phase B: 0.1% TFA, 5:95 water:acetonitrile

### Method D

Instrument: Agilent 1200/6140 LCMS single quadrupole
Linear gradient of 20% B to 100% B over 4.6 min and hold at 20% B for next 0.4 min UV visualization at 254 nm
Column: Kinetex XB C18 75×3 mm, 2.6 μM
Flow rate: 1 mL/min for first 4 min to 1.5 mL/min in 0.6 min and then to 1 mL/min in next 0.4 min
Mobile Phase A: 10 mM NH₄COOH in water
Mobile Phase B: ACN

### Method E

Instrument: Agilent 1290 infinity II with G6135B Mass spectrometer
Linear gradient of 0% B to 100% B over 3 min UV visualization at 220 nm
Column: Xbridge BEH XP C18 50×2.1 mm, 2.5 μM
Flow rate: 1.1 mL/min at 50° C.
Mobile Phase A: 10 mM NH₄OAc in Water:ACN (95:05)
Mobile Phase B: 10 mM NH₄OAc in Water:ACN (05:95)

### Method F

Instrument: Agilent 1290 infinity II with G6135B Mass spectrometer
Linear gradient of 0% B to 100% B over 3 min UV visualization at 220 nm
Column: Xbridge BEH XP C18 50×2.1 mm, 2.5 μM
Flow rate: 1.1 mL/min at 50° C.
Mobile Phase A: 0.1% TFA in Water:ACN (95:05)
Mobile Phase B: 0.1% TFA in Water:ACN (05:95)

### Method G

Instrument: Agilent 1200/6140 LCMS single quadrupole
Linear gradient of 60% B to 100% B over 4 min and hold at 100% B for next 0.6 min UV visualization at 220 nm
Column: Xbridge BEH XP C18 50×2.1 mm, 2.5 μM
Flow rate: 1.0 mL/min for first 4 min to 1.5 mL/min in next 0.6 min
Mobile Phase A: 0.1% HCOOH in Water
Mobile Phase B: ACN

NMR Employed in Characterization of Examples. 1H NMR spectra were obtained with Bruker or JEOL® Fourier transform spectrometers operating at frequencies as follows: 1H NMR: 400 MHz (Bruker or JEOL®) or 500 MHz (Bruker or JEOL®), Bruker Avance III 300 MHz, Bruker Avance Neo 400 MHz, Bruker Avance 111400 MHz. Spectra data are reported in the format: chemical shift (multiplicity, coupling constants, number of hydrogens). Chemical shifts are specified in ppm downfield of a tetramethylsilane internal standard (d units, tetramethylsilane=0 ppm) and/or referenced to solvent peaks, which in 1H NMR spectra appear at 2.51 ppm for DMSO-d6, 3.30 ppm for CD₃OD, 1.94 ppm for CD₃CN, and 7.24 ppm for CDCl₃.

### Intermediate 1: Preparation of 5-amino-N-(4-fluoro-3-(trifluoromethyl)phenyl)-2-methylbenzo[d]thiazole-6-carboxamide

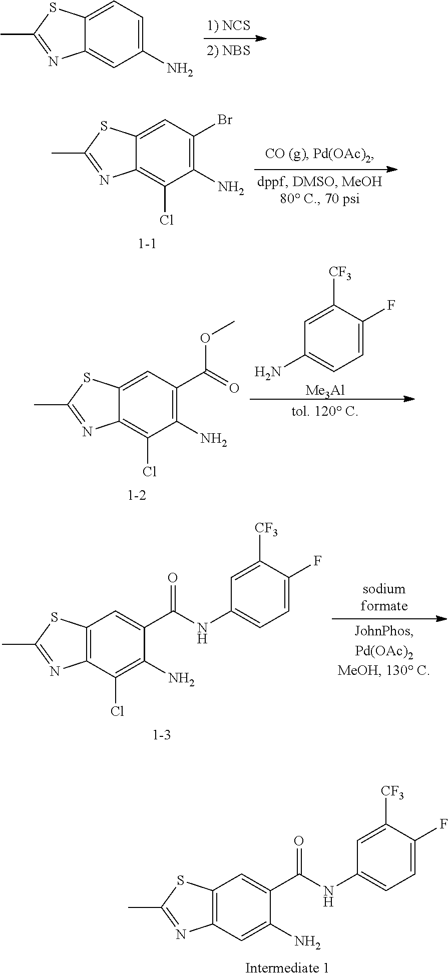

Intermediate 1-1: Preparation of 6-bromo-4-chloro-2-methylbenzo[d]thiazol-5-amine. Intermediate 1-1 (0.85 g, 3.10 mmol, 25% yield) was prepared by the procedure as described in Masuda, K, et al, PCT Int Appl, 2010044441, 2010. ¹H NMR (400 MHz, CDCl₃) δ 7.83 (s, 1H), 4.66 (br s, 2H), 2.86 (s, 3H). MS (ESI) m/z 279.1 (M+H, Br isotope peak seen).

Intermediate 1-2: Preparation of methyl 5-amino-4-chloro-2-methylbenzo[d]thiazole-6-carboxylate. To a steel reaction vessel, 1-1 (0.9 g, 3 mmol) was added, followed by TEA (0.5 mL, 3.2 mmol), PdOAc₂ (0.2 g, 0.6 mmol), dppf (0.5 g, 0.9 mmol), DMSO (12 mL) and MeOH (8 mL). The vessel was sealed and pressurized with CO gas at 70 psi and heated to 80° C. for 24 h. The reaction vessel was allowed to cool to rt, and the contents were partitioned with water (20 mL) and EtOAc (50 mL). The aqueous layer was further extracted with EtOAc (2×20 mL). The combined organic layers were washed with brine (15 mL), dried (MgSO₄), filtered and concentrated under reduced pressure to an oil. The residue was purified via normal phase chromatography, eluted with hexanes/EtOAc to afford 1-2 (0.7 g, 3 mmol, 90% yield) as a pale yellow solid. ¹H NMR (400 MHz, CDCl₃) δ 8.35 (s, 1H), 6.34 (br s, 2H), 3.96 (s, 3H), 2.89 (s, 3H). MS (ESI) m/z 257.0 (M+H)⁺.

Intermediate 1-3: 5-amino-4-chloro-N-(4-fluoro-3-(trifluoromethyl)phenyl)-2-methylbenzo[d]thiazole-6-carboxamide. Toluene (8 mL) was added to 4-fluoro-3-(trifluoromethyl)aniline (1.2 g, 7.0 mmol) in a microwave vial and the solution was cooled to 0° C. To this solution was added trimethylaluminum (3.5 mL, 7.0 mmol, 2 M in toluene). After 10 min, a toluene (6 mL) solution of 1-2 (0.6 g, 2.3 mmol) was added followed by additional toluene (32 mL). The reaction mixture was heated under microwave irradiation at 120° C. for 30 min. The cooled reaction mixture was quenched with 1N HCl (10 mL), extracted with EtOAc (3×30 mL). The combined organic portions were washed with brine (15 mL), dried (MgSO₄) and concentrated under reduced pressure. The residue was purified via normal phase chromatography, eluted with hexanes/EtOAc to afford 1-3 (0.8 g, 2 mmol, 90% yield) orange solid. ¹H NMR (400 MHz, CDCl₃) δ 7.94 (br d, J=1.8 Hz, 1H), 7.91 (s, 1H), 7.89 (dd, J=6.2, 2.6 Hz, 1H), 7.84 (dt, J=8.8, 3.5 Hz, 1H), 5.92 (br s, 2H), 2.92-2.89 (m, 3H). MS (ESI) m/z 404.1 (M+H)⁺.

Intermediate 1: In microwave vial was added palladium (II) acetate (56 mg, 0.2 mmol), [1,1′-biphenyl]-2-yldi-tert-butylphosphane (0.15 g, 0.50 mmol), sodium formate (0.2 g, 3 mmol) in degassed (under N₂) MeOH (10 mL). The mixture was degassed for 3 min under N₂ and stirred for 10 min, then 1-2 (0.3 g, 0.6 mmol) in THF (1 mL) was added. The vial was sealed and the reaction mixture was heated under microwave irradiation to 130° C. for 1 h. The solvent was removed under vacuum and the residue was purified via normal phase chromatography, elution with hexanes/EtOAc to afford intermediate 1 (0.14 g, 0.38 mmol, 61% yield) as a tan solid. ¹H NMR (500 MHz, CDCl₃) S 8.07 (br s, 1H), 7.99 (s, 1H), 7.89 (dd, J=6.1, 2.7 Hz, 1H), 7.83 (dt, J=8.9, 3.5 Hz, 1H), 7.33-7.30 (m, 1H), 7.25 (t, J=9.2 Hz, 1H), 5.31 (br s, 2H), 2.84 (s, 3H). MS (ESI) m/z 370.1 (M+H)⁺.

### Intermediate 2: Preparation of 5-(3-hydroxy-3-methylbutyl)-2-methoxybenzoic acid

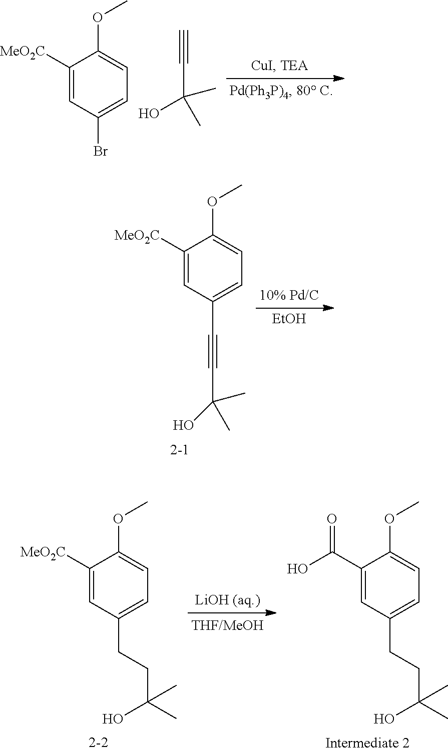

Intermediate 2-1: Methyl 5-(3-hydroxy-3-methylbut-1-yn-1-yl)-2-methoxybenzoate. In large microwave vial was added methyl 5-bromo-2-methoxybenzoate (1.7 g, 6.9 mmol), 2-methylbut-3-yn-2-ol (0.58 g, 6.9 mmol), Pd(Ph₃P)₄ (0.4 g, 0.35 mmol), copper(I) iodide (13 mg, 69 mmol) and TEA (15 mL). The reaction mixture was degassed (vac/N₂), sealed and heated at 80° C. for 18 h. The reaction mixture was partitioned with water (20 mL) and EtOAc (50 mL). The aqueous layer was further extracted with EtOAc (2×20 mL). The combined organic layers were washed with brine (15 mL), dried (MgSO₄) followed by purification via normal phase silica chromatography and eluting with hexanes/EtOAc to afford 2-1 (1.2 g, 4.8 mmol, 70% yield). MS (ESI) m/z: 249 (M+H)⁺.

Intermediate 2-2: Methyl 5-(3-hydroxy-3-methylbutyl)-2-methoxybenzoate. In a Parr bottle, 2-1 (1.2 g, 4.8 mmol) was added followed by EtOH (25 mL) and wet 10% Pd/C (0.2 g). The flask was sealed and hydrogenated at 20 psi for 18 h, the reaction mixture filtered through Celite®, and the filtrate was concentrated in vacuo. The residue was purified by normal phase silica chromatography eluted with hexanes/EtOAc to afford 2-2 (0.7 g, 3 mmol, 60% yield) as a pale yellow oil. ¹H NMR (400 MHz, CDCl₃) δ 7.67 (d, J=2.4 Hz, 1H), 7.33 (dd, J=8.4, 2.4 Hz, 1H), 6.94 (d, J=8.4 Hz, 1H), 3.92 (s, 3H), 3.92 (s, 3H), 2.88-2.59 (m, 2H), 1.86-1.69 (m, 2H), 1.32 (s, 6H), 1.25 (s, 1H).

Intermediate 2: To 2-2 (0.7 g, 3 mmol) in THF (21 ml), water (7 ml) was added followed by aq. LiOH (2M, 1.4 ml, 2.8 mmol). The reaction mixture was stirred at rt for 14 h. An additional equivalent of LiOH soln. was added and the reaction mixture stirred for 4 h. The reaction mixture was acidified with HCl (0.1 N) and extracted with EtOAc (2×25 mL). The combined organic portions were washed with brine (25 mL), dried (MgSO₄), and concentrated under reduced pressure to afford intermediate 2 (0.6 g, 3 mmol, 90% yield) which was used without further purification. MS (ESI) m/z: 239.2 (M+H)⁺.

### Intermediate 3-6 and 3-7: Preparation of 2-methoxy-5-(3a,4,6,6a-tetrahydrofuro[3,4-d]isoxazol-3-yl)benzoic acid (Homochiral analog)

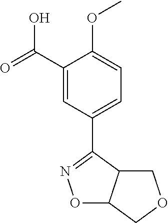 [SMILES: COc1ccc(C2=NOC3COCC23)cc1C(=O)O]

### Intermediate 3-6 & 3-7

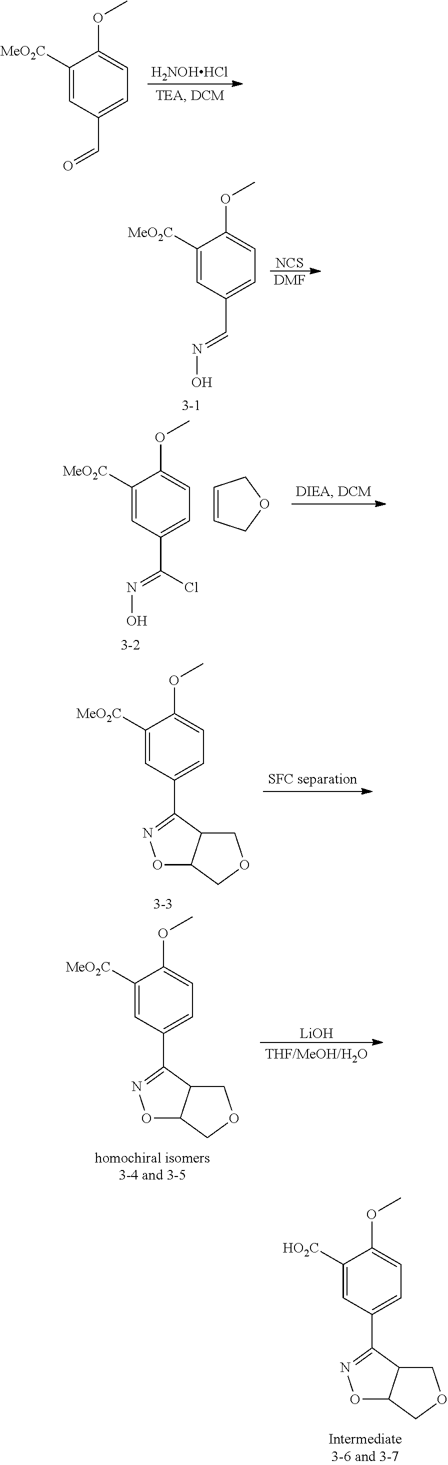

Intermediate 3-1: Preparation of methyl (E)-5-((hydroxyimino)methyl)-2-methoxybenzoate. Commercially available methyl 5-formyl-2-methoxybenzoate (1.16 g, 6 mmol) was dissolved in DCM (5 mL), and to this solution was added HONH₂·HCl (415 mg, 6 mmol) followed by TEA (1 mL) and the reaction mixture was stirred at rt for 14 h. The reaction mixture was quenched with water (100 mL) and extracted with EtOAc (2×25 mL). The combined organic portions were dried (MgSO₄) and concentrated under reduced pressure to afford 3-1 as a white solid (1.2 g, 5.7 mmol, 95% yield). ¹H NMR (400 MHz, CDCl₃) δ 8.13 (s, 1H), 8.03 (d, J=2.4 Hz, 1H), 7.78-7.67 (m, 1H), 7.03 (d, J=8.8 Hz, 1H), 3.97 (s, 3H), 3.93 (s, 3H). MS (ESI) m z: =210.1 (M+H)⁺.

Intermediate 3-2: Preparation of methyl (Z)-5-(chloro(hydroxyimino)methyl)-2-methoxybenzoate. Intermediate 3-1 (23 g, 0.10 mol) was dissolved in DMF (100 mL), and to this solution was added NCS (15 g, 0.10 mol) and the reaction mixture was stirred at rt for 24 h. The reaction mixture was quenched with water (300 mL) and solid formed was collected by filtration, dried under vacuum to afford 3-2 (23 g, 95 mmol, 86% yield) as a pale yellow solid. ¹H NMR (500 MHz, CDCl₃) δ 8.32-8.30 (m, 1H), 7.99-7.96 (m, 1H), 7.80-7.78 (m, 1H), 7.05-7.02 (d, 1H), 3.98 (s, 3H), 3.94 (s, 3H). MS (ESI) m/z: =244.1 (M+H)⁺.

Intermediate 3-3: Preparation of methyl 2-methoxy-5-(3a,4,6,6a-tetrahydrofuro[3,4-d]isoxazol-3-yl)benzoate. To intermediate 3-2 (0.4 g, 2 mmol) and 2,5-dihydrofuran (1.2 g, 17 mmol) in DCM (10 mL) was added TEA (0.7 mL, 5 mmol) and the reaction mixture was stirred 24 h. The solvents were removed under vacuum and the residue was directly purified by normal phase silica gel chromatography, eluting with hexanes/EtOAc, to afford 3-3 (0.4 g, 80% yield). ¹H NMR (500 MHz, CDCl₃) δ 7.98 (d, J=2.3 Hz, 1H), 7.86 (dd, J=8.8, 2.4 Hz, 1H), 7.04 (d, J=8.7 Hz, 1H), 5.38 (dd, J=9.2, 3.9 Hz, 1H), 4.34-4.26 (m, 2H), 4.20-4.09 (m, 1H), 3.96 (s, 3H), 3.91 (s, 3H), 3.83-3.76 (m, 1H), 2.92-2.70 (m, 1H). MS (ESI) m/z=278.3 (M+H)⁺.

Intermediate 3-4 and intermediate 3-5: The following chiral intermediates were separated by chiral SFC by the following preparative chromatographic methods from racemate 3-3: Instrument: PIC Solution SFC Column: Chiralpak IA, 30×250 mm, 5 micron, Mobile Phase: 20% MeOH/80% CO₂, Flow Conditions: 90 mL/min, 150 Bar, 40° C., Detector Wavelength: 220 nm; Analytical method: Intrument: Shimadzu Nexera SFC, Column Chiralpak IA 4.6×100 mm, 5 micron. Mobile Phase: 25% MeOH/75% CO₂. Flow Conditions: 2 mL/min, 150 Bar, 40° C., Detector Wavelength: 220 nm afford homochiral intermediate 3-4 (Peak-1, 28% yield, RT=1.83 min., >99% ee) and homochiral intermediate 3-5 (Peak-2, 28% yield, RT=3.83 min., >99% ee).

Intermediate 3-6: Preparation of 2-methoxy-5-(3a,4,6,6a-tetrahydrofuro[3,4-d]isoxazol-3-yl)benzoic acid. To a solution of intermediate 3-4 (75 mg, 0.3 mmol) in THF (3 mL) was added MeOH (0.6 mL) followed by LiOH (2M, 0.4 mL, 0.8 mmol). After 4 h, the reaction mixture was diluted with water and acidified with HCl (1 M) and extracted with EtOAc (2×25 mL). The combined organic portions were dried (MgSO₄) and concentrated under reduced pressure to afford intermediate 3-6 (71 mg, 100% yield). ¹H NMR (400 MHz, CD₃OD) δ 7.78 (d, J=2.3 Hz, 1H), 7.71 (dd, J=8.7, 2.3 Hz, 1H), 7.09 (d, J=8.8 Hz, 1H), 5.33 (dd, J=9.2, 3.6 Hz, 1H), 4.44 (ddd, J=8.9, 7.2, 1.3 Hz, 1H), 4.18 (d, J=10.6 Hz, 1H), 4.08 (dd, J=9.4, 1.0 Hz, 1H), 3.91-3.88 (m, 3H), 3.86 (dd, J=9.4, 6.9 Hz, 1H), 3.73 (dd, J=10.7, 3.7 Hz, 1H). MS (ESI) m/z: =264.1 (M+H)⁺.

Intermediate 3-7: Preparation of 2-methoxy-5-(3a,4,6,6a-tetrahydrofuro[3,4-d]isoxazol-3-yl)benzoic acid. Intermediate 3-7 (52 mg, 0.20 mmol, 100% yield) was prepared in a similar manner as intermediate 3-6, substituting intermediate 3-5 for intermediate 3-4. ¹H NMR (500 MHz, CD₃OD) δ 8.10 (d, J=2.4 Hz, 1H), 7.87 (dd, J=8.8, 2.4 Hz, 1H), 7.23 (d, J=8.7 Hz, 1H), 5.38 (dd, J=9.2, 3.7 Hz, 1H), 4.47 (ddd, J=8.9, 7.2, 1.2 Hz, 1H), 4.22 (d, J=10.7 Hz, 1H), 4.12 (q, J=7.1 Hz, 1H), 4.07 (dd, J=9.5, 0.9 Hz, 1H), 3.97 (s, 3H), 3.88 (dd, J=9.5, 6.9 Hz, 1H), 3.76 (dd, J=10.8, 3.6 Hz, 1H). MS (ESI) m/z: =264.1 (M+H)⁺

### Intermediate 4: Preparation of 5-(5-(hydroxymethyl)-3a,5,6,6a-tetrahydro-4H-cyclopenta[d]-isoxazol-3-yl)-2-methoxybenzoic acid

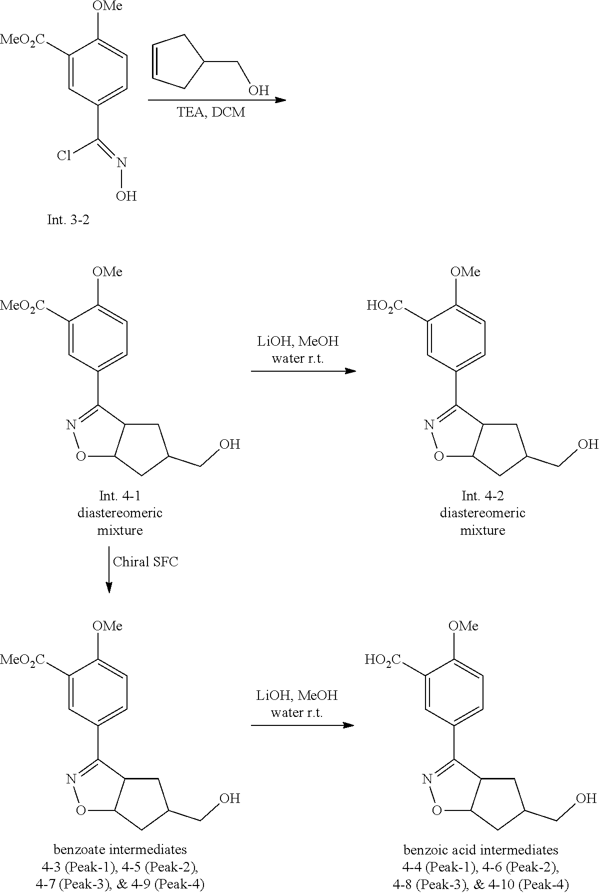

Intermediate 4-1: Preparation of methyl 5-(5-(hydroxymethyl)-3a,5,6,6a-tetrahydro-4H-cyclopenta[d]isoxazol-3-yl)-2-methoxybenzoate. Intermediate 3-2 (3.0 g, 12 mmol) was dissolved in DCM (123 mL) and to this was added cyclopent-3-en-1-ylmethanol (4.8 g, 49 mmol) followed by the addition of TEA (5.2 mL, 37 mmol) and the reaction mixture was stirred at rt. After stirring 14 h, the reaction mixture was concentrated under reduced pressure and the residue purified by normal phase chromatography by eluting with hexanes/EtOAc gave 4-1 (2.8 g, 9.2 mmol, 75% yield) as an oily mass. LC-MS RT=0.95 min; MS (ESI) m/z=306.3 (M+H)⁺. [Method A]

Diasteromeric intermediate 4-2: Preparation of 5-(5-(hydroxymethyl)-3a,5,6,6a-tetrahydro-4H-cyclopenta[d]isoxazol-3-yl)-2-methoxybenzoic acid. Intermediate 4-1 (88 mg, 0.29 mmol) was dissolved in THF (1 mL)/MeOH (1 mL) and treated with LiOH monohydrate (36 mg, 0.86 mmol) in H₂O (I mL) at rt. After 3 h, the reaction mixture was diluted with H₂O (5 mL) and the pH of the aq. layer was adjusted to pH 7 with 1M HCl solution and extracted with EtOAc (2×25 mL). The combined organic portions were washed with brine, dried (Na₂SO₄), filtered, and concentrated under reduced pressure to give 4-2 (62 mg, 74% yield). Intermediate 4-2 was used without further purification. LC-MS RT=0.85 min; MS (ESI) m/z=292.3 (M+H)⁺. [Method A]Intermediates 4-3 through 4-10

Intermediates 4-3, 4-5, 4-7, and 4-9 were obtained by chiral SFC separation of the diastereomeric mixture intermediate 4-1 (525 mg, 1.72 mmol). Chiral SFC Preparative chromatographic conditions: Instrument: Berger MG 11 (SFC); Column: Chiralpak AD-H, 21×250 mm, 5 micron; Mobile phase: 15% MeOH/85% CO₂; Flow conditions: 45 mL/min, 150 Bar, 40° C.: Detector wavelength: 210 nm; Injections details: 0.5 mL of ˜35 mg/mL in MeOH. Analytical chromatographic conditions: Instrument: Shimadzu Nexera SFC: Column: Chiralpak AD-H, 4.6×100 mm, 3 micron: Mobile phase: 15% MeOH/85% CO₂; Flow conditions: 2.0 mL/min, 150 Bar, 40° C.; Detector wavelength: 220 nm: Injection details: 5 μL of ˜1 mg/mL in MeOH.

Intermediate 4-3 (Peak-1, RT=4.07 min; >99% ee) was obtained as a film (150 mg, 29% yield). ¹H NMR (600 MHz, CDCl₃) δ 8.04 (d, J=2.3 Hz, 1H), 7.87 (dd, J=8.7, 2.3 Hz, 1H), 7.01 (d, J=8.8 Hz, 1H), 5.23 (dd, J=8.8, 5.1 Hz, 1H), 4.10 (t, J=8.7 Hz, 1H), 3.94 (s, 3H), 3.90 (s, 3H), 3.72-3.66 (m, 1H), 3.61 (dt, J=10.5, 5.2 Hz, 1H), 2.30-2.16 (m, 2H), 2.05 (dd, J=13.0, 6.1 Hz, 1H), 1.76 (ddd, J=12.9, 11.5, 9.4 Hz, 1H), 1.68-1.62 (m, 1H), 1.39 (br, J=4.8 Hz, 1H).

Intermediate 4-4: Preparation of 5-(5-(hydroxymethyl)-3a,5,6,6a-tetrahydro-4H-cyclopenta[d]isoxazol-3-yl)-2-methoxybenzoic acid. Intermediate 4-4 (100 mg, 78% yield) was prepared in a similar manner as intermediate 4-2 with the hydrolysis of intermediate 4-3. LC-MS RT=0.85 min; MS (ESI) m/z=292.3 (M+H)⁺. [Method A]Intermediate 4-5 (Peak-2, RT=4.55 min: >99% ee) was obtained as a film (33 mg, 6.3% yield). ¹H NMR (600 MHz, CDCl₃) δ 8.05 (d, J=2.3 Hz, 1H), 7.87 (dd, J=8.8, 2.3 Hz, 1H), 7.02 (d, J=8.8 Hz, 1H), 5.25 (ddd, J=10.1, 6.2, 4.2 Hz, 1H), 4.04-3.98 (m, 1H), 3.94 (s, 3H), 3.90 (s, 3H), 3.63-3.57 (m, 1H), 3.56-3.50 (m, 1H), 2.38-2.26 (m, 3H), 1.92-1.85 (m, 1H), 1.73-1.66 (m, 1H), 1.51 (t, J=5.3 Hz, 1H).

Intermediate 4-6: Preparation of 5-(5-(hydroxymethyl)-3a,5,6,6a-tetrahydro-4H-cyclopenta[d]isoxazol-3-yl)-2-methoxybenzoic acid. Intermediate 4-6 (20.2 mg, 92% yield) was prepared in a similar manner as intermediate 4-2 with the hydrolysis of intermediate 4-5. LC-MS RT=0.83 min; MS (ESI) m/z: 292.3 (M+H)⁺. [Method A]Intermediate 4-7 (Peak-3, RT=5.66 min: >99% ee) was obtained as a film (161 mg, 30.6% yield). ¹H NMR: (600 MHz, CDCl₃) δ 8.05-8.03 (m, 1H), 7.86 (dd, J=8.7, 2.3 Hz, 1H), 7.01 (d, J=8.8 Hz, 1H), 5.23 (dd, J=8.7, 5.2 Hz, 1H), 4.10 (t, J=8.7 Hz, 1H), 3.94 (s, 3H), 3.90 (s, 3H), 3.69 (br dd, J=10.6, 5.2 Hz, 1H), 3.63-3.58 (m, 1H), 2.28-2.17 (m, 2H), 2.05 (br dd, J=12.9, 6.2 Hz, 1H), 1.76 (ddd, J=13.0, 11.5, 9.4 Hz, 1H), 1.64-1.60 (m, 1H), 1.49 (br s, 1H).

Intermediate 4-8: Preparation of 5-(5-(hydroxymethyl)-3a,5,6,6a-tetrahydro-4H-cyclopenta[d]isoxazol-3-yl)-2-methoxybenzoic acid. Intermediate 4-8 (120 mg, 85% yield) was prepared in a similar manner as intermediate 4-2 with the hydrolysis of intermediate 4-7. LC-MS RT=0.83 min; MS (ESI) m/z: 292.3 (M+H)⁺. [Method A]Intermediate 4-9 (Peak-4, RT=9.81 min; >99% cc) was obtained as a film (47 mg, 9.0% yield). ¹H NMR: (600 MHz, CDCl₃) δ 8.04 (d, J=2.3 Hz, 1H), 7.87 (dd, J=8.7, 2.3 Hz, 1H), 7.02 (d, J=8.8 Hz, 1H), 5.24 (ddd, J=10.1, 6.2, 4.2 Hz, 1H), 4.03-3.98 (m, 1H), 3.94 (s, 3H), 3.90 (s, 3H), 3.63-3.57 (m, 11H), 3.56-3.49 (m, 1H), 2.38-2.25 (m, 3H), 1.91-1.85 (m, 1H), 1.72-1.66 (m, 1H), 1.55 (br s, 1H).

Intermediate 4-10. Preparation of 5-(5-(hydroxymethyl)-3a,5,6,6a-tetrahydro-4H-cyclopenta[d]isoxazol-3-yl)-2-methoxybenzoic acid. Intermediate 4-10 (18 mg, 52% yield) was prepared in a similar manner as intermediate 4-2 with the hydrolysis of intermediate 4-9. LC-MS RT=0.84 min; MS (ESI) m/z: 292.3 (M+H)⁺. [Method A]Intermediate 5: Preparation of 5-(5-(2-hydroxyacetyl)-3a,5,6,6a-tetrahydro-4H-pyrrolo[3,4-d]isoxazol-3-yl)-2-methoxybenzoic acid

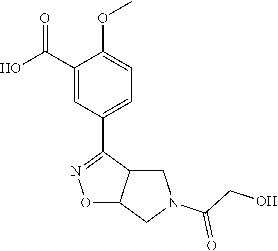 [SMILES: COc1ccc(C2=NOC3CN(C(=O)CO)CC23)cc1C(=O)O]

### Intermediate 5

### Homochiral

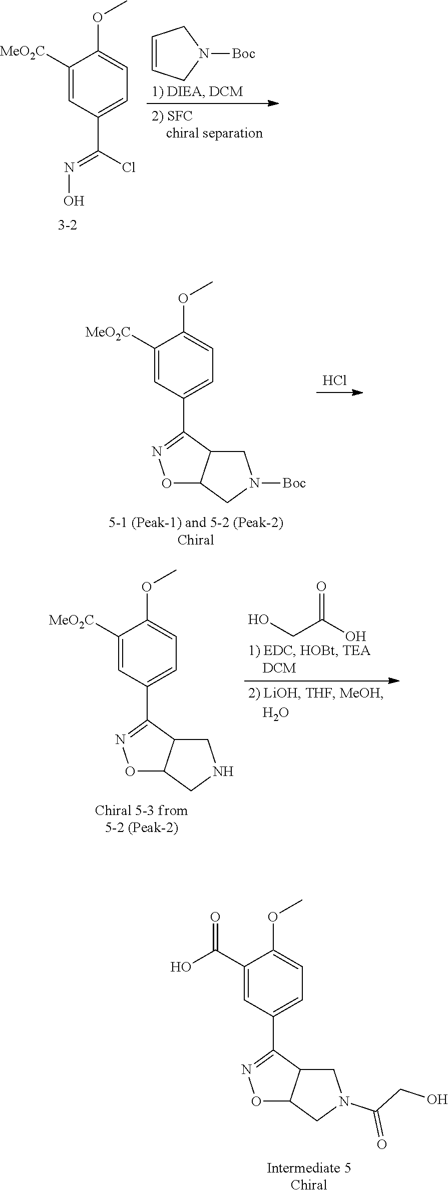

Intermediate 5-1: Preparation of methyl 2-methoxy-5-(3a,5,6,6a-tetrahydro-4H-pyrrolo[3,4-d]isoxazol-3-yl)benzoate. Intermediates 5-1 and 5-2 were prepared and isolated in a similar manner as that described for intermediate 3-3 by substituting tert-butyl 2,5-dihydro-1H-pyrrole-1-carboxylate for 2,5-dihydrofuran in the cycloaddition step, followed by SFC separation of the resulting diastereomer to the isomers by the following preparative chromatographic methods: Instrument: Jasco SFC Prep Column; Regis Whelk 21×250 mm ID, 5 μm. Temperature: 40° C., Flow rate: 45 mL/min, 150 Bar, Mobile Phase: gradient 80/20 CO₂/MeOH, Detector Wavelength: 220 nm, Injection Volume: 0.5 mL to afford intermediate 5-1 (peak 1, 0.36 g, 0.95 mmol, 23% yield, >99% ee, Analytical RT=8.80 min), intermediate 5-2 (peak-2, 0.36 g, 0.95 mmol, 23% yield, >99% ee, Analytical RT=9.86 min). Analytical Chromatographic Conditions: Instrument: Shimadzu Nexera SFC, Column: Regis Whelk-01, 4.6×100 mm ID, 3 μm, Temperature: 40° C., Flow rate: 2.0 mL/min, 150 Bar, Mobile Phase: gradient 80/20 CO₂/MeOH.

Intermediate 5-3: To intermediate 5-2 (Peak-2) (0.36 g, 0.96 mmol) in THF (10 mL) was added 4N HCl in dioxane (2.4 mL) and the reaction mixture was stirred for 14 h. The solvents were removed under reduced pressure to afford 5-3 (0.3 g, 0.96 mmol, 100% yield). ¹H NMR (500 MHz, DMSO-d) δ 7.95 (d, J=2.4 Hz, 1H), 7.85 (dd, J=8.8, 2.4 Hz, 1H), 7.26 (d, J=8.9 Hz, 1H), 5.43 (dd, J=9.3, 4.7 Hz, 1H), 4.67 (td, J=9.0, 1.7 Hz, 1H), 3.88 (s, 3H), 3.80 (s, 3H), 3.63 (br d, J=13.3 Hz, 1H), 3.54-3.42 (m, 2H), 3.39-3.33 (m, 2H). MS (ESI) m/z 277.1 (M+H)⁺.

Intermediate 5: Preparation of 5-(5-(2-hydroxyacetyl)-3a,5,6,6a-tetrahydro-4H-pyrrolo[3,4-d]isoxazol-3-yl)-2-methoxybenzoic acid. To intermediate 5-3 (78 mg, 0.30 mmol), was added glycolic acid (57 mg, 0.75 mmol), 1-HOBt (51 mg, 0.30 mmol), EDC (57 mg, 0.30 mmol) in DCM (5 ml) followed by TEA (70 μl, 0.5 mmol). After 1h, the reaction was partitioned with HCl (1N, 5 mL) and EtOAc (20 mL). The aqueous layer was extracted with EtOAc (2×20 mL). The combined organic layers were washed with brine (15 mL) and dried (MgSO₄) and concentrated under reduced pressure. The residue was dissolved in a solution of THF (2 mL)/MeOH (0.5 mL), cooled to 0° C., followed by the addition of aq. LiOH (2M, 0.4 ml, 0.7 mmol). The solvents were removed in vacuo, the residue was freeze-dried to afford intermediate 5 (80 mg, 0.25 mmol, 100% yield), which was used in next step without purification. MS (ESI) m/z 321.1 (M+H)˜.

### Intermediate 6: Preparation of 5-(5,5-dioxido-3a,4,6,6a-tetrahydrothieno[3,4-d]isoxazol-3-yl)-2-methoxybenzoic acid

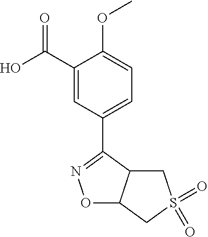 [SMILES: COc1ccc(C2=NOC3CS(=O)(=O)CC23)cc1C(=O)O]

### Intermediate 6

### Chiral

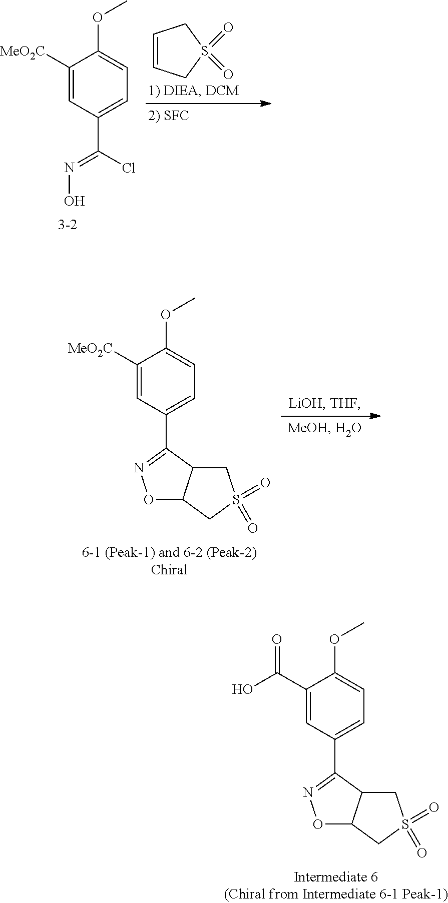

Intermediates 6-1 and 6-2: Preparation of methyl 5-(5,5-dioxido-3a,4,6,6a-tetrahydrothieno[3,4-d]isoxazol-3-yl)-2-methoxybenzoate. Intermediates 6-1 and 6-2 were prepared in a similar manner as that described for intermediate 3-3 by substituting 2,5-dihydrothiophene 1,1-dioxide for 2,5-dihydrofuran in the cycloaddition step followed by chiral SFC separation of the diastereomers by the following preparative chromatographic methods: Instrument: PIC Solution SFC Prep 200 Column: Chiralcel OD-H 21×250 mm ID, 5 μm, Temperature: 40° C., Flow rate: 45 mL/min, 150 Bar, Mobile Phase: gradient 75/25 CO₂/MeOH, Detector Wavelength: 271 nm, Injection Volume: 1 mL to afford Intermediate 6-1 (Peak 1, 0.1 g, 0.33 mmol, 18% yield, >99% ee, Analytical RT=3.79 min), intermediate 6-2 (Peak-2, 0.1 g, 0.33 mmol, 18% yield, >99% ee, Analytical RT=5.67 min). Analytical Chromatographic Conditions: Instrument: Shimadzu Nexera SFC, Column: Chiralcel OD-H, 4.6×100 mm ID, 3 μm, Temperature: 40° C., Flow rate: 2.0 mL/min, 150 Bar, Mobile Phase: gradient 85/15 CO₂/MeOH. Detector Wavelength: 220 nm. Intermediate 6-1: ¹H NMR (400 MHz, CDCl₃) δ 7.96 (d, J=2.4 Hz, 1H), 7.85 (dd, J=8.8, 2.2 Hz, 1H), 7.07 (d, J=9.0 Hz, 1H), 5.43 (ddd, J=10.1, 7.2, 4.1 Hz, 1H), 4.52-4.44 (m, 1H), 3.97 (s, 3H), 3.92 (s, 3H), 3.66-3.47 (m, 3H), 3.14 (dd, J=13.6, 8.1 Hz, 1H). Intermediate 6-2: ¹H NMR (400 MHz, CDCl₃) δ 7.96 (d, J=2.4 Hz, 1H), 7.85 (dd, J=8.8, 2.2 Hz, 1H), 7.06 (d, J=8.8 Hz, 1H), 5.42 (ddd, J=10.1, 7.2, 4.1 Hz, 1H), 4.52-4.44 (m, 1H), 3.97 (s, 3H), 3.91 (s, 3H), 3.66-3.46 (m, 3H), 3.14 (dd, J=13.6, 8.4 Hz, 1H).

Intermediate 6: Preparation of 5-(5,5-dioxido-3a,4,6,6a-tetrahydrothieno[3,4-d]isoxazol-3-yl)-2-methoxybenzoic acid. Intermediate 6 (82 mg, 0.26 mmol, 79% yield) was prepared from the intermediate 6-1 (Peak-1) in a similar procedure as that described for intermediate 3-6. MS (ESI) m/z 312.3 (M+H)⁺.

### Intermediate 7: Preparation of 5-((4S,6R)-4,6-bis((tert-butyldimethylsilyl)oxy)-3a,5,6,6a-tetrahydro-4H-cyclopenta[d]isoxazol-3-yl)-2-methoxybenzoic acid

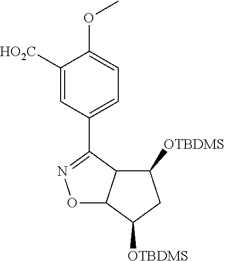 [SMILES: COc1ccc(C2=NOC3C2[C@@H](O[Si](C)(C)C(C)(C)C)C[C@H]3O[Si](C)(C)C(C)(C)C)cc1C(=O)O]

### Intermediate 7

### Chiral

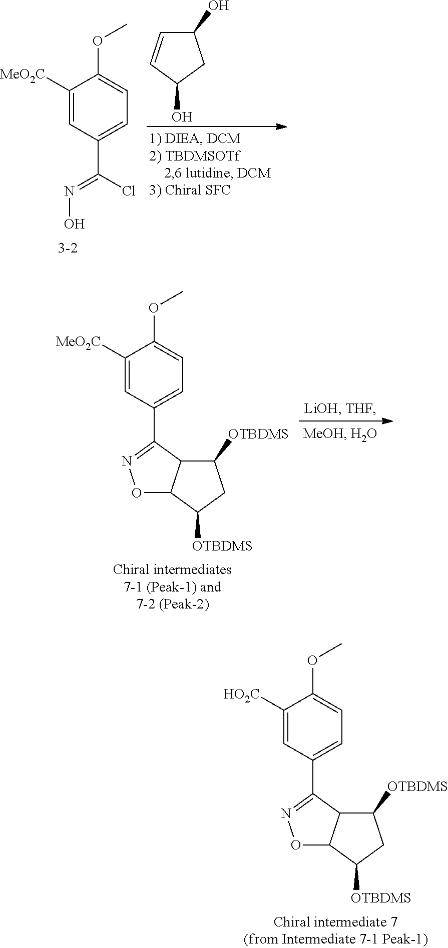

Intermediate 7-1: Preparation of methyl 5-((4S,6R)-4,6-bis((tert-butyldimethylsilyl)oxy)-3a,5,6,6a-tetrahydro-4H-cyclopenta[d]isoxazol-3-yl)-2-methoxybenzoate. To intermediate 3-2 (1.2 g, 5.0 mmol) in DCM (10 mL) was added (1R,3S)-cyclopent-4-ene-1,3-diol (0.5 g, 5 mmol), followed by TEA (0.5 g, 5 mmol) and the reaction mixture was stirred at rt for 24 h. The reaction mixture was quenched with water (50 mL) and extracted with EtOAc (2×25 mL). The combined organic portions were dried (MgSO₄) and concentrated under reduced pressure. Purification by normal phase silica using hexanes/EtOAc as eluents afforded the cycloadduct diol which was re-dissolved in DCM (10 mL), followed by the addition of tert-butyldimethylsilyl trifluoromethanesulfonate (2.6 g, 10 mmol) followed by 2,6-lutidine (1.6 g, 15 mmol). After 24 h, the reaction mixture was quenched with water (50 mL) and extracted with EtOAc (2×25 mL). The combined organic portions were dried (MgSO₄), concentrated under reduced pressure and the residuepurificatied via normal phase silica using hexanes/EtOAc as eluents to afford the a residue which was subjected to chiral SFC separation by the following preparative chromatographic methods: Instrument: PIC Solution SFC Prep 200 Column: Chiralcel OD-H 30×250 mm ID, 5 μm, Temperature: 40° C., Flow rate: 90 mL/min, 150 Bar. Mobile Phase: gradient 80/20 CO₂/IPA, Detector Wavelength: 270 nm, Injection Volume: 0.5 mL to afford intermediate 7-1 (peak-1, >99% cc, Analytical RT=4.69 min, 344 mg, 13% yield), intermediate 7-2 (peak-2, >99% ee, Analytical RT=6.32 min, 364 mg, 14% yield). Analytical Chromatographic Conditions: Instrument: Shimadzu Nexera SFC, Column: Chiralpak IC, 4.6×100 mm 1D, 3 μm, Temperature: 40° C., Flow rate: 2.0 mL/min, 150 Bar, Mobile Phase: gradient 90/10 CO₂/IPA. Detector Wavelength: 220 nm. Intermediate 7-1: ¹H NMR (600 MHz, CDCl₃) δ 8.16 (d, J=2.3 Hz, 1H), 7.89 (dd, J=8.8, 2.3 Hz, 1H), 6.98 (d, J=8.8 Hz, 1H), 5.01 (dd, J=10.4, 2.8 Hz, 1H), 4.29 (td, J=6.0, 2.8 Hz, 1H), 4.19 (td, J=5.7, 4.0 Hz, 1H), 4.06 (dd, J=10.3, 3.7 Hz, 1H), 3.95 (s, 3H), 3.89 (s, 3H), 2.06 (dt, J=13.2, 5.8 Hz, 1H), 1.80 (dt, J=12.9, 6.2 Hz, 1H), 0.93 (s, 9H), 0.91 (s, 9H), 0.14 (s, 3H), 0.12 (s, 3H), 0.02 (s, 3H), −0.05 (s, 3H). MS (ESI) m/z: 536.6 (M+H)⁺. Intermediate 7-2: ¹H NMR (600 MHz, CDCl₃) δ 8.16 (d, J=2.3 Hz, 1H), 7.89 (dd, J=8.8, 2.3 Hz, 1H), 6.98 (d, J=8.8 Hz, 1H), 5.01 (dd, J=10.4, 2.8 Hz, 1H), 4.29 (td, J=6.0, 2.8 Hz, 1H), 4.19 (td, J=5.7, 4.0 Hz, 1H), 4.06 (dd, J=10.3, 3.7 Hz, 1H), 3.95 (s, 3H), 3.89 (s, 3H), 2.06 (dt, J=13.2, 5.8 Hz, 1H), 1.80 (dt, J=12.9, 6.2 Hz, 1H), 0.93 (s, 9H), 0.91 (s, 9H), 0.14 (s, 3H), 0.12 (s, 3H), 0.02 (s, 3H), −0.05 (s, 3H). MS (ESI) m/z: 536.6 (M+H)˜.

Intermediate 7: To intermediate 7-1 (30 mg, 56 μmol) in THF (1 mL) was added MeOH (1 mL), H₂O (0.5 mL) followed by LiOH (4 mg, 0.17 mmol). After 4 h, the reaction mixture was diluted with water acidified with HCl (IN) and extracted with EtOAc (2×25 mL). The organic portions were combined and dried (MgSO₄) and concentrated under reduced pressure to afford intermediate 7 (18 mg, 34 μmol, 62% yield). ¹H NMR (500 MHz, CDCl₃) δ 8.48-8.46 (m, 1H), 8.04-8.00 (m, 1H), 7.10-7.05 (m, 1H), 5.06-5.02 (m, 1H), 4.33-4.29 (m, 1H), 4.23-4.18 (s, 3H), 4.15-4.13 (m, 1H), 4.12-4.10 (m, 1H), 4.00-3.94 (m, 1H), 1.29-1.24 (m, 1H), 0.93 (s, 18H), 0.12 (s, 3H), 0.12-0.03 (m, 3H), 0.03 (s, 1H), 0.02 (s, 3H), −0.05-0.06 (m, 3H). MS (ESI) m/z: 522.5 (M+H)⁺.

Intermediates 8-2 (chiral peak 1), 8-3 (chiral peak 2), 8-4 (chiral peak 3), 8-5 (chiral peak 4): 5-(5-hydroxy-3a,5,6,6a-tetrahydro-4H-cyclopenta[d]isoxazol-3-yl)-2-methoxybenzoic acid. The titled compounds were prepared following the scheme outlined below:

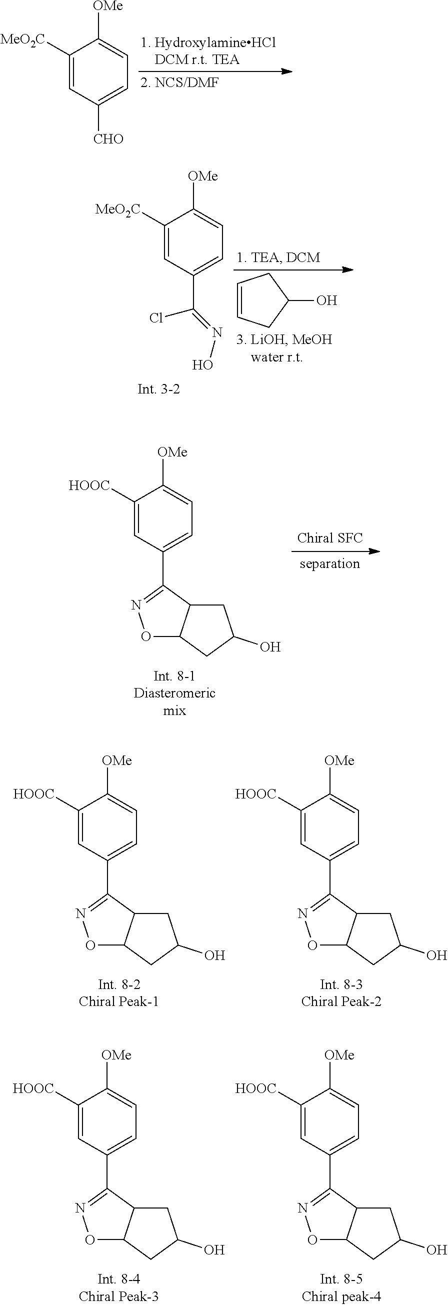

Intermediate 8-1. Preparation of 5-(5-hydroxy-3a,5,6,6a-tetrahydro-4H-cyclopenta[d]isoxazol-3-yl)-2-methoxybenzoic acid. Methyl 5-formyl-2-methoxybenzoate (24.9 g, 128 mmol) was dissolved in DCM (500 mL). To the solution was added triethyamine (17.9 mL, 128 mmol) followed by hydroxylamine hydrochloride (8.91 g, 128 mmol). The reaction mixture was stirred for 14 h at rt and concentrated under reduced pressure to yield a white solid. The solid was dissolved in water (200 mL) and the aqueous portion extracted with EtOAc (2×100 mL). The combined organic portion was, dried (MgSO₄), filtered and concentrated under reduced pressure to yield a white solid (27.1 g, 100% yield). The solid was re-dissolved in DMF (200 mL) and to the solution was added NCS (17.2 g, 128 mmol) and stirred at rt for 14 h. The reaction mixture was quenched by the addition of excess water and a white solid precipitated. The solid was separated by filtration and washed with excess water and dried under vacuum to afford a white solid (287 g, 89% yield) for intermediate 8-1. ¹H NMR (500 MHz, CDCl₃) δ 8.32-8.30 (m, 1H), 7.99-7.96 (m, 1H), 7.80-7.78 (m, 1H), 7.05-7.02 (d, 1H), 3.98 (s, 3H), 3.94 (s, 3H).

Intermediate 8-1 (diastereomeric mixture): Alternatively, (F)-5-((hydroxyimino)methyl)-2-methoxybenzoic acid (620 mg, 3.18 mmol) was dissolved in DMF (5 mL), to the solution was added NCS (424 mg, 3.18 mmol) and the reaction mixture was stirred at rt for 4 h. The reaction mixture was quenched with the addition of water (100 mL) and the solution extracted with EtOAc (2×25 mL), dried (MgSO₄) and concentrated under reduced pressure to an oil. The oil obtained was re-dissolved in DCM (10 mL) and cyclopent-3-ene-1-ol (2.67 g, 31.8 mmol) was added, followed by the addition of TEA (0.44 mL) and the reaction mixture stirred at it for 14h The resulting solution was filtered through a plug of silica gel and concentrated under reduced pressure to afford the diastereomeric mixture of intermediate 8-1 (227 mg, 26% yield). ¹H NMR (600 MHz, CDCl₃) δ 8.04 (d, J=2.3 Hz, 1H), 7.85 (dd, J=8.8, 2.3 Hz, 1H), 7.03 (d, J=8.8 Hz, 1H), 5.30 (ddd, J=9.4, 6.2, 2.9 Hz, 1H), 4.50 (quin, J=5.9 Hz, 1H), 4.19 (td, J=9.3, 4.7 Hz, 1H), 3.92 (s, 3H), 2.33-2.27 (m, 1H), 2.18-2.06 (m, 3H). LC-MS RT=0.83 min; MS (ESI) m/z: 278.1 (M+H)⁺. [Method A]

The chiral intermediates of 8-1 were separated by chiral SFC by the following preparative chromatographic methods: Instrument: Berger SFC; Column: IC 25×3 cm ID, 5 μm, Temperature: 40° C., Flow rate: 85 mL/min, Mobile Phase: gradient 75/25 CO₂/MeOH for 12 min then to 45% MeOH, Detector Wavelength: 235 nm, Injection Volume: 1000 μL to afford chiral 8-2 Peak-1, >99% cc, Analytical RT=8.80 min), chiral 8-3 (Peak-2, >95% cc, Analytical RT=9.86 min), chiral 8-4 (Peak-3, >99% cc, Analytical RT=13.53 min), chiral 8-5 (Peak-4, >99% ee, Analytical RT=16.67 min). Analytical Chromatographic Conditions: Instrument: Agilent SFC, Column: IC 250×4.6 mm ID, 5 μm, Temperature: Ambient, Flow rate: 2 mL/min, Mobile Phase: gradient 75/25 CO₂/MeOH 12 min then to 45% MeOH. Analytical data for peak-1-4: ¹H NMR (600 MHz, CD₃OD) δ 8.07 (d, J=2.2 Hz, 1H), 7.82 (dd, J=8.7, 2.1 Hz, 1H), 7.18 (d, J=8.8 Hz, 1H), 5.21 (ddd, J=9.2, 6.2, 2.5 Hz, 1H), 4.27 (m, 1H), 4.24 (td, J=9.4, 4.0 Hz, 1H), 3.94 (s, 3H), 2.16 (m, 1H), 2.05 (m, 1H), 2.00 (m, 1H), 1.99 (m, 1H), 13C NMR (151 MHz, CD₃OD) δ 169.5, 161.6, 160.0, 133.2, 131.4, 122.6 (2C), 113.9, 87.3, 72.7, 56.8, 51.5, 44.1, 40.3.

### Intermediate 9: Preparation of 5-(5-(((di-tert-butoxyphosphaneyl)oxy)methyl)-3a,5,6,6a-tetrahydro-4H-cyclopenta[d]isoxazol-3-yl)-2-methoxybenzoic acid

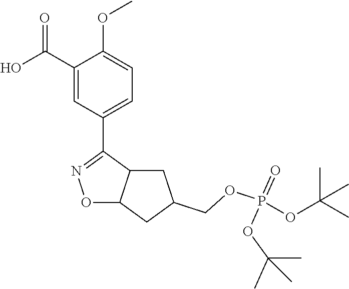 [SMILES: COc1ccc(C2=NOC3CC(COP(=O)(OC(C)(C)C)OC(C)(C)C)CC23)cc1C(=O)O]

### Intermediate 9

To intermediate 4 (50 mg, 0.16 mmol) in ACN (1.4 mL) and DCM (1 mL) was added 1H-tetrazole (23 mg, 0.32 mmol), followed by the dropwise addition of di-tert-butyl N,N-diisopropyl phosphoramidite (0.1 mL, 30 mmol). The reaction mixture was cooled to 0° C., and 2-hydroperoxy-2-methylpropane (47 μL, 0.32 mmol) was added and the reaction stirred at rt for 1.5 h. The reaction was concentrated under reduced pressure and EtOAc (20 mL) was added and washed with potassium phosphate dibasic solution (1.0 M, 20 mL). The organic layer was extracted with EtOAc (2×25 mL) and the combined organic layers were washed with H₂O (25 mL), brine (25 mL), dried (Na₂SO₄), and concentrated under reduced pressure. The residue was dissolved in a solution of THF/MeOH (1:1, 4 mL), cooled to 0° C., and LiOH (34 mg, 0.82 mmol) in H₂O (1 mL), was added. After 3 h, the reaction mixture was acidified with HCl (1.0 M) and extracted with EtOAc (2×25 mL). The combined organic extracts were washed with brine (25 mL). dried (Na₂SO₄>. and concentrated under reduced pressure to afford intermediate 9 (79 mg, 0.16 mmol, 100% yield) which was earned forward to the next reaction without further purification. MS (ESI) m/z: 484.5 (M+H)⁺.

### Intermediate 10: Preparation of 5-(3a,4,5,6,7,7a-hexahydro-4,7-methanobenzo[d]isoxazol-3-yl)-2-methoxybenzoic acid

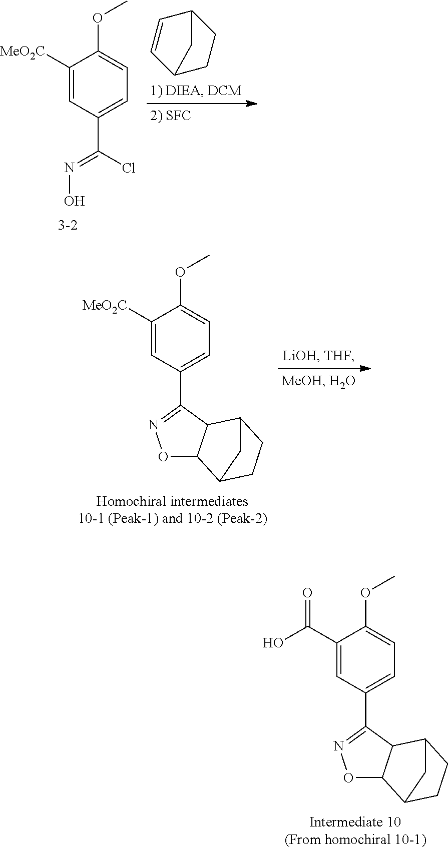

Intermediates 10-1 and 10-2: Preparation of methyl 5-(3a,4,5,6,7,7a-hexahydro-4,7-methanobenzo [d]isoxazol-3-yl)-2-methoxybenzoate: Intermediate 10-1 and 10-2 were prepared in a similar manner as that described for intermediate 3-3 by substituting bicyclo[2.2./]hept-2-ene for 2,5-dihydrofuran followed by chiral SFC separation of the diastereomer by the following preparative chromatographic methods: Instrument: Berger MG II Column: Chiralcel OD-H 21×250 mm ID, 5 μm, Temperature: 40° C., Flow rate: mL/min, 150 Bar, Mobile Phase: gradient 93/7 CO₂/MeOH, Detector Wavelength: 240 nm, Injection Volume: 0.5 mL to afford intermediate 10-1 (Peak-1, 0.16 g, 0.52 mmol, 38% yield >99% ee, Analytical RT=4.42 min), intermediate 10-2 (Peak-2, 0.16 g, 0.52 mmol, 38% yield, >99% ee, Analytical RT=5.52 min). Analytical Chromatographic Conditions: Instrument: Shimadzu Nexera SFC, Column: Chiralcel OD-H, 4.6×100 mm ID, 3 μm, Temperature: 40° C. Flow rate: 2.0 mL/min, 150 Bar, Mobile Phase: gradient 93/7 CO/MeOH. Detector Wavelength: 220 nm. Intermediate 10-1: ¹H NMR (500 MHz, CDCl₃) δ 8.09 (d, J=2.3 Hz, 1H), 7.89 (dd, J=8.8, 2.4 Hz, 1H), 7.03 (d, J=8.7 Hz, 1H), 4.66 (d, J=8.2 Hz, 1H), 3.96 (s, 3H), 3.93 (s, 3H), 3.50 (d, J=7.8 Hz, 1H), 2.65 (br d, J=3.7 Hz, 1H), 2.53 (br s, 1H), 1.71-1.59 (m, 2H), 1.56-1.49 (m, 1H), 1.45-1.34 (m, 1H), 1.26-1.13 (m, 2H). MS (ESI) m/z: 302 (M+H)⁺. Intermediate 10-2: ¹H NMR (500 MHz, CDCl₃) δ 8.09 (d, J=2.3 Hz, 1H), 7.89 (dd, J=8.9, 2.3 Hz, 1H), 7.03 (d, J=8.9 Hz, 1H), 4.66 (d, J=8.4 Hz, 1H), 3.96 (s, 3H), 3.93 (s, 3H), 3.50 (d, J=7.6 Hz, 1H), 2.65 (br d, J=3.8 Hz, 1H), 2.53 (br s, 1H), 1.65-1.58 (m, 2H), 1.54 (br d, J=10.5 Hz, 1H), 1.43-1.29 (m, 1H), 1.24-1.10 (m, 2H). MS (ESI) m/z: 302 (M+H)⁺.

Intermediate 10: To intermediate 10-1 (72 mg, 0.24 μmol) in THF (1.5 mL) was added MeOH (0.1 mL) followed by LiOH (2M, 0.4 mL, 0.7 mmol). After 4 h, the reaction mixture was diluted with water and acidified with HCl (1N) followed by extraction with EtOAc (2×25 mL). The combined organic portions were dried (MgSO₄) and concentrated under reduced pressure to afford intermediate 10 (68 mg, 0.23 μmol, 99% yield). ¹H NMR (400 MHz, CDCl₃) δ 11.11-9.94 (m, 1H), 8.38-8.27 (m, 1H), 8.16 (dd, J=8.8, 2.4 Hz, 1H), 7.14 (d, J=9.0 Hz, 1H), 4.69 (d, J=8.1 Hz, 1H), 4.20-4.03 (m, 3H), 3.55 (d, J=8.1 Hz, 1H), 2.66 (br d, J=3.3 Hz, 1H), 2.55 (br s, 1H), 1.67-1.59 (m, 2H), 1.52 (dt, J=10.7, 1.6 Hz, 1H), 1.47-1.40 (m, 1H), 1.27-1.06 (m, 2H)

### Intermediate 11-3: Preparation of 5-(5-(2-hydroxypropan-2-yl)-4,5-dihydroisoxazol-3-yl)-2-methoxybenzoic acid

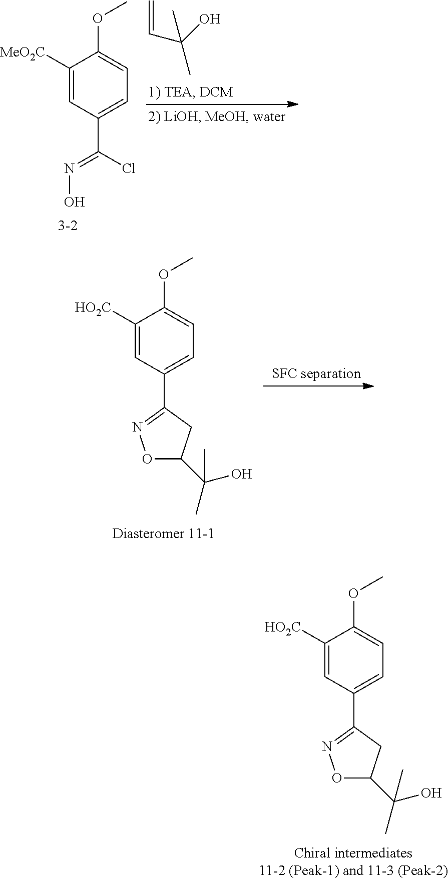

Intermediate 11-1: Preparation of 5-(5-(2-hydroxypropan-2-yl)-4,5-dihydroisoxazol-3-yl)-2-methoxybenzoic acid. Intermediate 11-1 was prepared (181 mg, 100% yield) in a similar manner as intermediate 4-1 by substituting 2-(cyclopent-3-en-1-yl)propan-2-ol for cyclopent-3-en-1-ylmethanol followed by hydrolysis as that described for intermediate 3-6. ¹H NMR (400 MHz, CDCl₃) δ 8.13 (d, J=2.2 Hz, 1H), 7.98 (dd, J=8.8, 2.2 Hz, 1H), 7.05 (d, J=9.0 Hz, 1H), 4.58 (dd, J=11.0, 8.8 Hz, 1H), 4.05 (s, 3H), 3.39 (dd, J=16.9, 8.8 Hz, 11H), 3.27 (dd, J=16.7, 11.0 Hz, 1H), 1.35 (s, 3H), 1.22 (s, 3H).

Intermediate 11-1 (0.18 g, 0.65 mmol) was separated by chiral SFC using the following preparative chromatographic methods: Preparative Chromatographic Conditions: Instrument: Berger MG II Column: Chiralpak AD-H, 21×250 mm, 5 micron, Mobile Phase: 40% MeOH/60% CO₂, Flow Conditions: 45 mL/min, 150 Bar, 40° C., Detector Wavelength: 220 nm, Injection Details: 0.5 mL of ˜65 mg/mL in MeOH. Chiral separation afforded chiral intermediate 11-2 (Peak 1, 64 mg, 0.23 mmol, 35% yield, >95% ee, Analytical RT=5.51 min), intermediate 11-3 (Peak 2, 71 mg, 0.25 mmol, 39% yield, >95% ee, Analytical RT=7.7 min), Analytical Chromatographic Conditions: Instrument: Shimadzu Nexera SFC, Column: Chiralpak AD-H, 4.6×100 mm, 3 micron, Mobile Phase: 35% MeOH/65% CO₂, Flow Conditions: 2.0 mL/min, 150 Bar, 40° C., Detector Wavelength: 220 nm, Injection Details: 5 μL of ˜1 mg/mL in MeOH. 111-2 (Peak-2) ¹H NMR (600 MHz, CD₃OD) δ 8.07 (d, J=2.2 Hz, 1H), 7.82 (dd, J=8.7, 2.1 Hz, 1H), 7.18 (d, J=8.8 Hz, 1H), 5.21 (ddd, J=9.2, 6.2, 2.5 Hz, 1H), 4.27 (m, 1H), 4.24 (td, J=9.4, 4.0 Hz, 1H), 3.94 (s, 3H), 2.16 (m, 1H), 2.05 (m, 1H), 2.00 (m, 1H), 1.99 (m, 1H). Homochiral intermediate 11-3 (Peak-2): ¹H NMR (600 MHz, CD₃OD) δ 8.10 (d, J=1.8 Hz, 1H), 7.86 (dd, J=8.7, 1.9 Hz, 1H), 7.19 (d, J=8.7 Hz, 1H), 5.21 (ddd, J=9.6, 6.4, 2.2 Hz, 1H), 4.32 (m, 1H), 4.18 (td, J=9.6, 3.1 Hz, 1H), 3.94 (s, 3H), 2.21 (m, 1H), 2.17 (m, 1H), 2.14 (m, 1H), 1.98 (dd, J=13.7, 1.5 Hz, 1H).

### Intermediates 12-2 and 12-3: Preparation of 2-methoxy-5-(5-(1-(2-methoxyethoxy)cyclopropyl)-4,5-dihydroisoxazol-3-yl)benzoic acid

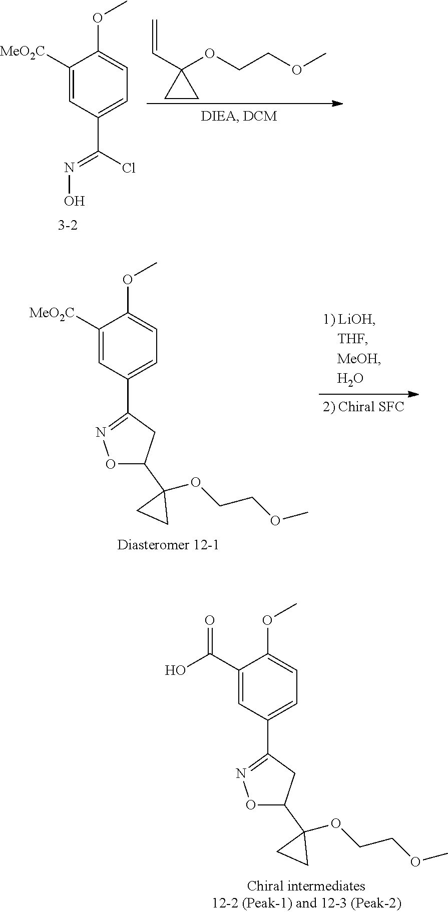

Intermediate 12-1 (0.27 g, 0.77 mmol, 87% yield) was prepared in a similar manner as that described for intermediate 3-3 by substituting 1-(2-methoxyethoxy)-1-vinylcyclopropane for 2,5-dihydrofuran followed by ester hydrolysis as described for intermediate 3-6. Chiral SFC separation of isomers was performed by the following preparative chromatographic methods: Instrument: Berger MG II Column: Chiralpak IC 21×250 mm 1D, 5 μm, Temperature: 40° C., Flow rate: 45 mL/min, 150 Bar, Mobile Phase: gradient 75/25 CO₂/MeOH, Detector Wavelength: 220 nm, Injection Volume: 0.5 mL. SFC separation afforded intermediate 12-2 (77 mg, 0.22 mmol, 30% yield) (peak-1, >99% ee, Analytical RT=5.04 min) and intermediate 12-3 (79 mg, 0.23 mmol, 31% yield) (peak-2, >99% ee, Analytical RT=6.48 min). Analytical Chromatographic Conditions: Instrument: Shimadzu Nexera SFC, Column: Chiralpak, 4.6×100 mm ID, 3 μm, Temperature: 40° C., Flow rate: 2.0 mL/min, 150 Bar, Mobile Phase: gradient 75/25 CO₂/MeOH. Detector Wavelength: 220 nm. Intermediate 12-2: ¹H NMR (500 MHz, CDCl₃) δ 10.61-10.34 (m, 1H), 8.35 (d, J=2.1 Hz, 1H), 8.12 (dd, J=8.8, 2.2 Hz, 1H), 7.15 (d, J=8.9 Hz, 1H), 4.59 (dd, J=10.9, 8.3 Hz, 1H), 4.16 (s, 3H), 3.91-3.83 (m, 1H), 3.80-3.74 (m, 1H), 3.69 (dd, J=16.9, 8.3 Hz, 1H), 3.48-3.39 (m, 2H), 3.30 (s, 3H), 1.13-1.02 (m, 1H), 1.00-0.93 (m, 1H), 0.90-0.79 (m, 2H), 0.78-0.69 (m, 1H). Intermediate 12-3: ¹H NMR (500 MHz, CDCl₃) δ 8.35 (d, J=2.3 Hz, 1H), 8.12 (dd, J=8.7, 2.3 Hz, 1H), 7.15 (d, J=8.9 Hz, 1H), 4.58 (dd, J=10.9, 8.3 Hz, 1H), 4.16 (s, 3H), 3.90-3.84 (m, 1H), 3.81-3.74 (m, 1H), 3.71-3.66 (m, 1H), 3.52 (s, 1H), 3.43-3.40 (m, 2H), 3.32-3.28 (m, 3H), 1.06 (ddd, J=10.7, 6.5, 5.3 Hz, 11H), 1.00-0.93 (m, 1H), 0.87-0.79 (m, 1H), 0.77-0.69 (m, 1H).

### Intermediate 13: Preparation of 5-(4-(hydroxymethyl)-2-oxopyrrolidin-1-yl)-2-methoxybenzoic acid

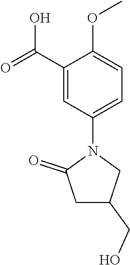 [SMILES: COc1ccc(N2CC(CO)CC2=O)cc1C(=O)O]

### Intermediate 13

To a solution containing 4-(hydroxymethyl)pyrrolidinone (50 mg, 0.43 mmol), methyl 5-iodo-2-methoxybenzoate (127 mg, 0.434 mmol), Xantphos (25 mg, 0.043 mmol), Cs₂CO₃ (0.28 g, 0.87 mmol) was added dioxane (2 mL) and the reaction mixture purged with N₂ for 10 min., followed by addition of Pd₂(dba)₃ (20 mg, 22 μmol). The resulting reaction mixture was heated at 100° C. for 15 h. MS (ESI) m/z: 280.2 (M+H)⁺. To the cooled reaction flask was added a silicycle Pd trap and the reaction mixture was filtered. The filtrate was diluted with water (10 mL) and extracted with EtOAc (2×25 mL). The combined organic portions were washed with brine (15 mL), dried (MgSO₄) and concentrated under reduced pressure. To the residue was added in THF (3 mL) followed by a solution of MeOH (0.5 mL)/water (1 mL) and the reaction mixture cooled to 0° C. Aq. LiOH (2M, 0.7 mL, 1.3 mmol) was added and the reaction mixture was stirred for 3 h, acidified (1N HCl) and concentrated under reduced pressure. The residual was freeze-dried to afford intermediate 13 as a brown solid, which was used in the next step without further purification. MS (ESI) m/z: 266.1 (M+H)⁺.

### Intermediate 14: Preparation of 2-methoxy-5-(5-oxo-3a,4,5,6,7,7a-hexahydro-4,7-methanoisoxazolo[5,4-c]pyridin-3-yl)benzoic acid

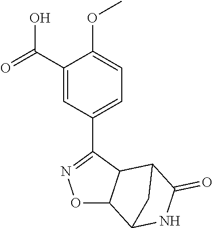 [SMILES: COc1ccc(C2=NOC3C4CC(C(=O)N4)C23)cc1C(=O)O]

### Intermediate 14

Intermediate 14 (0.17 g, 0.60 mmol, 86% yield) was prepared in a similar manner as intermediate 3-3 by substituting (1S,4R)-2-azabicyclo[2.2.1]hept-5-en-3-one for 2,5-dihydrofuran and (Z)-5-(chloro(hydroxyimino)methyl)-2-methoxybenzoic acid for intermediate 3-2 and was used without purification. MS (ESI) m/z: 303.3 (M+H)⁺.

### Intermediate 15: Preparation of 6-amino-N-(4-fluoro-3-(trifluoromethyl)phenyl)-2-methylbenzo[d]thiazole-5-carboxamide

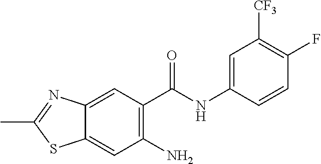 [SMILES: Cc1nc2cc(C(=O)Nc3ccc(F)c(C(F)(F)F)c3)c(N)cc2s1]

### Intermediate 15

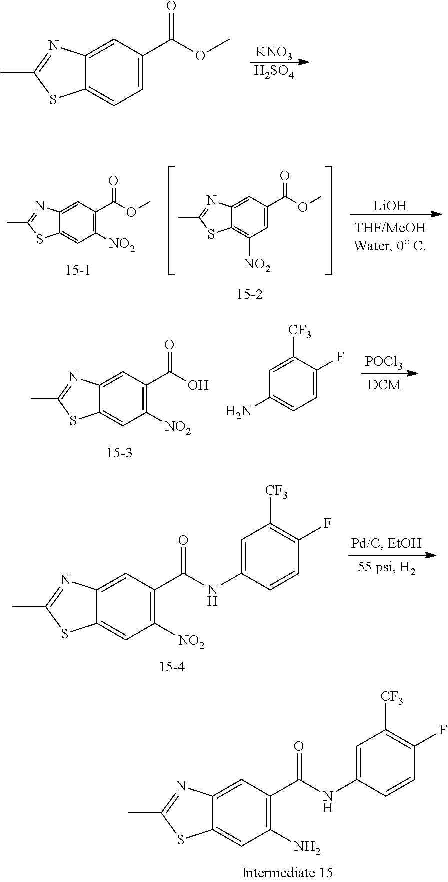

Intermediate 15-1: Preparation of methyl 2-methyl-6-nitrobenzo[d]thiazole-5-carboxylate. To methyl 2-methylbenzo[d]thiazole-5-carboxylate (0.1 g, 0.5 mmol) was added H₂SO₄ (0.5 mL) and the reaction mixture cooled to 0° C. To this solution was added a cold (0° C.) solution of KNO₃ (59 mg, 0.60 mmol) in H₂SO₄ (1 mL). After 24 h, ice water was added to the reaction mixture and the solution extracted with (4×20 mL) DCM. Hexanes were added to the organic layer and solids precipitated which were collected by filtration, but the majority of the desired product was in the aqueous layer. The aqueous layers were concentrated in vacuo and residue obtained was purified by normal phase chromatography eluted with hexanes/EtOAc to afford 15-1 (46 mg, 38% yield), ¹H NMR (400 MHz, CDCl₃) δ 8.49 (s, 1H), 8.26 (s, 1H), 4.00 (s, 3H), 2.96 (s, 3H) and 15-2 (15 mg, 12% yield), ¹H NMR (400 MHz, CDCl₃) δ 9.04 (d, J=1.3 Hz, 1H), 8.93 (d, J=1.3 Hz, 1H), 4.07 (s, 3H), 2.96 (s, 3H).

Intermediate 15-3: Preparation of 2-methyl-6-nitrobenzo[d]thiazole-5-carboxylic acid. Hydrolysis of intermediate 15-1 (46 mg, 0.18 mmol) was done in THF (2 mL)/MeOH (0.5 mL)/water (0.5 mL), and LiOH (2M, 0.5 ml, 0.5 mmol) in 24 h. The solvents were reduced in vacuo and the residue dissolved in dil. HCl (2 mL). The solution was extracted with EtOAc (2×10 mL) and the combined organic portions dried (MgSO₄) and concentrated under reduced pressure to yield intermediate 15-3 (39 mg, 0.16 mmol, 90% yield) as a yellow solid. ¹H NMR (400 MHz, DMSO-d₆) δ 14.44-13.28 (m, 1H), 8.87 (s, 1H), 8.28 (s, 1H), 2.92 (s, 3H).

Intermediate 15-4: Preparation of N-(4-fluoro-3-(trifluoromethyl)phenyl)-2-methyl-6-nitrobenzo[d]thiazole-5-carboxamide. To 15-3 (49 mg, 0.21 mmol) and 4-fluoro-3-(trifluoromethyl)aniline (37 mg, 0.21 mmol) and pyridine (166 μL, 2.00 mmol) in DCM (1 mL), cooled to 0° C., was added POCl₃ (19 μL, 0.21 mmol). After 24 h, the reaction mixture was quenched with NaHCO₃(aq.) (20 mL) and extracted with EtOAc (3×20 mL). The combined organic layers were washed with brine (15 mL) and dried (MgSO₄). Purification by normal phase silica gel chromatography afforded intermediate 15-4 (41 mg, 0.10 mmol, 50% yield). ¹H NMR (400 MHz, DMSO-d6) δ 11.10 (s, 1H), 9.08 (s, 1H), 8.34 (s, 1H), 8.21 (dd, J=6.6, 2.4 Hz, 1H), 8.01-7.93 (m, 1H), 7.58 (t, J=9.8 Hz, 1H), 2.94 (s, 3H). MS (ESI) m/z 399.8 (M+H)⁺.

Intermediate 15: Intermediate 15-4 (41 mg, 0.1 mmol) was hydrogenated in EtOH (4 mL) in the presence of 10% Pd/C (10 mg) at 55 psi. After 4 h, the catalyst was removed by filtration and the reaction mixture concentrated under reduced pressure to afford intermediate 15 (34 mg, 92 μmol, 90% yield). ¹H NMR (400 MHz, DMSO-d6) δ 10.54 (s, 1H), 8.29-8.26 (m, 1H), 8.07 (m, 2H), 7.57-7.49 (m, 2H), 6.39 (s, 2H), 2.72 (s, 3H). MS (ESI) m/z 370 (M+H)⁺.

### Intermediate 16-6: 5′-(2-(tert-butoxy)-1-((tert-butoxycarbonyl)amino)-2-oxoethyl)-2′-fluoro-4-methoxy-[1,1′-biphenyl]-3-carboxylic acid

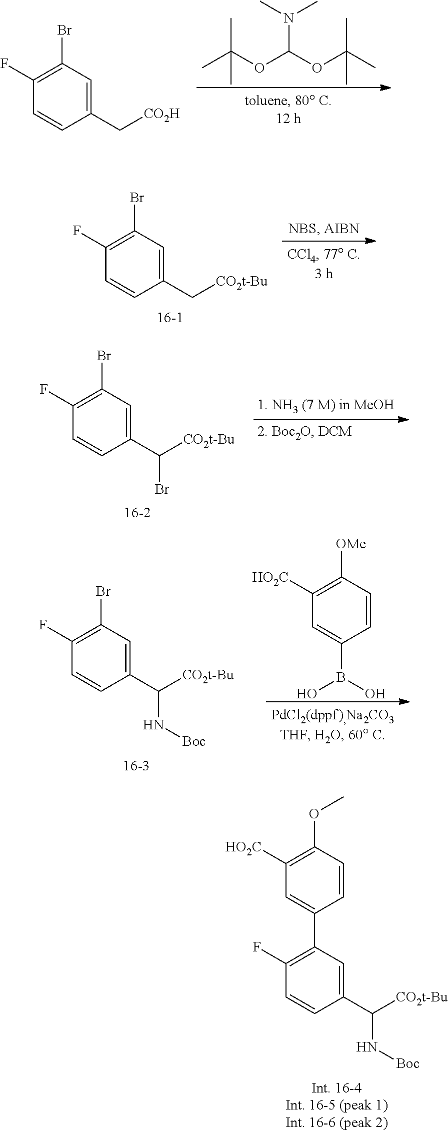

Intermediate 16-1: Preparation of tert-butyl 2-(3-bromo-4-fluorophenyl)acetate. Intermediate 16-1 was prepared by the general procedures described Ludwig, J.: Lehr, M. Syn. Comm. 2004, 34, 3691-3695, except that the reaction temperature was maintained at 80° C. for 12 h. ¹H NMR (500 MHz, CDCl₃) δ 7.49 (dd, J=6.6, 2.2 Hz, 1H), 7.20 (ddd, J=8.3, 4.6, 2.2 Hz, 1H), 7.13-7.03 (m, 1H), 3.49 (s, 2H), 1.46 (s, 9H).

### Intermediate 16-2: Preparation of tert-butyl 2-bromo-2-(3-bromo-4-fluorophenyl)acetate

To a 20 mL reaction vial charged with intermediate 16-1 (266 mg, 0.92 mmol) was added NBS (0.2 g, 1 mmol), carbon tetrachloride (10 mL), and AIBN (15 mg, 90 mmol). The solution was stirred at 77° C., for 3 h. The solution was concentrated under reduced pressure and purified by normal phase silica gel chromatography to afford intermediate 16-2 (0.3 g, 0.8 mmol, 91% yield). ¹H NMR (500 MHz, CDCl₁) δ 7.79 (dd, J=6.5, 2.3 Hz, 1H), 7.55-7.46 (m, 1H), 7.18-7.10 (m, 1H), 5.18 (s, 1H), 1.50 (s, 9H).

Intermediate 16-3: Preparation of tert-butyl 2-(3-bromo-4-fluorophenyl)-2-((tert-butoxycarbonyl) amino) acetate. To intermediate 16-2 (60 mg, 0.16 mmol) was added ammonia (0.5 mL, 4 mmol, in MeOH). After stirring at rt for 12 h, the reaction mixture was concentrated under vacuum. DCM (1 mL) was then added to the residue followed by BOC-anhydride (0.11 mL, 0.49 mmol) and DIEA (57 μL, 0.33 mmol) and the reaction mixture was stirred at rt for 1 h. The reaction mixture was concentrated under vacuum and silica gel chromatography purification produced 16-3 (42 mg, 0.1 mmol, 63% yield). LC-MS RT=1.14 min; MS (ESI) m/z: 406.0 (M+H)⁺. [Method A]

Intermediates 16-5 and 16-6: To a reaction vessel containing intermediate 16-3 (330 mg, 0.816 mmol) was added 5-borono-2-methoxybenzoic acid (208 mg, 1.061 mmol), PdCl₂(dppf)-DCM Adduct (100 mg, 0.122 mmol), and Na₂CO₃ (346 mg, 3.27 mmol) in THF (12 mL). The reaction mixture was degassed by bubbling N₂ for 10 min, sealed, and stirred at 60° C. for 18 h. After allowing to cool to rt, the reaction mixture was concentrated under reduced pressure and subjected to reverse phase-HPLC purification to produce intermediate 16-4 (25 mg, 53 mmol, 52% yield). ¹H NMR (500 MHz, CDCl₃) δ 8.38 (d, J=1.9 Hz, 1H), 7.80 (dt, J=8.7, 2.0 Hz, 1H), 7.46 (dd, J=−7.4, 2.5 Hz, 1H), 7.36 (dddd, J=8.8, 4.4, 2.2, 1.1 Hz, 1H), 7.19-7.13 (m, 2H), 5.67 (br d, J=5.2 Hz, 1H), 5.25 (br d, J=6.3 Hz, 1H), 4.16 (s, 3H), 1.46 (br s, 9H), 1.44 (s, 9H). Intermediate 16-4 was separated into enantiomers using chiral SFC. Preparative chromatographic conditions: Instrument: Berger MG II; Column: Chiralpak ID, 21×250 mm, 5 micron; Mobile phase: 20% MeOH/80% CO₂; Flow conditions: 45 mL/min, 120 Bar, 40° C.: Detector wavelength: 209 nm; Injection details: 49 injections in MeOH. Analytical chromatographic conditions: Instrument: Waters UPC2 analytical SFC; Column: Chiralpak IC, 4.6×100 mm, 3 micron; Mobile phase: 25% MeOH/75% CO₂; Flow conditions: 2 mL/min, 150 Bar, 40° C.; Detector wavelength: 220 nm. Intermediate 16-5: (peak 1, RT=4.22 min, 95.7% ee). Intermediate 16-6 (peak 2, RT=5.11 min, >99% ee).

### Intermediate 17: Preparation of (S)-5′-(1-((cyclobutylcarbamoyl)oxy)-2,2,2-trifluoroethyl)-2′-fluoro-4-methoxy-[1,1′-biphenyl]-3-carboxylic acid

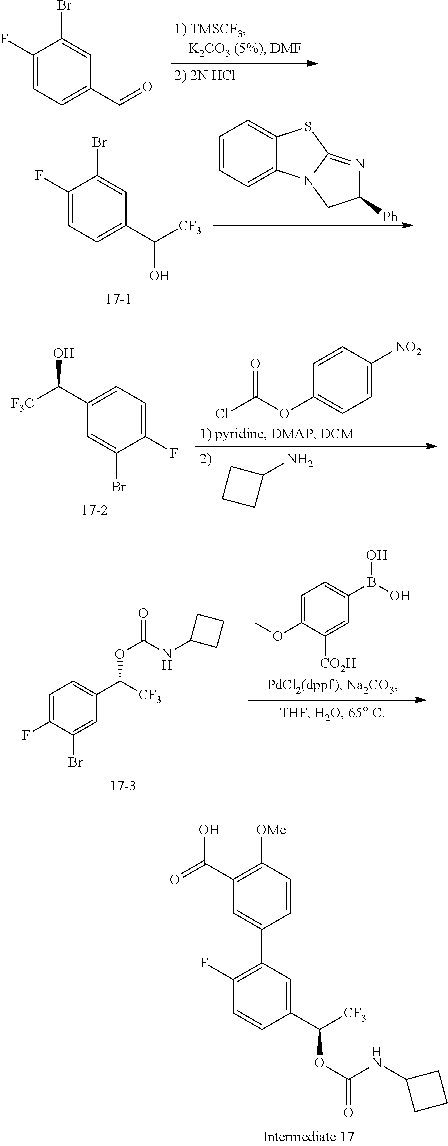

Intermediate 17-1: Preparation of 1-(3-bromo-4-fluorphenyl)-2,2,2-trifluoroethan-1-ol. 3-Bromo-4-fluorobenzaldehyde (0.2 g, 1 mmol) was dissolved in DMF (3.5 mL), and to this solution was added (trifluoromethyl)trimethylsilane (0.3 mL, 2 mmol), and K₂CO₃ (8.0 mg, 58 μmol). The reaction mixture was stirred at rt for 60 min and HCl (2N, 3 mL) was added. After stirring at it for an additional 1 h, the reaction mixture was diluted with EtOAc (15 mL), and the solution washed with sat. NH₄C₁ (25 mL). The aqueous phase was extracted with EtOAc (2×10 mL), the combined organic portions dried (Na₂SO₄), concentrated under reduced pressure and purified the residue by silica gel chromatography using hexanes/EtOAc as eluents to produce intermediate 17-1 (0.2 g, 0.8 mmol, 70% yield). ¹H NMR (500 MHz, CDCl₃) δ 7.74 (dd, J=6.5, 2.1 Hz, 1H), 7.43 (ddd, J=8.4, 4.8, 2.2 Hz, 1H), 7.19 (t, J=8.4 Hz, 1H), 5.11-4.98 (m, 1H), 2.69 (d, J=4.4 Hz, 1H).

Intermediate 17-2: Preparation of (S)-1-(3-bromo-4-fluorophenyl)-2,2,2-trifluoroethan-1-ol. (S)-2-phenyl-2,3-dihydrobenzo[d]imidazo[2,1-b]thiazole (0.4 g, 2 mmol) and intermediate 17-1 (11 g, 40 mmol) were dissolved in diisopropyl ether (134 mL) and chilled between 0° C. to −20° C. The solution was treated with isobutyl anhydride (4 mL, mmol) and transferred to a freezer (−20° C.) for 18 h. The reaction was quenched with MeOH (1 mL) and phosphate buffer. The resulting solution was extracted with EtOAc (2×25 mL) dried (MgSO₄) concentrated under reduced pressure to yield a residue which was purified via normal phase chromatography using hexanes/EtOAc as eluents to afford intermediate 17-2 (5 g, 20 mmol, 44% yield. >99% ee). ¹H NMR (500 MHz, CDCl₃) δ 7.74 (dd, J=6.3, 1.9 Hz, 1H), 7.50-7.39 (m, 1H), 7.18 (t, J=8.4 Hz, 1H), 6.71-5.53 (m, 1H), 5.03 (q, J=6.5 Hz, 1H).

Intermediate 17-3: Preparation of (S)-1-(3-bromo-4-fluorophenyl)-2,2,2-trifluoroethyl cyclobutylcarbamate. Intermediate 17-2 (0.3 g, 1 mmol), pyridine (0.4 mL, 6 mmol), and DMAP (13 mg, 0.10 mmol) were dissolved in DCM (20 mL) and 4-nitrophenyl carbonochloridate (Ig, 6 mmol) was added. The reaction mixture was stirred for 1 h, followed by the addition of cyclobutanamine (0.78 g, 11 mmol). After 2 h, the reaction mixture was concentrated under reduced pressure and the residue purified by normal phase chromatography using hexanes/EtOAc as eluents to afford 17-3 (0.35 g, 0.90 mmol, 85% yield) as a white solid. MS (ESI) m/z: 371.7 (M+H, Br isotope peak seen).⁺

Intermediate 17: Preparation of (S)-5′-(1-((cyclobutylcarbamoyl)oxy)-2,2,2-trifluoroethyl)-2′-fluoro-4-methoxy-[1,1′-biphenyl]-3-carboxylic acid. In a vial was added 17-3 (30 mg, 0.080 mmol), 5-borono-2-methoxybenzoic acid (21 mg, 0.10 mmol), PdCl₂(dppf)-DCM (9 mg, 0.01 mmol), Na₂CO₃ (34 mg, 0.32 mmol), THF (1 mL) and H₂O (0.25 mL). The reaction mixture was degassed by bubbling N₂ for 10 min, sealed. and stirred at 65° C. for 3 h. After allowing to cool to rt the reaction was quenched with HCl (IN), and extracted with EtOAc (2×25 mL). The combined organic portions were dried (Na₂SO₄), concentrated under reduced pressure and purified by reverse phase chromatography using a gradient of solvent A: 20% ACN-80% H₂O—0.1% TFA; solvent B: 80% ACN-20% H₂O—0.1% TFA to afford intermediate 17 (24 mg, 0.050 mmol, 66% yield). ¹H NMR (400 MHz, CDCl₃) δ 8.39 (d, 0.1=1.5 Hz, 1H), 7.83 (br d, J=8.6 Hz, 1H), 7.56 (br d, J=6.4 Hz, 1H), 7.45 (br s, 1H), 7.25-7.10 (m, 2H), 7.02-6.44 (m, 1H), 6.10 (q, J=6.5 Hz, 1H), 5.26 (br d, J=7.5 Hz, 1H), 4.17 (s, 3H), 2.54-2.25 (m, 2H), 2.08-1.88 (m, 2H), 1.83-1.46 (m, 2H) MS (ESI) m/z: 442.0 (M+H)⁺.

### Intermediate 18: Preparation of 6-amino-N-(4-fluoro-3-(trifluoromethyl)phenyl)benzo [d]thiazole-5-carboxamide

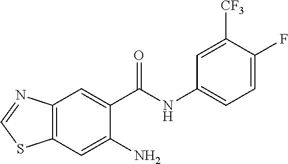 [SMILES: Nc1cc2scnc2cc1C(=O)Nc1ccc(F)c(C(F)(F)F)c1]

### Intermediate 18

Intermediate 18 (33 mg, 93 μmol, 80% yield) was prepared in a similar manner as intermediate 15 by substituting methyl benzo[d]thiazole-5-carboxylate for methyl 2-methylbenzo[d]thiazole-5-carboxylate. ¹H NMR (400 MHz, CDCl₃) δ 8.72 (s, 1H), 8.61-8.47 (m, 1H), 8.30 (s, 1H), 8.00 (dd, J=6.2, 2.6 Hz, 1H), 7.82 (dt, J=8.6, 3.5 Hz, 1H), 7.24 (s, 1H), 7.20 (s, 1H), 5.55 (s, 2H). MS (ESI) m/z: 356.2 (M+H)⁺.

### Intermediate 19: Preparation of 5-amino-N-cyclopentyl-2-methylbenzo[d]thiazole-6-carboxamide

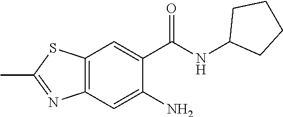 [SMILES: Cc1nc2cc(N)c(C(=O)NC3CCCC3)cc2s1]

### Intermediate 19

Intermediate 19 (51 mg, 190 μmol, 73% yield) was prepared in a similar manner as intermediate 1, substituting cyclopentanamine for 4-fluoro-3-(trifluoromethyl)aniline. ¹H NMR (500 MHz, CDCl₃) δ 7.86-7.69 (m, 1H), 7.23 (s, 1H), 5.42-5.34 (m, 2H), 4.45-4.34 (m, 1H), 2.80 (s, 3H), 2.16-2.08 (m, 2H), 1.79-1.73 (m, 2H), 1.71-1.63 (m, 3H), 1.61-1.50 (m, 2H). MS (ESI) m/z: 276 (M+H)⁺.

### Intermediate 20: Preparation of 6-amino-N-(4-fluoro-3-(trifluoromethyl)phenyl)-2-(trifluoromethyl)benzo[d]thiazole-5-carboxamide

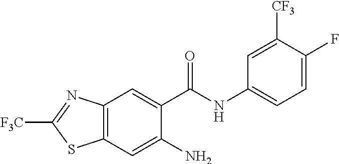 [SMILES: Nc1cc2sc(C(F)(F)F)nc2cc1C(=O)Nc1ccc(F)c(C(F)(F)F)c1]

### Intermediate 20

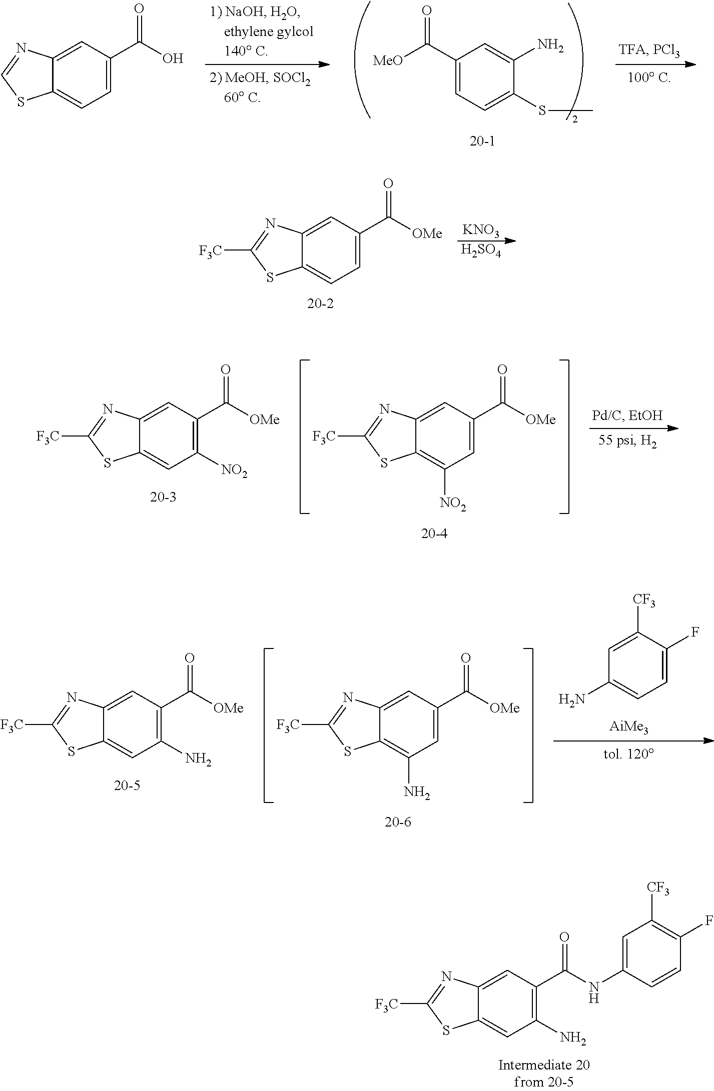

Intermediate 20-1: Preparation of dimethyl 4,4′-disulfanediylbis(3-aminobenzoate). Benzo[d]thiazole-5-carboxylic acid (1 g, 5.58 mmol) and NaOH (0.67 g, 17 mmol) in ethylene glycol (25 mL), water (25 mL) were heated to 140° C. for 4 h. The reaction mixture was allowed to cool to rt and partitioned with water (20 mL) and EtOAc (50 mL). The aqueous layer pH was adjusted to 4 with HCl and was extracted with EtOAc (2×20 mL). The combined organic layers were washed with brine (15 mL) and dried (MgSO₄). The organic layer was concentrated under vacuum and the recovered solid in MeOH (50 mL) was added thionyl chloride (0.4 mL, 6 mmol) and the reaction mixture was heated to 60° C. for 24 h. The reaction mixture was allowed to cool, triturated with DCM and hexanes and solid was filtered. The solid was treated with saturated aqueous sodium bicarbonate solution (30 mL) and extracted with EtOAc (3×30 mL). The combined organic layers were washed with brine (15 mL), dried (MgSO₄) and concentrated under reduced pressure. The residue was purified via silica gel chromatography eluting with DCM/0-10% MeOH to generate intermediate 20-1 (0.9 g, 2.5 mmol, 32% yield) as a bright yellow solid, which was used without further purification. MS (ESI) m/z: 365.2 (M+H)⁺.

Intermediate 20-2: Preparation of methyl 2-(trifluoromethyl)benzo[d]thiazole-5-carboxylate (Du, G. et. al. Het. 2015, 9, 1723). To intermediate 20-1 (0.28 g, 0.77 mmol) in toluene (4 mL), was added TFA (0.6 mL, 8 mmol), PCl₃ (3.8 mL, 7.7 mmol) and the reaction mixture was heated to 100° C. for 2 h, then stirred at rt for 18 h. The solvents were removed under reduced pressure and the residue was purified via silica gel chromatography eluting with hexanes/EtOAc to afford intermediate 20-2 (0.2 g, 0.8 mmol, 100% yield) as a white solid. ¹H NMR (400 MHz, CDCl₃) δ 8.91 (d, J=1.3 Hz, 1H), 8.27 (dd, J=8.6, 1.5 Hz, 11H), 8.09 (d, J=8.6 Hz, 1H), 4.04 (s, 3H). MS (ESI) m/z: 262.1 (M+H)⁺.

Intermediate 20-3: Preparation of methyl 6-nitro-2-(trifluoromethyl)benzo[d]thiazole-5-carboxylate. Intermediate 20-3 (98 mg, 0.32 mmol, 42% yield) was prepared in a similar manner as 15-1, substituting 20-2 for methyl 2-methylbenzo[d]thiazole-5-carboxylate. For 20-4 ¹H NMR (600 MHz, CDCl₃) δ 8.25 (d, J=8.6 Hz, 1H), 8.20 (d, J=8.6 Hz, 1H), 3.99 (s, 3H). MS (ESI) m/z: 307.0 (M+H)⁺. The material obtain thus also contained regioisomeric 20-4 (30 mg, 0.1 mmol, 26% yield)

Intermediates 20-5 and 20-6: Preparation of methyl 6-amino-2-(trifluoromethyl)benzo[d]thiazole-5-carboxylate and methyl 7-amino-2-(trifluoromethyl)benzo[d]thiazole-5-carboxylate. Intermediates 20-3 and 20-4 (98 mg, 0.32 mmol) were hydrogenated at 55 psi in the presence of 10% Pd/C (15 mg) for 5 h. The reaction mixture was filtered to afford a yellow film containing 20-5 and 20-6 (80 mg, 0.29 mmol, 90% yield) that was used without purification. MS (ESI) m-z: 277.1 (M+H)⁺.

Intermediate 20 (24 mg, 0.056 mmol, 77% yield) was prepared in the same general manner as 1-3 substituting intermediates 20-5 and 20-6 for 1-2. The material was purified by reverse phase HPLC. ¹H NMR (400 MHz, CDCl₃) δ 8.33 (s, 1H), 8.05-7.93 (m, 2H), 7.78 (dt, J=9.1, 3.5 Hz, 1H), 7.29-7.24 (m, 1H), 7.22 (s, 1H). Protons not visible in the NMR, likely due to overlap with the solvent peak. MS (ESI) m/z: 424.2 (M+H)⁺.

### Intermediate 21: Preparation of 5-(4-(((tert-butoxycarbonyl)amino)methyl)-2-oxopyrrolidin-1-yl)-2-methoxybenzoic acid

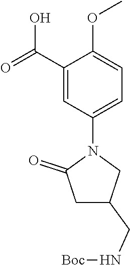 [SMILES: COc1ccc(N2CC(CNC(=O)OC(C)(C)C)CC2=O)cc1C(=O)O]

### Intermediate 21

Intermediate 21 (0.17 g, 0.46 mmol, 81% yield) was prepared in a similar manner as intermediate 13 substituting tert-butyl ((5-oxopyrrolidin-2-yl)methyl)carbamate for 4-(hydroxymethyl)pyrrolidinone and was used without further purification. MS (ESI) m/z: 309.3 (M+H-tBu).

### Intermediate 22: Preparation of 5-amino-N-(4-fluoro-3-(trifluoromethyl)phenyl)-3-methylbenzo[d]isoxazole-6-carboxamide

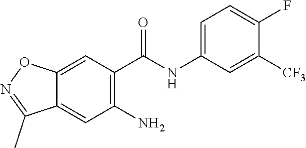 [SMILES: Cc1noc2cc(C(=O)Nc3ccc(F)c(C(F)(F)F)c3)c(N)cc12]

### Intermediate 22

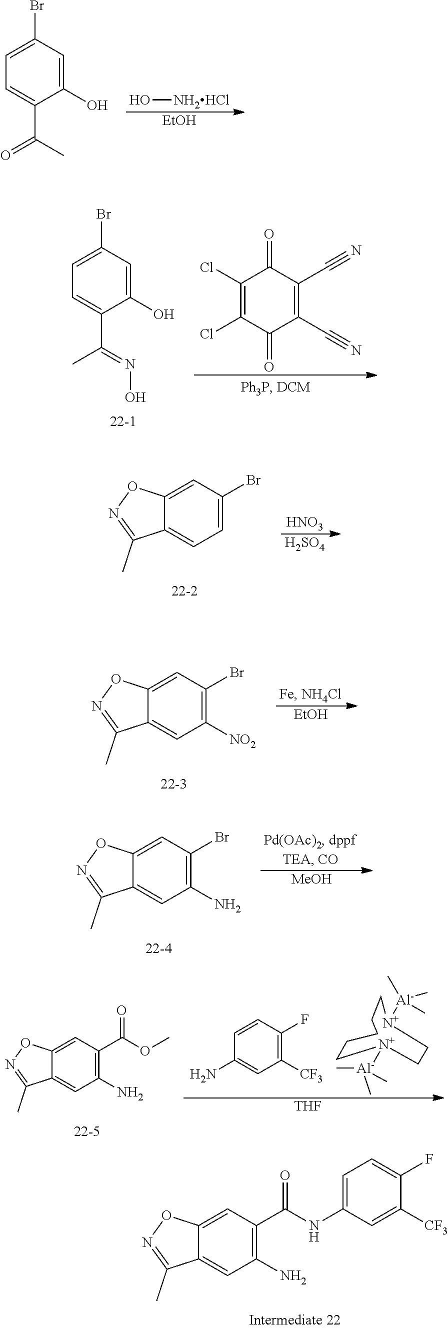

### Intermediate 22-1: Preparation of (Z)-1-(4-bromo-2-hydroxyphenyl)ethan-1-one oxime

To a solution of 1-(4-bromo-2-hydroxyphenyl)ethan-1-one (6 g, 30 mmol) in EtOH (8 mL) and water (2 mL). was added, hydroxylamine hydrochloride (5.8 g, 84 mmol) and sodium acetate (3.4 g, 42 mmol). The reaction mixture was then refluxed for 2 h under inert atmosphere before allowing to cool to rt. The solvents were then concentrated under vacuum to obtain a residue which was subjected to silica gel column chromatography using hexane and EtOAc as eluants to yield 22-1 (6.0 g, 93% yield). MS (ESI) m/z: 229.9 (M+H)⁺.

Intermediate 22-2: Preparation of 6-bromo-3-methylbenzo[d]isoxazole. To a mixture of 4,5-dichloro-3,6-dioxocyclohexa-1,4-diene-1,2-dicarbonitrile (11.8 g, 52.0 mmol) and triphenylphosphine (13.7 g, 52.0 mmol) in dry DCM (100 mL) at rt, was added, 22-1 (6 g, mmol) and the reaction mixture was stirred for 1 h. The reaction mixture was concentrated under vacuum and the residue subjected to silica gel column chromatography using hexane and EtOAc as eluents to yield intermediate 22-2 (2.0 g, 58% yield). MS (ESI) m/z: 211.9 (M+H)⁺.

### Intermediate 22-3: Preparation of 6-bromo-3-methyl-5-nitrobenzo[d]isoxazole

Intermediate 22-2 (1.4 g, 6.6 mmol) was added to a precooled sulfuric acid (0.53 ml, 9.9 mmol) at 0° C. and the reaction mixture was stirred for 10 min. To this mixture was then added nitric acid (0.4 ml, 10 mmol) and the reaction mixture was stirred for 2 h. The reaction mixture was then poured onto crushed ice to form a precipitate which was extracted by EtOAc (20 mL). The organic layer was separated, dried over sodium sulfate and concentrated under vacuum to yield a residue which was subjected to silica gel column chromatography using hexane and EtOAc as eluents to yield intermediate 22-3 (1.3 g, 77% yield). MS (ESI) m/z: 359.3 (M+2H)⁺.

Intermediate 22-4: Preparation of 6-bromo-3-methylbenzo[d]isoxazol-5-amine. To a stirred solution of intermediate 22-3 (500 mg, 1.9 mmol) in EtOH (3 mL) was added, a solution of ammonium chloride (312 mg, 5.80 mmol) in water (3 mL). The reaction mixture was then heated to 50° C. and iron (543 mg, 9.70 mmol) was added. The reaction temperature was then increased to 80° C. and the reaction mixture stirred for 14 h. The reaction mixture was allowed to cool to rt and then filtered through Celite® and washed with EtOAc. The solution was concentrated under reduced pressure. The resulting residue was dissolved in EtOAc (20 mL) and washed with water (20 mL). The organic layers were then dried over sodium sulfate, concentrated under reduced pressure to get a residue which was purified by silica gel column chromatography using hexane and EtOAc as eluents to yield intermediate 22-4 (400 mg, 91% yield). MS (ESI) m/z: 227.0 (M+H)⁺.

Intermediate 22-5: Preparation of methyl 5-amino-3-methylbenzo[d]isoxazole-6-carboxylate. A solution of intermediate 22-4 (1.1 g, 4.8 mmol) in MeOH (100 mL) was added into the reactor followed by DMF (100 mL). The reaction mixture was purged with nitrogen gas after which diacetoxypalladium (0.22 g, 0.97 mmol), dppf (0.8 g, 1 mmol) and TEA (10.1 mL, 73.0 mmol) were added. After purging the reaction mixture with nitrogen gas, the reaction mixture was stirred for 18 h under atmosphere of carbon monoxide at 100° C. The reaction mixture was then allowed to cool to rt and concentrated under vacuum to give a residue which was subjected to silica gel chromatography using hexane and EtOAc as eluents to yield intermediate 22-5 (700 mg, 70% yield). MS (ESI) m/z: 207.1 (M+H)⁺.

Intermediate 22: To a stirred solution of intermediate 22-5 (50 mg, 0.24 mmol) in THF (2 mL) was added bis(trimethylaluminium)-1,4-diazabicyclo[2,2,2]octane adduct (62 mg, 0.24 mmol) and the reaction mixture was stirred for 10 min. Then 4-fluoro-3-(trifluoromethyl)aniline (0.03 mL. 0.24 mmol) was added and the reaction mixture was stirred for 12 h at rt. To this mixture was added a saturated solution of sodium potassium tartrate and the solution was stirred for 10 min after which it was extracted with EtOAc (5 mL). The organic layers were dried over sodium sulfate, concentrated under vacuum to give a residue which was purified by silica gel chromatography to yield intermediate 22 (60 mg, 70% yield). MS (EST) m/z: 354.1 (M+H)⁺.

### Intermediate 23: Preparation of 5′-(tert-butoxycarbonyl)-2′-fluoro-4-methoxy-[1,1′-biphenyl]-3-carboxylic acid

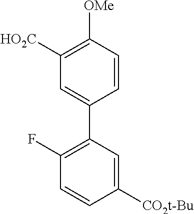 [SMILES: COc1ccc(-c2cc(C(=O)OC(C)(C)C)ccc2F)cc1C(=O)O]

### Intermediate 23

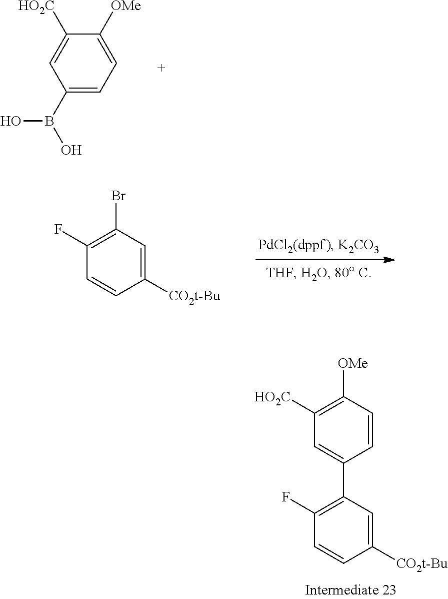 [SMILES: COc1ccc(B(O)O)cc1C(=O)O ; CC(C)(C)OC(=O)c1ccc(F)c(Br)c1 ; COc1ccc(-c2cc(C(=O)OC(C)(C)C)ccc2F)cc1C(=O)O]

Intermediate 23: To a vial was added 5-borono-2-methoxybenzoic acid (500 mg, 2.55 mmol), tert-butyl 3-bromo-4-fluorobenzoate (842 mg, 3.06 mmol), tert-butyl 3-bromo-4-fluorobenzoate (842 mg, 3.06 mmol), K₂CO₃ (1.76 g, 12.8 mmol), PdCl₂(dppf)-CH₂Cl₂ adduct (313 mg, 0.380 mmol), and THF (22 mL). The reaction mixture was degassed for 2 min with nitrogen, then heated at 80° C. for 18 h. After cooling to rt, the reaction mixture was diluted with IN HCl (25 mL) and the solution extracted with EtOAc (3×25 mL). The combined organic portions were dried over Na₂SO₄, filtered, concentrated under reduced pressure, and the resulting residue was dissolved in DMF and purified by preparative RP-HPLC to afford intermediate 23 (586 mg, 1.69 mmol, 66% yield). ¹H NMR (500 MHz, DMSO-d6) δ 12.78 (br s, 1H), 7.98 (dd, J=7.7, 2.2 Hz, 1H), 7.93 (ddd, J=8.5, 4.8, 2.3 Hz, 1H), 7.84-7.79 (m, 1H), 7.73 (dt, J=8.7, 1.7 Hz, 1H), 7.45 (dd, J=10.5, 8.5 Hz, 1H), 7.28 (d, J=8.8 Hz, 1H), 3.89 (s, 3H), 1.57 (s. 9H): MS (ESI) m/z: 347.1 (M+H)⁺.

### Intermediate 24: Preparation of 6-amino-2,2-difluoro-N-(4-fluoro-3-(trifluoromethyl)phenyl)benzo[d][1,3]dioxole-5-carboxamide

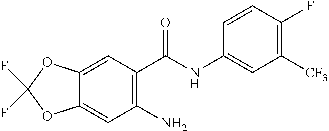 [SMILES: Nc1cc2c(cc1C(=O)Nc1ccc(F)c(C(F)(F)F)c1)OC(F)(F)O2]

### Intermediate 24

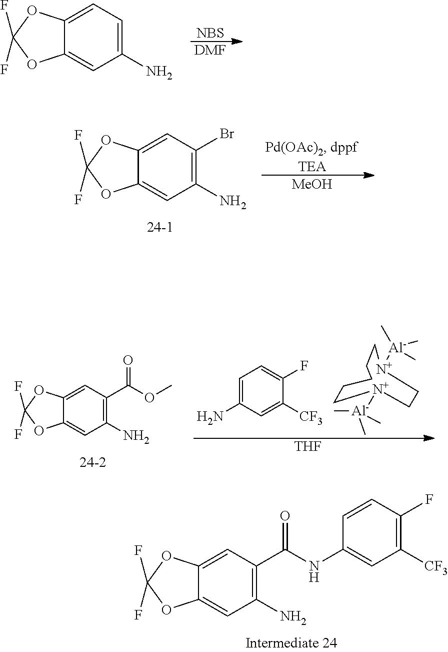

Intermediate 24-1: Preparation of 6-bromo-2,2-difluorobenzo[d][1,3]dioxol-5-amine. To a stirred suspension of 5-amino-2,2-difluoro-1,3-benzodioxole (9.4 g, 54 mmol) in DMF (100 mL) was added NBS (10.63 g, 59.70 mmol). The reaction mixture was then stirred at rt for 2 h. The reaction mixture was partitioned between EtOAc and water. The organic layer was separated then washed with brine, dried over anhydrous sodium sulfate, filtered and concentrated under vacuum to yield a brown gum which was subjected to silica gel chromatography to yield 24-1 (11.3 g, 43.9 mmol, 81% yield). MS (E⁻) m/z=252.2 (M+2H)⁺.

Intermediate 24-2: Preparation of methyl 6-amino-2,2-difluorobenzo[d][1,3]dioxole-5-carboxylate. To a degassed solution of 24-1 (5.5 g, 22 mmol) in MeOH (120 mL) and DMF (120 mL) was added TEA (30.4 mL, 218 mmol), palladium(II) acetate (1.47 g, 6.55 mmol) and dppf (4.84 g, 8.73 mmol). The reaction mixture was then stirred at 100° C. for 16 h under an atmosphere of carbon monoxide gas. The reaction mixture was concentrated under vacuum and the obtained residue was partitioned between EtOAc (40 mL) and water (40 mL). The organic layer was further washed with brine (40 mL), dried over anhydrous sodium sulfate, filtered and concentrated under vacuum to yield a brown gum which was subjected to silica gel chromatography using hexane and EtOAc as eluants to yield 24-2 (4.0 g, 17 mmol, 78% yield). MS (E⁻) m/z=232.2 (M+H)⁺.

Intermediate 24: To a solution of 24-2 (1.7 g, 7.4 mmol) in toluene (20 mL) was added 4-fluoro-3-(trifluoromethyl)aniline (1.32 g, 7.35 mmol) and bis(trimethylaluminium)-1,4-diazabicyclo[2.2.2]octane adduct (1.89 g, 7.35 mmol). The reaction mixture was then heated at 100° C. for 8 h. The reaction mixture was allowed to cool and concentrated under vacuum and the obtained residue was dissolved in saturated ammonium chloride (25 mL), EtOAc (25 mL) and 0.5 N HCl solution (15 mL) and stirred for 5 h. The organic layer was further washed with brine, dried over anhydrous sodium sulfate, filtered and concentrated under reduced pressure to provide a brown gum which was subjected to silica gel chromatography using hexane and EtOAc as eluents to yield intermediate 24 (1.1 g, 2.8 mmol, 38% yield). MS (ESI) m/z: 379.1 (M+H)⁺.

### Intermediate 25: Preparation of 5-(5-(tert-butoxycarbonyl)-3a,5,6,6a-tetrahydro-4H-pyrrolo[3,4-d]isoxazol-3-yl)-2-methoxybenzoic acid

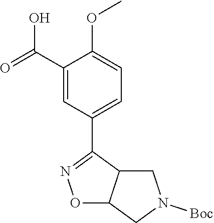 [SMILES: COc1ccc(C2=NOC3CN(C(=O)OC(C)(C)C)CC23)cc1C(=O)O]

### Intermediate 25

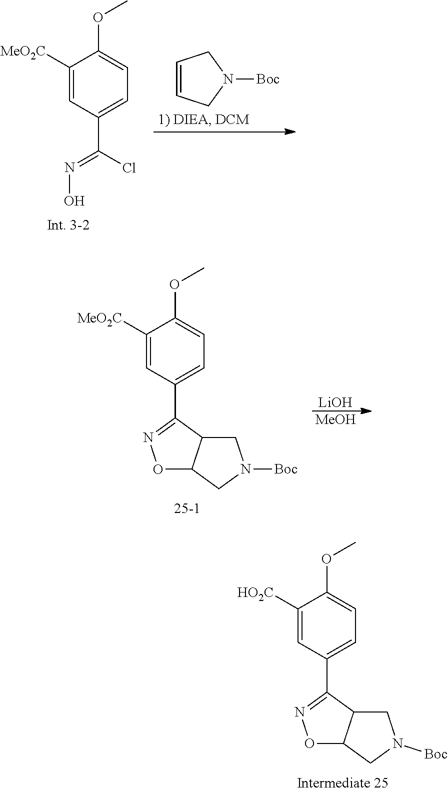

Intermediate 25-1: Preparation of tert-butyl 3-(4-methoxy-3-(methoxycarbonyl)phenyl)-3a,4,6,6a-tetrahydro-5H-pyrrolo[3,4-d]isoxazole-5-carboxylate. 25-1 (500 mg, 46% yield) was prepared in a similar manner as intermediate 3-3 by substituting tert-butyl 2,5-dihydro-1H-pyrrole-1-carboxylate for 2,5-dihydrofuran. MS (ESI) m-.: 277.1 (M+H-Boc).

Intermediate 25: To a solution of 25-1 (2.1 g, 5.6 mmol) in MeOH (20 mL) was added LiOH (0.40 g, 17 mmol) as 2N aq solution at 0° C. and the reaction mixture stirred for 3 h. The reaction mixture was concentrated under vacuum, acidified to pH 4 using 1.5N HCl and the solid was collected by filtration and dried under high vacuum to afford intermediate 25 (1.7 g, 4.7 mmol, 84% yield). MS (ESI) m/z: 361.2 (M+H)⁺.

### Intermediate 26: Preparation of 5-(1-(tert-butoxycarbonyl)pyrrolidin-3-yl)-2-methoxybenzoic acid

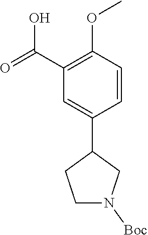 [SMILES: COc1ccc(C2CCN(C(=O)OC(C)(C)C)C2)cc1C(=O)O]

### Intermediate 26

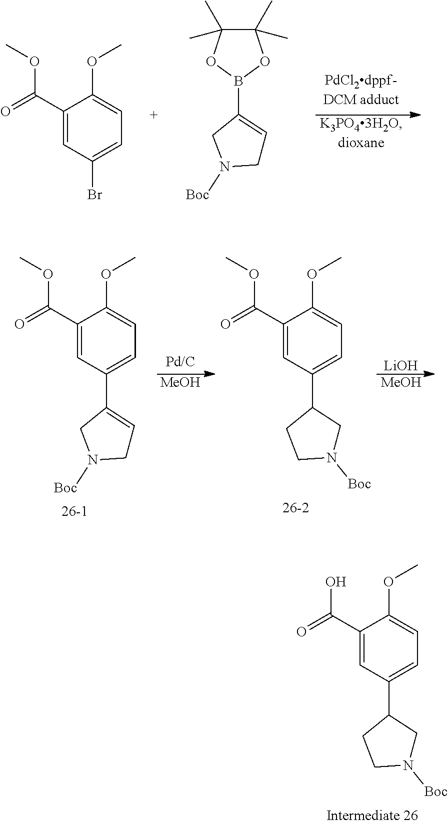

Intermediate 26-1: Preparation of tert-butyl 3-(4-methoxy-3-(methoxycarbonyl)phenyl)-2,5-dihydro-1H-pyrrole-1-carboxylate. To a degassed solution of methyl 5-bromo-2-methoxybenzoate (2 g, 8 mmol) and tert-butyl 3-(4,4,5,5-tetramethyl-1,3,2-dioxaborolan-2-yl)-2,5-dihydro-1H-pyrrole-1-carboxylate (2.65 g, 8.98 mmol) in dioxane (55 mL) and water (10 mL) was added PdCl₂(dppf)-DCM adduct (0.66 g, 0.80 mmol) and potassium phosphate tribasic monohydrate (5.64 g, 25.0 mmol). The reaction mixture was stirred at 100° C. for 4 h in a sealed tube. The reaction mixture was allowed to cool to rt and diluted with EtOAc (25 mL). The organic layers were then washed with brine (20 mL) and dried over sodium sulfate to give a residue that was subjected to silica gel chromatography using to 35% EtOAc in petroleum ether to yield 26-1 (2.2 g, 6.6 mmol, 81% yield). MS (E⁻) m/z=278.1 (M+H-tBu).

Intermediate 26-2: Preparation of tert-butyl 3-(4-methoxy-3-(methoxycarbonyl)phenyl)pyrrolidine-1-carboxylate. To a degassed solution of 26-1 (2.2 g, 6.6 mmol) in MeOH (100 mL) was added Pd—C (1.7 g, 16.5 mmol) and stirred at rt for 12 h under hydrogen balloon. The reaction mixture was then filtered over a pad of Celite® which was washed with excess MeOH. The combined layers were concentrated in vacuum to get 26-2 (1.7 g, 5.0 mmol, 77% yield). MS (E⁻) m/z=280.2 (M+H-tBu)

Intermediate 26: To a solution of 26-2 (2.0 g, 5.96 mmol) in MeOH (10 mL) and THF (10 mL) was added a solution of LiOH (1.43 g, 59.6 mmol) in H₂O (5 mL). The reaction mixture was stirred for 5h then concentrated under vacuum and acidified using HCl. The precipitated solids were then collected by filtration and dried under vacuum to yield intermediate 26 (1.5 g, 4.7 mmol, 78% yield). MS (ESI) m, z: 320.2 (M−H)⁻.

### Intermediate 27: Preparation of 5-(3-hydroxy-3-methylbut-1-yn-1-yl)-2-methoxybenzoic acid

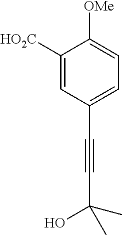 [SMILES: COc1ccc(C#CC(C)(C)O)cc1C(=O)O]

### Intermediate 27

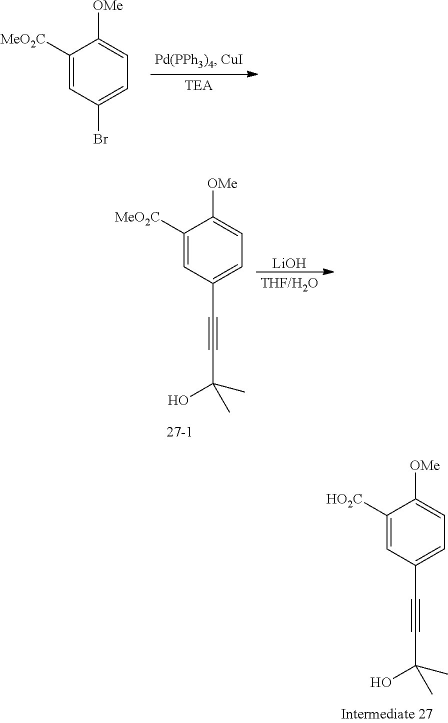

Intermediate 27-1: Preparation of methyl 5-(3-hydroxy-3-methylbut-1-yn-1-yl)-2-methoxybenzoate. A slurry of methyl 5-bromo-2-methoxybenzoate (1.7 g. 6.9 mmol), 2-methylbut-3-yn-2-ol (0.58 g, 6.9 mmol), Pd(Ph₃p)₄ (401 mg, 0.350 mmol) and CuI (13 mg, 0.070 mmol) in TEA (15 mL) was degassed, blanketed under nitrogen and heated to 80° C. for 14 h. The reaction mixture was dissolved in water (20 mL) and the solution extracted with EtOAc (20 mL). The combined organic layers were then dried over sodium sulfate and concentrated under vacuum. The residue was then subjected to silica gel chromatography using hexane and EtOAc to yield intermediate 27-1 (1.2 g, 4.83 mmol, 70% yield). MS (ESI) m/z: 248.9 (M+H)⁺.

Intermediate 27: To a solution of intermediate 27-1 (158 mg, 0.640 mmol) in THF (4.8 mL) and water (1.5 mL) was added LiOH (27 mg, 0.64 mmol) and stirred at rt for 2 h. The reaction mixture was then concentrated and acidified with IN HCl. The residue was then extracted with EtOAc (6 mL) and concentrated under reduced pressure to yield intermediate 27 (149 mg, 0.64 mmol, 100% yield). MS (EST) m/z: 248.9 (M+H)⁺.

### Intermediate 28: Preparation of 5-(5-(tert-butoxycarbonyl)-4,5,6,7-tetrahydrothiazolo[5,4-c]pyridin-2-yl)-2-methoxybenzoic acid

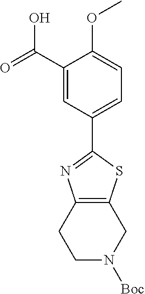 [SMILES: COc1ccc(-c2nc3c(s2)CN(C(=O)OC(C)(C)C)CC3)cc1C(=O)O]

### Intermediate 30

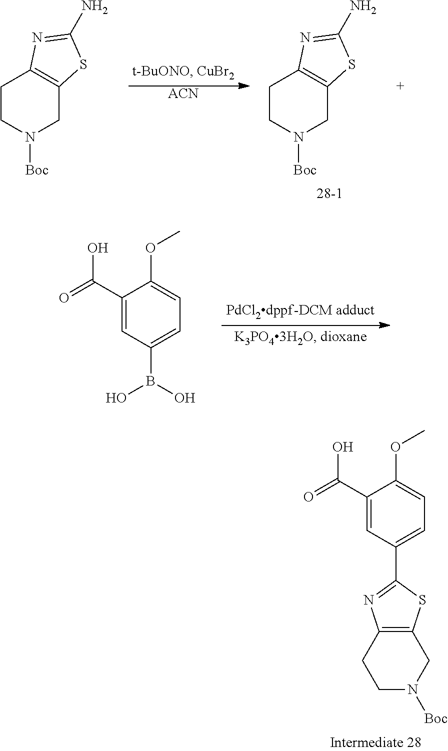 [SMILES: CC(C)(C)OC(=O)N1CCc2nc(N)sc2C1 ; "ACN" ; CC(C)(C)OC(=O)N1CCc2nc(N)sc2C1 ; COc1ccc(B(O)O)cc1C(=O)O ; COc1ccc(-c2nc3c(s2)CN(C(=O)OC(C)(C)C)CC3)cc1C(=O)O]

Intermediate 28-1: Preparation of tert-butyl 2-bromo-6,7-dihydrothiazolo[5,4-c]pyridine-5(4H)-carboxylate. To a slurry of CuBr₂ (297 mg, 1.33 mmol) in anhydrous ACN (3 mL) under argon was added tert-butyl nitrite (0.181 mL, 1.371 mmol). The reaction mixture was stirred at rt for 10 min. A suspension of tert-butyl 2-amino-6,7-dihydrothiazolo[5,4-c]pyridine-5(4H)-carboxylate (200 mg, 0.78 mmol) in anhydrous ACN (4 mL) was added dropwise. The reaction mixture was again stirred at rt for 1 h. The reaction mixture was concentrated under vacuum and the residue was diluted with EtOAc (8 mL) and 1N HCl added. The organic layer was collected, washed with 0.5N HCl (2×), sat. NaHCO₃, brine and dried over sodium sulfate. After concentration under reduced pressure, the residue was dissolved in a chloroform and purified using silica gel chromatography using hexane and EtOAc as eluents to yield 28-1 (85 mg, 0.26 mmol, 34% yield). ¹H NMR (500 MHz, CD₃CN) δ 4.56 (t, J=0.8 Hz, 2H), 3.70 (t, J=5.8 Hz, 2H), 2.81-2.76 (m, 2H), 1.47 (s, 9H).

Intermediate 28: Intermediate 30 is prepared in the same way as intermediate 26-1 by replacing methyl 5-bromo-2-methoxybenzoate with intermediate 28-1 to yield intermediate 28 (750 mg, 1.92 mmol. 38% yield). MS (ESI) m/z: 391.3 (M+H)⁺.

### Intermediate 29: Preparation of 5-(7-(tert-butoxycarbonyl)-5,6,7,8-tetrahydroimidazo[1,2-a]pyrazin-3-yl)-2-methoxybenzoic acid

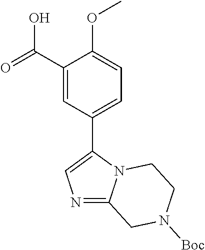 [SMILES: COc1ccc(-c2cnc3n2CCN(C(=O)OC(C)(C)C)C3)cc1C(=O)O]

### Intermediate 29

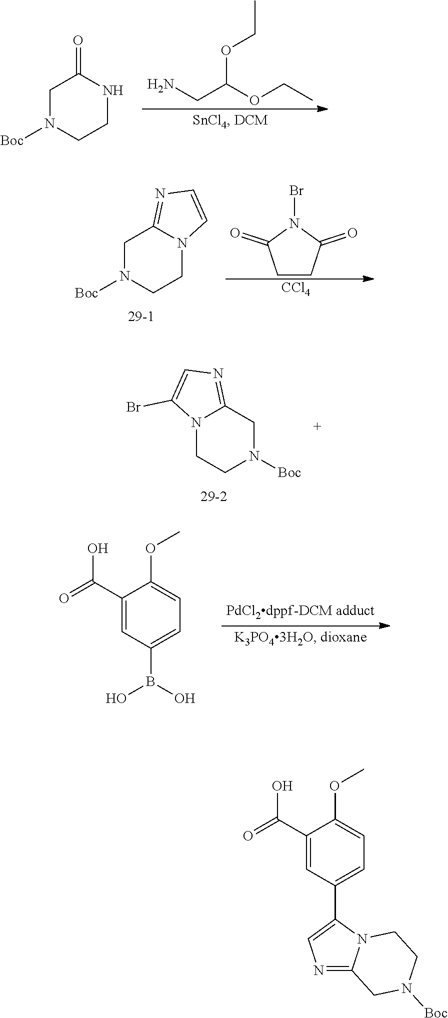

Intermediate 29-1: Preparation of tert-butyl 5,6-dihydroimidazo[1,2-a]pyrazine-7(8H)-carboxylate. A round bottom flask was charged with tert-butyl 3-oxopiperazine-1-carboxylate (5.06 g, 25.3 mmol), DCM (10 mL), mesitylene (10 mL) and tin (IV) chloride (0.89 mL, 7.6 mmol). The reaction mixture was brought to reflux then aminoacetaldehyde diethyl acetal (7.4 mL, 51 mmol) was added slowly. The reaction mixture was heated at reflux for 84 h and then allowed to cool to rt. The reaction mixture was then diluted with DCM (50 mL) and filtered through activated charcoal. The filtrate was concentrated under vacuum and subjected to silica gel chromatography using EtOAc and MeOH as eluants to yield 29-1 (0.768 g, 3.44 mmol, 14% yield). MS (EST) m/z: 224.1 (M+H)⁺.

Intermediate 29-2: Preparation of tert-butyl 3-bromo-5,6-dihydroimidazo[1,2-a]pyrazine-7(8H)-carboxylate. A round bottom flask was charged with intermediate 29-1 (768 mg, 3.44 mmol)], N-bromosuccinimide (612 mg, 3.44 mmol) and CCl₄ (10 mL). The reaction mixture was heated to reflux for 30 min and then allowed to cool to rt. The organic layers were concentrated under vacuum and the residue subjected to silica gel chromatography using hexane and EtOAc as eluents to yield 29-2 (513 mg, 1.70 mmol, 49% yield). MS (ESI) m/z: (M+H)⁺.

Intermediate 29: Intermediate 29 is prepared in the same way as intermediate 26-1 by replacing methyl 5-bromo-2-methoxybenzoate with intermediate 29-2 to yield intermediate 29 (350 mg, 0.94 mmol, 37% yield). MS (ESI) m/z: (M+H)⁺.

### Intermediate 30: Preparation of 2-methoxy-5-((oxetan-3-yloxy)methyl)benzoic acid

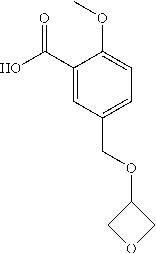 [SMILES: COc1ccc(COC2COC2)cc1C(=O)O]

### Intermediate 30

To a solution of methyl 5-(bromomethyl)-2-methoxybenzoate (20 mg, 0.080 mmol) and oxetan-3-ol (17 mg, 0.23 mmol) in DMSO (1 mL) was added KOH (13 mg, 0.23 mmol) and the reaction mixture stirred at rt for 3 h. The reaction mixture was then acidified with 1N HCl and extracted with EtOAc (4 mL). The organic layers were concentrated under vacuum to yield intermediate 30 which was used without further purification (90 mg, 0.37 mmol, 98% yield). MS (ESI) m/z: 237.2 (M+H)⁺.

### Intermediate 31: Preparation of 2-methoxy-5-(3-morpholino-1,2,4-oxadiazol-5-yl)benzoic acid

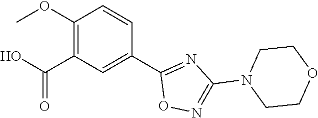 [SMILES: COc1ccc(-c2nc(N3CCOCC3)no2)cc1C(=O)O]

### Intermediate 31

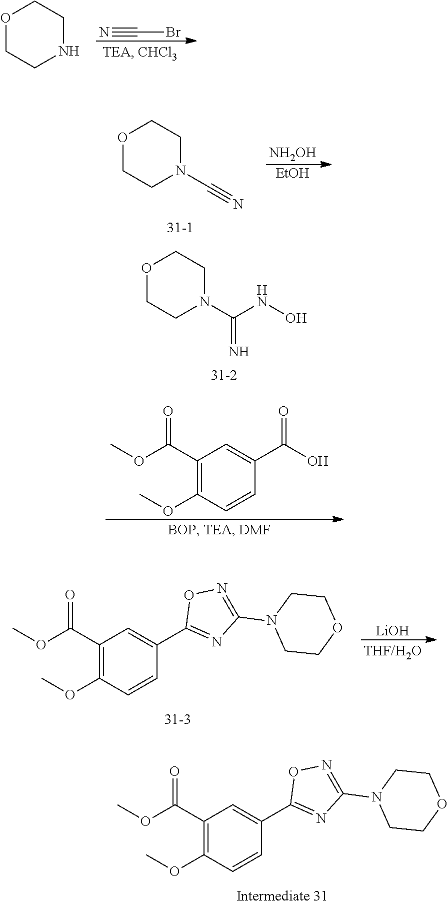

Intermediate 31-1: Preparation of morpholine-4-carbonitrile. To a stirred solution of morpholine (2 mL, 20 mmol) in CHCl₃ (20 mL) was added cyanic bromide (2.92 g, 27.5 mmol) followed by TEA (6.4 mL. 46 mmol) and the reaction mixture was stirred at rt for 12 h. The reaction mixture was then concentrated under vacuum and diluted with water. The aqueous layer was extracted with EtOAc (30 mL) and the organic layers were concentrated under vacuum to yield 31-1 which was used without further purification (2.44 g, 21.8 mmol, 95% yield. MS (ESI) m/z: 130.2 (M+18).

Intermediate 31-2: Preparation of N-hydroxymorpholine-4-carboximidamide. To a 50 mL round bottom flask was added 31-1 (250 mg, 2.23 mmol), followed by EtOH (20 mL) and hydroxylamine (295 mg, 4.46 mmol) (50% solution in water). The reaction mixture was then heated to reflux for 3 h. The cooled reaction mixture was concentrated under vacuum to yield 31-2 which was used without further purification (341 mg, 2.23 mmol, 100% yield). ¹H NMR (400 MHz. DMSO-d6) δ 8.33 (s, 1H), 5.18 (s, 2H), 3.63-3.54 (m, 4H), 2.97-2.89 (m, 4H).

Intermediate 31-3: Preparation of methyl 2-methoxy-5-(3-morpholino-1,2,4-oxadiazol-5-yl)benzoate. To a stirred solution of 4-methoxy-3-(methoxycarbonyl)benzoic acid (250 mg, 1.19 mmol) in DMF (5 mL) was added intermediate 31-2 (207 mg, 1.43 mmol) followed by BOP (1.05 g, 2.38 mmol) and TEA (0.5 mL, 4 mmol). The reaction mixture was stirred at rt for 16 h. The reaction mixture was quenched with water and extracted with EtOAc (6 mL). The organic layers were then purified by silica gel chromatography using hexane and EtOAc as eluents to yield 31-3 (300 mg, 0.94 mmol, 79% yield). LCMS (EST) m/z 320.3 (M+H)⁺.

### Intermediate 31: Preparation of 2-methoxy-5-(3-morpholino-1,2,4-oxadiazol-5-yl)benzoic acid

Intermediate 31 (160 mg, 0.52 mmol, 80% yield) was prepared in the same general way as previously described using LiOH hydrolysis protocol as described in intermediate 27. MS (ESI) m/z: 306.2 (M+H)⁺.

### Intermediate 32: Preparation of (S)-5-((3-(hydroxymethyl)morpholino)sulfonyl)-2-methoxybenzoic acid

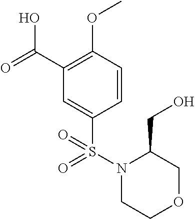 [SMILES: COc1ccc(S(=O)(=O)N2CCOC[C@@H]2CO)cc1C(=O)O]

### Intermediate 32

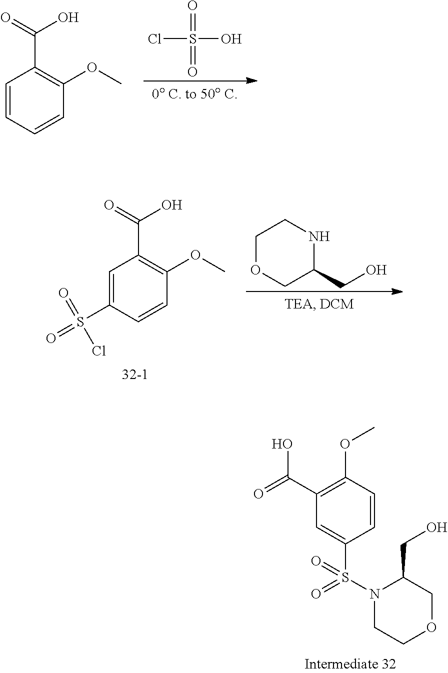

Intermediate 32-1: Preparation of 5-(chlorosulfonyl)-2-methoxybenzoic acid. 2-methoxybenzoic acid (5 g, 30 mmol) was dissolved in chlorosulfonic acid (11 mL, 160 mmol) at 0° C. The reaction mixture was heated to 50° C. for 1 h then poured to ice cold water. The precipitate formed was collected by filtration to yield intermediate 32-1 which was used without further purification (3.42 g, 41.0% yield). ¹H NMR (500 MHz, CDCl₃) δ 8.81 (d, J=2.6 Hz, 1H), 8.25 (dd, J=8.9, 2.7 Hz, 1H), 7.28 (s, 1H), 4.20 (s, 3H).

Intermediate 32: Preparation of (S)-5-((3-(hydroxymethyl)morpholino)sulfonyl)-2-methoxybenzoic acid. To a solution of intermediate 32-1 (50 mg, 0.20 mmol) in DCM (2.5 mL) at 0° C. was added (S)-morpholin-3-ylmethanol (24 mg, 0.20 mmol) followed by TEA (0.11 mL, 0.80 mmol). After completion of addition the reaction mixture was stirred at rt for 1 h. The reaction mixture was concentrated under vacuum to yield intermediate 32 which was used without further purification (66 mg, 100% yield). MS (ESI) m/z: 332.6 (M+H)⁺.

### Example 2: Preparation of N-(4-fluoro-3-(trifluoromethyl)phenyl)-5-(2-methoxy-5-(3a,4,6,6a-tetrahydrofuro[3,4-d]isoxazol-3-yl)benzamido)-2-methylbenzo[d]thiazole-6-carboxamide

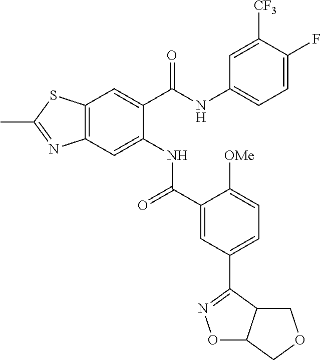 [SMILES: COc1ccc(C2=NOC3COCC23)cc1C(=O)Nc1cc2nc(C)sc2cc1C(=O)Nc1ccc(F)c(C(F)(F)F)c1]

To a mixture of intermediate 1 (2.53 mg, 6.84 μmol), intermediate 3-6 (1.8 mg, 6.84 μmol), and TCFH (1.9 mg, 6.84 μmol) was added ACN (1 mL) and 1-methyl-1H-imidazole (0.5 mg, 6.84 μmol). After 24 h, the reaction mixture was purified by reverse phase HPLC (5:95 ACN, water with 0.05% TFA to 95:5 ACN, water with 0.05% TFA) to afford example 2 (2.6 mg, 4.2 μmol, 62% yield). ¹H NMR (500 MHz, DMSO-d6) δ 11.73-11.50 (m, 1H), 11.02 (s, 1H), 9.08 (s, 1H), 8.51 (s, 1H), 8.38-8.29 (m, 1H), 8.11-8.02 (m, 1H), 7.91-7.83 (m, 1H), 7.57 (br t, J=9.8 Hz, 1H), 7.33 (br d, J=8.9 Hz, 1H), 5.41-5.33 (m, 1H), 4.55 (br d, J=8.1 Hz, 1H), 4.11 (br d, J=10.8 Hz, 1H), 4.06 (s, 3H), 3.94-3.86 (m, 1H), 3.83-3.77 (m, 1H), 3.68-3.65 (m, 1H), 3.17 (d, J=5.2 Hz, 1H), 2.86 (s, 3H); MS (ESI) m/z: 615.1 (M+H)-; HPLC purity 100% with retention time 2.2 min.

### Method B

Compounds listed in Table-1 are compounds of this invention that were prepared, purified and isolated according to the methods described above.

TABLE 1

| | | | | | LC RT |
|---|---|---|---|---|---|
| Ex. | | | MS (ESI) | | (min) |
| No. | Structure | Name | (M + H)⁺ | ¹H NMR | Meth. |
| 3 | 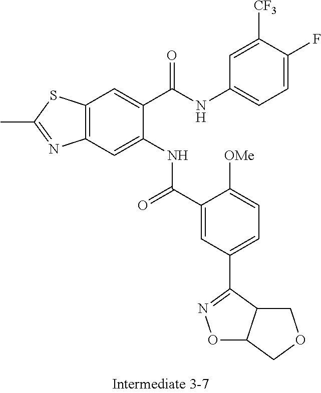 [SMILES: COc1ccc(C2=NOC3COCC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (2-methoxy-5- (3a,4,6,6a- tetrahydrofuro [3,4-d]isox- azol-3-yl)- benzamido)- 2-methylbenzo [d]thiazole-6- carboxamide | 615.3 | ¹H NMR (400 MHz, DMSO- d₆) δ 11.89- 11.49 (m, 1H), 11.02 (s, 1H), 9.11 (s, 1H), 8.54 (s, 1H), 8.40-8.24 (m, 2H), 8.06 (td, J = 4.7, 3.0 Hz, 1H), 7.88 (dd, J = 8.6, 2.3 Hz, 1H), 7.57 (t, J = 9.7 Hz, 1H), 7.34 (d, J = 8.8 Hz, 1H), 5.37 (dd, J = 9.3, 3.4 Hz, 1H), 4.56 (dd, J = 8.6, 6.7 Hz, 1H), 4.12 (d, J = 10.8 Hz, 1H), 4.06 (s, 3H), 3.92 (s, 1H), 3.83-3.77 | 2.22 C |
| | | | | (m, 1H), 3.71- | |
| | | | | 3.64 (m, 1H), | |
| | | | | 2.87 (s, 3H) | |
| 4 | 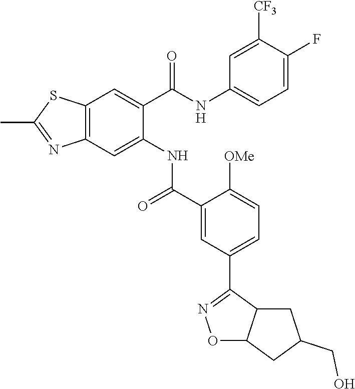 [SMILES: COc1ccc(C2=NOC3CC(CO)CC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-(hy- droxymethyl)- 3a,5,6,6a-tetra- hydro-4H- cyclopenta[d] isoxazol-3-yl)- 2-methoxy- benzamido)-2- methylbenzo [d]thiazole-6- carboxamide | 643.2 | ¹H NMR (500 MHz, CD₃OD) δ 11.81 (s, 1H), 10.68 (s, 1H), 9.19 (d, J = 2.7 Hz, 1H), 8.46 (d, J = 2.4 Hz, 1H), 8.39 (s, 1H), 8.30 (dd, J = 6.1, 2.7 Hz, 1H), 8.06-7.97 (m, 1H), 7.95 (dd, J = 8.8, 2.4 Hz, 1H), 7.39 (t, J = 9.6 Hz, 1H), 7.30 (d, J = 8.9 Hz, 1H), 5.23 (dd, J = 8.6, 5.0 Hz, 1H), 4.37- 4.25 (m, 1H), 4.13 (s, 3H), 3.61-3.51 (m, 3H), 2.90 (s, 3H), 2.25-2.17 | 2.07 C |
| | | | | (m, 1H), 2.13 | |
| | | | | (dd, J = 11.7, 5.8 | |
| | | | | Hz, 1H), 2.07- | |
| | | | | 2.00 (m, 1H), | |
| | | | | 1.88-1.76 (m, | |
| | | | | 1H), 1.67 (ddd, | |
| | | | | J = 13.4, 11.7, | |
| | | | | 5.0 Hz, 1H) | |
| 5 | 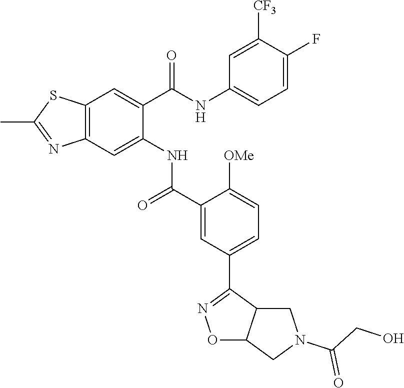 [SMILES: COc1ccc(C2=NOC3CN(C(=O)CO)CC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-(2-hy- droxyacetyl)- 3a,5,6,6a-tetra- hydro-4H-pyr- rolo[3,4-d] isoxazol-3-yl)- 2-methoxy- benzamido)-2- methylbenzo [d]thiazole-6- carboxamide | 672.2 | ¹H NMR (500 MHz, DMSO- d₆) δ 9.24-9.01 (m, 1H), 8.51 (s, 1H), 8.41-8.23 (m, 2H), 8.16- 8.02 (m, 1H), 7.94-7.85 (m, 1H), 7.61-7.54 (m, 1H), 7.34 (d, J = 8.8 Hz, 1H), 5.49-5.27 (m, 1H), 4.79- 4.49 (m, 1H), 4.07 (s, 3H), 4.01-3.77 (m, 3H), 3.72-3.58 (m, 1H), 3.56- 3.46 (m, 5H), 2.86 (s, 3H) | 2.04 B |
| 6 | 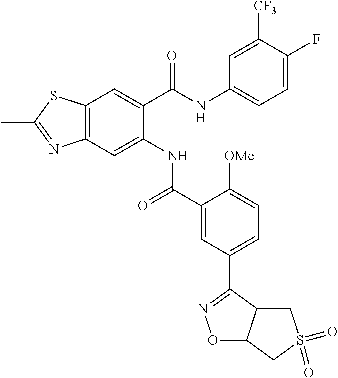 [SMILES: COc1ccc(C2=NOC3CS(=O)(=O)CC23)cc1C(=O)Nc1cc2nc(C)sc2cc1C(=O)Nc1ccc(F)c(C(F)(F)F)c1] | 5-(5-(5,5-diox- ido-3a,4,6,6a- tetrahydro- thieno[3,4-d] isoxazol-3-yl)- 2-methoxy- benzamido)-N- (4-fluoro-3- (trifluorometh- yl)phenyl)-2- methylbenzo [d]thiazole-6- carboxamide | 663.08 | ¹H NMR (500 MHz, DMSO- d₆) δ 11.75- 11.54 (m, 1H), 11.01 (s, 1H), 9.26-8.90 (m, 1H), 8.52-8.43 (m, 1H), 8.32 (br s, 2H), 8.09- 8.00 (m, 1H), 7.83 (dd, J = 8.5, 2.1 Hz, 1H), 7.55 (br t, J = 9.7 Hz, 1H), 7.33 (d, J = 8.7 Hz, 1H), 5.44 (br dd, J =10.7, 6.7 Hz, 1H), 4.79 (td, J = 10.3, 3.2 Hz, 1H), 4.05 (s, 3H), 3.19 (brs, 2H), 3.17 (s, | 2.1 C |
| | | | | 2H), 2.85 (s, | |
| | | | | 3H) | |
| 7 | 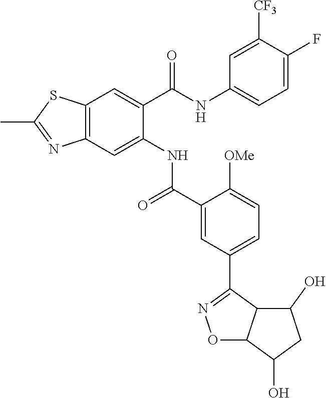 [SMILES: COc1ccc(C2=NOC3C(O)CC(O)C23)cc1C(=O)Nc1cc2nc(C)sc2cc1C(=O)Nc1ccc(F)c(C(F)(F)F)c1] | 5-(5-(4,6- dihydroxy- 3a,5,6,6a-tetra- hydro-4H- cyclopenta[d] isoxazol-3-yl)- 2-methoxy- benzamido)-N- (4-fluoro-3- (trifluorometh- yl)phenyl)-2- methylbenzo [d]thiazole-6- carboxamide | 645.0 | ¹H NMR (500 MHz, DMSO- d₆) δ 11.64 (s, 1H), 11.03 (s, 1H), 9.08 (s, 1H), 8.50 (br s, 1H), 8.33 (br d, J = 4.6 Hz, 1H), 8.10-8.03 (m, 1H), 8.00-7.91 (m, 1H), 7.64- 7.46 (m, 1H), 7.46-7.17 (m, 1H), 5.04-4.92 (m, 1H), 4.15- 4.09 (m, 1H), 4.06 (s, 3H), 3.66-3.49 (m, 3H), 2.87 (s, 3H), 1.95-1.83 (m, 1H), 1.77- 1.66 (m, 1H) | 2.12 B |
| 8 | 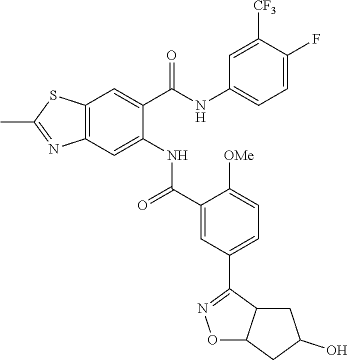 [SMILES: COc1ccc(C2=NOC3CC(O)CC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-hydroxy- 3a,5,6,6a-tetra- hydro-4H- cyclopenta[d] isoxazol-3-yl)- 2-methoxy- benzamido)-2- methylbenzo [d]thiazole-6- carboxamide | 627.2 | ¹H NMR (500 MHz, DMSO- d6) δ 11.65 (s, 1H), 11.01 (s, 1H), 9.26-8.93 (m, 1H), 8.54- 8.45 (m, 1H), 8.33 (br d, J = 2.1 Hz, 2H), 8.15-8.01 (m, 1H), 7.84 (dd, J = 8.7, 1.9 Hz, 1H), 7.56 (br t, J = 9.7 Hz, 1H), 7.31 (d, J = 8.8 Hz, 1H), 5.22- 5.06 (m, 1H), 4.51 (br d, J = 2.8 Hz, 1H), 4.21-4.09 (m, 2H), 4.05 (s, 3H), 2.86 (s, | 2.06 B |
| | | | | 3H), 2.18-2.03 | |
| | | | | (m, 2H), 1.91 | |
| | | | | (br d, J = 14.9 | |
| | | | | Hz, 1H), 1.76 | |
| | | | | (br d, J = 13.4 | |
| | | | | Hz, 1H) | |
| 9 | 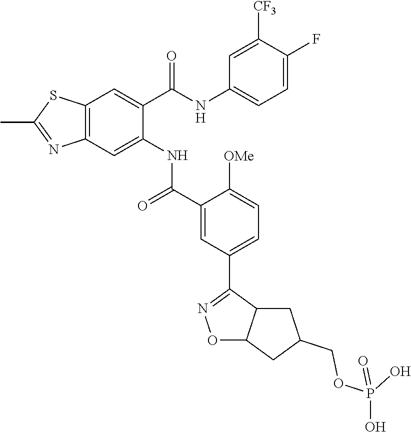 [SMILES: COc1ccc(C2=NOC3CC(COP(=O)(O)O)CC23)cc1C(=O)Nc1cc2nc(C)sc2cc1C(=O)Nc1ccc(F)c(C(F)(F)F)c1] | (3-(3-((6-((4- fluoro-3- (trifluorometh- yl)phenyl)car- bamoyl)-2- methylbenzo [d]thiazol-5- yl)carbamo- yl)-4-meth- oxyphenyl)- 3a,5,6,6a-tetra- hydro-4H- cyclopenta[d] isoxazol-5- yl)methyldi- hydrogen phosphate | 723.1 | ¹H NMR (500 MHz, DMSO- d₆) δ 11.67 (s, 1H), 11.03 (s, 1H), 9.12 (s, 1H), 8.53 (s, 1H), 8.38-8.29 (m, 2H), 8.16- 8.02 (m, 1H), 7.88 (dd, J = 8.7, 2.4 Hz, 1H), 7.59 (t, J = 9.8 Hz, 1H), 7.34 (d, J = 8.9 Hz, 1H), 5.17 (dd, J = 8.8, 5.1 Hz, 1H), 4.30 (br t, J = 8.7 Hz, 1H), 4.07 (s, 3H), 3.89-3.78 (m, 3H), 2.88 (s, 3H), 2.18-2.03 (m, 3H), 1.92- 1.86 (m, 1H), 1.78-1.72 (m, 1H), 1.66-1.59 (m, 1H) | 1.93 C |
| 10 | 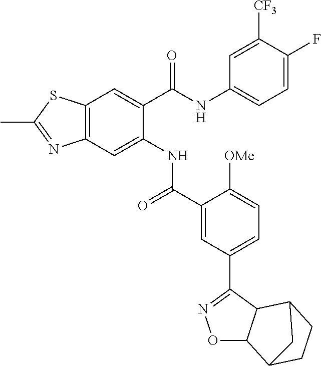 [SMILES: COc1ccc(C2=NOC3C4CCC(C4)C23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(3a,4,5,6,7, 7a-hexahydro- 4,7-methano- benzo[d]isox- azol-3-yl)-2- methoxybenz- amido)-2- methylbenzo [d]thiazole-6- carboxamide | 637.0 | ¹H NMR (500 MHz, DMSO- d6) δ 11.65 (br s, 1H), 11.02 (br s, 1H), 9.09 (br s, 1H), 8.67-8.46 (m, 1H), 8.35 (br s, 2H), 8.07 (br dd, J = 4.6, 2.4 Hz, 1H), 7.89 (br d, J = 8.2 Hz, 1H), 7.57 (br t, J = 9.6 Hz, 1H), 7.39-7.12 (m, 1H), 4.61 (br d, J = 8.2 Hz, 1H), 4.06 (br s, 3H), 3.74-3.60 (m, 1H), 3.48 (br d, J = 10.4 Hz, 1H), 2.87 (br s, 3H), 2.40 (br s, | 2.55 B |
| | | | | 1H), 1.51 (br d, | |
| | | | | J = 1.5 Hz, 2H), | |
| | | | | 1.44-1.35 (m, | |
| | | | | 1H), 1.29 (br d, | |
| | | | | J = 9.2 Hz, 1H), | |
| | | | | 1.24-1.09 (m, | |
| | | | | 2H) | |
| 11 | 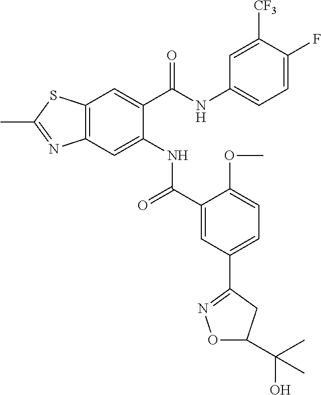 [SMILES: COc1ccc(C2=NOC(C(C)(C)O)C2)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-(2- hydroxypro- pan-2-yl)-4,5- dihydroisox- azol-3-yl)-2- methoxybenz- amido)-2- methylbenzo [d]thiazole-6- carboxamide | 629.2 | ¹H NMR (500 MHz, DMSO- d6) δ 9.08 (s, 1H), 8.49 (s, 1H), 8.37-8.20 (m, 2H), 8.14- 7.95 (m, 1H), 7.86 (dd, J = 8.7, 2.0 Hz, 1H), 7.56 (br t, J = 9.9 Hz, 1H), 7.32 (d, J = 8.9 Hz, 1H), 4.53- 4.39 (m, 1H), 4.05 (s, 3H), 3.60-3.45 (m, 1H), 3.42-3.28 (m, 1H), 2.86 (s, 3H), 2.56 (s, 2H), 1.12 (br s, 3H), 1.12 (br s, 3H) | 2.17 B |
| 12 | 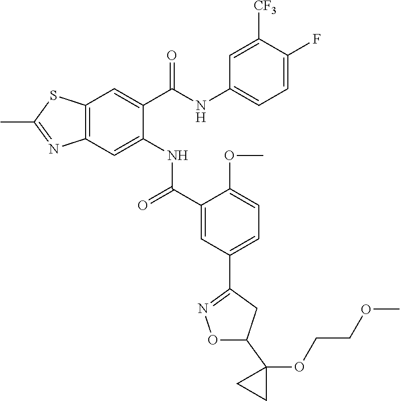 | N-(4-fluoro-3- (trifluorometh- yl)phenyl)-5- (2-methoxy-5- (5-(1-(2-meth- oxyethoxy)- cyclopropyl)- 4,5-dihydro- isoxazol-3-yl)- benzamido)- 2-methylbenzo [d]thiazole-6- carboxamide | 687.3 | ¹H NMR (500 MHz, DMSO-d₆) δ 11.67 (s, 1H), 11.03 (s, 1H), 9.11 (s, 1H), 8.54 (s, 1H), 8.39- 8.32 (m, 2H), 8.13-8.05 (m, 1H), 7.89 (dd, J = 8.6, 2.4 Hz, 1H), 7.59 (t, J = 9.6 Hz, 1H), 7.34 (d, J = 8.9 Hz, 1H), 4.59 (dd, J = 10.8, 8.4 Hz, 1H), 4.07 (s, 3H), 3.75-3.68 (m, 1H), 3.66- 3.60 (m, 1H), 3.59-3.50 (m, 4H), 3.14 (s, 3H), 2.88 (s, 3H), 0.98-0.89 (m, 1H), 0.83- 0.75 (m, 1H), | 2.34 C |
| | | | | 0.70 (s, 2H) | |
| 13 | 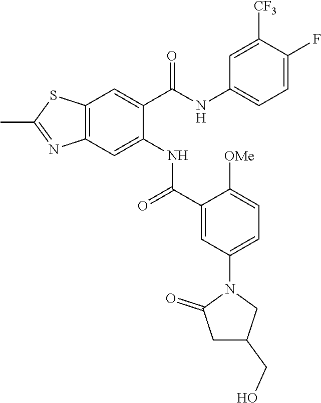 [SMILES: COc1ccc(N2CC(CO)CC2=O)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(4-(hy- droxymethyl)- 2-oxopyrroli- din-1-yl)-2- methoxybenz- amido)-2- methylbenzo [d]thiazole-6- carboxamide | 617.0 | ¹H NMR (500 MHz, DMSO- d₆) δ 11.67 (s, 1H), 11.04 (s, 1H), 9.11 (s, 1H), 8.51 (s, 1H), 8.36 (br d, J = 3.7 Hz, 1H), 8.29 (d, J = 2.4 Hz, 1H), 8.13- 8.03 (m, 1H), 7.88 (dd, J = 9.2, 2.4 Hz, 1H), 7.58 (br t, J = 9.8 Hz, 1H), 7.26 (br d, J = 8.9 Hz, 1H), 4.01 (s, 3H), 3.93 (br t, J = 8.7 Hz, 1H), 3.66 (br dd, J = 9.2, 5.5 Hz, 1H), 3.55-3.25 (m, 1H), 3.01 (s, 1H), 2.88 (s, | 1.98 C |
| | | | | 3H), 2.69-2.58 | |
| | | | | (m, 2H), 2.32 | |
| | | | | (br dd, J = 16.0, | |
| | | | | 5.0 Hz, 1H), | |
| | | | | 1.18 (br t, J = | |
| | | | | 7.2 Hz, 1H) | |
| 14 | 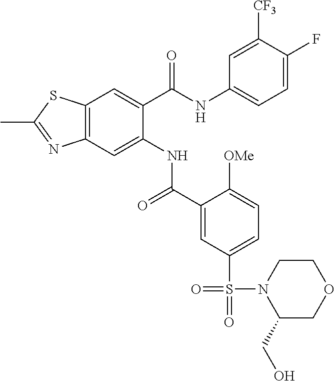 [SMILES: COc1ccc(S(=O)(=O)N2CCOC[C@@H]2CO)cc1C(=O)Nc1cc2nc(C)sc2cc1C(=O)Nc1ccc(F)c(C(F)(F)F)c1] | (S)-N-(4- fluoro-3-(tri- fluoromethyl)- phenyl)-5-(5- ((3-(hydroxy- methyl)morph- olino)sulfon- yl)-2-methoxy- benzamido)-2- methylbenzo [d]thiazole-6- carboxamide | 681.2 | ¹H NMR (500 MHz, DMSO- d₆) δ 11.87- 11.64 (m, 1H), 11.21-10.89 (m, 1H), 9.22-9.01 (m, 1H), 8.57 (s, 1H), 8.49 (d, J = 1.8 Hz, 1H), 8.37 (br d, J = 4.0 Hz, 1H), 8.10 (br dd, J = 4.9, 3.7 Hz, 1H), 8.07-8.01 (m, 1H), 7.60 (br t, J = 9.8 Hz, 1H), 7.54-7.35 (m, 1H), 4.15 (s, 3H), 3.88 (br d, J = 11.6 Hz, 1H), 3.82 (s, 1H), 3.71 (br d, J = 8.2 Hz, 1H), 3.69-3.59 (m, | 2.08 B |
| | | | | 1H), 3.49 (br d, | |
| | | | | J = 10.4 Hz, | |
| | | | | 1H), 3.42-3.31 | |
| | | | | (m, 1H), 3.23 | |
| | | | | (br d, J = 12.2 | |
| | | | | Hz, 1H), 3.18 | |
| | | | | (br d, J = 10.4 | |
| | | | | Hz, 1H), 2.90 (s, | |
| | | | | 3H), 1.44-1.02 | |
| | | | | (m, 2H) | |
| 15 | 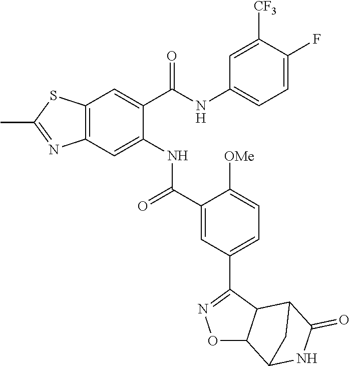 [SMILES: COc1ccc(C2=NOC3C4CC(C(=O)N4)C23)cc1C(=O)Nc1cc2nc(C)sc2cc1C(=O)Nc1ccc(F)c(C(F)(F)F)c1] | N-(4-fluoro- 3-(trifluoro- methyl)phen- yl)-5-(2-meth- oxy-5-(5-oxo- 3a,4,5,6,7,7a- hexahydro- 4,7-methano- isoxazolo[5,4- c]pyridin-3- yl)benzami- do)-2-methyl- benzo[d]thi- azole-6-car- boxamide (Diastereo- meric mixture) | 654.1 | ¹H NMR (500 MHz, DMSO- d₆) δ 11.66 (br d, J = 3.4 Hz, 1H), 11.02 (s, 1H), 9.09 (br s, 1H), 8.50 (br d, J = 2.7 Hz, 1H), 8.38 (dd, J = 10.9, 1.8 Hz, 1H), 8.34-8.28 (m, 1H), 8.06 (br dd, J = 4.8, 3.7 Hz, 1H), 7.96-7.90 (m, 1H), 7.89-7.83 (m, 1H), 7.56 (br t, J = 9.7 Hz, 1H), 7.35 (br d, J = 8.8 Hz, 1H), 7.29-6.86 (m, 2H), 5.28-4.83 (m, 1H), 4.31 | 2.12 C |
| | | | | (br d, J = 8.2 Hz, | |
| | | | | 1H), 4.12-4.00 | |
| | | | | (m, 3H), 2.86 (s, | |
| | | | | 3H), 1.90 (br t, | |
| | | | | J = 9.8 Hz, 1H), | |
| | | | | 1.70-1.37 (m, | |
| | | | | 1H) | |
| 16 | 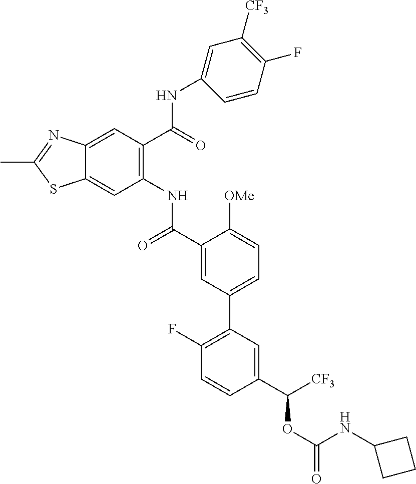 [SMILES: COc1ccc(-c2cc([C@H](OC(=O)NC3CCC3)C(F)(F)F)ccc2F)cc1C(=O)Nc1cc2sc(C)nc2cc1C(=O)Nc1ccc(F)c(C(F)(F)F)c1] | (S)-2,2,2- trifluoro-1-(6- fluoro-3′-((5- ((4-fluoro-3- (trifluorometh- yl)phenyl)car- bamoyl)-2- methylbenzo [d]thiazol-6- yl)carbamoyl)- 4′-methoxy- [1,1′-biphen- yl]-3-ylethyl cyclobutylcar- bamate | 793.27 | ¹H NMR (500 MHz, DMSO- d₆) δ 9.34-9.08 (m, 1H), 8.44- 8.33 (m, 2H), 8.22 (br s, 1H), 8.16 (br d, J = 7.6 Hz, 1H), 8.13-8.06 (m, 1H), 7.82-7.75 (m, 1H), 7.72 (br d, J = 7.3 Hz, 1H), 7.63-7.54 (m, 2H), 7.49- 7.42 (m, 1H), 7.39 (s, 1H), 6.48-6.25 (m, 1H), 4.08 (s, 3H), 4.00-3.89 (m, 1H), 3.01 (s, 1H), 2.85 (s, 3H), 2.25-2.05 (m, 2H), 2.00- 1.80 (m, 2H), 1.71-1.50 (m, 2H), 1.23 (br s, 1H) | 2.82 B |
| 19 | 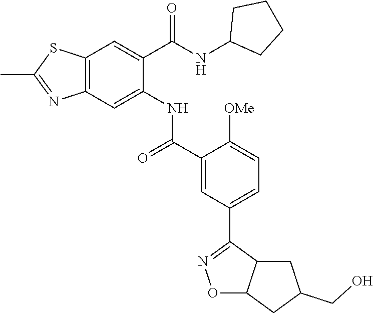 [SMILES: COc1ccc(C2=NOC3CC(CO)CC23)cc1C(=O)Nc1cc2nc(C)sc2cc1C(=O)NC1CCCC1] | N-cyclopent- yl-5-(5-(5- (hydroxymeth- yl)-3a,5,6,6a- tetrahydro-4H- cyclopenta[d] isoxazol-3-yl)- 2-methoxy- benzamido)-2- methylbenzo [d]thiazole-6- carboxamide | 549.3 | ¹H NMR (500 MHz, CD₃OD) δ 9.15 (s, 1H), 8.43 (d, J = 2.4 Hz, 1H), 8.15 (s, 1H), 7.96 (dd, J = 8.7, 2.3 Hz, 1H), 7.32 (d, J = 8.9 Hz, 1H), 5.24 (dd, J = 8.7, 5.0 Hz, 1H), 4.42 (t, J = 6.7 Hz, 1H), 4.29 (t, J = 8.6 Hz, 1H), 4.19 (s, 3H), 3.68- 3.45 (m, 3H), 2.88 (s, 3H), 2.31-2.12 (m, | 1.78 C |
| | | | | 2H), 2.11-1.98 | |
| | | | | (m, 4H), 1.88- | |
| | | | | 1.76 (m, 4H), | |
| | | | | 1.73-1.51 (m, | |
| | | | | 5H) | |
| 21 | 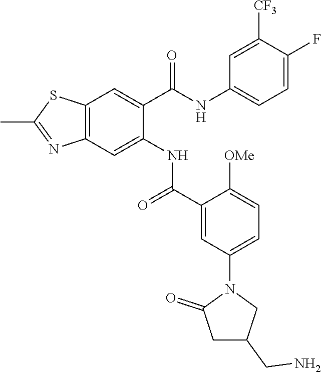 [SMILES: COc1ccc(N2CC(CN)CC2=O)cc1C(=O)Nc1cc2nc(C)sc2cc1C(=O)Nc1ccc(F)c(C(F)(F)F)c1] | 5-(5-(4-(ami- nomethyl)-2- oxopyrrolidin- 1-yl)-2-meth- oxybenzami- do)-N-(4-fluoro-3-(tri- fluoromethyl)- phenyl)-2- methylbenzo [d]thiazole-6- carboxamide, TFA | 616.1 | ¹H NMR (500 MHz, DMSO- d₆) δ 9.08 (s, 1H), 8.50 (s, 1H), 8.35 (br d, J = 4.0 Hz, 1H), 8.11 (s, 1H), 8.06 (br d, J = 8.5 Hz, 1H), 7.62 (br d, J = 8.5 Hz, 1H), 7.59-7.51 (m, 1H), 7.33-7.23 (m, 1H), 4.26 (br d, J = 2.1 Hz, 1H), 4.02 (s, 3H), 3.64- 3.51 (m, 1H), 2.86 (s, 3H), 2.70-2.59 (m, 2H), 2.45-2.35 (m, 1H), 2.29- 2.16 (m, 1H), | 1.79 B |
| | | | | 2.06-1.97 (m, | |
| | | | | 1H), 1.90 (s, | |
| | | | | 3H) (One proton | |
| | | | | is not visible in | |
| | | | | the NMR, likely | |
| | | | | due to overlap | |
| | | | | with the solvent | |
| | | | | peak) | |
| 22 | 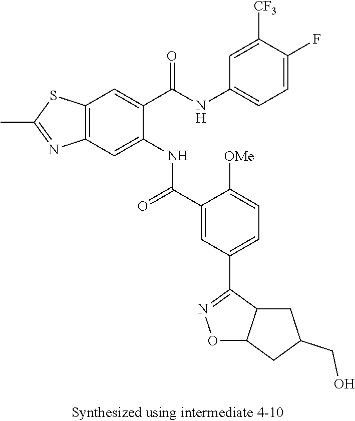 [SMILES: COc1ccc(C2=NOC3CC(CO)CC23)cc1C(=O)Nc1cc2nc(C)sc2cc1C(=O)Nc1ccc(F)c(C(F)(F)F)c1] | N-(4-fluoro-3- (trifluoro- methyl)phen- yl)-5-(5-(5- (hydroxymeth- yl)-3a,5,6,6a- tetrahydro-4H- cyclopenta[d] isoxazol-3-yl)- 2-methoxy- benzamido)-2- methylbenzo [d]thiazole-6- carboxamide | 642.9 | ¹H NMR (500 MHz, DMSO- d₆) δ 11.68 (s, 1H), 11.03 (s, 1H), 9.11 (s, 1H), 8.53 (s, 1H), 8.37 (br s, 2H), 8.14-8.03 (m, 1H), 7.88 (dd, J = 9.0, 1.4 Hz, 1H), 7.59 (br t, J = 9.8 Hz, 1H), 7.34 (d, J = 8.5 Hz, 1H), 5.18 (br dd, J = 9.5, 6.4 Hz, 1H), 4.24-4.12 (m, 1H), 4.07 (s, 3H), 3.47-3.36 (m, 1H), 3.34- 3.30 (m, 1H), 3.28-3.19 (m, 1H), 2.88 (s, 3H), 2.33-2.22 (m, 1H), 2.20- 2.06 (m, 2H), | 2.13 C |
| | | | | 1.71-1.58 (m, | |
| | | | | 1H), 1.50 (dt, | |
| | | | | J = 12.4, 6.3 Hz, | |
| | | | | 1H | |
| 23 | 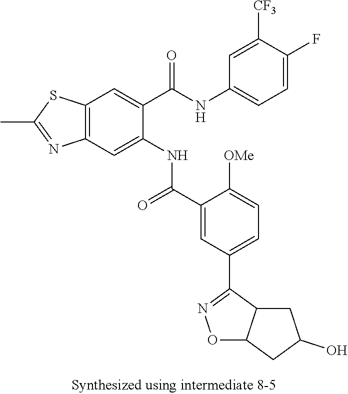 [SMILES: COc1ccc(C2=NOC3CC(O)CC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-hydroxy- 3a,5,6,6a- tetrahydro- 4H-cyclopenta [d]isoxazol-3- yl)-2-methoxy- benzamido)-2- methylbenzo [d]thiazole-6- carboxamide | 628.9 | ¹H NMR (500 MHz, DMSO- d₆) δ 11.65 (s, 1H), 11.01 (s, 1H), 9.08 (s, 1H), 8.55-8.43 (m, 1H), 8.32 (br s, 2H), 8.14- 8.02 (m, 1H), 7.84 (dd, J = 8.7, 2.0 Hz, 1H), 7.56 (br t, J = 9.6 Hz, 1H), 7.31 (d, J = 8.6 Hz, 1H), 5.19- 5.03 (m, 1H), 4.51 (br s, 1H), 4.23-4.11 (m, 2H), 4.04 (s, 3H), 2.85 (s, 3H), 2.18-2.03 (m, 2H), 1.91 (br d, J = 13.7 Hz, 1H), 1.76 | 2.07 B |
| | | | | (br d, J = 13.2 | |
| | | | | Hz, 1H) | |
| 24 | 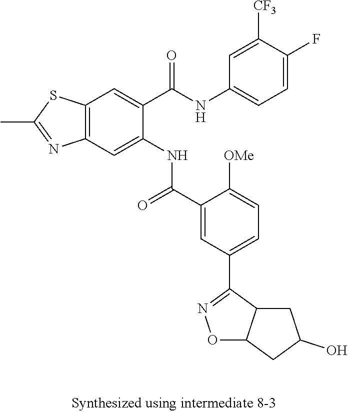 [SMILES: COc1ccc(C2=NOC3CC(O)CC23)cc1C(=O)Nc1cc2nc(C)sc2cc1C(=O)Nc1ccc(F)c(C(F)(F)F)c1] | N-(4-fluoro- 3-(trifluoro- methyl)phen- yl)-5-(5-(5- hydroxy- 3a,5,6,6a- tetrahydro- 4H-cyclopenta [d]isoxazol-3- yl)-2-methoxy- benzamido)- 2-methylbenzo [d]thiazole-6- carboxamide | 627.2 | ¹H NMR (500 MHz, DMSO- d₆) δ 11.80- 11.53 (m, 1H), 11.02 (s, 1H), 9.24-8.93 (m, 1H), 8.55-8.47 (m, 1H), 8.39- 8.27 (m, 2H), 8.14-8.02 (m, 1H), 7.85 (dd, J = 8.7, 1.7 Hz, 1H), 7.65-7.50 (m, 1H), 7.33 (d, J = 8.7 Hz, 1H), 5.23-5.11 (m, 1H), 4.35- 4.20 (m, 1H), 4.17-4.08 (m, 1H), 4.05 (s, 3H), 2.86 (s, 3H), 2.04-1.86 (m. 3H), 1.85- 1.72 (m, 1H), 1.62-1.60 (m, 1H) | 2.08 B |
| 26 | 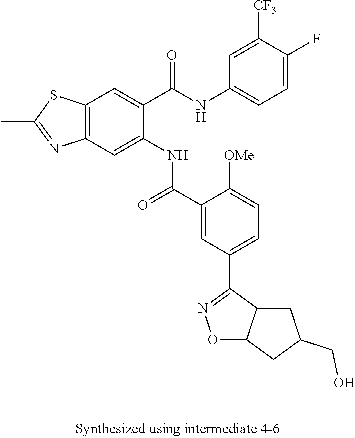 [SMILES: COc1ccc(C2=NOC3CC(CO)CC23)cc1C(=O)Nc1cc2nc(C)sc2cc1C(=O)Nc1ccc(F)c(C(F)(F)F)c1] | N-(4-fluoro- 3-(trifluoro- methyl)phen- yl)-5-(5-(5- (hydroxymeth- yl)-3a,5,6,6a- tetrahydro-4H- cyclopenta[d] isoxazol-3-yl)- 2-methoxy- benzamido)-2- methylbenzo [d]thiazole-6- carboxamide | 643.3 | ¹H NMR (500 MHz, DMSO- d₆) δ 11.65 (s, 1H), 11.01 (s, 1H), 9.08 (s, 1H), 8.49 (s, 1H), 8.33 (s, 2H), 8.09-8.01 (m, 1H), 7.86 (dd, J = 8.8. 2.2 Hz, 1H), 7.56 (br t, J = 9.7 Hz, 1H), 7.32 (d, J = 8.9 Hz, 1H), 5.23-5.08 (m, 1H), 4.19-4.09 (m, 1H), 4.07- 3.99 (m, 3H), 3.79-3.64 (m, 1H), 3.30 (dt, J = 10.2, 5.1 Hz, 1H), 3.26-3.18 (m, 1H), 2.92- 2.82 (m, 3H), 2.31-2.19 (m, 1H), 2.18-2.06 (m, 2H), 1.70- | 2.15 B |
| | | | | 1.60 (m, 1H), | |
| | | | | 1.53-1.39 (m, | |
| | | | | 1H) | |
| 27 | 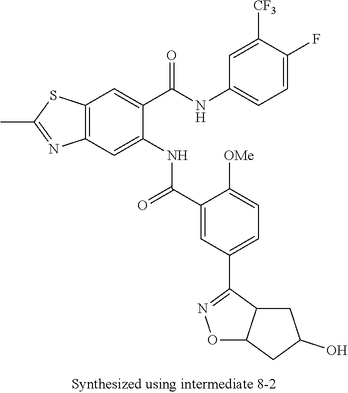 [SMILES: COc1ccc(C2=NOC3CC(O)CC23)cc1C(=O)Nc1cc2nc(C)sc2cc1C(=O)Nc1ccc(F)c(C(F)(F)F)c1] | N-(4-fluoro-3- (trifluorometh- yl)phenyl)-5- (5-(5-hydroxy- 3a,5,6,6a-tetra- hydro-4H- cyclopenta[d] isoxazol-3-yl)- 2-methoxy- benzamido)-2- methylbenzo [d]thiazole-6- carboxamide | 629.1 | ¹H NMR (500 MHz, DMSO- d₆) δ 11.80- 11.52 (m, 1H), 11.15-10.88 (m, 1H), 9.08 (s, 1H), 8.50 (s, 1H), 8.36-8.24 (m, 1H), 8.14- 7.99 (m, 1H), 7.94-7.72 (m, 1H), 7.67-7.49 (m, 1H), 7.33 (d, J = 9.0 Hz, 1H), 5.22-5.07 (m, 1H), 4.43- 4.25 (m, 1H), 4.13-4.09 (m, 1H), 4.05 (s, 3H), 3.85-3.65 (m, 2H), 2.94- 2.71 (m, 3H), 2.09-1.69 (m, 3H) One proton | 2.12 C |
| | | | | is not visible in | |
| | | | | the NMR, likely | |
| | | | | due to overlap | |
| | | | | with the solvent | |
| | | | | peak | |
| 28 | 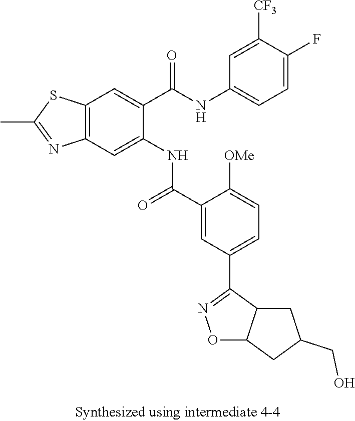 [SMILES: COc1ccc(C2=NOC3CC(CO)CC23)cc1C(=O)Nc1cc2nc(C)sc2cc1C(=O)Nc1ccc(F)c(C(F)(F)F)c1] | N-(4-fluoro- 3-(trifluoro- methyl)phen- yl)-5-(5-(5- (hydroxymeth- yl)-3a,5,6,6a- tetrahydro-4H- cyclopenta[d] isoxazol-3-yl)- 2-methoxy- benzamido)-2- methylbenzo [d]thiazole-6- carboxamide | 641.2 | ¹H NMR (500 MHz, DMSO- d₆) δ 11.66 (s, 1H), 11.02 (s, 1H), 9.09 (s, 1H), 8.51 (s, 1H), 8.42-8.30 (m, 2H), 8.14- 8.02 (m, 1H), 7.88 (dd, J = 8.7, 2.0 Hz, 1H), 7.57 (br t, J = 9.6 Hz, 1H), 7.34 (d, J = 8.9 Hz, 1H), 5.16 (br dd, J = 8.4, 5.0 Hz, 1H), 4.61 (br t, J = 5.2 Hz, 1H), 4.24 (br t, J = 8.7 Hz, 1H), 4.06 (s, 3H), 3.50 (br s, 1H), 3.44-3.28 (m, 1H), 2.87 (s, 3H), 2.01 (br dd, | 2.17 B |
| | | | | J = 13.3, 5.6 Hz, | |
| | | | | 1H), 1.92 (br dd, | |
| | | | | J = 11.6, 5.8 Hz, | |
| | | | | 1H), 1.82 (br dd, | |
| | | | | J = 13.7, 5.2 Hz, | |
| | | | | 1H), 1.75-1.65 | |
| | | | | (m, 1H), 1.63- | |
| | | | | 1.51 (m, 1H) | |
| 29 | 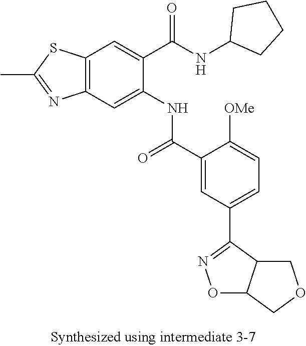 [SMILES: COc1ccc(C2=NOC3COCC23)cc1C(=O)Nc1cc2nc(C)sc2cc1C(=O)NC1CCCC1] | N-cyclopentyl- 5-(2-methoxy- 5-(3a,4,6,6a- tetrahydrofuro [3,4-d]isox- azol-3-yl)- benzamido)-2- methylbenzo [d]thiazole-6- carboxamide (5.9 mg, 0.011 mmol, 36.7% yield) | 521.1 | ¹H NMR (500 MHz, DMSO- d6) δ 12.08- 11.83 (m, 1H), 9.13 (s, 1H), 8.72 (d, J = 7.3 Hz, 1H), 8.42- 8.21 (m, 2H), 7.89 (dd, J = 8.7, 2.4 Hz, 1H), 7.34 (d, J = 8.8 Hz, 1H), 5.38 (dd, J = 9.1, 3.5 Hz, 1H), 4.57 (t, J = 7.7 Hz, 1H), 4.31 (br d, J = 7.3 Hz, 1H), 4.13 (s, 1H), 4.11 (s, 3H), 3.95 (d, J = 9.1 Hz, 1H), 3.81 (dd, J = 9.5, 6.9 Hz, 1H), 3.68 (dd, J = 10.7, 3.8 Hz, 1H), 2.84 (s, 3H), 2.02-1.88 (m, 2H), 1.79-1.66 (m, 2H), 1.63- 1.51 (m, 4H) | 1.831 C |

### Example 30: Preparation of 2-(6-fluoro-3′-((5-((4-fluoro-3-(trifluoromethyl)phenyl)carbamoyl)-2-methylbenzo[d]thiazol-6-yl)carbamoyl)-4′-methoxy-[1,1′-biphenyl]-3-yl)-2-(tetrahydro-2H-pyran-4-carboxamido)acetic acid

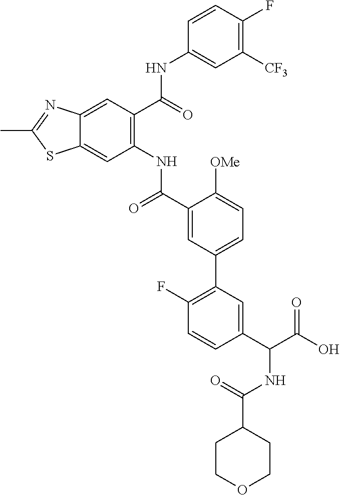 [SMILES: COc1ccc(-c2cc(C(NC(=O)C3CCOCC3)C(=O)O)ccc2F)cc1C(=O)Nc1cc2sc(C)nc2cc1C(=O)Nc1ccc(F)c(C(F)(F)F)c1]

Example 30. To intermediate 15 (34 mg, 92 μmol) and intermediate 16-6 (44 mg, 92 μmol) in ACN (2 mL), was added DIEA (0.4 mL, 2.1 mmol) followed by HATU (42 mg, 0.11 mmol). After 24 h, the reaction mixture was purified by reverse phase HPLC. MS(ESI) m/z: 827.2 (M+H)⁺. The solvents were removed and the residue was partially deprotected with HCl (4N/EtOAc) to afford 2-amino-2-(6-fluoro-3′-((5-((4-fluoro-3-(trifluoromethyl)phenyl)carbamoyl)-2-methylbenzo[d]thiazol-6-yl)carbamoyl)-4′-methoxy-[1,1′-biphenyl]-3-yl)acetic acid. The ensuing intermediate residue was dissolved in DCM (1 mL) followed by the addition of DIEA (0.4 ml, 2.1 mmol), and tetrahydro-2H-pyran-4-carbonyl chloride (14 mg, 92 μmol) and stirred 15 min. The solvents were removed in vacuo and the residue was treated with 50% TFA/DCM (1 mL). After 1 h, the solvents were removed in vacuo and the residue was purified by reverse phase HPLC to afford example 30 (2.6 mg, 3% yield). ¹H NMR (500 MHz, DMSO-d6) δ 11.76 (s, 1H), 11.07 (s, 1H), 9.20 (s, 1H), 8.67 (br d, J=7.8 Hz, 1H), 8.38 (s, 2H), 8.23 (s, 1H), 8.14-8.00 (m, 1H), 7.77 (br d, J=8.6 Hz, 1H), 7.62-7.53 (m, 2H), 7.47-7.22 (m, 3H), 5.41 (d, J=7.5 Hz, 1H), 4.07 (s, 3H), 3.92-3.77 (m, 2H), 3.73-3.61 (m, 1H), 3.39-3.23 (m, 2H), 2.99-2.88 (m, 1H), 2.84 (s, 3H), 1.68-1.60 (m, 2H), 1.59-1.48 (m, 2H); MS (ESI) m/z 804.87 (M+H)⁺; HPLC purity 86%. Analytical LC-MS: 2.12 min. [Method C]

### Example 31: Preparation of 6-fluoro-3′-((6-((4-fluoro-3-(trifluoromethyl)phenyl) carbamoyl)-3-methylbenzo[d]isoxazol-5-yl)carbamoyl)-4′-methoxy-[1,1′-biphenyl]-3-carboxylic acid

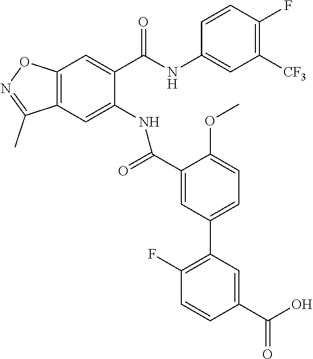 [SMILES: COc1ccc(-c2cc(C(=O)O)ccc2F)cc1C(=O)Nc1cc2c(C)noc2cc1C(=O)Nc1ccc(F)c(C(F)(F)F)c1]

To a stirred solution of intermediate 22 (100 mg, 0.28 mmol). Intermediate 23 (98 mg, 0.28 mmol) in ACN (2 mL). was added 1-methylimidazole (23 mg, 0.28 mmol) followed by N-(chloro(dimethylamino)methylene)-N-methylmethanaminium hexafluorophosphate(V) (79 mg, 0.28 mmol) and the reaction mixture was stirred for 15 h at rt. The reaction mixture was concentrated under vacuum and purified using silica gel chromatography to yield tert-butyl 6-fluoro-3′-((6-((4-fluoro-3-(trifluoromethyl)phenyl)carbamoyl)-3-methylbenzo[d]isoxazol-5-yl)carbamoyl)-4′-methoxy-[1,1′-biphenyl]-3-carboxylate that was treated with TFA at 0° C. and the reaction mixture was stirred for 5 h at rt. The reaction mixture was concentrated under vacuum to yield a residue which was subjected to PREP HPLC to yield example 31 (16 mg, 14% yield). 1H NMR (400 MHz, DMSO-d6) δ 11.69-11.57 (m, 1H), 10.99-10.87 (m, 1H), 8.82-8.73 (m, 1H), 8.38-8.29 (m, 1H), 8.26-8.21 (m, 1H), 8.19-8.14 (m, 1H), 8.11-8.02 (m, 2H), 8.01-7.92 (m, 1H), 7.85-7.77 (m, 1H), 7.60-7.56 (m, 1H), 7.48-7.41 (m, 1H), 7.39-7.35 (m, 1H), 4.10-4.02 (m, 3H), 2.70-2.64 (m, 3H), 2.72-2.63 (m, 3H): MS (ESI) m/z: 626.0 (M+H)⁺; HPLC RT=2.24 min. [Method D]

Examples 32 to 34 were prepared using the general procedures described for Example 31.

TABLE 2

| | | | MS | | LC RT |
|---|---|---|---|---|---|
| Ex. | | | (ESI) | | (min) |
| No. | Structure | Name | (M + H)⁺ | ¹H NMR | Meth. |
| 32 | 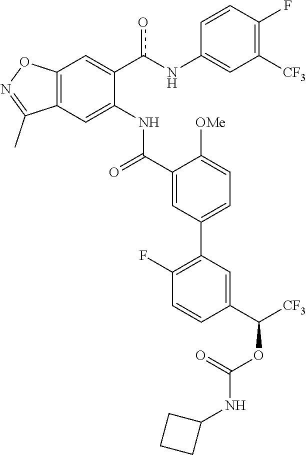 | (S)-2,2,2-trifluoro-1- (6-fluoro-3′-((6-((4- fluoro-3- (trifluoromethyl)phenyl) carbamoyl)-3- methylbenzo[d]isoxazol- 5-yl)carbamoy])-4- methoxy-[1,1′- biphenyl]-3-yl)ethyl cyclobutylcarbamate | 777 | ¹H NMR (400 MHz, DMSO-d₆) δ 11.77- 11.55 (m, 1H), 11.03- 10.87 (m, 1H), 8.91- 8.70 (m, 1H), 8.38-8.32 (m, 1H), 8.22-8.15 (m, 3H), 8.10-8.04 (m, 1H), 7.80-7.70 (m, 2H), 7.58 (s, 2H), 7.39 (d, J = 8.5 Hz, 3H), 6.45-6.35 (m, 1H), 4.07 (s, 3H), 4.02- 3.89 (m, 2H), 2.73-2.60 (m, 3H), 2.23-2.03 (m, 2H), 2.00-1.81 (m, 2H), 1.67-1.51 (m, 2H) | 3.23 D |
| 34 | 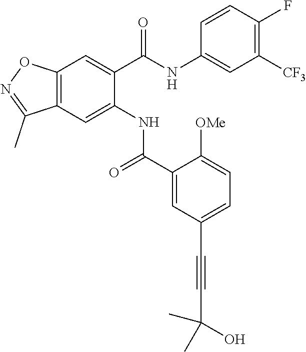 [SMILES: COc1ccc(C#CC(C)(C)O)cc1C(=O)Nc1cc2c(C)noc2cc1C(=O)Nc1ccc(F)c(C(F)(F)F)c1] | N-(4-fluoro-3- (trifluoromethyl)phenyl)- 5-(5-(3-hydroxy- 3-methylbut-1-yn-1- yl)-2- methoxybenzamido)- methylbenzo[d]isoxazole- 6-carboxamide | 570.2 | ¹H NMR (400 MHz, DMSO-d₆) δ ppm 11.81- 11.48 (m, 1H), 11.00- 10.84 (m, 1H), 8.79- 8.71 (m, 1H), 8.36-8.26 (m, 1H), 8.23-8.14 (m, 1H), 8.08-7.97 (m, 2H), 7.60-7.52 (m, 2H), 7.27- 7.19 (m, 1H), 5.50- 5.38 (m, 1H), 4.01 (s, 3H), 2.69 (s, 3H), 1.47 (s, 6H) | 3.27 D |

### Example 35: Preparation of N3-(2,2-difluoro-6-((4-fluoro-3-(trifluoromethyl)phenyl)carbamoyl)benzo[d][1,3]dioxol-5-yl)-4-methoxy-N1-((1-(methylsulfonyl)cyclopropyl)methyl)isophthalamide

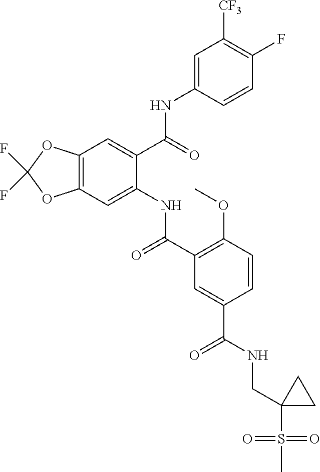 [SMILES: COc1ccc(C(=O)NCC2(S(C)(=O)=O)CC2)cc1C(=O)Nc1cc2c(cc1C(=O)Nc1ccc(F)c(C(F)(F)F)c1)OC(F)(F)O2]

Example 35-1: Preparation of tert-butyl 3-((2,2-difluoro-6-((4-fluoro-3-(trifluoromethyl)phenyl)carbamoyl)benzo[d][1,3]dioxol-5-yl)carbamoyl)-4-methoxybenzoate. To a solution of intermediate 24 (250 mg, 0.66 mmol), 5-(tert-butoxycarbonyl)-2-methoxybenzoic acid (333 mg, 1.32 mmol) in ACN (40 mL) at 0° C. was added chloro-N,N,N′,N′-tetramethylformamidinium hexafluorophosphate (556 mg, 1.98 mmol) and 1-methylimidazole (326 mg, 3.97 mmol) and stirred at rt for 14 h. The reaction mixture was diluted with EtOAc (6 mL) and washed with brine (6 mL) and dried over sodium sulfate. The solution was concentrated under vacuum and purified using silica gel chromatography using hexane and EtOAc as eluents to yield example 35-1 (280 mg, 0.46 mmol, 69% yield). MS (EST) m/z: 611.3 (M−H).

Example 35-2: Preparation of 3-((2,2-difluoro-6-((4-fluoro-3-(trifluoromethyl)phenyl)carbamoyl)benzo[d][1,3]dioxol-5-yl)carbamoyl)-4-methoxybenzoic acid. To a solution of example 35-1 (280 mg, 0.46 mmol) in DCM (8 mL) at 0° C. was added TFA (1 mL, 10 mmol) and the reaction mixture stirred at rt for 4 h. The solution was concentrated under vacuum and the residue triturated with diethyl ether to generate a solid which was dried to yield example 35-2 (210 mg, 0.37 mmol, 83% yield) which was used without further purification. MS (ESI) m/z: 555.2 (M−H)⁻.

Example 35: To a solution of example 35-2 (15 mg, 0.03 mmol), (1-(methylsulfonyl)cyclopropyl)methanamine hydrochloride (6 mg, 0.03 mmol) in DMF (2 mL) was added TEA (0.01 mL, 0.08 mmol) and HATU (12 mg, 0.03 mmol) at 0° C. The reaction mixture was stirred at rt for 14 h. The reaction mixture was concentrated under vacuum and subjected to PREP HPLC purification to yield example 35 (10 mg, 0.020 mmol, 53% yield). 1H NMR (400 MHz, DMSO-d6) δ 11.79 (s, 1H), 10.86 (s, 1H), 8.75 (t, J=6.0 Hz, 1H), 8.63-8.52 (m, 2H), 8.30 (dd, J=2.6, 6.5 Hz, 1H), 8.07 (dd, J=2.4, 8.8 Hz, 1H), 8.04-7.89 (m, 2H), 7.56 (t, J=9.7 Hz, 1H), 7.33 (d, J=8.8 Hz, 1H), 4.06 (s, 3H), 3.83 (d, J=6.1 Hz, 2H), 3.07 (s, 3H), 1.29-1.19 (m, 2H), 1.18-1.08 (m. 2H): MS (ESI) m/z: 688.2 (M+H); HPLC RT=2.18 min. [Method E]

Examples 36 to 42 were prepared using the general procedures described for Example 35.

TABLE 3

| | | | MS | | LC RT |
|---|---|---|---|---|---|
| Ex. | | | (ESI) | | (min) |
| No. | Structure | Name | (M + H)⁺. | ¹H NMR | Meth. |
| 36 | 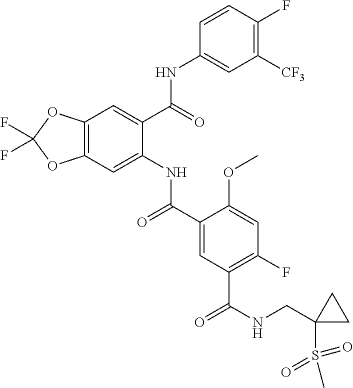 [SMILES: COc1cc(F)c(C(=O)NCC2(S(C)(=O)=O)CC2)cc1C(=O)Nc1cc2c(cc1C(=O)Nc1ccc(F)c(C(F)(F)F)c1)OC(F)(F)O2] | N1-(2,2-difluoro-6- ((4-fluoro-3- (trifluoromethyl)phenyl) carbamoyl)benzo[d] [1,3]dioxol-5-yl)-4- fluoro-6-methoxy-N3- ((1- (methylsulfonyl)cyclo propyl)methyl)iso- phthalamide | 706.1 | 1H NMR (400 MHz, DMSO-d₆) δ 11.74 (br s, 1H), 10.84 (br s, 1H), 8.55 (s, 1H), 8.49-8.41 (m, 1H), 8.40 (d, J = 8.8 Hz, 1H), 8.30 (dd, J = 6.5, 2.6 Hz, 1H), 8.01 (dt, J = 8.6. 3.8 Hz, 1H), 7.94 (s, 1H), 7.55 (t, J = 9.8 Hz, 1H), 7.25 (d, J = 12.7 Hz, 1H), 4.07 (s, 4H), 3.83 (d, J = 6.1 Hz, 2H), 3.09 (s, 3H), 1.30-1.21 (m, 2H), 1.16-1.09 (m, 2H) | 2.26 E |
| 37 | 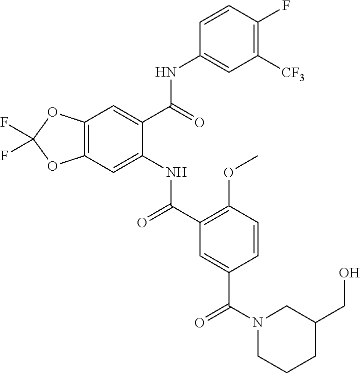 [SMILES: COc1ccc(C(=O)N2CCCC(CO)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(5-(3- (hydroxymethyl) piperidine-1-carbonyl)- 2-methoxybenzamido) benzo[d][1,3]dioxole- 5-carboxamide | 654.2 | 1H NMR (400 MHz, DMSO-d₆) δ 11.84- 11.72 (m, 1H), 10.87 (br s, 1H), 8.56 (s, 1H), 8.30 (dd, J = 2.4, 6.4 Hz, 1H), 8.08-7.98 (m, 2H), 7.95 (s, 1H), 7.64-7.48 (m, 2H), 7.28 (d, J = 8.6 Hz, 1H), 4.62-4.24 (m, 2H), 4.05 (s, 3H), 3.77-3.43 (m, 1H), 3.08-2.71 (m, 2H), 1.77-1.69 (m, 2H), 1.67-1.27 (m, 3H), 1.25-1.14 (m, 2H) | 2.17 E |
| 38 | 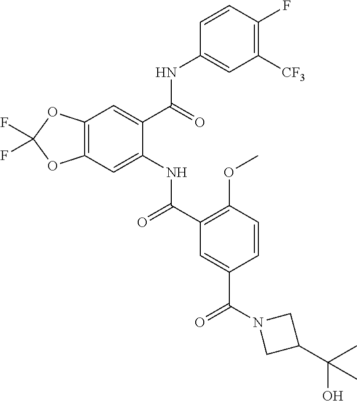 [SMILES: COc1ccc(C(=O)N2CC(C(C)(C)O)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(5-(3-(2- hydroxypropan-2- yl)azetidine-1- carbonyl)-2- methoxybenzamido) benzo[d][1,3]dioxole- 5-carboxamide | 654.2 | 1H NMR (400 MHz, DMSO-d₆) δ 11.82- 11.69 (m, 1H), 10.85 (s, 1H), 8.57 (s, 1H), 8.33- 8.23 (m, 2H), 8.07-7.99 (m, 1H), 7.96 (s, 1H), 7.86 (dd, J = 2.3, 8.7 Hz, 1H), 7.56 (t, J = 9.8 Hz, 1H), 7.29 (d, J = 8.8 Hz, 1H), 4.55 (s, 1H), 4.29- 4.12 (m, 2H), 4,06 (s, 3H), 3.98-3.88 (m, 2H), 2.65-2.56 (m, 1H), 1.03 (brd, J = 12.2 Hz, 5H) | 2,21 E |
| 39 | 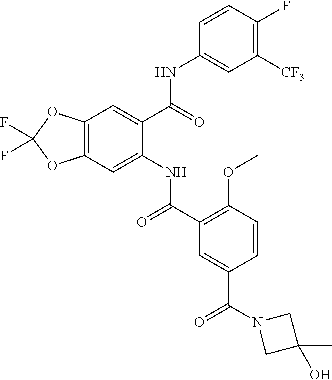 [SMILES: COc1ccc(C(=O)N2CC(C)(O)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(3-hydroxy- 3-methylazetidine-1- carbonyl)-2- methoxybenzamido) benzo[d][1,3]dioxole- 5-carboxamide | 626.2 | 1H NMR (400 MHz, DMSO-d₆) δ 11.75 (br s, 1H), 10.85 (s, 1H), 8.56 (s, 1H), 8.35-8.21 (m, 2H), 8.08-7.99 (m, 1H), 7.96 (s, 1H), 7.85 (dd, J = 2.4, 8.6 Hz, 1H), 7.56 (t, J = 10.0 Hz, 1H), 7.29 (d, J = 8.8 Hz, 1H), 5.68 (s, 1H), 4.21-4.10 (m, 2H), 4.06 (s, 3H), 3.98- 3.83 (m, 2H), 1.39 (s, 3H) | 2.12 E |
| 40 | 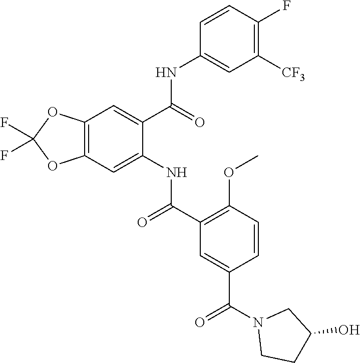 [SMILES: COc1ccc(C(=O)N2CC[C@@H](O)C2)cc1C(=O)Nc1cc2c(cc1C(=O)Nc1ccc(F)c(C(F)(F)F)c1)OC(F)(F)O2] | (R)-2,2-difluoro-N-(4- fluoro-3- (trifluoromethyl) phenyl)-6-(5-(3- hydroxypyrrolidine-1- carbonyl)-2- methoxybenzamido) benzo[d][1,3]dioxole- 5-carboxamide | 626.2 | 1H NMR (400 MHz, DMSO-d₆) δ 11.77 (br dd, J = 3.4, 4.6 Hz, 1H), 10.87 (s, 1H), 8.56 (s, 1H), 8.30 (dd, J = 1.8, 6.5 Hz, 1H), 8.24-8.16 (m, 1H), 8.07-7.99 (m, 1H), 7.95 (s, 1H), 7.81-7.72 (m, 1H), 7.57 (t, J = 9.7 Hz, 1H), 7.28 (d, J = 8.8 Hz, 1H), 5.05-4.90 (m, 1H), 4.36-4.19 (m, 1H), 4.05 (s, 3H), 3.65- 3.43 (m, 3H), 3.22 (br d, J = 10.5 Hz, 1H), 2.00- 1.71 (m, 2H) | 2.02 E |
| 41 | 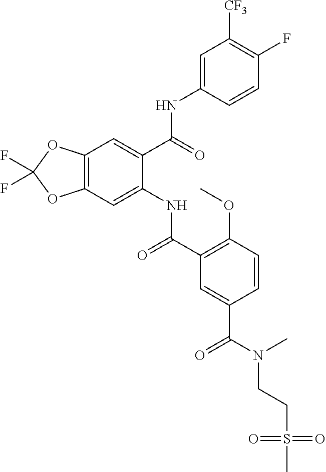 [SMILES: COc1ccc(C(=O)N(C)CCS(C)(=O)=O)cc1C(=O)Nc1cc2c(cc1C(=O)Nc1ccc(F)c(C(F)(F)F)c1)OC(F)(F)O2] | N3-(2,2-difluoro-6- ((4-fluoro-3- (trifluoromethyl)phenyl) carbamoyl)benzo[d] [1,3]dioxol-5-yl)-4- methoxy-N1-methyl- N1-(2- (methylsulfony])ethyl) isophthalamide | 676.2 | 1H NMR (400 MHz, DMSO-d₆) δ 11.80 (br s, 1H), 10.86 (br s, 1H), 8.56 (s, 1H), 8.30 (dd, J = 2.4, 6.4 Hz, 1H), 8.09 (d, J = 1.5 Hz, 1H), 8.06- 7.98 (m, 1H),7.95 (s, 1H), 7.65 (dd, J = 2.0, 8.6 Hz, 1H), 7.56 (t, J = 9.8 Hz, 1H), 7.29 (d, J = 8.6 Hz, 1H), 4.05 (s, 3H), 3.91-3.56 (m, 3H), 3.49 (br t, J = 7.0 Hz, 2H), 3.14- 3.02 (m, 2H), 2.97 (s, 3H) | 2.11 E |
| 42 | 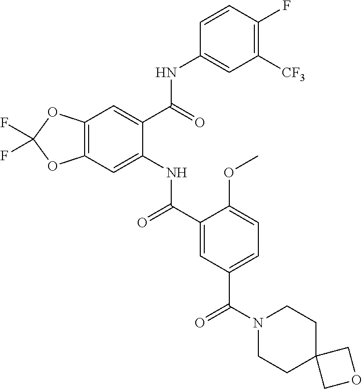 [SMILES: COc1ccc(C(=O)N2CCC3(CC2)COC3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5- (2-oxa-7- azaspiro[3.5]nonane- 7- carbonyl)benzamido) benzo[d][1,3]dioxole- 5-carboxamide | 666.2 | 1H NMR (400 MHz, DMSO-d₆) δ 10.95- 10.77 (m, 2H), 8.55 (s, 1H), 8.32-8.22 (m, 1H), 7.95-7.95 (m, 1H), 8.03- 7.88 (m, 2H), 7.64- 7.48 (m,2H), 7.28 (d, J = 8.6 Hz, 1H), 4.32 (s, 4H), 4.02 (s, 3H), 1.87- 1.70 (m, 4H), 1.23 (s, 4H) | 2.23 E |

### Example 43: Preparation of 2,2-difluoro-N-(4-fluoro-3-(trifluoromethyl)phenyl)-6-(2-methoxy-5-(3a,5,6,6a-tetrahydro-4H-pyrrolo[3,4-d]isoxazol-3-yl)benzamido)benzo[d][1,3]dioxole-5-carboxamide

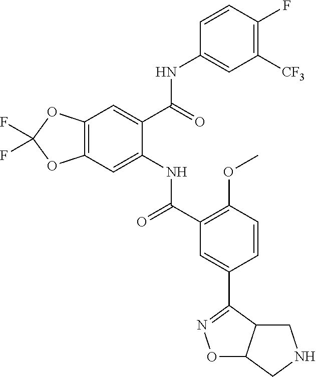 [SMILES: COc1ccc(C2=NOC3CNCC23)cc1C(=O)Nc1cc2c(cc1C(=O)Nc1ccc(F)c(C(F)(F)F)c1)OC(F)(F)O2]

### From Peak 2

Example 43-1: Preparation of tert-butyl 3-(3-((2,2-difluoro-6-((4-fluoro-3-(trifluoromethyl)phenyl)carbamoyl)benzo[d][1,3]dioxol-5-yl)carbamoyl)-4-methoxyphenyl)-3a,4,6,6a-tetrahydro-5H-pyrrolo[3,4-d]isoxazole-5-carboxylate. To a solution of intermediate 24 (400 mg, 1.06 mmol) and intermediate 25 (575 mg, 1.58 mmol) in ACN (25 mL) at 0° C. were added chloro-N,N,N′,N′-tetramethylformamidinium hexafluorophosphate (890 mg, 3.17 mmol) and 1-methylimidazole (521 mg, 6.35 mmol). The reaction mixture was stirred at rt for 14 h then concentrated under vacuum to yield a residue which was subjected to silica gel chromatography using 40 to 50% EtOAc in petroleum ether to yield example 43-1 (450 mg, 0.6 mmol, 59% yield). The isomers were separated by SFC with Example 43-1 being Peak-2, (RT=6.1 min., >95% ee) Instrument: PIC Solution, Prep SFC Column: Whelk (R,R) 250×30 mm, 5 micron, Mobile phase: 60% CO₂ with 40% of 0.2% of methanolic ammonia in MeOH, Flow conditions: 100 g/min, Back Pressure: 100 bar, Temperature: 35° C., Detector wavelength: 272 nm, Analytical method: Column: Whelk (R,R) 250×4.6 mm, 5 micron, Mobile phase: 60% CO₂ with 40% of 0.2% of methanolic ammonia in MeOH, Flow conditions: 4.0 g/min, Back Pressure: 100 bar, Temperature: 35° C., Detector wavelength: 272 nm, to afford Peak-1, RT=5.2 min., >95% ee) and Peak-2, RT=6.1 min., >95% ee). SFC purification yielded example-43-1 peak-1 (140 mg, 0.19 mmol, 18% yield) and example-43-1 peak-2 (150 mg, 0.21 mmol, 20% yield). MS (ESI) m/z: 721.3 (M−H)⁻.

Example 43: To a solution of Example 43-1 peak-2 (130 mg, 0.18 mmol) in DCM (10 mL) at 0° C. was added TFA (0.28 mL, 3.6 mmol) and the reaction mixture stirred at it for 4 h. The reaction mixture was then concentrated under vacuum and the residue subjected to reverse phase HPLC purification using Waters XBridge C18 column, 19×150 mm, 5 microns; Mobile Phase A: 10-mM NH₄OAc; Mobile Phase B: ACN; Gradient: 21-65% B over 25 min, then a 5 minute hold at 100% B at a flow rate of 15 mL/min to yield example 43 (15 mg, 0.020 mmol, 13% yield). ¹H NMR (400 MHz, DMSO-d₆) δ 11.77 (s, 1H), 10.87 (s, 1H), 8.57 (s, 1H), 8.34-8.25 (m, 2H), 8.06-7.98 (m, 1H), 7.96 (s, 1H), 7.86 (dd, J=8.7, 2.3 Hz, 1H), 7.57 (t, J=9.8 Hz, 1H), 7.32 (d, J=8.8 Hz, 1H), 5.26-5.18 (m, 1H), 4.30-4.23 (m, 1H), 4.05 (s, 3H), 3.21-3.16 (m, 1H), 3.04-2.88 (m, 2H), 2.83-2.74 (m, 1H). MS (ESI) m/z: 623.2 (M+H)⁺; HPLC RT=2.3 min. [Method E]

### Example 44: Preparation of 2,2-difluoro-N-(4-fluoro-3-(trifluoromethyl)phenyl)-6-(5-(5-(2-hydroxyacetyl)-3a,5,6,6a-tetrahydro-4H-pyrrolo[3,4-d]isoxazol-3-yl)-2-methoxybenzamido)benzo[d][1,3]dioxole-5-carboxamide

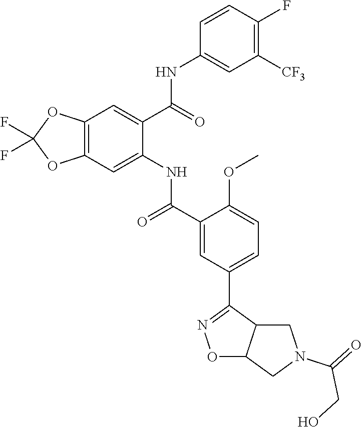 [SMILES: COc1ccc(C2=NOC3CN(C(=O)CO)CC23)cc1C(=O)Nc1cc2c(cc1C(=O)Nc1ccc(F)c(C(F)(F)F)c1)OC(F)(F)O2]

To a solution of example 43 (20 mg, 0.03 mmol) and 2-hydroxyacetic acid (2.5 mg, 0.03 mmol) in DMF (2 mL) was added HATU (12 mg, 0.03 mmol) and TEA (0.02 mL, 0.16 mmol) and the reaction mixture was stirred at rt for 15 h. The reaction mixture was concentrated under vacuum and the residue subjected to reverse phase HPLC purification using Waters XBridge C18 column, 19×150 mm, 5 microns; Mobile Phase A: 10-mM NH₄OAc; Mobile Phase B: ACN; Gradient: 15-65% B over 25 min, then a 5 minute hold at 100% B at a flow rate of 15 mL/min to yield example 44 (8 mg, 0.01 mmol, 40% yield). ¹H NMR (400 MHz, DMSO-d₆) δ 11.77 (br s, 1H), 10.87 (s, 1H), 8.60-8.51 (m, 1H), 8.35-8.25 (m, 2H), 8.08-7.99 (m, 1H), 7.96 (s, 1H), 7.93-7.81 (m, 1H), 7.57 (t, J=9.9 Hz, 1H), 7.34 (d, J=8.8 Hz, 1H), 5.42-5.28 (m, 1H), 4.77-4.63 (m, 1H), 4.60-4.46 (m, 1H), 4.10-4.01 (m, 3H), 3.99-3.93 (m, 1H), 3.89-3.73 (m, 2H), 3.69-3.55 (m, 2H), 1.23 (s, 2H). MS (ESI) m/z: 695.2 (M+H)⁺; HPLC RT=2.25 min. [Method E]

### Example 45: Preparation of 2,2-difluoro-N-(4-fluoro-3-(trifluoromethyl)phenyl)-6-(5-(5-(2-hydroxy-2-methylpropyl)-3a,5,6,6a-tetrahydro-4H-pyrrolo[3,4-d]isoxazol-3-yl)-2-methoxybenzamido)benzo[d][1,3]dioxole-5-carboxamide

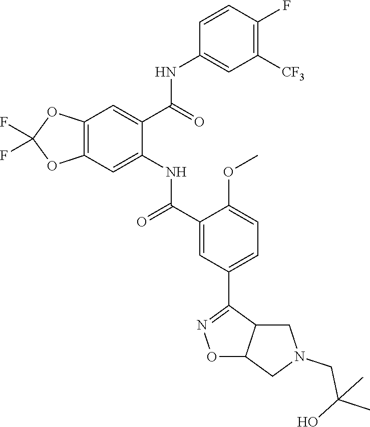 [SMILES: COc1ccc(C2=NOC3CN(CC(C)(C)O)CC23)cc1C(=O)Nc1cc2c(cc1C(=O)Nc1ccc(F)c(C(F)(F)F)c1)OC(F)(F)O2]

A solution of example 43-1peak2 (20 mg, 0.03 mmol) and 2,2-dimethyloxirane (6 mg, 0.08 mmol) in EtOH (2 mL) was stirred at 55° C. for 15 h. The reaction mixture was then concentrated under vacuum which was then subjected to reverse phase HPLC purification using Waters XBridge C18 column, 19×150 mm, 5 microns; Mobile Phase A: 10-mM NH₄OAc; Mobile Phase B: ACN; Gradient: 30-75% B over 25 min, then a 5 minute hold at 100% B at a flow rate of 15 mL/min to yield example 45 (18 mg, 0.010 mmol, 81% yield). ¹H NMR (400 MHz, DMSO-d₆) δ 11.78 (s, 1H), 10.86 (s, 1H), 8.57 (s, 1H), 8.34-8.23 (m, 2H), 8.07-7.99 (m, 1H), 7.95 (s, 1H), 7.85 (dd, J=8.8, 2.2 Hz, 1H), 7.56 (t, J=9.7 Hz, 1H), 7.31 (d, J=9.0 Hz, 1H), 5.10 (dd, J=9.5, 4.4 Hz, 1H), 4.34-4.22 (m, 1H), 4.05 (s, 4H), 3.21 (d, J=10.8 Hz, 1H), 3.09 (br d, J=9.5 Hz, 1H), 2.45 (br dd, J=11.5, 4.6 Hz, 11H), 2.34-2.27 (m, 11H), 2.26-2.19 (m, 1H), 1.01 (s, 3H), 0.94 (s, 3H). MS (ESI) m/z: 695.3 (M+H)⁺; HPLC RT=2.52 min. [Method E]

### Example 46: Preparation of 2,2-difluoro-N-(4-fluoro-3-(trifluoromethyl)phenyl)-6-(5-(1-(2-hydroxy-2-methylpropanoyl)pyrrolidin-3-yl)-2-methoxybenzamido)benzo[d][1,3]dioxole-5-carboxamide

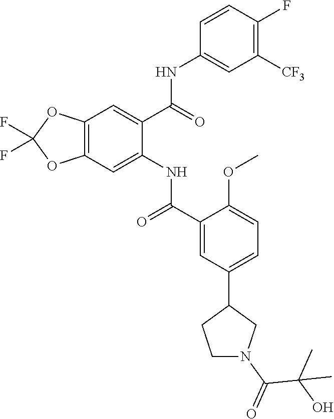 [SMILES: COc1ccc(C2CCN(C(=O)C(C)(C)O)C2)cc1C(=O)Nc1cc2c(cc1C(=O)Nc1ccc(F)c(C(F)(F)F)c1)OC(F)(F)O2]

### From Isomer 1

To a solution of example 54 (20 mg, 0.03 mmol) and 2-hydroxy-2-methylpropanoic acid (4 mg, 0.04 mmol) in DMF (2 mL) was added HATU (13 mg, 0.03 mmol) followed by TEA (0.03 mL, 0.2 mmol) and the reaction mixture was stirred at rt for 15 h. The reaction mixture was then concentrated under vacuum and the residue subjected to reverse phase HPLC purification using Waters XBridge C18 column, 19×150 mm, 5 micron; Mobile Phase A: 10-mM NH₄OAc; Mobile Phase B: ACN; Gradient: 15-55% B over 25 min, then a 5 minute hold at 100% B at a flow rate of 15 mL/min to yield example 46 (14 mg, 0.020 mmol, 55% yield). ¹H NMR (400 MHz, DMSO-d6) δ 11.75-11.69 (m, 11H), 10.85 (s, 1H), 8.56 (d, J=2.7 Hz, 1H), 8.34-8.27 (m, 1H), 8.06-7.99 (m, 1H), 7.97-7.90 (m, 2H), 7.61-7.55 (m, 1H), 7.55-7.46 (m, 1H), 7.23-7.16 (m, 1H), 5.16 (d, J=3.4 Hz, 1H), 4.35-4.27 (m, 1H), 4.11-4.05 (m, 1H), 3.99 (s, 3H), 3.57-3.51 (m, 1H), 3.17 (d, J=5.1 Hz, 2H), 2.27-2.12 (m, 2H), 1.97-1.75 (m, 1H), 1.36-1.26 (m, 4H), 1.23 (s, 1H). MS (ESI) m/z: 668.2 (M+H)⁺; HPLC RT=2.34 min. [Method E]

### Example 47: Preparation of N3-(2,2-difluoro-6-((4-fluoro-3-(trifluoromethyl)phenyl)carbamoyl)benzo[d][1,3]dioxol-5-yl)-6′-fluoro-4-methoxy-N3′-(methylsulfonyl)-[1,1′-biphenyl]-3,3′-dicarboxamide

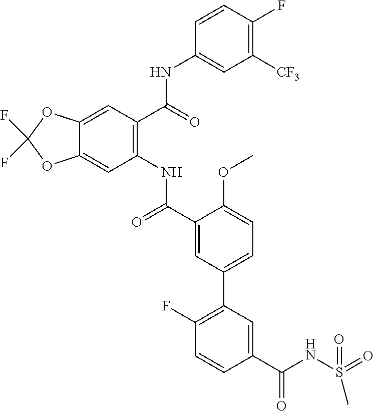 [SMILES: COc1ccc(-c2cc(C(=O)NS(C)(=O)=O)ccc2F)cc1C(=O)Nc1cc2c(cc1C(=O)Nc1ccc(F)c(C(F)(F)F)c1)OC(F)(F)O2]

To a solution of example 57 (130 mg, 0.2 mmol) in THF (6.0 mL) at 0° C. was added CDI (65 mg, 0.40 mmol) followed by TEA (0.14 mL, 0.99 mmol) and the reaction mixture was stirred at 80° C. for 4 h. The reaction mixture was then concentrated under vacuum and the residue (40 mg, 0.006 mmol) was dissolved in THF (2 mL) and cooled to 0° C. To this cooled solution was added methanesulfonamide (16 mg, 0.17 mmol) followed by TEA (0.04 mL, 0.29 mmol) and the reaction mixture was heated to 80° C. and stirred at that temperature for 2 h. The reaction mixture was then concentrated under vacuum and the residue subjected to reverse phase HPLC purification using Waters XBridge C18 column, 19×150 mm, 5 micron; Mobile Phase A: 10-mM NH₄OAc; Mobile Phase B: ACN; Gradient: 15-55% B over 25 min, then a 5 minute hold at 100% B at a flow rate of mL/min to yield example 47 (18 mg, 0.080 mmol, 41% yield). ¹H NMR (400 MHz, DMSO-d6) δ 12.41-12.21 (m, 1H), 11.83 (s, 1H), 10.87 (s, 1H), 8.59 (s, 1H), 8.38-8.27 (m, 2H), 8.17 (dd, J=2.2, 7.6 Hz, 1H), 8.09-7.93 (m, 3H), 7.88 (br d, J=8.8 Hz, 1H), 7.57 (t, J=9.7 Hz, 1H), 7.49 (dd, J=8.9, 10.4 Hz, 1H), 7.39 (d, J=8.8 Hz, 1H), 4.08 (s, 3H), 3.39 (s, 3H). MS (ESI) m/z: 728.2 (M+H)⁻; HPLC RT=2.05 min. [Method E]

### Example 48: Preparation of N3-(2,2-difluoro-6-((4-fluoro-3-(trifluoromethyl)phenyl)carbamoyl)benzo[d][1,3]dioxol-5-yl)-6′-fluoro-4-methoxy-N3′-(methylsulfonyl)-[1,1′-biphenyl]-3,3′-dicarboxamide

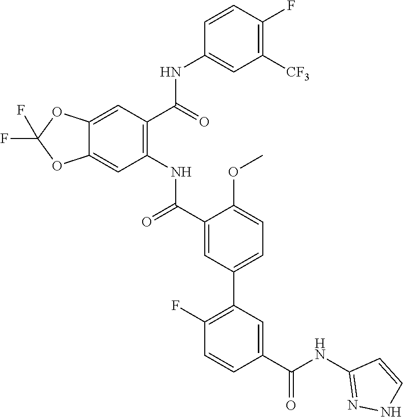 [SMILES: COc1ccc(-c2cc(C(=O)Nc3cc[nH]n3)ccc2F)cc1C(=O)Nc1cc2c(cc1C(=O)Nc1ccc(F)c(C(F)(F)F)c1)OC(F)(F)O2]

To a solution of intermediate 24 (40 mg, 0.06 mmol) in THF (2 mL) at 0° C. was added 1H-pyrazol-3-amine (14 mg, 0.17 mmol) followed by TEA (0.04 mL, 0.3 mmol) and the reaction mixture was stirred at 80° C. for 2 h. The reaction mixture was then concentrated under vacuum and the residue subjected to reverse phase HPLC purification using Waters XBridge C18 column, 19×150 mm, 5 micron; Mobile Phase A: 10-mM NH₄OAc; Mobile Phase B: ACN; Gradient: 20-65% B over 25 min, then a 5 minute hold at 100% B at a flow rate of 15 mL/min to yield example 48 (6 mg, 0.01 mmol, 10% yield). ¹H NMR (400 MHz, DMSO-d₆) δ 11.83 (s, 1H), 11.04 (s, 1H), 10.88 (s, 1H), 8.60 (s, 1H), 8.38-8.29 (m, 2H), 8.24 (dd, J=2.2, 7.8 Hz, 1H), 8.04 (dq, J=2.7, 5.3 Hz, 2H), 7.96 (s, 1H), 7.91 (br d, J=8.6 Hz, 1H), 7.67 (d, J=2.0 Hz, 1H), 7.57 (t, J=9.9 Hz, 2H), 7.49-7.34 (m, 2H), 6.65 (d, J=2.0 Hz, 1H), 4.08 (s, 3H). MS (ESI) m/z: 716.2 (M+H)⁺; HPLC RT=2.42 min. [Method E]

### Example 49: Preparation of 2,2-difluoro-N-(4-fluoro-3-(trifluoromethyl)phenyl)-6-(2-methoxy-5-(1-(methylsulfonyl)piperidin-3-yl)benzamido)benzo[d][1,3]dioxole-5-carboxamide

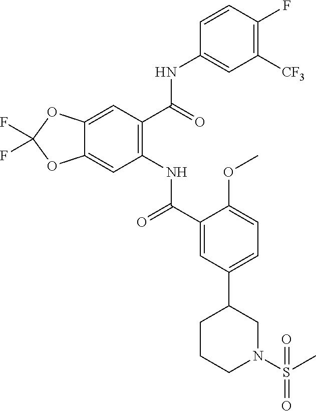 [SMILES: COc1ccc(C2CCCN(S(C)(=O)=O)C2)cc1C(=O)Nc1cc2c(cc1C(=O)Nc1ccc(F)c(C(F)(F)F)c1)OC(F)(F)O2]

Example 49-1: Preparation of 6-(5-(1-benzyl-1,4,5,6-tetrahydropyridin-3-yl)-2-methoxybenzamido)-2,2-difluoro-N-(4-fluoro-3-(trifluoromethyl)phenyl)benzo[d][1.3]dioxole-5-carboxamide. Example 49-1 (400 mg, 0.56 mmol, 74% yield) is prepared in the same way as example 43-1 by replacing intermediate 25 with 5-(1-benzyl-1,4,5,6-tetrahydropyridin-3-yl)-2-methoxybenzoic acid made similar to intermediate 27. MS (ESI) m/z=684.3 (M+H)⁺.

Example 49-2: Preparation of 2,2-difluoro-N-(4-fluoro-3-(trifluoromethyl)phenyl)-6-(2-methoxy-5-(piperidin-3-yl)benzamido)benzo[d][1,3]dioxole-5-carboxamide. To a solution of example 49-1 (400 mg, 0.58 mmol) in MeOH (20 mL) was added Pd—C (156 mg, 1.46 mmol) and the reaction mixture stirred at rt for 12 h under hydrogen balloon. The reaction mixture was then filtered over a pad of Celite® and concentrated under vacuum to yield a residue that was subjected to chiral separation. Isomers separated by SFC with Example 49-2 being Peak-2 (RT=7.8 min, >95% ee) Instrument: Thar SFC-80, Prep SFC Column: Luxcellulose-4 250×21.5 mm, 5 micron, Mobile phase: 75% CO₂ with 25% of 4M methanolic ammonia in MeOH, Flow conditions: 80 g/min, Back Pressure: 100 bar, Temperature: 30° C., Detector wavelength: 220 nm: Analytical method: Luxcellulose-4 250×4.6 mm, 5 micron, Mobile phase: 75% CO₂ with 25% of 4M methanolic ammonia in MeOH, Flow conditions: 4 g/min, Back Pressure: 100 bar, Temperature: 30° C., Detector wavelength: 220 nm: example 49-1 peak1 (Peak-1, RT=5.4 min., >95% ee)(40 mg, 0.006 mmol, 11% yield) and example 49-2 peak2 (Peak-2, RT=7.8 min., >95% ee) (41 mg, 0.06 mmol, 11% yield).

### Example 49: Preparation of 2,2-difluoro-N-(4-fluoro-3-(trifluoromethyl)phenyl)-6-(2-methoxy-5-(1-(methylsulfonyl)piperidin-3-yl)benzamido)benzo[d][1,3]dioxole-5-carboxamide

To a solution of Example 49-2 peak2 (10 mg, 0.02 mmol) and TEA (5 μL. 0.05 mmol) in DCM (2 mL) at 0° C. was added methanesulfonyl chloride (2.3 mg, 0.02 mmol) and the reaction mixture stirred at rt for 12 h. The reaction mixture was then concentrated under vacuum and the residue subjected to reverse phase HPLC purification using Waters XBridge C18 column, 19×150 mm. 5 microns; Mobile Phase A: 10-mM NH₄OAc: Mobile Phase B: ACN; Gradient: 15-65% B over 25 min, then a 5 minute hold at 100% B at a flow rate of 15 mL/min to yield Example 49 (2 mg, 0.003 mmol, 20% yield). ¹H NMR (400 MHz, DMSO-<6) δ 11.73 (s, 1H), 10.87 (s, 1H), 8.57 (s, 1H), 8.31 (dd, J=2.7, 6.4 Hz, 1H), 8.07-7.99 (m, 11H), 7.99-7.89 (m, 2H), 7.63-7.49 (m, 2H), 7.20 (d, J=8.6 Hz, 1H), 3.99 (s, 3H), 3.61-3.54 (m, 2H), 2.88 (s, 3H), 2.86-2.70 (m, 3H), 1.93-1.77 (m, 2H), 1.66-1.50 (m, 2H). MS (ESI) m/z: 672.2 (M+H)⁺; HPLC RT=2.48 min. [Method F]

### Example 50: Preparation of 6-(5-(5-amino-3a,5,6,6a-tetrahydro-4H-cyclopenta[d]isoxazol-3-yl)-2-methoxybenzamido)-2,2-difluoro-N-(4-fluoro-3-(trifluoromethyl)phenyl)benzo[d][1,3]dioxole-5-carboxamide

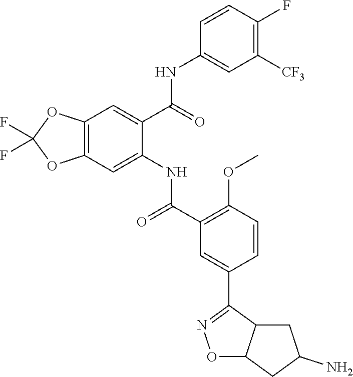 [SMILES: COc1ccc(C2=NOC3CC(N)CC23)cc1C(=O)Nc1cc2c(cc1C(=O)Nc1ccc(F)c(C(F)(F)F)c1)OC(F)(F)O2]

Example 50-1 (70 mg, 0.11 mmols, 8% yield) was prepared in the same way as example 43-1 by replacing intermediate 25 with the racemic 5-(5-((tert-butoxycarbonyl)amino)-3a,5,6,6a-tetrahydro-4H-cyclopenta[d]isoxazol-3-yl)-2-methoxybenzoic acid prepared as intermediate 3-3. The reaction mixture was diluted with EtOAc (6 mL) and washed with brine. The organic layers are then dried over sodium sulfate and purified using silica gel chromatography using hexane and petroleum ether as eluants to yield the diastereomeric mixture which was then subjected to SFC purification Instrument: Make/Model: Thor SFC-350, Column/dimensions: Luxcellulose-2 (250×30) mm, 5u, % CO₂: 50%, % Co-solvent: 50% of 4M methanolic ammonia in MeOH, Total Flow: 150.0 g/min, Back Pressure: 100 bar, Temperature: 30° C., UV: 272 nm, Analytical SFC Conditions, Column/dimensions: Luxcellulose-2 (250×4.6) mm, 5u, % CO₂: 50%, % Co solvent: 50% of 4M methanolic ammonia in MeOH, Total Flow: 4.0 g/min, Back Pressure: 100 bar, Temperature: 30° C., UV: 272 nm, to afford chiral (Peak-1, RT=2.6 min., >95% ee) and chiral (Peak-2. RT=5.4 min., >95% ee). Peak 1 from the above SFC separation was further subjected to a second SFC purification using the below SFC conditions—Instrument: Make/Model: WATERS SFC-350, Column/dimensions: Chiralpak AS-H (250×30) mm, 5u, % CO₂: 80%, % Co solvent: 20% of 0.2% of 4 M methanolic ammonia in MeOH, Total Flow: 150 g/min, Back Pressure: 100 bar, Temperature: 30° C., UV: 271 nm, Analytical SC Conditions, Column/dimensions: Chiralpak AS-H (250×4.6) mm, 5u, % CO₂: 80%, % Co solvent: 20% of 0.2% of 4M methanolic ammonia in MeOH, Total Flow: 4 g/min, Back Pressure: 100 bar, Temperature: 30° C., UV: 271 nm, to afford chiral (Peak-1, RT=3.8 min., >95% ee) and chiral (Peak-2, RT=5.9 min., >95% cc). Peak 1 from second SFC purification is the desired peak 50-1. MS (ESI) m/z=735.0 (M−H)⁻.

### Example 50: Preparation of 6-(5-(5-amino-3a,5,6,6a-tetrahydro-4H-cyclopenta[d]isoxazol-3-yl)-2-methoxybenzamido)-2,2-difluoro-N-(4-fluoro-3-(trifluoromethyl)phenyl)benzo[d][1,3]dioxole-5-carboxamide

To a solution of example 50-1 (Isomer 3) (95 mg, 0.13 mmol) in DCM (10 mL) was added TFA (0.2 mL, 3 mmol) at 0° C. and stirred at rt for 4 h. The reaction mixture was then concentrated under vacuum and the residue subjected to reverse phase HPLC purification using Waters XBridge C18 column, 19×150 mm, 5 micron: Mobile Phase A: 10-mM NH₄OAc; Mobile Phase B: ACN; Gradient: 18-65% B over 25 min, then a 5 minute hold at 100% B at a flow rate of 15 mL/min to yield example 50 (8 mg, 0.01 mmol, 9% yield). ¹H NMR (400 MHz, DMSO-d6) δ 11.93-11.64 (m, 1H), 8.57 (s, 1H), 8.36-8.24 (m, 2H), 8.07-7.98 (m, 1H), 7.96 (s, 1H), 7.87 (dd, J=2.3, 8.9 Hz, 1H), 7.56 (t, J=9.9 Hz, 1H), 7.33 (d, J=8.6 Hz, 1H), 5.15-5.06 (m, 1H), 4.15-3.99 (m, 5H), 2.38-2.21 (m, 3H), 1.90 (s, 2H), 1.73-1.60 (m, 1H), 1.53-1.41 (m, 1H). MS (ESI) m/z: 637.2 (M+H)⁺; HPLC RT=2.08 min. [Method E]

### Example 51: Preparation of 2,2-difluoro-N-(4-fluoro-3-(trifluoromethyl)phenyl)-6-(5-(5-(2-hydroxyacetamido)-3a,5,6,6a-tetrahydro-4H-cyclopenta[d]isoxazol-3-yl)-2-methoxybenzamido)benzo[d][1,3]dioxole-5-carboxamide

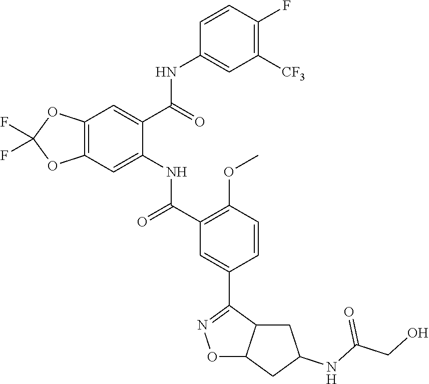 [SMILES: COc1ccc(C2=NOC3CC(NC(=O)CO)CC23)cc1C(=O)Nc1cc2c(cc1C(=O)Nc1ccc(F)c(C(F)(F)F)c1)OC(F)(F)O2]

To a solution of example 51 (20 mg, 0.03 mmol) and 2-hydroxyacetic acid (5 mg, 0.06 mmol) in DMF (2 mL) was added HATU (12 mg, 0.03 mmol) followed by TEA (0.02 mL, 0.16 mmol) and stirred at rt for 15 h. The reaction mixture was then concentrated under vacuum and the residue subjected to reverse phase HPLC purification using Waters XBridge C18 column, 19×150 mm, 5 microns; Mobile Phase A: 10-mM NH₄OAc; Mobile Phase B: ACN; Gradient: 25-55% B over 25 min, then a 5 minute hold at 100% B at a flow rate of 15 ml/min to yield example 51 (3 mg, 0.004 mmol, 10% yield). ¹H NMR (400 MHz, DMSO-d6) δ 11.94-11.73 (m, 1H), 10.86 (br d, J=17.9 Hz, 1H), 8.57 (s, 1H), 8.34-8.21 (m, 2H), 8.06-7.97 (m, 1H), 7.95 (s, 1H), 7.87 (dd, J=2.4, 8.8 Hz, 1H), 7.82 (d, J=8.3 Hz, 1H), 7.60-7.46 (m, 1H), 7.32 (d, J=9.0 Hz, 1H), 5.50-5.38 (m, 1H), 5.18-5.04 (m, 1H), 4.32-4.22 (m, 1H), 4.13-3.98 (m, 4H), 3.75 (br s, 2H), 2.16-2.06 (m, 1H), 2.01-1.82 (m, 3H). MS (ESI) m/z: 695.1 (M+H)⁺; HPLC RT=2.24 min. [Method E]

### Example 52: Preparation of 2,2-difluoro-N-(4-fluoro-3-(trifluoromethyl)phenyl)-6-(2-methoxy-5-(4,5,6,7-tetrahydrothiazolo[5,4-c]pyridin-2-yl)benzamido)benzo[d][1,3]dioxole-5-carboxamide

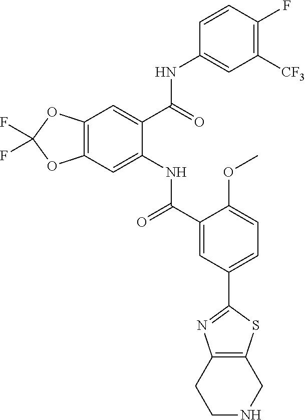 [SMILES: COc1ccc(-c2nc3c(s2)CNCC3)cc1C(=O)Nc1cc2c(cc1C(=O)Nc1ccc(F)c(C(F)(F)F)c1)OC(F)(F)O2]

Example 52 (8 mg, 0.01 mmol, 4% yield) was prepared in the same way as example 43-1 by replacing intermediate 25 with intermediate 28 followed by deprotection with 20% TFA in DCM. ¹H NMR (400 MHz, DMSO-d6) δ 11.77 (br s, 1H), 10.84 (br d, J=1.2 Hz, 1H), 8.56 (s, 1H), 8.49 (d, J=2.4 Hz, 1H), 8.30 (dd, J=2.6, 6.5 Hz, 1H), 8.08-7.98 (m, 2H), 7.93 (s, 1H), 7.55 (t, J=9.9 Hz, 2H), 7.32 (d, J=8.8 Hz, 1H), 4.05 (s, 3H), 3.93 (s, 2H), 3.02 (t, J=5.6 Hz, 2H), 2.79-2.68 (m, 2H). MS (ESI) m/z: 651.2 (M+H)⁺; HPLC RT=2.31 min. [Method E]

### Example 53: Preparation of 6-(5-(5-(2-amino-2-oxoethyl)-4,5,6,7-tetrahydrothiazolo[5,4-c]pyridin-2-yl)-2-methoxybenzamido)-2,2-difluoro-N-(4-fluoro-3-(trifluoromethyl)phenyl)benzo[d][1,3]dioxole-5-carboxamide

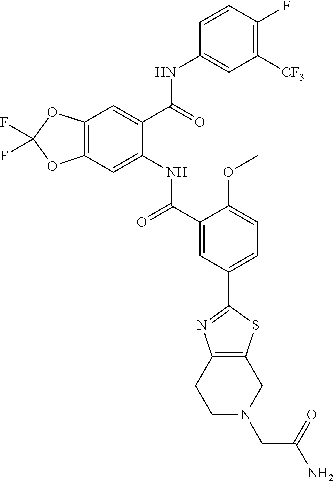 [SMILES: COc1ccc(-c2nc3c(s2)CN(CC(N)=O)CC3)cc1C(=O)Nc1cc2c(cc1C(=O)Nc1ccc(F)c(C(F)(F)F)c1)OC(F)(F)O2]

To a solution of example 52 (20 mg, 0.03 mmol) and 2-bromoacetamide (7 mg, 0.05 mmol) in DMF (0.5 mL) and THF (1.5 mL) was added TEA (0.021 mL, 0.15 mmol) and the reaction mixture stirred at rt for 15 h. The reaction mixture was then concentrated under vacuum and the residue subjected to reverse phase HPLC purification using Waters Xbridge C18 column, 19×150 mm. 5 microns, Mobile Phase A: 10-mM NH₄OAc; Mobile Phase B: ACN; Gradient: 15-65% B over 25 min, then a 5 minute hold at 100% B at a flow rate of 15 mL/min to yield example 53 (1.2 mg, 0.0010 mmol) 5.0% yield). ¹H NMR (400 MHz, DMSO-d₆) δ 11.87-11.71 (in. 1H), 10.87 (br s, 1H), 8.58 (s, 1H) 8.52 (d, J=2.5 Hz, H), 8.36-8.25 (in, 1H), 8.11-7.99 (in. 2H), 7.97 (s, 1H), 7.57 (t, J=9.8 Hz, 1H), 7.33-7.32 (in. 1H), 7.40-7.25 (in. 11H₁) 7.15 (brs, H), 4.07 (s, 3=), 3.81 (, 2H), 3.14 (s, 2H), 2.96-2.81 (m, 4H). MS (ESI) m/z: 708.4 (M+Hd; HPLC RT=2.3 min. [Method E]

Examples 54 to 97 were prepared by the general procedures described for Example 52.

TABLE 4

| | | | MS | | LC RT |
|---|---|---|---|---|---|
| Ex. | | | (ESI) | | (min) |
| No. | Structure | Name | (M + H)⁺. | ¹H NMR | Meth. |
| 54 | 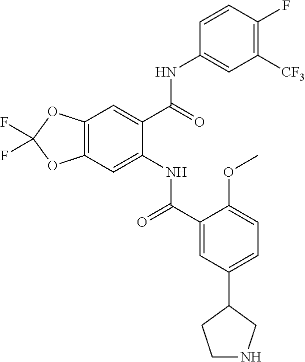 [SMILES: COc1ccc(C2CCNC2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5- (pyrrolidin-3- yl)benzamido)benzo[d] [1,3]dioxole-5- carboxamide | 582.2 | ¹H NMR (400 MHz, DMSO- d₆) δ 11.74 (s, 2H), 8.56 (s, 1H), 8.29 (dd, J = 6.5, 2.8 Hz, 1H), 8.05-7.96 (m, 1H), 7.94 (s, 1H), 7.90 (d, J = 2.2 Hz, 1H), 7.55 (t, J = 9.8 Hz, 1H), 7.48 (dd, J = 8.7, 2.3 Hz, 1H), 7.16 (d, J = 8.6 Hz, 1H), 4.00-3.94 (m, 3H), 3.28- 3.25 (m, 2H), 3.19 (br d, J = 7.6 Hz, 1H), 3.09-3.04 (m, 1H), 3.00-2.94 (m, 1H), 2.71- 2.63 (m, 1H), 2.24-2.12 (m, 1H), 1.87 (s, 1H) SFC Conditions: Isomers separated by SFC with Example 46 being Peak I (RT = 6.6 min., >95% ee) Instrument: Thar SFC 350 Prep SFC Column: Chiralpak IG 250 × 30 mm, 5 micron, Mobile phase: 70% CO₂ with | 2.11 E |
| | | | | 30% of 0.2% ammonia in | |
| | | | | MeOH, Flow conditions: | |
| | | | | 150 g/min, Back Pressure: | |
| | | | | 100 bar, Temperature: 30° C., | |
| | | | | Detector wavelength: 220 nm, | |
| | | | | Analytical method: Column: | |
| | | | | Chiralpak IG 250 × 4.6 mm, | |
| | | | | 5 micron, Mobile phase: 70% | |
| | | | | CO₂ with 30% of 0.2% | |
| | | | | ammonia in MeOH, Flow | |
| | | | | conditions: 4 g/min, Back | |
| | | | | Pressure: 100 bar, | |
| | | | | Temperature: 30° C., Detector | |
| | | | | wavelength: 220 nm, to afford | |
| | | | | homochiral (Peak-1, RT = | |
| | | | | 6.6 min., >95% ee) (90 mg, | |
| | | | | 0.15 mmol, 33% yield) and | |
| | | | | homochiral (Peak-2, RT = | |
| | | | | 11 min., >95% ee) (50 mg, | |
| | | | | 0.09 mmol, 18% yield). | |
| 55 | 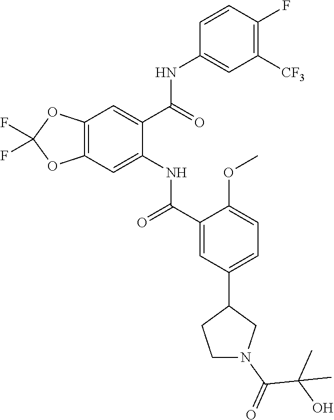 [SMILES: COc1ccc(C2CCN(C(=O)C(C)(C)O)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(1-(2-hydroxy-2- methylpropanoyl) pyrrolidin-3-yl)-2- methoxybenzamido) benzo[d][1,3]dioxole-5- carboxamide | 668.2 | ¹H NMR (400 MHz, DMSO- d₆) δ 11.73 (d, J = 3.2 Hz, 1H), 10.86 (s, 1H), 8.56 (d, J = 2.7 Hz, 1H), 8.36-8.24 (m, 1H), 8.08-7.98 (m, 1H), 7.94 (s, 2H), 7.62-7.43 (m, 2H), 7.20 (dd, J = 8.4, 3.8 Hz, 1H), 5.23- 5.15 (m, 1H), 4.35-4.07 (m, 1H), 3.99 (s, 3H), 3.83-3.68 (m, 1H), 3.53 (br dd, J = 19.7, 9.9 Hz, 1H), 3.36 (br d, J = 1.2 Hz, 1H), 3.29-3,21 (m, 1H), 2.28-2.09 (m, 1H), 1.97- 1.79 (m, 1H), 1.38-1.28 (m, 6H) | 2.48 E |
| | From Isomer 2 | | | | |
| 57 | 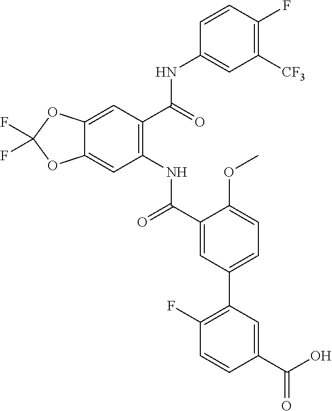 [SMILES: COc1ccc(-c2cc(C(=O)O)ccc2F)cc1C(=O)Nc1cc2c(cc1C(=O)Nc1ccc(F)c(C(F)(F)F)c1)OC(F)(F)O2] | 3′-((2,2-difluoro-6-((4- fluoro-3- (trifluoromethyl)phenyl) carbamoyl)benzo[d][1,3] dioxol-5- yl)carbamoyl)-6-fluoro- 4′-methoxy-[1,1′- biphenyl]-3-carboxylic acid | 649.2 | ¹H NMR (400 MHz, DMSO- d₆) δ 11.80 (s, 1H), 11.94 (s, 1H), 8.55 (s, 1H), 8.42-8.31 (m, 1H), 8.31-8.22 (m, 1H), 8.14-8.05 (m, 2H), 7.90 (s, 2H), 7.90-7.77 (m, 1H), 7.74- 7.51 (m, 1H), 7.48-7.35 (m, 2H), 3.93 (s, 3H) | 3.43 D |
| 58 | 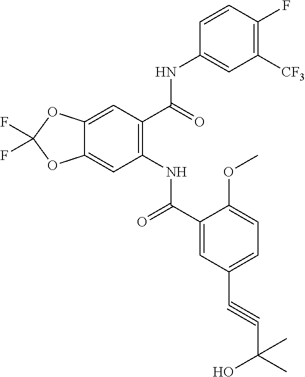 [SMILES: COc1ccc(C#CC(C)(C)O)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(3-hydroxy-3- methylbut-1-yn-1-yl)-2- methoxybenzamido) benzo[d][1,3]dioxole-5- carboxamide | 595.3 | ¹H NMR (400 MHz, DMSO- d₆) δ 11.74 (s, 1H), 10.85 (s, 1H), 8.53 (s, 1H), 8.30 (dd, J = 2.4, 6.6 Hz, 1H), 8.08- 7.97 (m, 2H), 7.94 (s, 1H), 7.63-7.49 (m, 2H), 7.23 (d, J = 8.8 Hz, 1H), 5.47 (s, 1H), 4.02 (s, 3H), 1.46 (s, 6H) | 2.49 E |
| 59 | 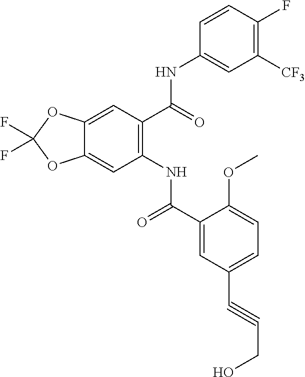 [SMILES: COc1ccc(C#CCO)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(3-hydroxyprop-1- yn-1-yl)-2- methoxybenzamido) benzo[d][1,3]dioxole-5- carboxamide | 567.2 | ¹H NMR (400 MHz, DMSO- d₆) δ 11.76 (br s, 1H), 10.90- 10.80 (m, 1H), 8.54 (s, 1H), 8.29 (dd, J = 2.6, 6.2 Hz, 1H), 8.07-7.97 (m, 2H), 7.95 (s, 1H), 7.67-7.51 (m, 2H), 7.49- 7.28 (m, 1H), 7.24 (d, J = 8.8 Hz, 1H), 5.32 (t, J = 5.9 Hz, 1H), 4.29 (d, J = 6.1 Hz, 2H), 4.02 (s, 3H) | 2.33 F |
| 61 | 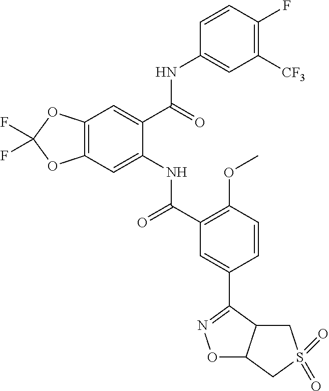 [SMILES: COc1ccc(C2=NOC3CS(=O)(=O)CC23)cc1C(=O)Nc1cc2c(cc1C(=O)Nc1ccc(F)c(C(F)(F)F)c1)OC(F)(F)O2] | 6-(5-(5,5-dioxido- 3a,4,6,6a- tetrahydrothieno[3,4- d]isoxazol-3-yl)-2- methoxybenzamido)- 2,2-difluoro-N-(4- fluoro-3- (trifluoromethyl)phenyl) benzo[d][1,3]dioxole-5- carboxamide | 670.2 | ¹H NMR (400 MHz, DMSO- d₆) δ 11.80-11.88 (m, 1H), 11.69-11.79 (m, 1H), 10.80- 10.96 (m, 1H), 8.54-8.63 (m, 1H), 8.21-8.35 (m, 1H), 7.91- 8.07 (m, 2H), 7.80-7.90 (m, 1H), 7.48-7.66 (m, 1H), 7.21- 7.37 (m, 1H), 5.38-5.53 (m, 1H), 4.74-4.85 (m, 1H), 4.05 (s, 3H), 3.60-3.73 (m, 1H), 3.32 (s, 1H), 2.05-2.25 (m, 2H) SFC Conditions: Isomers separated by SFC with Example ?? being Peak-1 (RT = 6.65 min., >95% ee) Instrument: Thor SFC-350 Prep SFC Column: Luxcellulose-4 250 × 30 mm, 5 micron, Mobile Phase: 65% CO₂ with 35% of 4 M methanolic ammonia in MeOH, Flow Conditions: 140 g/min, Back Pressure: | 2.41 D |
| | | | | 100 bar, Temperature: 30° C., | |
| | | | | Detector wavelength: 271 nm; | |
| | | | | Analytical method: Column: | |
| | | | | Luxcellulose-4 250 × 4.6 mm, | |
| | | | | 5 micron, Mobile Phase: 65% | |
| | | | | CO₂ with 35% of 4 M | |
| | | | | methanolic ammonia in | |
| | | | | MeOH, Flow Conditions: | |
| | | | | 4 g/min, Back Pressure: | |
| | | | | 100 bar, Temperature: 30° C., | |
| | | | | Detector wavelength: 271 nm, | |
| | | | | to afford homochiral (Peak-1, | |
| | | | | RT = 6.65 min., >95% ee) and | |
| | | | | homochiral (Peak-2, RT = | |
| | | | | 11.59 min., >95% ee). | |
| 62 | 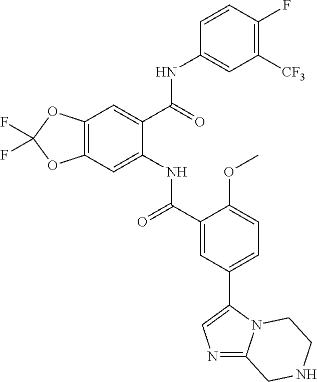 [SMILES: COc1ccc(-c2cnc3n2CCNC3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5- (5,6,7,8- tetrahydroimidazo[1,2- a]pyrazin-3- yl)benzamido)benzo[d] [1,3]dioxole-5- carboxamide | 634.2 | ¹H NMR (400 MHz, DMSO- d₆) δ 11.77 (s, 1H), 10.87 (s, 1H), 8.58 (s, 1H), 8.31 (td, J = 1.3, 7.7 Hz, 1H), 8.07- 8.00 (m, 2H), 7.96 (s, 1H), 7.67 (dd, J = 2.2, 8.8 Hz, 1H), 7.60-7.53 (m, 1H), 7.32 (d, J = 8.3 Hz, 2H), 6.98 (s, 1H), 4.04 (s, 3H), 3.91-3.86 (m, 4H), 3.05-3.01 (m, 2H) | 2.11 E |
| 64 | 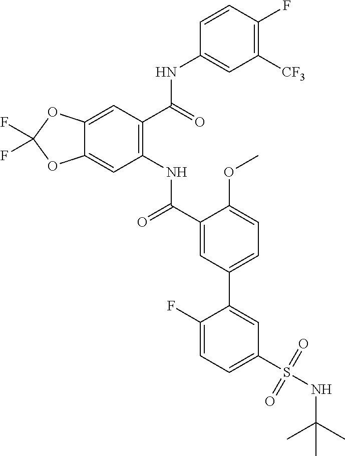 [SMILES: COc1ccc(-c2cc(S(=O)(=O)NC(C)(C)C)ccc2F)cc1C(=O)Nc1cc2c(cc1C(=O)Nc1ccc(F)c(C(F)(F)F)c1)OC(F)(F)O2] | 6-(5′-(N-(tert- butyl)sulfamoyl)-2′- fluoro-4-methoxy-{1,1′- biphenyl]-3- carboxamido)-2,2- difluoro-N-(4-fluoro-3- (trifluoromethyl)phenyl) benzo[d][1,3]dioxole-5- carboxamide | 742.2 | 1H NMR (400 MHz, DMSO- d6) δ 11.82 (br s, 1H), 10.87 (s, 1H), 8.60-8.49 (m, 1H), 8.34-8.26 (m, 1H), 8.22 (d, J = 1.2 Hz, 1H), 8.05-7.96 (m, 2H), 7.96-7.90 (m, 1H), 7.89- 7.75 (m, 2H), 7.65 (s, 1H), 7.60-7.47 (m, 2H), 7.40 (d, J = 9.0 Hz, 1H), 4.07 (s, 3H), 1.11 (s, 9H) | 2.71 E |
| 65 | 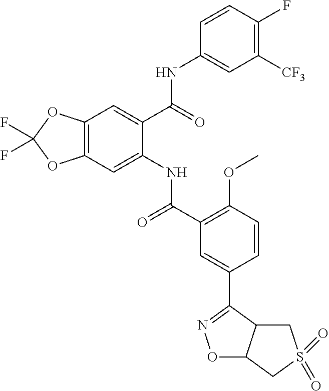 [SMILES: COc1ccc(C2=NOC3CS(=O)(=O)CC23)cc1C(=O)Nc1cc2c(cc1C(=O)Nc1ccc(F)c(C(F)(F)F)c1)OC(F)(F)O2] | 6-(5-(5,5-dioxido- 3a,4,6,6a- tetrahydrothieno[3,4- d]isoxazol-3-yl)-2- methoxybenzamido)- 2,2-difluoro-N-(4- fluoro-3- (trifluoromethyl)phenyl) benzo[d][1,3]dioxole-5- carboxamide | 672.1 | 1H NMR (400 MHz, DMSO- d6) δ 11.77 (s, 1H), 10.87 (s, 1H), 8.57 (s, 1H), 8.39-8.24 (m, 2H), 8.04 (td, J = 4.4, 7.3 Hz, 1H), 7.97 (s, 1H), 7.88-7.82 (m, 1H), 7.58 (t, J = 9.8 Hz, 1H), 7.34 (d, J = 8.5 Hz, 1H), 5.44 (ddd, J = 2.5, 6.5, 11.0 Hz, 1H), 4.80 (dt, J = 4.3, 10.4 Hz, 1H), 4.07 (s, 3H), 3.73-3.61 (m, 2H), 3.55-3.44 (m, 1H), 3.23-3.12 (m, 1H) | 2.28 E |
| 66 | 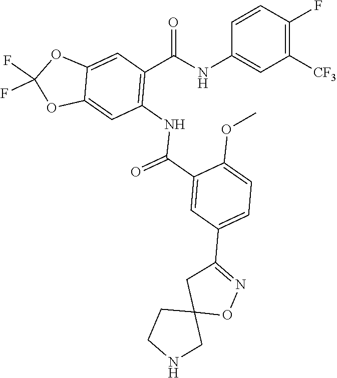 [SMILES: COc1ccc(C2=NOC3(CCNC3)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(1-oxa- 2,7-diazaspiro[4.4]non- 2-en-3- yl)benzamido)benzo[d] [1,3]dioxole-5- carboxamide | 637.2 | 1H NMR (400 MHz, DMSO- d6) δ 11.82 (s, 1H), 8.56 (s, 1H), 8.33-8.23 (m, 2H), 8.06- 7.99 (m, 1H), 7.96 (s, 1H), 7.84 (dd, J = 2.3, 8.7 Hz, 1H), 7.56 (t, J = 9.8 Hz, 1H), 7.32 (d, J = 8.8 Hz, 1H), 4.05 (s, 3H), 3.48 (br s, 2H), 3.11- 2.89 (m, 4H), 2.17-1.93 (m, 2H), 1.90 (s, 1H) | 2.09 E |
| 67 | 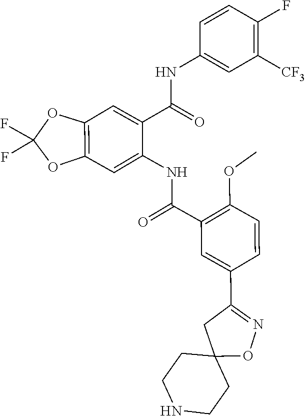 [SMILES: COc1ccc(C2=NOC3(CCNCC3)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(1-oxa- 2,8-diazaspiro[4.5]dec- 2-en-3- yl)benzamido)benzo[d] [1,3]dioxole-5- carboxamide | 651.2 | 1H NMR (400 MHz, DMSO- d6) δ 11.77 (s, 1H), 10.87 (s, 1H), 8.56-8.58 (m, 1 H) 8.27- 8.32 (m, 1 H) 7.99-8.06 (m, 2 H) 7.93-7.98 (m, 1 H) 7.83- 7.88 (m, 1 H) 7.52-7.61 (m, 1 H) 7.28-7.35 (m, 1 H) 4.03 (s, 3H) 3.40 (br s, 1 H) 3.23 (s, 2 H) 2.93-3.00 (m, 1 H) 2.75-2.82 (m, 2 H) 1.89- 1.92 (m, 2 H) 1.74 (br s, 2 H) | 2.09 E |
| 68 | 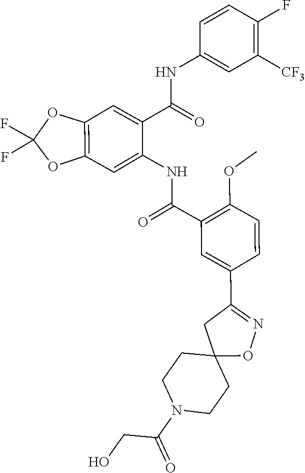 [SMILES: COc1ccc(C2=NOC3(CCN(C(=O)CO)CC3)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(8-(2- hydroxyacetyl)-1-oxa- 2,8-diazaspiro[4.5]dec- 2-en-3-yl)-2- methoxybenzamido) benzo[d][1,3]dioxole-5- carboxamide | 709.2 | 1H NMR (400 MHz, DMSO- d6) δ 11.77 (s, 1H), 10.86 (s, 1H), 8.57 (s, 1H), 8.32-8.25 (m, 2H), 8.07-8.00 (m, 1H), 7.96 (s, 1H), 7.86 (dd, J = 2.7, 8.8 Hz, 1H), 7.57 (t, J = 9.4 Hz, 1H), 7.33 (d, J = 8.8 Hz, 1H), 7.22-6.92 (m, 1H), 4.55 (t, J = 5.7 Hz, 1H), 4.12 (d, J = 5.4 Hz, 2H), 4.05 (s, 3H), 3.66- 3.62 (m, 1H), 3.54-3.50 (m, 1H), 3.44-3.40 (m, 1H), 3.28 (s, 3H), 1.80-1.71 (m, 4H) | 2.31 E |
| 69 | 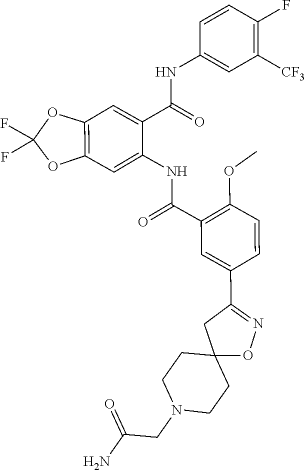 [SMILES: COc1ccc(C2=NOC3(CCN(CC(N)=O)CC3)C2)cc1C(=O)Nc1cc2c(cc1C(=O)Nc1ccc(F)c(C(F)(F)F)c1)OC(F)(F)O2] | 6-(5-(8-(2-amino-2- oxoethyl)-1-oxa-2,8- diazaspiro[4.5]dec-2-en- 3-yl)-2- methoxybenzamido)- 2,2-difluoro-N-(4- fluoro-3- (trifluoromethyl)phenyl) benzo[d][1,3]dioxole-5- carboxamide | 708.3 | 1H NMR (400 MHz, DMSO- d6) δ 11.77 (br s, 1 H) 10.87 (s, 1 H) 8.26-8.32 (m, 2 H) 8.00-8.08 (m, 1 H) 7.85- 7.89 (m, 1 H) 7.57-7.93 (m, 1 H) 7.37-7.69 (m, 1 H) 7.06- 7.37 (m, 2 H) 5.08 (t, J = 6.02 Hz, 2 H) 4.05 (s, 3 H) 3.60 (br t, J = 6.53 Hz, 2 H) 3.05 (d, J = 16.56 Hz, 4 H) 2.66-2.70 (m, 4 H) 1.76 (dt, J = 6.78, 3.14 Hz, 2 H) | 2.3 E |
| 70 | 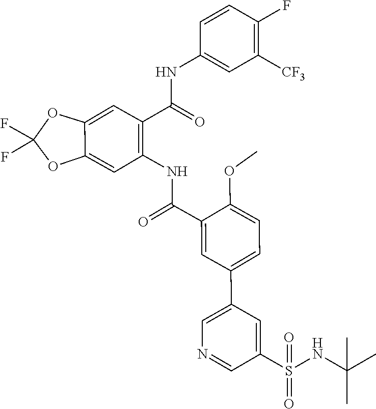 [SMILES: COc1ccc(-c2cncc(S(=O)(=O)NC(C)(C)C)c2)cc1C(=O)Nc1cc2c(cc1C(=O)Nc1ccc(F)c(C(F)(F)F)c1)OC(F)(F)O2] | 6-(5-(5-(N-(tert- butyl)sulfamoyl)pyridin- 3-yl)-2- methoxybenzamido)- 2,2-difluoro-N-(4- fluoro-3- (trifluoromethyl)phenyl) benzo[d][1,3]dioxole-5- carboxamide | 725.2 | 1H NMR (DMSO-d6, 400 MHz) δ 11.83 (s, 1H), 10.88 (s, 1H), 9.12 (d, J = 2.0 Hz, 1H), 8.94 (d, J = 2.2 Hz, 1H), 8.59 (s, 1H), 8.45 (t, J = 2.2 Hz, 1H), 8.38 (d, J = 2.4 Hz, 1H), 8.32 (dd, J = 6.7, 2.6 Hz, 1H), 8.00-8.10 (m, 2H), 7.97 (s, 1H), 7.88 (s, 1H), 7.57 (t, J = 9.8 Hz, 1H), 7.42 (d, J = 8.6 Hz, 1H), 4.08 (s, 3H), 1.14 ppm (s, 9H) | 2.55 E |
| 71 | 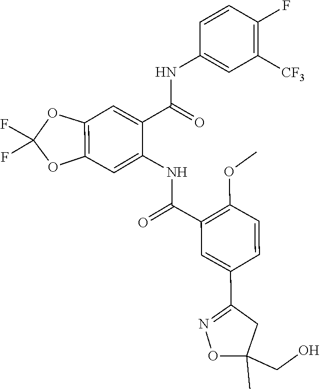 [SMILES: COc1ccc(C2=NOC(C)(CO)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5- (hydroxymethyl)-5- methyl-4,5- dihydroisoxazol-3-yl)-2- methoxybenzamido) benzo[d][1,3]dioxole-5- carboxamide | 624.0 (M − H) | 1H NMR (400 MHz, DMSO- d6) δ 11.77 (br s, 1 H) 10.87 (s, 1 H) 8.26-8.32 (m, 2 H) 8.00-8,08 (m, 1 H) 7.85- 7.89 (m, 1 H) 7.57-7.93 (m, 1 H) 7.37-7.69 (m, 1 H) 7.06- 7.37 (m, 2 H) 5.08 (t, J = 6.02 Hz, 1 H) 4.05 (s, 3 H) 3.60 (br t, J = 6.53 Hz, 2 H) 3.05 (d, J = 16.56 Hz, 1 H) 2.66-2.70 (m, 3 H) 1.76 (dt, J = 6.78, 3.14 Hz, 1 H) SFC conditions: Isomers separated by SFC with Example 71 being Peak- 2 (RT = 8.21 min., >95% ee). Instrument: Thor SFC-350 Prep SFC Column: Chiralpak IC 250 × 30 mm, 5 micron, Mobile Phase: 70% CO₂ with 30% of methanolic ammonia in MeOH, Flow Conditions: 140 g/min, Back Pressure: 100 bar, Temperature: 30° C., | 3.51 D |
| | | | | Detector Wavelength: 220 nm, | |
| | | | | Analytical method: | |
| | | | | Chiralpak IC 250 × 4.6 mm, | |
| | | | | 5 micron, Mobile Phase: 75% | |
| | | | | CO₂ with 25% of 4 M | |
| | | | | methanolic ammonia in | |
| | | | | MeOH, Flow Conditions: | |
| | | | | 4 g/min, Back Pressure: | |
| | | | | 100 bar, Temperature: 35° C., | |
| | | | | Detector Wavelength: 220 nm | |
| | | | | to afford homochiral (Peak-1, | |
| | | | | RT = 7.51 min., >95% ee) and | |
| | | | | homochiral (Peak-2, RT = | |
| | | | | 8.21 min, >95% ee). | |
| 72 | 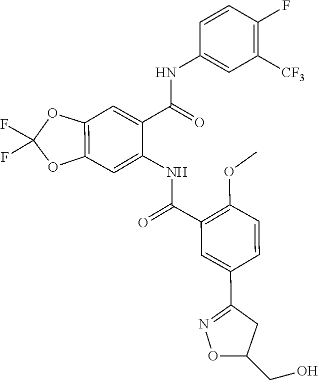 [SMILES: COc1ccc(C2=NOC(CO)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5- (hydroxymethyl)-4,5- dihydroisoxazol-3-yl)-2- methoxybenzamido) benzo[d][1,3]dioxole-5- carboxamide | 612.2 | 1H NMR (400 MHz, DMSO- d6) δ 11.78 (s, 1 H) 10.88 (s, 1 H) 8.58 (s, 1 H) 8.29-8.33 (m, 2 H) 8.02-8.07 (m, 1 H) 7.97 (s, 1 H) 7.85-7.89 (m, 1 H) 7.58 (t, J = 9.79 Hz, 1 H) 7.33 (d, J = 8.53 Hz, 1H) 4.96- 5.03 (m, 1H) 4.71 (br d, J = 3.51 Hz, 1 H) 4.06 (s, 3 H) 3.31 (br d, J = 2.51 Hz, 1 H) 3.18-3.27 (m, 1H) 2.69- 2.72 (m, 2 H) SFC conditions: Isomers separated by SFC with Example 72 being Peak- 2 (RT = 4.71 min., >95% ee), Instrument: Thar SFC-80 Prep SFC Column: Luxcellulose-4 250 × 21.5 mm, 5 micron, Mobile Phase: 60% CO₂ with 40% of 0.2% of 4 M methanolic ammonia in MeOH, Flow Conditions: | 3.33 D |
| | | | | 80 g/min, Back Pressure: | |
| | | | | 100 bar, Temperature: 30° C. | |
| | | | | Detector Wavelength: 272 nm, | |
| | | | | Analytical method: | |
| | | | | Column: Luxcellulose-4 | |
| | | | | 250 × 4.6 mm, 5 micron, | |
| | | | | Mobile Phase: 60% CO₂ with | |
| | | | | 40% of 0.2% of 4 M | |
| | | | | methanolic ammonia in | |
| | | | | MeOH, Flow Conditions: | |
| | | | | 4 g/min, Back Pressure: | |
| | | | | 100 bar, Temperature: | |
| | | | | 30° C., Detector Wavelength: | |
| | | | | 272 nm, to afford homochiral | |
| | | | | (Peak-2, 4.71 min., >95% ee) | |
| 73 | 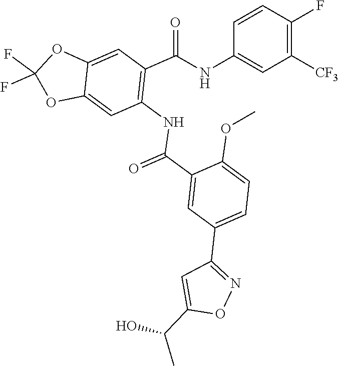 [SMILES: COc1ccc(-c2cc([C@H](C)O)on2)cc1C(=O)Nc1cc2c(cc1C(=O)Nc1ccc(F)c(C(F)(F)F)c1)OC(F)(F)O2] | (S)-2,2-difluoro-N-(4- fluoro-3- (trifluoromethyl)phenyl)- 6-(5-(5-(1- hydroxyethyl)isoxazol- 3-yl)-2- methoxybenzamido) benzo[d][1,3]dioxole-5- carboxamide | 624.3 | 1H NMR (400 MHz, DMSO- d6) δ 11.78 (s, 1H), 10.86 (s, 1H), 8.58 (s, 1H), 8.50 (d, J = 2.4 Hz, 1H), 8.30 (dd, J = 6.6. 2.4 Hz, 1H), 8.11- 7.98 (m, 2H), 7.96 (s, 1H), 7.56 (t, J = 9.9 Hz, 1H), 7.37 (d, J = 8.8 Hz, 1H), 6.89 (s, 1H), 5.77 (d, J = 5.6 Hz, 1H), 4.88 (quin, J = 6.3 Hz, 1H), 4.07 (s, 3H), 1.46(d, J = 6.8 Hz, 3H) | 2.37 E |
| 74 | 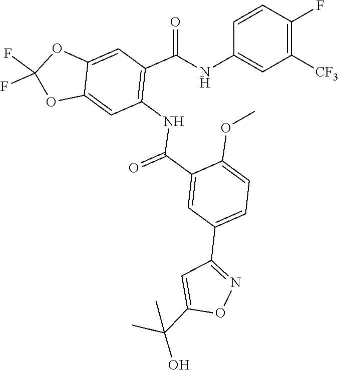 [SMILES: COc1ccc(-c2cc(C(C)(C)O)on2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5-(2- hydroxypropan-2- ylisoxazol-3-yl)-2- methoxybenzamido) benzo[d][1,3]dioxole-5- carboxamide | 638.2 | 1H NMR (400 MHz, DMSO- d6) δ 11.79 (s, 1H), 10.86 (s, 1H), 8.58 (s, 1H), 8.50 (s, 1H), 8.31 (br d, J = 6.6 Hz, 1H), 8.12-8.00 (m, 2H), 7.96 (s, 1H), 7.64-7.50 (m, 1H), 7.37 (d, J = 8.6 Hz, 1H), 6.86 (d, J = 1.2 Hz, 1H), 5,66 (s, 1H), 4.07 (s, 3H), 1.52 (s, 6H) | 2.42 E |
| 75 | 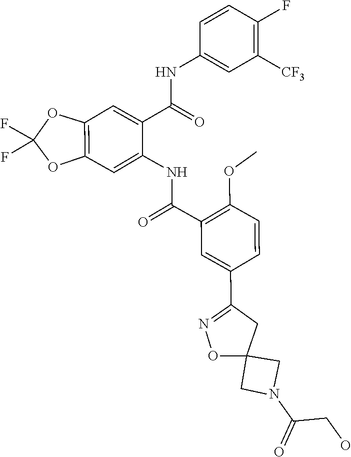 [SMILES: COc1ccc(C2=NOC3(C2)CN(C(=O)CO)C3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(2-(2- hydroxyacetyl)-5-oxa- 2,6-diazaspiro[3.4]oct- 6-en-7-yl)-2- methoxybenzamido) benzo[d][1,3]dioxole-5- carboxamide | 681.2 | 1H NMR (400 MHz, DMSO- d6) δ 11.78 (s, 1H), 10.86 (s, 1H), 8.57 (s, 1H), 8.33-8.25 (m, 2H), 8.06-8.00 (m, 1H), 7.96 (s, 1H), 7.85 (dd, J = 2.2, 8.3 Hz, 1H), 7.57 (t, J = 9.8 Hz, 1H), 7.34 (d, J = 8.8 Hz, 1H), 5.01 (t, J = 6.1 Hz, 1H), 4.52- 4.46 (m, 1H), 4.44-4.39 (m, 1H), 4.22 (br dd, J = 1.2, 10.0 Hz, 1H), 4.12-4.02 (m, 4H), 3.94 (d, J = 6.1 Hz, 2H), 3.80 (s, 2H), 2.90 (s, 1H) | 2.2 E |
| 76 | 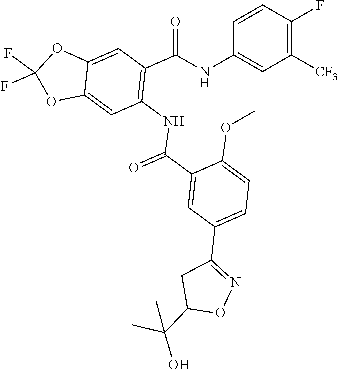 [SMILES: COc1ccc(C2=NOC(C(C)(C)O)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5-(2- hydroxypropan-2-yl)- 4,5-dihydroisoxazol-3- yl)-2- methoxybenzamido) benzo[d][1,3]dioxole-5- carboxamide | 640.3 | 1H NMR (400 MHz, DMSO- d6) δ 10.82-11.06 (m, 1 H) 10.65-10.79 (m, 1 H) 8.58- 8.60 (m, 1 H) 8,56-8.57 (m, 1 H) 8.28-8.33 (m, 1 H) 8.00- 8.06 (m, 1 H) 7.96-7.98 (m, 1 H) 7.84-7.89 (m, 1 H) 7.54- 7.61 (m, 1 H) 7.28-7.35 (m, 1 H) 4.58 (s, 1 H) 4.39-4.54 (m, 1 H) 4.08 (s, 3 H) 3.12- 3.64 (m, 2H) 1.21 (d, J = 2.51 Hz, 6H) SFC Conditions: Isomers separated by SFC with Example 76 being Peak-2 (RT = 4.5 min., >95% ee). Instrument: Thar SFC-80, Prep SFC Column: Luxcellulose-4 250 × 21.5 mm, 5 micron, Mobile Phase: 65% CO₂ with 35% of 0.2% of 4 M methanolic ammonia in MeOH, Flow Conditions: | 3.59 D |
| | | | | 80 g/min, Back Pressure: | |
| | | | | 100 bar, Temperature: 30°C., | |
| | | | | Detector Wavelength: 272 | |
| | | | | Analytical method: | |
| | | | | Instrument: Thar SFC-80, | |
| | | | | Column: Luxcellulose-4 | |
| | | | | 250 × 4.6 mm, 5 micron, | |
| | | | | Mobile Phase: 65% CO₂ with | |
| | | | | 35 % of 0.2% of 4 M | |
| | | | | methanolic ammonia in | |
| | | | | MeOH, Flow Conditions: | |
| | | | | 4 g/min, Back Pressure: | |
| | | | | 100 bar, Temperature: 30 °C., | |
| | | | | Detector Wavelength: | |
| | | | | 272 nm, to afford homochiral | |
| | | | | (Peak-1, RT = 3.2 min., | |
| | | | | % ee) and homochiral (Peak-2, | |
| | | | | RT = 4.5 min., >95% ee) | |
| 77 | 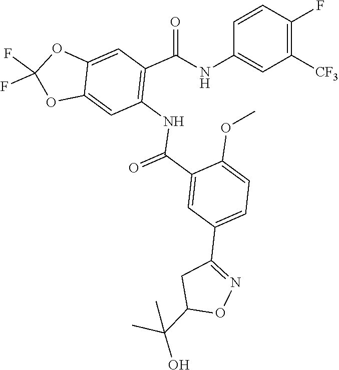 [SMILES: COc1ccc(C2=NOC(C(C)(C)O)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5-(2- hydroxypropan-2-yl)- 4,5-dihydroisoxazol-3- yl)-2- methoxybenzamido) benzo[d][1,3]dioxole-5- carboxamide | 640.2 | 1H NMR (400 MHz, DMSO- d6) δ 10.82-11.06 (m, 1 H) 10.65-10.79 (m, 1 H) 8.58- 8.60 (m, 1 H) 8.56-8.57 (m, 1 H) 8.28-8.33 (m, 1 H) 8.00- 8.06 (m, 1 H) 7.96-7.98 (m, 1 H) 7.84-7.89 (m, 1 H) 7.54- 7.61 (m, 1 H) 7.28-7.35 (m, 1 H) 4.58 (s, 1 H) 4.39-4.54 (m, 1 H) 4.08 (s, 3 H) 3.12- 3.64 (m, 2H) 1.21 (d, J = 2.51 Hz, 6H) SFC Conditions: Isomers separated by SFC with Example 77 being Peak-1 (RT = 3.2 min., >95% ee). Instrument: Thar SFC-80, Prep SFC Column: Luxcellulose-4 250 × 21.5 mm, 5 micron, Mobile Phase: 65% CO₂ with 35% of 0.2% of 4 M methanolic ammonia MeOH, Flow Conditions: | 3.6 D |
| | | | | 80 g/min, Back Pressure: | |
| | | | | 100 bar, Temperature: | |
| | | | | 30° C., Detector Wavelength: | |
| | | | | 272 nm. | |
| | | | | Analytical method: | |
| | | | | Instrument: Thar SFC-80, | |
| | | | | Column: Luxcellulose-4 | |
| | | | | 250 × 4.6 mm, 5 micron, | |
| | | | | Mobile Phase: 65% CO₂ with | |
| | | | | 35% of 0.2% of 4 M methanolic | |
| | | | | ammonia in MeOH, Flow | |
| | | | | Conditions: g/min, Back | |
| | | | | Pressure: 100 bar, | |
| | | | | Temperature: 30° C., Detector | |
| | | | | Wavelength: 272 nm, to afford | |
| | | | | homochiral (Peak-1, RT = | |
| | | | | 3.2 min., % ee) and | |
| | | | | homochiral (Peak-2, RT = | |
| | | | | 4.5 min., >95% ee) | |
| 78 | 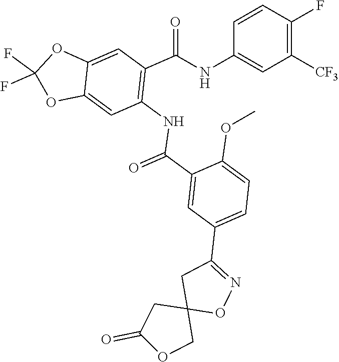 [SMILES: COc1ccc(C2=NOC3(COC(=O)C3)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(8- oxo-1,7-dioxa-2- azaspiro[4.4]non-2-en- 3- yl)benzamido)benzo[d] [1,3]dioxole-5- carboxamide | 652.1 | 1H NMR (400 MHz, DMSO- d6) δ 11.86-11.69 (m, 1H), 10.85 (s, 1H), 8.56 (s, 1H), 8.37-8.22 (m, 2H), 8.07- 7.99 (m, 1H), 7.95 (s, 1H), 7.88 (dd, J = 2.3, 8.7 Hz, 1H), 7.56 (t, J = 9.8 Hz, 1H), 7.35 (d, J = 8.8 Hz, 1H), 4.46-4.36 (m, 2H), 4.06 (s, 3H), 3.74 (s, 2H), 2.66-2.53 (m, 2H) | 2.45 E |
| 79 | 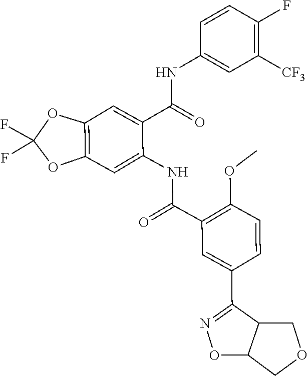 [SMILES: COc1ccc(C2=NOC3COCC23)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5- (3a,4,6,6a- tetrahydrofuro[3,4- dlisoxazol-3- yl)benzamido)benzo[d] [1,3]dioxole-5- carboxamide | 625.2 | 1H NMR (400 MHz, DMSO- d6) δ 10.06-10.12 (m, 1 H) 9.22-9.29 (m, 1 H) 8.82- 8.87 (m, 1 H) 8.69-8.73 (m, 1 H) 8.63-8.75 (m, 1 H) 8.63- 8.68 (m, 1 H) 8.48-8.55 (m, 1 H) 8.34-8.44 (m, 1 H) 8.02- 8.13 (m, 1 H) 7.40-7.44 (m, 1 H) 6.50-6.59 (m, 1 H) 5.96- 6.01 (m, 1 H) 4.73-4.78 (m, 4H) 4.05 (s, 3 H) SFC Conditions: Isomers separated by SFC with Example 78 being Peak- 2 (RT = 4.49 min., >95% ee). Instrument details: Make/Model: Thor SFC-350, Preparative SFC Conditions, Column/dimensions: Chiralpak IC (250 × 30) mm, 5 u, % CO₂: 70%, % Co solvent: 30% of 4 M methanolic ammonia in | 3.04 D |
| | | | | MeOH, Total Flow: 140 g/min, | |
| | | | | Back Pressure: 100 bar, | |
| | | | | Temperature: 30° C., UV: | |
| | | | | 220 nm, Analytical SFC | |
| | | | | Conditions, | |
| | | | | Column/dimensions: | |
| | | | | Chiralpak IC (250 × 4.6) mm, | |
| | | | | 5 u, % CO₂: 75%, % Co | |
| | | | | solvent: 25% of 4 M | |
| | | | | methanolic ammonia in | |
| | | | | MeOH, Total Flow: 4.0 g/min, | |
| | | | | Back Pressure: 100 bar, | |
| | | | | Temperature: 35° C., | |
| | | | | UV: 220 nm | |
| 80 | 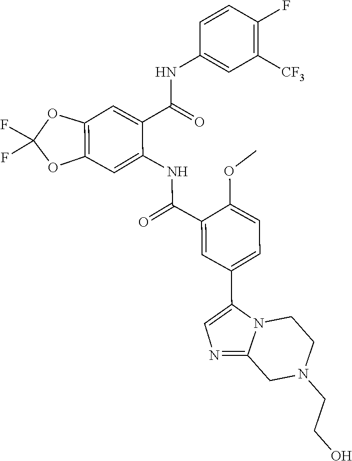 [SMILES: COc1ccc(-c2cnc3n2CCN(CCO)C3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(7-(2- hydroxyethyl)-5,6,7,8- tetrahydroimidazo[1,2- a]pyrazin-3-yl)-2- methoxybenzamido) benzo[d][1,3]dioxole-5- carboxamide | 678.2 | 1H NMR (400 MHz, DMSO- d6) δ 10.88-10.83 (m, 1H), 10.85 (s, 1H), 8.66 (s, 1H), 8.73-8.54 (m, 1H), 8.15 (dd, J = 2.6, 6.5 Hz, 1H), 8.18- 8.11 (m, 1H), 8.08-7.97 (m, 1H), 7.81 (dd, J = 1.7, 8.7 Hz, 1H), 7.54 (t, J = 9.8 Hz, 1H), 7.39 (s, 1H), 7.17 (s, 1H), 4.22 (s, 1H), 4.30-4.12 (m, 1H), 3.91 (s, 3H), 3.99-3.85 (m, 1H), 3.67-3.59 (m, 1H), 3.41-3.40 (m, 1H), 3.49- 3.39 (m, 1H), 3.32 (s, 1H), 2.69-2.66 8080 (m, 1H), 2.38- 2.28 (m, 1H), 1.61-1.49 (m, 1H), 1.15 (s, 1H) | 2.11 F |
| 81 | 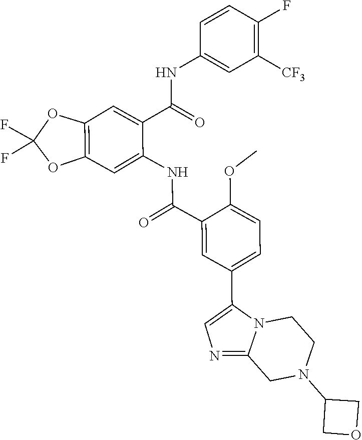 [SMILES: COc1ccc(-c2cnc3n2CCN(C2COC2)C3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(7- (oxetan-3-yl)-5,6,7,8- tetrahydroimidazo[1,2- a]pyrazin-3- yl)benzamido)benzo[d] [1,3]dioxole-5- carboxamide | 690.2 | 1H NMR (400 MHz, DMSO- d6) δ 11.88-11.76 (m, 1H), 10.92-10.83 (m, 1H), 8.58 (s, 1H), 8.37-8.24 (m, 1H), 8.20- 8.13 (m, 1H), 8.07-7.94 (m, 1H),7.86-7.77 (m, 1H), 7.76- 7.69 (m, 1H), 7.63-7.50 (m, 1H), 7.45-7.36 (m, 1H), 7.14- 7.04 (m, 1H), 4.71-4.60 (m, 1H), 4.58-4.47 (m, 2H), 4.17- 3.98 (m, 5H), 3.94-3.78 (m, 2H), 3.21-3.05 (m, 2H), 2.96- 2.76 (m, 21) | 1.93 E |
| 82 | 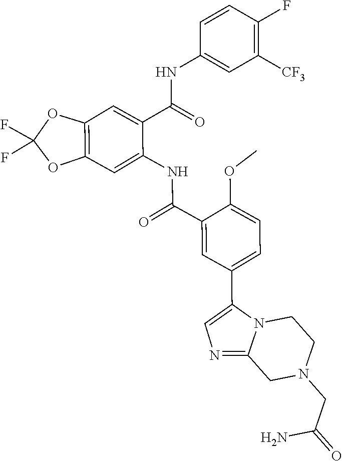 [SMILES: COc1ccc(-c2cnc3n2CCN(CC(N)=O)C3)cc1C(=O)Nc1cc2c(cc1C(=O)Nc1ccc(F)c(C(F)(F)F)c1)OC(F)(F)O2] | 6-(5-(7-(2-amino-2- oxoethyl)-5,6,7,8- tetrahydroimidazo[1,2- a]pyrazin-3-yl)-2- methoxybenzamido)- 2,2-difluoro-N-(4- fluoro-3- (trifluoromethyl)phenyl) benzo[d][1,3]dioxole-5- carboxamide | 691.2 | 1H NMR (400 MHz, DMSO- d6) δ 11.88-11.70 (m, 1H), 10.93-10.78 (m, 1H), 8.66- 8.53 (m, 1H), 8.36-8.25 (m, 1H), 8.11-8.00 (m, 1H), 7.99- 7.92 (m, 1H), 7.75-7.65 (m, 1H), 7.61-7.51 (m, 1H), 7.44- 7.36 (m, 1H), 7.35-7.29 (m, 1H), 7.23-7.13 (m, 1H), 4.13- 3.96 (m, 5H), 3.85-3.55 (m, 2H), 3.21-3.05 (m, 2H), 2.97- 2.83 (m, 2H) | 2.07 E |
| 83 | 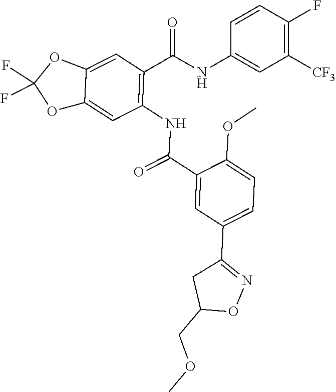 [SMILES: COCC1CC(c2ccc(OC)c(C(=O)Nc3cc4c(cc3C(=O)Nc3ccc(F)c(C(F)(F)F)c3)OC(F)(F)O4)c2)=NO1] | 2,2-difluoro-N-(4- fluoro-3- (trifluoromethyl)phenyl)- 6-(2-methoxy-5-(5- (methoxymethyl)-4,5- dihydroisoxazol-3- yl)benzamido)benzo[d] [1,3]dioxole-5- carboxamide | 626.2 | 1H NMR (400 MHz, DMSO- d6) δ 10.84-10.91 (m, 2 H) 8.57-8.61 (m, 1 H) 8.24- 8.31 (m, 2 H) 7.95-8.04 (m, 1 H) 7.83-7.88 (m, 2 H) 7.51- 7.58 (m, 1 H) 7.29-7.33 (m, 1 H) 4.78-4.88 (m, 1 H) 4.06 (s, 3 H) 3.42-3.51 (m, 2 H) 3.28-3.31 (m, 2 H) 3.23 (s, 3H) | 3.73 D |
| 84 | 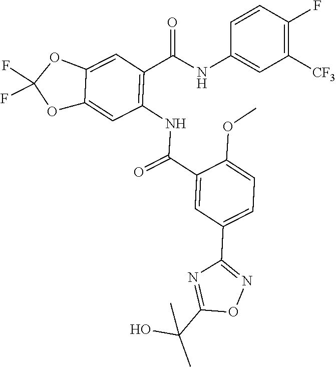 [SMILES: COc1ccc(-c2noc(C(C)(C)O)n2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(5-(5-(2- hydroxypropan-2-yl)- 1,2,4-oxadiazol-3-yl)-2- methoxybenzamido) benzo[d][1,3]dioxole-5- carboxamide | 639.2 | 1H NMR (400 MHz, DMSO- d6) δ 11.81 (s, 1H), 10.88 (s, 1H), 8.69 (d, J = 2.2 Hz, 1H), 8.57 (s, 1H), 8.35-8.28 (m, 1H), 8.19 (dd, J = 8.7, 2.3 Hz, 1H), 8.07-8.00 (m, 1H), 7.97 (s, 1H), 7.57 (t, J = 9.8 Hz, 1H), 7.43 (d, J = 8.8 Hz, 1H), 6.09 (s, 1H), 4.10 (s, 3H), 1.62 (s, 6H) | 2.47 E |
| 87 | 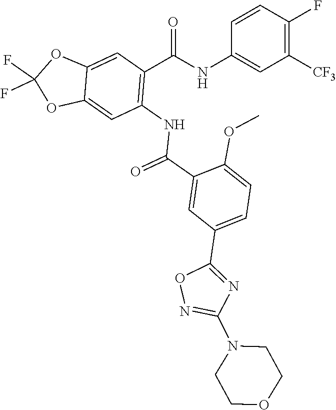 [SMILES: COc1ccc(-c2nc(N3CCOCC3)no2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(2-methoxy- 5-(3-morpholino-1,2,4- oxadiazol-5- yl)benzamido)benzo[d] [1,3]dioxole-5- carboxamide | 666.2 | 1H NMR (400 MHz, DMSO- d6) δ 11.80 (br s, 1H), 10.85 (br s, 1H), 8.65 (br s, 1H), 8.55 (s, 1H), 8.34-8.26 (m, 1H), 8.22-8.15 (m, 1H), 8.09- 7.98 (m, 1H), 7.98-7.89 (m, 1H), 7.60-7.51 (m, 1H), 7.48- 7.39 (m, 1H), 4.11 (s, 3H), 3.76-3.66 (m, 4H), 3.42- 3.38 (m, 4H) | 2.66 E |
| 88 | 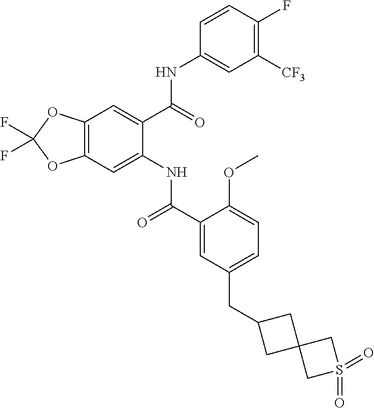 [SMILES: COc1ccc(CC2CC3(C2)CS(=O)(=O)C3)cc1C(=O)Nc1cc2c(cc1C(=O)Nc1ccc(F)c(C(F)(F)F)c1)OC(F)(F)O2] | 6-(5-((2,2-dioxido-2- thia-6- azaspiro[3.3]heptan-6- yl)methyl)-2- methoxybenzamido)- 2,2-difluoro-N-(4- fluoro-3- (trifluoromethyl)phenyl) benzo[d][1,3]dioxole-5- carboxamide | 672.2 | 1H NMR (400 MHz, DMSO- d6) δ 10.85 (s, 2H), 8.57 (s, 1H), 8.34-8.24 (m, 1H), 8.02 (ddd, J = 3.3, 5.7, 6.8 Hz, 1H), 7.94 (s, 1H), 7.91 (d, J = 2.4 Hz, 1H), 7.56 (t, J = 9.8 Hz, 1H), 7.45 (dd, J = 1.8, 8.7 Hz, 1H), 7.17 (d, J = 8.6 Hz, 1H), 4.02 (s, 4H), 4.31 (s, 4H), 3.98 (s, 3H), 3.54 (s, 2H) | 2.25 E |
| 92 | 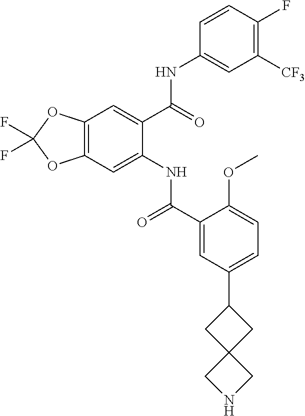 [SMILES: COc1ccc(C2CC3(CNC3)C2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(2- azaspiro[3.3]heptan-6- yl)benzamido)benzo[d] [1,3]dioxole-5- carboxamide | 608.3 | 1H NMR (400 MHz, DMSO- d6) δ 11.71 (s, 1H), 10.83 (s, 1H), 8.57 (s, 1H), 8.25 (dd, J = 2.1, 6.0 Hz, 1H), 8.02- 7.90 (m, 2H), 7.79 (d, J = 1.7 Hz, 1H), 7.52 (t, J = 10.0 Hz, 1H), 7.38 (dd, J = 2.4, 8.6 Hz, 1H), 7.13 (d, J = 8.6 Hz, 2H), 3.94 (s, 3H), 3.71 (br d, J = 0.7 Hz, 1H), 3.49 (br d, J = 1.5 Hz, 4H), 2.18-2.05 (m, 2H), 1.83 (s, 2H) | 2.13 E |
| 93 | 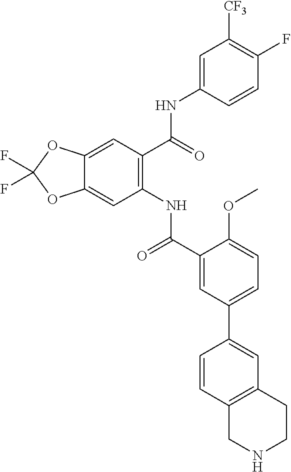 [SMILES: COc1ccc(-c2ccc3c(c2)CCNC3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5- (1,2,3,4- tetrahydroisoquinolin-6- yl)benzamido)benzo[d] [1,3]dioxole-5- carboxamide | 644.2 | 1H NMR (DMSO-d6, 400 MHz) δ 11.73-11.94 (m, 1H), 10.71-11.00 (m, 1H), 8.59 (s, 1H), 8.32 (dd, J = 6.5, 2.6 Hz, 1H), 8.25 (d, J = 2.7 Hz, 1H), 7.99-8.07 (m, 1H), 7.95 (s, 1H), 7.85 (dd, J = 8.7, 2.6 Hz, 1H), 7.57 (t, J = 9.8 Hz, 1H), 7.33-7.41 (m, 2H), 7.30 (d, J = 8.8 Hz, 1H), 7.10 (d, J = 8.1 Hz, 1H), 4.04 (s, 3H), 3.85-3.93 (m, 2H), 2.93-3.03 (m, 2H), 2.73-2.81 ppm (m, 2H) | 2.23 E |
| 94 | 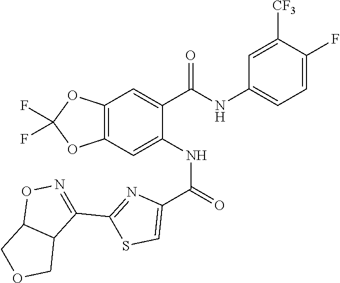 [SMILES: O=C(Nc1cc2c(cc1C(=O)Nc1ccc(F)c(C(F)(F)F)c1)OC(F)(F)O2)c1csc(C2=NOC3COCC23)n1] | N-(2,2-difluoro-6-((4- fluoro-3- (trifluoromethyl)phenyl) carbamoyl)benzo[d][1,3] dioxol-5-yl)-2- (3a,4,6,6a- tetrahydrofuro[3,4- d]isoxazol-3-yl)thiazole- 4-carboxamide | 601.2 | 1H NMR (400 MHz, DMSO- d6) δ 11.56 (s, 1H), 10.85 (s, 1H) 8.65-8.68 (m, 1H) 8.54- 8.57 (m, 1H) 8.17-8.22 (m, 1H) 8.02-8.06 (m, 1H) 7.92- 7.99 (m, 1H) 7.48-7.56 (m, 1H) 4.36-4.44 (m, 1H) 4.36- 4.45 (m, 1H) 4.13-4.19 (m, 1H) 3.58-3.70 (m, 1H) 3.57- 3.71 (m, 1H) 1.85-1.87 (m, 1H) SFC Conditions: Isomers separated by SFC with Example 94 being Peak- I (RT = 1.73 min., >95% ee), Instrument: PIC 175, Prep SFC Column: Chiralpak IG | 3.21 D |
| | | | | 250 × 30 mm, 5 micron, | |
| | | | | Mobile Phase: 50% CO₂ with | |
| | | | | 50% of 4 M methanolic | |
| | | | | ammonia in MeOH, Flow | |
| | | | | Conditions: 140 g/min, Back | |
| | | | | Pressure: 100 bar, | |
| | | | | Temperature: 40° C., Detector | |
| | | | | Wavelength: 285 nm, | |
| | | | | Analytical method: Column: | |
| | | | | Chiralpak IG 250 × 4.6 mm, | |
| | | | | 5 micron, Mobile Phase: 50% | |
| | | | | CO₂ with 50% of 4 M | |
| | | | | methanolic ammonia in | |
| | | | | MeOH, Flow Conditions: | |
| | | | | 4 g/min, Back Pressure: | |
| | | | | 100 bar, Temperature: 30° C., | |
| | | | | Detector Wavelength: 285 nm, | |
| | | | | to afford homochiral | |
| | | | | (Peak-1, RT = 1.73 min., | |
| | | | | >95% ee) | |
| 95 | 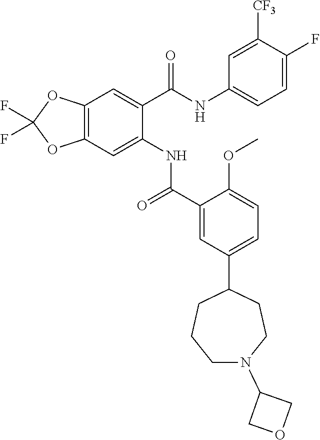 [SMILES: COc1ccc(C2CCCN(C3COC3)CC2)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(2-methoxy-5-(1- (oxetan-3-yl)azepan-4- yl)benzamido)benzo[d] [1,3]dioxole-5- carboxamide | 666.2 | 1H NMR (400 MHz, DMSO- d6) δ 11.70 (s, 1H), 10.84 (s, 1H), 8.56 (s, 1H), 8.30 (dd, J = 6.6, 2.4 Hz, 1H), 8.06- 7.98 (m, 1H), 7.93 (s, 1H), 7.87 (d, J = 2.2 Hz, 1H), 7.56 (t, J = 9.7 Hz, 1H), 7.43 (dd, J = 8.4. 2.3 Hz, 1H), 7.14 (d, J = 8.3 Hz, 1H), 4.55 (br t, J = 6.4 Hz, 2H), 4.46-4.34 (m, 2H), 3.96 (s, 3H), 3.77-3.63 (m, 1H), 2.89-2.80 (m, 91H), 2.62-2.55 (m, 1H), 1.88- 1.61 (m, 6H) | 2.69 E |
| 96 | 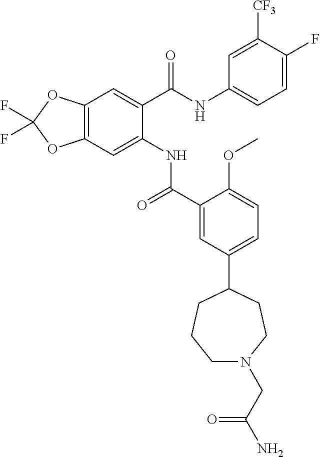 [SMILES: COc1ccc(C2CCCN(CC(N)=O)CC2)cc1C(=O)Nc1cc2c(cc1C(=O)Nc1ccc(F)c(C(F)(F)F)c1)OC(F)(F)O2] | 6-(5-(1-(2-amino-2- oxoethyl)azepan-4-yl)- 2-methoxybenzamido)- 2,2-difluoro-N-(4- fluoro-3- (trifluoromethyl)phenyl) benzo[d][1,3]dioxole-5- carboxamide | 667.2 | 1H NMR (400 MHz, DMSO- d6) δ 11.70 (s, 1H), 10.86 (s, 1H), 8.55 (s, 1H), 8.31 (dd, J = 6.2, 2.3 Hz, 1H), 8.07- 7.98 (m, 1H), 7.93 (s, 1H), 7.88 (d, J = 2.4 Hz, 1H), 7.56 (t, J = 9.8 Hz, 1H), 7.44 (dd, J = 8.6. 2.2 Hz, 1H), 7.27- 7.19 (m, 1H), 7.17-7.08 (m, 2H), 3.96 (s, 3H), 3.90 (s, 1H), 3.05 (br s, 2H), 2.89- 2.80 (m, 1H), 2.73 (br d, J = 2.4 Hz, 4H), 1.87-1.59 (m, 6H) | 2.53 E |
| 97 | 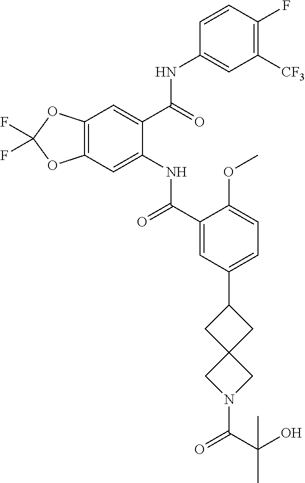 [SMILES: COc1ccc(C2CC3(C2)CN(C(=O)C(C)(C)O)C3)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(2-(2-hydroxy-2- methylpropanoyl)-2- azaspiro[3.3]heptan-6- yl)-2- methoxybenzamido) benzo[d][1,3]dioxole-5- carboxamide | 694.3 | 1H NMR (400 MHz, DMSO- d6) δ 11.72 (br s, 1H), 10.89- 10.79 (m, 1H), 8.56 (s, 1H), 8.30 (dd, J = 2.4, 6.4 Hz, 1H), 8.06-7.97 (m, 1H), 7.93 (s, 1H), 7.85(d, J = 2.2 Hz, 1H), 7.56 (t, J = 9.8 Hz, 1H), 7.42 (dd, J = 2.2, 8.6 Hz, 1H), 7.16 (d, J = 8.8 Hz, 1H), 5.03 (d, J = 16.6 Hz, 1H), 4.51 (s, 1H), 4.29 (s, 1H), 4.00-3.92(m, 4H), 3.76 (s, 1H), 3.41-3.37 (m, 1H), 2.57-2.53 (m, 1H), 2.26-2.14 (m, 2H), 1.23 (d, J = 6.6 Hz, 6H) | 2.38 E |
| 98 | 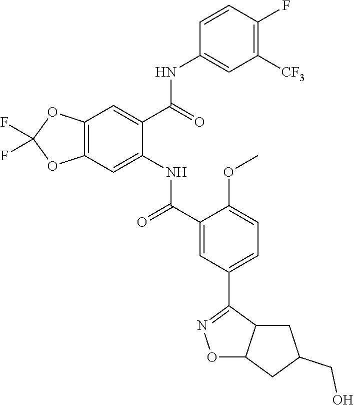 [SMILES: COc1ccc(C2=NOC3CC(CO)CC23)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl)phenyl)- 6-(5-(5- (hydroxymethyl)- 3a,5,6,6a-tetrahydro-4H- cyclopentafd]isoxazol- 3-yl)-2- methoxybenzamido) benzo[d][1,3]dioxole-5- carboxamide | 652.2 | ¹H NMR (400 MHz, DMSO- d6) δ ppm 11.71-11.84 (m, 1 H) 10.83-10.91 (m, 1 H) 8.58 (s, 1 H) 8.30-8.34 (m, 2 H) 8.01-8.07 (m, 1 H) 7.96- 7.98 (m, 1 H) 7.86-7.91 (m, 1 H) 7.54-7.61 (m, 1 H) 7.31- 7.35 (m, 1 H) 5.12-5.18 (m, 1 H) 4.47-4.52 (m, 1 H) 4.21- 4.27 (m, 1 H) 4.04-4.08 (m, 3 H) 3.34-3.44 (m, 2 H) 1.97- 2.05 (m, 1 H) 1.86-1.95 (m, 1 H) 1.78-1.85 (m, 2 H) 1.64- 1.72 (m, 1 H) 1.48-1.62 (m, 1 H) | 3.45 C |
| 99 | 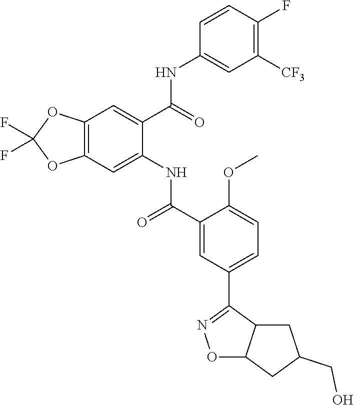 [SMILES: COc1ccc(C2=NOC3CC(CO)CC23)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(5-(5- (hydroxymethyl)- 3a,5,6,6a-tetrahydro-4H- cyclopenta[d]isoxazol- 3-yl)-2- methoxybenzamido) benzo[d][1,3]dioxole-5- carboxamide | 652.2 | ¹H NMR (400 MHz, DMSO- d6) δ ppm 11.74-11.91 (m, 1 H) 10.84-10.91 (m, 1 H) 8.58 (s, 1 H) 8.30-8.37 (m, 2 H) 8.02-8.06 (m, 1 H) 7.96- 7.98 (m, 1 H) 7.86-7.90 (m, 1 H) 7.57 (t, J = 9.79 Hz, 1 H) 7.31-7.35 (m, 1 H) 5.13- 5.20 (m, 1 H) 4.47-4.51 (m, 1 H) 4.09-4.18 (m, 1 H) 4.03- 4.06 (m, 3 H) 3.16-3.28 (m, 2 H) 2.09-2.29 (m, 4 H) 1.58- 1.73 (m, 1 H) 1.39-1.54 (m, 1 H) | 3.46 C |
| 100 | 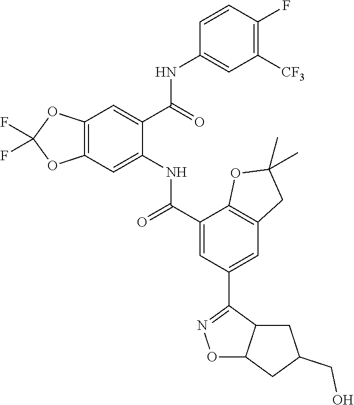 [SMILES: CC1(C)Cc2cc(C3=NOC4CC(CO)CC34)cc(C(=O)Nc3cc4c(cc3C(=O)Nc3ccc(F)c(C(F)(F)F)c3)OC(F)(F)O4)c2O1] | 2,2-difluoro-N-(4- fluoro-3- (trifluoromethyl)phenyl)- 6-(5-(5- (hydroxymethyl)- 3a,5,6,6a-tetrahydro-4H- cyclopentafdlisoxazol- 3-yl)-2,2-dimethyl-2,3- dihydrobenzofuran-7- carboxamido)benzo[d] [1,3]dioxole-5- carboxamide | 692.2 | ¹H NMR (400 MHz, DMSO- d6) δ ppm 11.35-11.57 (m, 1 H) 10.83-10.92 (m, 1 H) 8.54-8.59 (m, 1 H) 8.41- 8.48 (m, 1 H) 7,98-8.03 (m, 1 H) 7.93-7.97 (m, 1 H) 7.85- 7.92 (m, 1 H) 7.76-7.80 (m, 1 H) 7.51-7.61 (m, 1 H) 5.12 (dd, J = 8.82, 5.07 Hz, 1 H) 4.48 (t, J = 5.32 Hz, 1 H) 4.20 (br t, J = 8.75 Hz, 1 H) 3.37-3.44 (m, 2 H) 3.07- 3.19 (m, 3 H) 2.19 (s, 1 H) 1.96-2.03 (m, 1 H) 1.87- 1.94 (m, 1 H) 1.78-1.86 (m, 1 H) 1.64-1.72 (m, 1 H) 1.54- 1.64 (m, 2 H) 1.47-1.52 (m, 6 H) | 0.86 C |
| 101 | 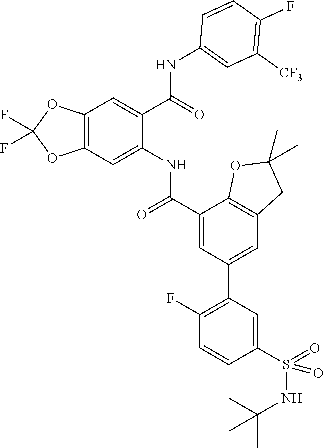 [SMILES: CC(C)(C)NS(=O)(=O)c1ccc(F)c(-c2cc3c(c(C(=O)Nc4cc5c(cc4C(=O)Nc4ccc(F)c(C(F)(F)F)c4)OC(F)(F)O5)c2)OC(C)(C)C3)c1] | 6-(5-(5-(N-(tert- butyl)sulfamoyl)-2- fluorophenyl)-2,2- dimethyl-2,3- dihydrobenzofuran-7- carboxamido)-2,2- difluoro-N-(4-fluoro-3- (trifluoromethyl)phenyl) benzo[d][1,3]dioxole-5- carboxamide | 782.2 | ¹H NMR (400 MHz, DMSO- d6) δ = 11.40 (s, 1H), 10.87 (s, 1H), 8.55 (s, 1H), 8.46 (dd, J = 2.4, 6.6 Hz, 1H), 8.01- 7.93 (m, 3H), 7.92-7.80 (m, 2H), 7.67 (s, 1H), 7.62 (s, 1H), 7.60-7.45 (m, 2H), 3.18 (s, 2H), 1.55 (s, 6H), 1.12 (s, 9H) | 2.87 A |
| 102 | 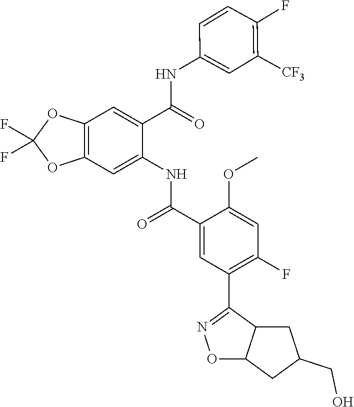 [SMILES: COc1cc(F)c(C2=NOC3CC(CO)CC23)cc1C(=O)Nc1cc2c(cc1C(=O)Nc1ccc(F)c(C(F)(F)F)c1)OC(F)(F)O2] | 2,2-difluoro-N-(4- fluoro-3- (trifluoromethyl) phenyl)-6-(4-fluoro- 5-(5-(hydroxymethyl)- 3a,5,6,6a-tetrahydro-4H- cyclopenta[d]isoxazol- 3-yl)-2- methoxybenzamido) benzo[d][1,3]dioxole-5- carboxamide | 670 | ¹H NMR (400 MHz, DMSO- d6) δ = 11.72 (s, 1H), 10.86 (s, 1H), 8.55 (s, 1H), 8.39 (d, J = 8.6 Hz, 1H), 8.31 (dd, J = 2.6, 6.5 Hz, 1H), 8.07-7.99 (m, 1H), 7.96 (s, 1H), 7.57 (t, J = 9.8 Hz, 1H), 7.30 (d, J = 13.0 Hz, 1H), 5.14 (dd, J = 5.1, 8.8 Hz, 1H), 4.49 (t, J = 5.3 Hz, 1H), 4.26-4.15 (m, 1H), 4.07 (s, 3H), 3.44-3.34 (m, 2H), 2.00 (dd, J = 5.5, 13.6 Hz, 1H), 1.90 (td, J = 5.7, 11.6 Hz, 1H), 1.78-1.69 (m, 1H), 1.67-1.50 (m, 2H) | 1.07 A |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of Formula (I): 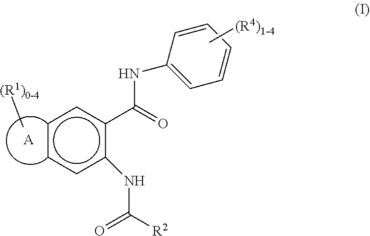
or a pharmaceutically acceptable salt thereof, wherein:
Ring A is a 5-membered heterocyclyl comprising 1-3 heteroatoms selected from O, S(═O)ₚ, N, and NR^{1a}, and substituted with 0-4 R¹;
R¹ is halo or C₁₋₄ alkyl substituted with 0-5 halo or —OH;
R^{1a} is H or C₁₋₃ alkyl;
R² is phenyl substituted with 0-3 R³ and 1 R⁵, bicyclic carbocyclyl substituted with 0-3 R³ and 0-1 R⁵, or a 5 to 12-membered heterocyclyl comprising 1-5 heteroatoms selected from O, S(═O)ₚ, N, and NR²a and substituted with 0-3 R³ and 0-1 R⁵;
R^{2a} is H, C₁₋₃ alkyl, or C(═O)R^{b};
R³ is halo, CN, OH, C₄a alkyl, or —OC₁₋₄ alkyl substituted with 0-5 halo, OH, —OC₁₋₄ alkyl, aryl, or heterocycly;
R⁴ is halo, CN, C₁₋₄ alkyl substituted with 0-5 halo, OH, or —OC₁₋₄ alkyl substituted with 0-5 halo;
R⁵ is —C(═O)NR¹³R¹³, —S(═O)ₚNR¹³R¹³, C₂₋₈ alkenyl substituted with 0-3 R⁶ and 0-2 R⁵, C₂₋₈ alkynyl substituted with 0-3 R⁶ and 0-2 R⁵, C₃₋₆ carbocyclyl substituted with 0-3 R⁶ and 0-2 R⁵, or a 3- to 12-membered heterocyclyl comprising 1-4 heteroatoms selected from O, S(═O)ₚ, N and NR¹⁰ and substituted with 0-3 R⁶ and 0-1 R⁷; provided when R² is phenyl and R⁷ is heterocyclyl, said heterocyclyl is bonded to the phenyl ring through a carbon or nitrogen atom;
R⁶ is halo, CN, ═O, —OH, —OC₁-a alkyl, or C₁₋₄ alkyl substituted with 0-2 halo or OH;
R⁷ is C₁₋₅ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR^{c}, C₃₋₆ carbocyclyl substituted with 0-5 R¹, or a 4- to 6-membered heterocyclyl comprising 1-4 heteroatoms selected from O, S(═O)ₚ, N and NR^{d}, and substituted with 0-5 R^{c};
R⁸ is halo, —C(═O)OR, —C(═O)NR^{a}R^{a}, —C(═O)NR^{a}OR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{e}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c}, —OP(═O)(OH)₂, —(CH₂)ₙ—C₃₋₆ carbocyclyl substituted with 0-3 R^{e}, or —(CH₂)ₙ-heterocyclyl comprising 1-4 heteroatoms selected from O, S(═O)ₚ, and N, and NR^{d}, and substituted with 0-5 R^{e};
R¹⁰ is H, C₁₋₄ alkyl substituted with 0-2 R¹¹, —C(═O)R^{b}, —C(═O)OR^{b}, —C(═O)NR^{a}R^{a}, —S(O)ₚR^{c}, C₃₋₆ carbocyclyl substituted with 0-5 R^{e}, or a 4- to 6-membered heterocyclyl comprising 1-4 heteroatoms selected from O, S(═O)ₚ, N and NR¹² and substituted with 0-5 R^{e};
R¹¹ is —OH, —C(═O)OH, —C(═O)NR^{a}R^{a}, or aryl;
R¹² is H, C₁₋₃ alkyl, or aryl;
R¹³ is H or C₁₋₃ alkyl substituted with 0-2 R⁶ and 0-2 R⁷; or R¹³ and R¹³ together with the nitrogen atom to which they are both attached form a heterocyclyl comprising additional 0-5 heteroatoms selected from O, S(═O)ₚ, N, and NR¹¹), and substituted with 0-3 R and 0-2 R:
R^{a} is H, C₁₋₆ alkyl substituted with 0-5 Rr, C₂₋₆ alkenyl substituted with 0-5 R^{g}, C₂₋₆ alknyl substituted with 0-5 R^{c}, —(CH₂)ₙ—C₃₋₆ carbocyclyl substituted with 0-5 R^{e}, or —(CH₂)ₙ-heterocyclyl substituted with 0-5 R^{c}; or R^{a} and R^{a} together with the nitrogen atom to which they are both attached form a heterocyclyl substituted with 0-5 R^{e};
R^{b} is H, C₁₋₆ alkyl substituted with 0-5 R⁵, C₂₋₆ alkenyl substituted with 0-5 R^{c}, C₂₋₆ alkynyl substituted with 0-5 R^{c}, —(CH₂)—C₃₋₁₀ carbocyclyl substituted with 0-5 R^{c}, or —(CH₂)ₙ-heterocyclyl substituted with 0-5 R^{e};
R^{c} is C₁₋₆ alkyl substituted with 0-5 R^{e}, C₂₋₆alkenyl substituted with 0-5 R^{e}, C₂₋₆ alkynyl substituted with 0-5 R^{e}, C₃₋₆ carbocyclyl, or heterocyclyl;
R^{d} is H or C₁₋₄ alkyl;
R^{c} is halo, CN, NO₂, ═O, C₁₋₆ alkyl substituted with 0-5 R⁹, C₂₋₆ alkenyl substituted with 0-5 RR, C₂₋₆ alkynyl substituted with 0-5 R^{E}, —(CH₂)ₙ-carbocyclyl substituted with 0-5 R^{g}, —(CH₂)ₙ-heterocyclyl substituted with 0-5 R⁵, —(CH₂)ₙOR^{f}, —C(═O)OR^{f}, —C(═O)NR^{f}R^{f}, —NR^{f}C(═O)R^{f}, —S(═O)ₚR^{f}, —S(═O)ₚNR^{f}R^{f}, —NR^{a}S(═O)ₚR^{f}, —NR^{f}C(═O)OR^{f}, —OC(═O)NR^{f}R^{f}, or —(CH₂)ₙNR^{f}R^{f};
R^{f} is H, C₁₋₆ alkyl substituted with 0-1 —OC₁₋₄ alkyl, C₃₋₆ cycloalkyl, aryl, or heterocyclyl; or R^{f} and R^{f} together with the nitrogen atom to which they are both attached form a heterocyclyl;
R^{g} is halo, CN, OH, S(═O)₂C₁₋₃ alkyl, C₁₋₆alkyl, C₃₋₆ cycloalkyl, or aryl;
n is zero, 1, 2, or 3; and
p is zero, 1, or 2.

2. The compound of claim 1, having Formula (II): 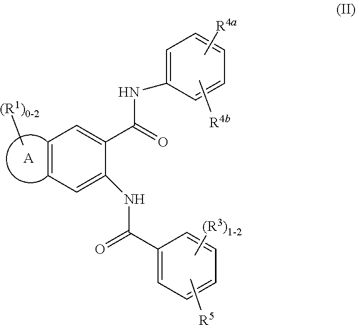
or a pharmaceutically acceptable salt thereof, wherein: 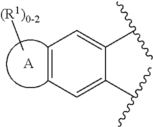
is 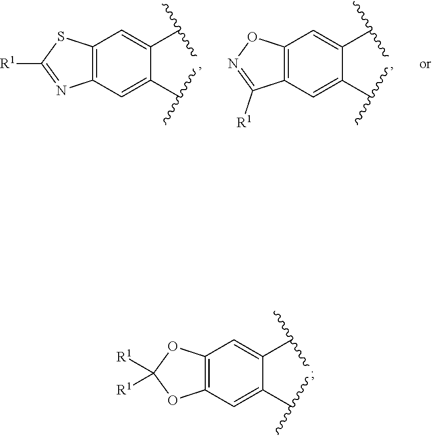
R¹ is halo or C₁₋₃ alkyl;
R³ is halo, C₁₋₃ alkyl, or —OC₁₋₄ alkyl substituted with 0-4 halo:
R^{4a} is halo;
R^{4b} is C₁₋₄ alkyl substituted with 0-4 halo;
R⁵ is —C(═O)NR¹³R¹³, —S(═O)ₚNR¹³R¹³, C₂₋₇ alkenyl substituted with 0-3 R⁶ and 0-2 R⁷, C₂₋₇ alkynyl substituted with 0-3 R⁶ and 0-2 R⁷, phenyl substituted with 0-3 R⁶ and 0-2 R⁷, or a 3- to 10-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₄ alkyl substituted with 0-1 R⁶ and 0-1 R⁵, —OR^{b}, —NR^{a}R^{a}, —NR³C(═O)R^{b}, —NR³C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)_{P}R^{c}, —OC(═O)R^{b}, —S(═O)ₚR^{c}, —S(═O)NR^{a}R^{a}, C₃₋₆ cycloalkyl substituted with 0-5 R^{C}, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{c};
R¹¹ is —OH, —C(═O)OH, or aryl:
R¹² is H, C₁₋₃ alkyl, or aryl;
R¹³ is H or C₁₋₃ alkyl substituted with 0-1 R⁶ and 0-1 R⁷; or R¹³ and R¹³ together with the nitrogen atom to which they are both attached form a heterocyclyl comprising additional 0-5 heteroatoms selected from O, S(═O)ₚ, N, and NR¹⁰, and substituted with 0-3 R⁶ and 0-2 R⁷:
R^{a} is H, C₁₋₅ alkyl substituted with 0-5 R^{e}, C₂₋₅ alkenyl substituted with 0-5 R^{e}, C₂₋₅ alkynyl substituted with 0-5 R^{c}, —(CH₂)ₙ—C₃₋₁₀ carbocyclyl substituted with 0-5 R^{e}, or —(CH₂)ₙ-heterocyclyl substituted with 0-5 R^{c}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₈ alkenyl substituted with 0-5 R^{c}, C₂₋₅ alkynyl substituted with 0-5 R^{c}, —(CH₂)ₙ—C₃₋₁₀ carbocyclyl substituted with 0-5 R⁵, or —(CH₂)ₙ-heterocyclyl substituted with 0-5 R^{c};
R^{c} is C₁₋₅ alkyl substituted with 0-5 R^{g}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₃ alkyl;
R^{c} is halo, CN, ═O, C₁₋₆ alkyl substituted with 0-5 R^{g}, C₂₋₆ alkenyl substituted with 0-5 R⁵, C₂₋₆ alkynyl substituted with 0-5 R⁵, —(CH₂)ₙ—C₃₋₆ cycloalkyl, —(CH₂)ₙ-aryl, —(CH₂)ₙ-heterocyclyl, —(CH₂)ₙOR^{f}, —C(═O)OR^{f}, or —S(═O)ₚR^{f};
R^{f} is H or C₁₋₃ alkyl substituted with 0-1 —OC₁₋₄ alkyl;
R⁸ is halo, CN, OH, C₁₋₆ alkyl, C₃₋₆ cycloalkyl, or aryl;
n is zero, 1, 2, or 3; and
p is zero, 1, or 2.

3. The compound of claim 2, having Formula (III): 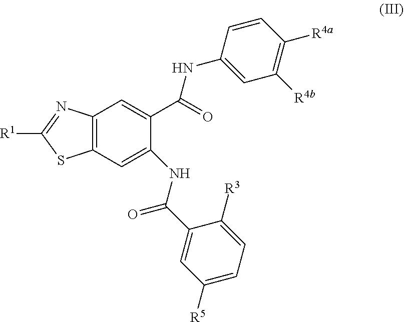
or a pharmaceutically acceptable salt thereof, wherein:
R¹ is C₁₋₂ alkyl;
R³ is —OC₁₋₄ alkyl;
R^{4a} is halo;
R^{4b} is C₁₋₃ alkyl substituted with 0-4 F;
R⁵ is —S(═O)ₚNR¹³R¹³, phenyl substituted with 0-3 R⁶ and 0-2 R⁵, or a 3- to 10-membered heterocyclyl comprising 1-4 heteroatoms selected from O, S(═O)ₚ, N and NR¹⁰ substituted with 0-3 R^{b} and 0-1 R⁷;
R⁶ is halo, CN, C₁₋₃ alkyl, —OH, or —OC₁₋₄ alkyl;
R⁷ is C₁₋₄ alkyl substituted with 0-1 R⁸ and 0-1 R⁹, OR^{b}, —NR^{a}R^{a}, —NR^{f}C(═O)R^{c}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, or C₁₋₄ alkyl substituted with 0-3 halo or OH;
R⁶ is —OR^{b}, —NR^{a}R^{a}, —NR^{a}C(═O)R^{b}, —NR^{a}C(═O)OR^{b}, —NR^{a}S(═O)ₚR, —NR^{a}S(O)ₚNR^{a}R^{a}, —OC(═O)NR^{a}R^{a}, —OC(═O)NR^{a}OR^{b}, or —S(═O)ₚNR^{a}R^{a};
R¹¹ is H, C₁₋₄ alkyl substituted with 0-2 R¹¹, or —C(═O)R^{b};
R¹³ and R¹³ together with the nitrogen atom to which they are both attached form a heterocyclyl comprising additional 0-5 heteroatoms selected from O, S(═O)ₚ, N, and NR¹⁰, and substituted with 0-3 R⁵ and 0-2 R⁷;
R^{a} is H, C₁₋₅ alkyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{c}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₃ carbocyclyl, or heterocyclyl;
R^{d} is H or C₁₋₂ alkyl;
R^{e} is halo, CN, ═O, C₁₋₆ alkyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₅ alkyl substituted with 0-1 —OC₁₋₄ alkyl;
R^{g} is halo, CN, OH, C₁₋₆ alkyl, or C₃₋₆ cycloalkyl;
n is zero, 1, 2, or 3; and
p is zero, 1, or 2.

4. The compound of claim 3, or a pharmaceutically acceptable salt thereof, wherein:
R¹ is —CH₃;
R³ is —OCH:
R^{4a} is F;
R^{4b} is CF;
R⁵ is —S(═O)₂NR¹³R¹³, 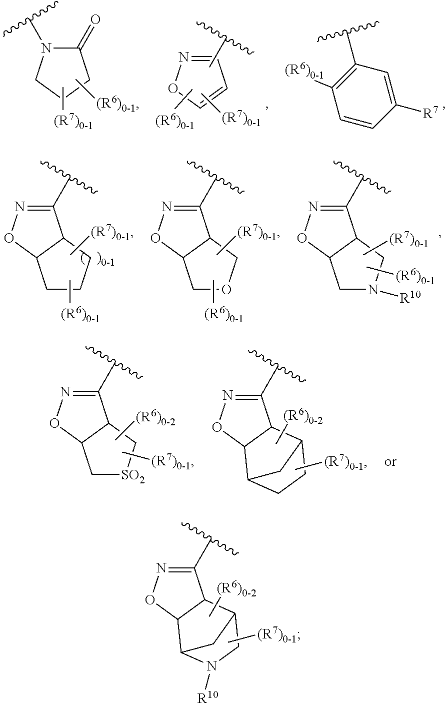
R⁶ is halo, C₁₋₄ alkyl, —OH, or —OC₁₋₄ alkyl;
R⁷ is C₁₋₄ alkyl substituted with 0-1 R⁸ and 0-1 R⁹, —C(═O)NR^{a}R^{a}, or C₃₋₆ cycloalkyl substituted with 0-2 R^{e};
R⁸ is halo, —C(═O)OC₁₋₄ alkyl, or C₁₋₄ alkyl;
R⁹ is —OR^{b}, —NR^{a}R^{a}, —NR^{a}C(═O)R^{b}, or —OC(═O)NR^{a}R^{a};
R¹⁰ is H, C₁₋₃ alkyl substituted with 0-2 R¹¹, or —C(═O)R^{b};
R¹¹ is —OH;
R¹³ and R¹³ together with the nitrogen atom to which they are both attached form a heterocyclyl comprising additional 0-5 heteroatoms selected from O, S(═O)ₚ, and N, and substituted with 0-3 R⁶ and 0-2 R⁷;
R^{a} is H, C₁₋₃ alkyl, or C₃₋₆ cycloalkyl;
R^{b} is H, C₁₋₄ alkyl substituted with 0-1 R^{c}, or heterocyclyl;
R^{c} is —OR^{f}, and
R^{f} is H or C₁₋₃ alkyl substituted with —OC₁₋₃alkyl.

5. The compound of claim 4, or a pharmaceutically acceptable salt thereof, wherein:
R⁵ is 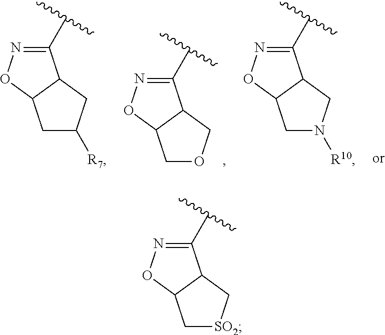
R⁷ is C₁₋₄ alkyl substituted with 0-1 R⁹;
R⁹ is —OR^{b};
R¹⁰ is H or —C(═O)R^{b};
R^{b} is H or C₁₋₄ alkyl substituted with 0-1 R^{e};
R^{c} is —OR^{f};
R^{f} is H or C₁₋₂ alkyl.

6. The compound of claim 2, having Formula (IV): 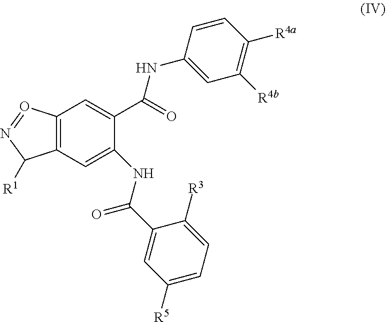
or a pharmaceutically acceptable salt thereof, wherein:
R¹ is C₁₋₂ alkyl;
R³ is —OC₁₋₄ alkyl;
R^{4a} is halo;
R^{4b} is C₁₋₃ alkyl substituted with 0-4 F;
R⁵ is C₂₋₄ alkynyl substituted with OH, or phenyl substituted with 0-3 R⁸ and 0-2 R⁷;
R⁶ is halo, CN, C₁₋₃ alkyl, —OH, or —OC₁₋₄ alkyl;
R⁷ is C₁₋₂ alkyl substituted with 0-1 R⁸ and 0-1 R⁹;
R⁸ is halo or C₁₋₄ alkyl substituted with 0-3 halo or OH;
R⁹ is —OR^{b}, —OC(═O)NR^{a}R^{a}, or —OC(═O)NR^{a}OR^{b};
R¹ is H, C₁₋₃ alkyl, or C₃₋₆ cycloalkyl; and
R^{b} is H or C₁₋₃ alkyl.

7. The compound of claim 2, having Formula (V): 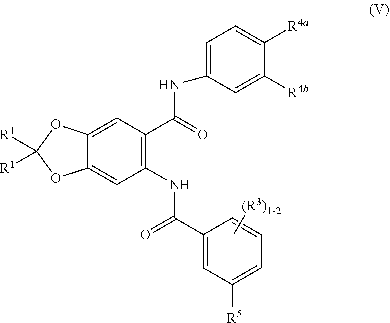
or a pharmaceutically acceptable salt thereof, wherein:
R¹ is halo;
R³ is halo, C₁₋₃ alkyl, or —OC₁₋₃ alkyl;
R^{4a} is halo;
R^{4b} is C₁₋₃ alkyl substituted with 0-3 halo;
R⁸ is —C(═O)NR¹³R¹³, C₂₋₆ alkenyl substituted with 0-3 R⁶ and 0-2 R⁷, C₂₋₆ alkynyl substituted with 0-3 R⁶ and 0-2 R⁵, phenyl substituted with 0-3 R⁶ and 0-2 R⁵, or a 3- to 10-membered heterocyclyl comprising 1-4 heteroatoms selected from O, S(═O)ₚ, N and NR¹⁰, and substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₄ alkyl substituted with 0-1 R⁸ and 0-1 R^{e}, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, —C(═O)NR^{a}S(═O)ₚR^{c}, C₃₋₄ cycloalkyl substituted with 0-4 R^{e}, or a 4- to 6-membered heterocyclyl comprising 1-4 heteroatoms selected from O, S(═O)_{P}, N and NR^{d}, and substituted with 0-4 R^{c};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, —S(O)ₚR^{c}, C₃₋₆ cycloalkyl substituted with 0-5 R^{c}, or a 4- to 6-membered heterocyclyl comprising 1-4 heteroatoms selected from O, S(═O)ₚ, N and NR¹² and substituted with 0-5 R^{e};
R¹¹ is —OH, —C(═O)OH, —C(═O)NR^{a}R^{a}, or aryl:
R¹² is H, C₁₋₃ alkyl, or aryl;
R¹³ is H or C₁₋₄ alkyl substituted with 0-3 R⁶ and 0-2 R⁷; or R¹¹ and R¹³ together with the nitroen atom to which they are both attached form a heterocyclyl comprising additional 0-4 heteroatoms selected from O, S(═O)ₚ, N, and NR¹⁰, and substituted with 0-3 R⁶ and 0-2 R¹;
R^{a} is H, C₁₋₅ alkyl substituted with 0-4 R^{c}, —(CH₂)ₙ—C₃₋₁₀ carbocyclyl substituted with 0-4 R^{e}, or —(CH₂)ₙ-heterocyclyl substituted with 0-4 R: or R^{a} and R^{a} together with the nitrogen atom to which they are both attached form a heterocyclyl substituted with 0-4 R¹;
R^{b} is H, C₁₋₅ alkyl substituted with 0-4 R^{e}, —(CH₂)ₙ—C₃₋₁₀ carbocyclyl substituted with 0-4 R⁵, or —(CH₂)ₙ-heterocyclyl substituted with 0-4 R¹;
R^{c} is C₁₋₅ alkyl substituted with 0-4 R^{c}, C₃₋₆ carbocyclyl, or heterocyclyl;
R^{d} is H or C₁₋₂ alkyl:
R^{c} is halo, CN, ═O, C₁₋₆ alkyl substituted with 0-5 R⁵, —(CH₂)—C₃₋₆ cycloalkyl, —(CH₂)ₙ-aryl, —(CH₂)ₙ-heterocyclyl, —(CH₂)ₙOR¹, or —C(═O)OR^{f};
R^{f} is H or C₁₋₃ alkyl substituted with 0-1 —OC₁₋₄ alkyl:
R^{g} is halo, CN, OH, C₁₋₆ alkyl, or C₃₋₆ cycloalkyl;
n is zero, 1, 2, or 3; and
p is zero, 1, or 2.

8. The compound of claim 7, having Formula (VI): 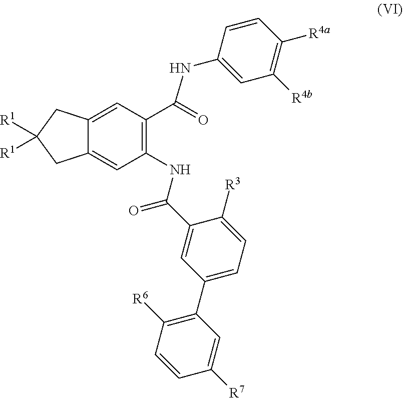
or a pharmaceutically acceptable salt thereof, wherein:
R¹ is F;
R³ is —OCH:
R^{4a} is F;
R^{4b} is CF₃;
R⁶ is halo;
R⁷ is —C(═O)OR^{b}, —C(═O)NR^{a}R^{a}, —S(═O)ₚNR^{a}R^{a}, or —C(═O)NR^{a}S(═O)ₚC₁₋₄alkyl;
R^{a} is H, C₁₋₃ alkyl substituted with 0-5 R^{e}, or heterocyclyl substituted with 0-4 R^{e};
R^{b} is H or C₁₋₃ alkyl substituted with 0-5 R^{e}; and
R^{c} is halo, CN, ═O, or C₁₋₆ alkyl.

9. The compound of claim 7, or a pharmaceutically acceptable salt thereof, wherein:
R¹ is F;
R³ is —OCH₃;
R^{4a} is F;
R^{4b} is CF₃;
R⁵ is 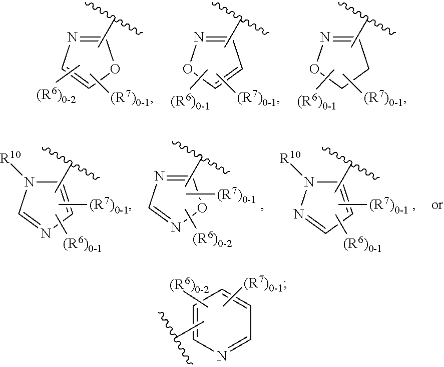
R⁶ is halo, —OH, or C₁₋₄ alkyl substituted with 0-1 OH;
R⁷ is —NR^{a}R^{a} or —S(═O)₂NR^{a}R^{a};
R¹⁰ is H, —C(═O)R^{b}, or C₁₋₄ alkyl substituted with 0-1 R¹¹;
R¹¹ is —OH, —C(═O)OH, or aryl;
R^{a} is H or C₁₋₃ alkyl; or R^{a} and R^{a} together with the nitrogen atom to which they are both attached form a heterocyclyl; and
R^{b} is H or C₁₋₃ alkyl.

10. The compound of claim 7, or a pharmaceutically acceptable salt thereof, wherein:
R¹ is F;
R³ is —OCH₃;
R^{4a} is F;
R^{4b} is CF₃:
R⁵ is 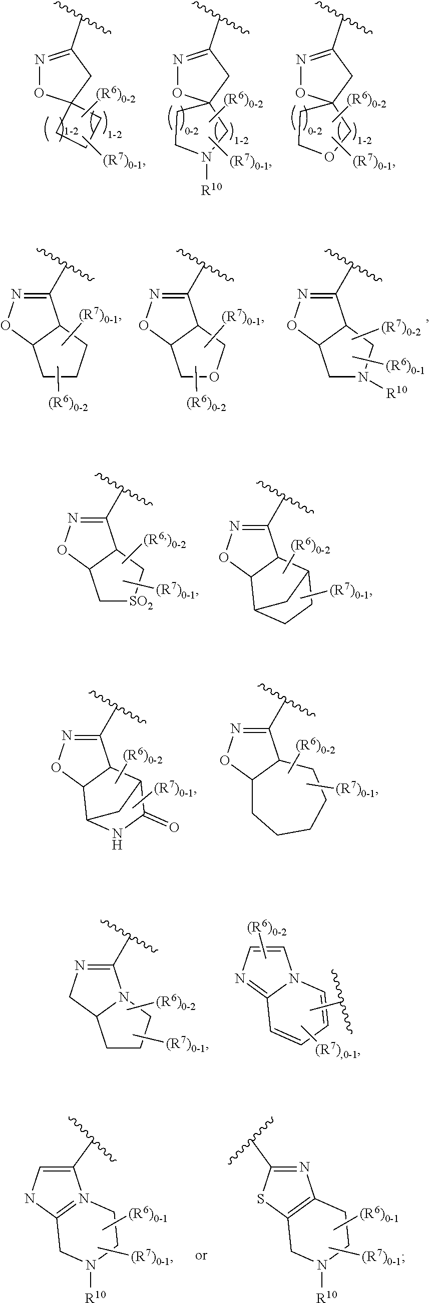
R⁶ is halo, ═O, —OH, —OC₁₋₄ alkyl, or C₁₋₄ alkyl;
R⁷ is C₁₋₃ alkyl substituted with 0-1 R⁸ and 0-1 R⁹, —NR^{a}R^{a}, or —NR^{a}C(═O)R^{b};
R⁸ is —C(═O)OR^{b};
R⁹ is OH;
R¹⁰ is H, C₁₋₃ alkyl substituted with 0-2 R¹¹, —C(═O)R^{b}, —C(O)OR, —C(═O)NR^{a}R^{a}, or a 4- to 6-membered heterocyclyl comprising 1-4 heteroatoms selected from O, S(═O)ₚ, N and NR¹², and substituted with 0-4 R^{e};
R¹¹ is —OH, —C(═O)OH, or C(═O)NR^{a}R^{a};
R¹¹ is H and C₁₋₃ alkyl;
R^{a} is H or C₁₋₃ alkyl;
R^{b} is H or C₁₋₃ alkyl substituted with 0-1 R^{e}; and
R^{e} is OH.

11. The compound of claim 10, or a pharmaceutically acceptable salt thereof, wherein:
R⁵ is 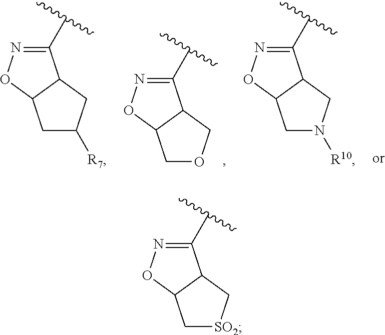
R⁷ is C₁₋₃ alkyl substituted with 0-1 R⁹;
R⁹ is —OH;
R¹⁰ is H, C₁₋₃ alkyl substituted with 0-2 R¹¹, or —C(═O)R^{b};
R¹¹ is —OH;
R^{b} is H or C₁₋₃ alkyl substituted with 0-1 R^{e}; and
R^{e} is OH.

12. The compound of claim 7, or a pharmaceutically acceptable salt thereof, wherein:
R¹ is F;
R³ is —OCH;
R^{4a} is F;
R^{4b} is CF₃;
R⁵ is 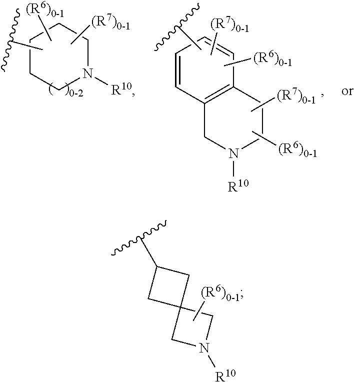
R⁶ is halo, ═O, —OH, —OC₁₋₄ alkyl, or C₁₋₄ alkyl;
R⁷ is C₁₋₂ alkyl substituted with 0-1 R⁸ and 0-1 R⁹, —NR^{a}R^{a}, —NR^{a}C(═O)R^{b}, —NR^{a}C(═O)OR^{b}, or—C(═O)OR^{b};
R⁶ is —C(═O)OR^{b}, —C(═O)NHR^{a}, —C(═O)NHOR^{b}, or C₁₋₄ alkyl substituted with 0-3 halo or OH;
R⁹ is —NR^{a}C(═O)R^{b};
R¹⁰ is H, C₁₋₄ alkyl substituted with 0-2 R¹⁰, —C(═O)R^{b}, —C(═O)OR^{b}, —C(═O)NR^{a}R^{a}, —S(═O)₂C₁₋₃ alkyl, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or C(═O)NR^{a}R^{a};
R¹² is H, C₁₋₃ alkyl, or aryl;
R^{a} is H or C₁₋₃ alkyl;
R^{b} is H, or C₁₋₃ alkyl substituted with 0-1 R^{e}; and
R^{e} is OH.

13. The compound of claim 7, or a pharmaceutically acceptable salt thereof, wherein:
R¹ is F;
R³ is —OCH₃;
R^{4a} is F;
R^{4b} is CF₃;
R⁵ is —C(═O)NR^{a}R^{a};
R⁶ is halo, —OH, or C₁₋₄ alkyl substituted with 0-1 OH;
R⁷ is —S(═O)₂C₁₋₃ alkyl or C₃₋₆ cycloalkyl substituted with 0-2 R^{e}:
R¹⁰ is H, —C(═O)R^{b}, or C₁₋₄ alkyl substituted with 0-1R¹¹;
R¹¹ is —OH, —C(═O)OH, or aryl;
R¹³ is H or C₁₋₃ alkyl substituted with 0-1 R⁶ and 0-1 R⁷; or R¹³ and R¹³ together with the nitrogen atom to which they are both attached form a heterocyclyl selected from 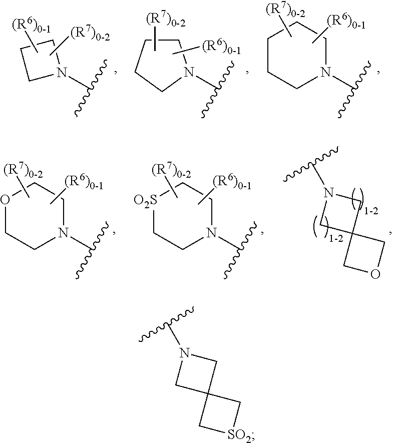
R^{b} is H or C₁₋₆ alkyl; and
R^{c} is —S(═O)₂C₁₋₃ alkyl.

14. The compound of claim 13, or a pharmaceutically acceptable salt thereof, wherein:
R⁵ is —C(═O)NHR¹³;
R¹³ is C₁₋₃ alkyl substituted with 0-1 R⁷;
R⁷ is C₃₋₆ cycloalkyl substituted with 0-2 R^{e}; and
R^{e} is —S(═O)₂C₁₋₃ alkyl.

15. The compound of claim 2, or a pharmaceutically acceptable salt thereof, wherein: 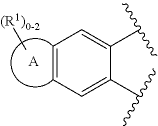
is 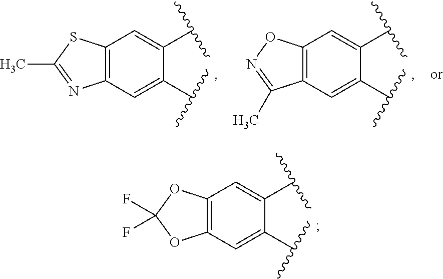 [SMILES: *c1cc2nc(C)sc2cc1* ; *c1cc2onc(C)c2cc1* ; *c1cc2c(cc1*)OC(F)(F)O2]
R³ is —OC₁₋₃ alkyl;
R^{4a} is F;
R^{4b} is CF₃;
R⁵ is —C(═O)NHR¹³, 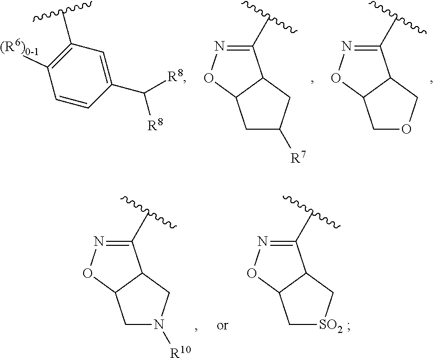
R⁷ is C₁₋₃ alkyl substituted with 0-1 R⁹ or C₃₋₆ cycloalkyl substituted with 0-5 R^{e};
R⁸ is halo, —C(═O)OR^{b}, or C₁₋₄ alkyl substituted with 0-3 halo;
R⁹ is —OH, —NR^{a}C(═O)R^{b} or —OC(═O)NR^{a}R^{a};
R¹⁰ is H, C₁₋₃ alkyl substituted with 0-2 R¹¹, or —C(═O)R^{b};
R¹¹ is —OH;
R¹³ is H or C₁₋₃ alkyl substituted with 0-1 R⁷;
R^{b} is H, C₁₋₃ alkyl substituted with 0-5 R^{e}, or heterocyclyl:
R^{e} is —OR^{f} or —S(═O)₂C₁₋₃ alkyl; and
R^{f} is H or C₁₋₃ alkyl.

16. A composition comprising a compound of claim 1, or a pharmaceutically acceptable salt thereof, and a pharmaceutically acceptable carrier.

17. A method for treating a disease associated with relaxin comprising administering a therapeutically effective amount of the composition of claim 16 to a patient in need thereof.

18. The method of claim 17 wherein the disease is selected from the group consisting of angina pectoris, unstable angina, myocardial infarction, heart failure, acute coronary disease, acute heart failure, chronic heart failure, and cardiac iatrogenic damage.

19. The method of claim 18 wherein the disease is heart failure.

20. The method of claim 18 wherein the disease is fibrosis.
